(19) United States
(12) Patent Application Publication
(10) Pub. No.: US 2014/0303031 A1
(43) Pub. Date: Oct. 9, 2014

(54) METHODS AND COMPOSITIONS FOR CHARACTERIZING AUTISM SPECTRUM DISORDER BASED ON GENE EXPRESSION PATTERNS

(71) Applicant: Children's Medical Center Corporation, Boston, MA (US)

(72) Inventors: Louis M. Kunkel, Westwood, MA (US); Isaac S. Kahane, Newton, MA (US); Sek Won Kong, Lexington, MA (US); Christian D. Collins, Brookline, MA (US)

(73) Assignee: Children's Medical Center Corporation, Boston, MA (US)

(21) Appl. No.: 14/355,017

(22) PCT Filed: Oct. 31, 2012
(86) PCT No.: PCT/US2012/062735

§ 371 Date: Apr. 29, 2014

Related U.S. Application Data
(60) Provisional application No. 61/710,646, filed on Oct. 5, 2012.
(60) Provisional application No. 61/553,914, filed on Oct. 31, 2011.

(51) Int. Cl. C12Q 1/68; G01N 33/543

(52) U.S. Cl. CPC C12Q 1/6883; G01N 33/543
USPC 506/9; 506/17; 506/18; 435/6.11; 702/19

(57) ABSTRACT

The invention relates to methods and kits for characterizing and diagnosing autism spectrum disorder in an individual based on gene expression levels.

### RELATED APPLICATIONS

This application claims priority under 35 U.S.C. §119 to U.S. provisional patent application, U.S. Ser. No. 61/553,914, filed Oct. 31, 2011, entitled “Methods and Compositions for Characterizing Autism Spectrum Disorder Based on Gene Expression Patterns,” and U.S. provisional patent application, U.S. Ser. No. 61/710,646, filed Oct. 5, 2012, entitled “Methods and Compositions for Characterizing Autism Spectrum Disorder Based on Gene Expression Patterns,” the entire contents of which are incorporated herein by reference.

### FEDERALLY SPONSORED RESEARCH

This invention was made with United States Government support under grants R01MH085143 and P30HD018655 awarded by, respectively, the National Institute of Mental Health and the National Institute of Child Health & Human Development of the National Institutes of Health. The United States government has certain rights in the invention.

### BACKGROUND OF INVENTION

Autism Spectrum Disorders (ASD) cover a broad spectrum of neurocognitive and social developmental delays with typical onset before 3 years of age including Autistic Disorder, Pervasive Developmental Disorder—Not Otherwise Specified and Asperger's Disorder as subclassified in the Diagnostic and Statistical Manual of Psychiatric Disorders, 4th edition, Text Revision (DSM-IV-TR). Prevalence of ASD has been increasing during last decades, and current estimation is 1 in 91 to 3.7 in 1000. There are waiting lists for evaluation by most centers with expertise, and despite the progress made in adopting instruments such as the Autism Diagnostic Interview-Revised (ADI-R) and the Autism Diagnostic Observation Schedule (ADOS) there remains significant debate regarding the prognostic value and accuracy of existing instruments.

### SUMMARY OF INVENTION

It has been discovered that a variety of genes are differentially expressed in individuals having autism spectrum disorder compared with individuals free of autism spectrum disorder. Such genes are identified herein as “autism spectrum disorder-associated genes”. It has also been discovered that the autism spectrum disorder status of an individual can be classified with a high degree of accuracy, sensitivity, and/or specificity based on expression levels of these autism spectrum disorder-associated genes. Accordingly, methods and related kits are provided herein for characterizing and/or diagnosing autism spectrum disorder in an individual. In some embodiments, methods are provided for subclassifying individuals by molecular endophenotypes (e.g., gene expression profiles).

According to some aspects of the invention, methods are provided for characterizing the autism spectrum disorder status of an individual in need thereof. In some embodiments, the methods involve subjecting a clinical sample obtained from the individual to a gene expression analysis, in which the gene expression analysis comprises determining expression levels of a plurality of autism spectrum disorder-associated genes in the clinical sample using an expression level determining system. In some embodiments, the methods further involve determining the autism spectrum disorder status of the individual based on the expression levels of the plurality of autism spectrum disorder-associated genes. In some embodiments, the methods further involve a step of obtaining the clinical sample from the individual. In some embodiments, the methods further involve a step of diagnosing autism spectrum disorder in the individual based on the autism spectrum disorder status. In some embodiments, the clinical sample is a sample of peripheral blood, brain tissue, or spinal fluid.

In some embodiments, methods are provided that involve applying an autism spectrum disorder-classifier to autism spectrum disorder gene expression levels to determine the autism spectrum disorder status of the individual. For example, according to some aspects of the invention, methods of characterizing the autism spectrum disorder status in an individual in need thereof are provided that involve (a) subjecting a clinical sample obtained from the individual to a gene expression analysis, in which the gene expression analysis comprises determining expression levels of a plurality of autism spectrum disorder-associated genes in the clinical sample using an expression level determining system, in which the autism spectrum disorder-associated genes comprise at least ten genes selected from Table 4, 5, 6, 8, 9, 10, or 11; and (b) applying an autism spectrum disorder-classifier to the expression levels, in which the autism spectrum disorder-classifier characterizes the autism spectrum disorder status of the individual based on the expression levels. In some embodiments, the methods comprise diagnosing autism spectrum disorder in the individual based on the autism spectrum disorder status.

In certain embodiments, the autism spectrum disorder-classifier is based on an algorithm selected from logistic regression, partial least squares, linear discriminant analysis, quadratic discriminant analysis, neural network, naïve Bayes, C4.5 decision tree, k-nearest neighbor, random forest, and support vector machine. In certain embodiments, the autism spectrum disorder-classifier has an accuracy of at least 65%. In certain embodiments, the autism spectrum disorder-classifier has an accuracy in a range of about 65% to 90%. In certain embodiments, the autism spectrum disorder-classifier has a sensitivity of at least 65%. In certain embodiments, the autism spectrum disorder-classifier has a sensitivity in a range of about 65% to about 95%. In certain embodiments, the autism spectrum disorder-classifier has a specificity of at least 65%. In certain embodiments, the autism spectrum disorder-classifier has a specificity in range of about 65% to about 85%.

In some embodiments, the autism spectrum disorder-classifier is trained on a data set comprising expression levels of the plurality of autism spectrum disorder-associated genes in clinical samples obtained from a plurality of individuals identified as having autism spectrum disorder. In certain embodiments, the interquartile range of ages of the plurality of individuals identified as having autism spectrum disorder is from about 2 years to about 10 years. In some embodiments, the autism spectrum disorder-classifier is trained on a data set comprising expression levels of the plurality of autism spectrum disorder-associated genes in clinical samples obtained from a plurality of individuals identified as not having autism spectrum disorder. In certain embodiments, the interquartile range of ages of the plurality of individuals identified as not having autism spectrum disorder is from about 2 years to about 10 years. In some embodiments, the autism spectrum disorder-classifier is trained on a data set consisting of expression levels of the plurality of autism spectrum disorder-associated genes in clinical samples obtained from a plurality of male individuals. In some embodiments, the autism spectrum disorder-classifier is trained on a data set comprising expression levels of the plurality of autism spectrum disorder-associated genes in clinical samples obtained from a plurality of individuals identified as having autism spectrum disorder. In certain embodiments, the individuals were identified as having autism spectrum disorder based on DSM-IV-TR criteria.

In some embodiments, the autism spectrum disorder-associated genes comprise at least one, at least two, at least three, at least four, at least five, at least six, at least seven, at least eight, at least nine, at least ten, at least twenty, at least 30, at least 40, at least 50, at least 60, at least 70, at least 80, or at least 90 genes selected from Table 4, 5, 6, 8, 9, 10 or 11. In some embodiments, the autism spectrum disorder-associated genes comprise at least one of: LRRC6, SULF2, and YES1.

In some embodiments, the autism spectrum disorder genes comprise at least one, at least two, at least three, at least four, at least five, at least six, at least seven, or at least eight genes selected from Tables 13, 14, 15, 16, 17, 18, 19, 20, 21, 23, or 24.

In some embodiments, the autism spectrum disorder-associated gene is selected from the group consisting of: ADAM10, ARFGEF1, CAB39, COL4A3BP, CREBBP, DDX42, DNAJC3, HNRNPA2B1, IVNS1ABP, KIAA0247, KIDINS220, MGAT4A, MTMR10, MYO5A, NBEAL2, NCOA6, NUP50, PNN, PTPRE, RBL2, RNF145, ROCK1, RPS6KA3, SERINC3, SIRPA, SLA, SNRK, STK38, SULF2, TBC1D14, TMEM2, TRIP12, UTY, ZDHHC17, ZFP36L2, ZMAT1, ZNF12, and ZNF292. In some embodiments, the autism spectrum disorder-associated gene is selected from the group consisting of: AHNAK, BOD1L, CD9, CNTRL, IFNAR2, KBTBD11, KCNE3, KLHL2, MAN2A2, MAPK14, MEGF9, MIR223, PNISR, RMND5A, SSH2, ZNF516, and ZNF548.

In some embodiments, the methods involve comparing each expression level of the plurality of autism spectrum disorder-associated genes with an appropriate reference level, and the autism spectrum disorder status of the individual is determined based on the results of the comparison. In some embodiments, a higher level of at least one autism spectrum disorder-associated gene selected from: ZNF12, RBL2, ZNF292, IVNS1ABP, ZFP36L2, ARFGEF1, UTY, SLA, KIAA0247, HNRNPA2B1, RNF145, PTPRE, SFRS18, ZNF238, TRIP12, PNN, ZDHHC17, MLL3, MTMR10, STK38, SERINC3, NIPBL, TIGD1, DDX42, NUP50, CAB39, ROCK1, SULF2, FABP2, KIDINS220, NCOA6, SIRPA, PCSK5, ADAM10, ZNF33A, ZMAT1, C10orf28, MGAT4A, CEP110, ZZEF1, CREBZF, DOCK11, ATRN, COL4A3BP, FAM133A, TTC14, TMEM30A, MYO5A, KDM2A, ZCCHC14, RNF44, ZBTB44, CLTC, UTRN, ATXN7, PPP1R12A, LBR, TBC1D14, SPATA13, HK2, CREBBP, MED23, ZFYVE16, PAN3, RBBP6, AVL9, ZNF354A, ACTR2, TMBIM1, RPS6KA3, DNMBP, NBEAL2, MYSM1, TMEM2, SNRK, KIAA1109, HECA, DNAJC3, KIF5B, POLR2B, ANTXR2, VPS13C, MANBA, NIN, LRRC6, and YES1 compared with an appropriate reference level indicates that the individual has autism spectrum disorder. In some embodiments, a lower level of STXBP6 compared with an appropriate reference level indicates that the individual has autism spectrum disorder.

In some embodiments, the autism spectrum disorder-associated genes comprise at least one gene selected from each of at least two of the following KEGG pathways: Neurotrophin signaling pathway, Long-term potentiation, mTOR signaling pathway, Progesterone-mediated oocyte maturation, Regulation of actin cytoskeleton, Fc gamma R-mediated phagocytosis, Renal cell carcinoma, Chemokine signaling pathway, Type II diabetes mellitus, Non-small cell lung cancer, Colorectal cancer, ErbB signaling pathway, Prostate cancer, and Glioma. In some embodiments, the autism spectrum disorder-associated genes comprise at least one gene selected from each of the foregoing KEGG pathways.

In some embodiments, the autism spectrum disorder-associated genes comprise at least two different genes selected from at least two of the following sets: (i) MAPK1, RPS6KA3, YWHAG, CRKL, MAP2K1, PIK3CB, PIK3CD, SH2B3, MAPK8, KIDINS220; (ii) MAPK1, RPS6KA3, GNAQ, MAP2K1, CREBBP, PPP3CB, PPP1R12A; (iii) MAPK1, RPS6KA3, PIK3CB, PIK3CD, CAB39, RICTOR; (iv) IGF1R, MAPK1, RPS6KA3, MAP2K1, PIK3CB, PIK3CD, MAPK8; (v) GNA13, MAPK1, CRKL, ROCK1, MAP2K1, PIK3CB, PIK3CD, SSH2, PPP1R12A, IQGAP2, ITGB2; (vi) MAPK1, PTPRC, DOCK2, CRKL, MAP2K1, PIK3CB, PIK3CD; (vii) MAPK1, CRKL, MAP2K1, PIK3CB, PIK3CD, CREBBP; (viii) MAPK1, DOCK2, CRKL, ROCK1, MAP2K1, PIK3CB, PREX1, PIK3CD, CCR2, CCR10; (ix) MAPK1, PIK3CB, PIK3CD, HK2, MAPK8; (x) MAPK1, RASSF5, MAP2K1, PIK3CB, PIK3CD; (xi) IGF1R, MAPK1, MAP2K1, PIK3CB, PIK3CD, MAPK8; (xii) MAPK1, CRKL, MAP2K1, PIK3CB, PIK3CD, MAPK8; (xiii) IGF1R, MAPK1, MAP2K1, PIK3CB, PIK3CD, CREBBP; and (xiv) IGF1R, MAPK1, MAP2K1, PIK3CB, PIK3CD.

In some embodiments, the autism spectrum disorder genes comprise at least two, at least three, at least four, at least five, at least six, at least seven, at least eight, at least nine, at least ten, at least twenty, at least 30, at least 40, at least 50, at least 60, at least 70, or at least 80 genes selected from Table 6. In some embodiments, the autism spectrum disorder genes comprise all of the genes Table 6.

In some embodiments, the autism spectrum disorder genes comprise at least two, at least three, at least four, at least five, at least six, at least seven, at least eight, at least nine, at least ten, at least twenty, at least 30, at least 40, at least 50, at least 60, at least 70, at least 80, or at least 90 genes selected from Table 9. In some embodiments, the autism spectrum disorder genes comprise all of the genes Table 9. In certain embodiments, the autism spectrum disorder is autistic disorder (AUT).

In some embodiments, the autism spectrum disorder genes comprise at least two, at least three, at least four, at least five, at least six, at least seven, at least eight, at least nine, at least ten, at least twenty, at least 30, or at least 40 genes selected from Table 10. In certain embodiments, the autism spectrum disorder is pervasive developmental disorder—not otherwise specified (PDDNOS).

In some embodiments, the autism-spectrum disorder-associated gene is not AFF2, CD44, CNTNAP3, CREBBP, DAPK1, JMJD1C, NIPBL, PTPRC, SH3KBP1, STK39, DOCK8, RPS6KA3, or ATRX.

In some embodiments, the autism spectrum disorder genes comprise at least two, at least three, at least four, at least five, at least six, at least seven, or at least eight genes selected from Table 11. In certain embodiments, the autism spectrum disorder is Asperger's disorder (ASP).

In some embodiments, each expression level is a level of an RNA encoded by an autism spectrum disorder-associated gene. In certain embodiments, the expression level determining system comprises a hybridization-based assay for determining the level of the RNA in the clinical sample. In certain embodiments, the hybridization-based assay is an oligonucleotide array assay, an oligonucleotide conjugated bead assay, a molecular inversion probe assay, a serial analysis of gene expression (SAGE) assay, or an RT-PCR assay.

In some embodiments, each expression level is a level of a protein encoded by an autism spectrum disorder-associated gene. In certain embodiments, the expression level determining system comprises an antibody-based assay for determining the level of the protein in the clinical sample. In certain embodiments, the antibody-based assay is an antibody array assay, an antibody conjugated-bead assay, an enzyme-linked immuno-sorbent (ELISA) assay, or an immunoblot assay.

In some embodiments, the expression levels of autism spectrum disorder associated genes used in the methods comprise a combination of proteins levels and RNA levels.

According to some aspects of the invention, arrays are provided that comprise, or consist essentially of, oligonucleotide probes that hybridize to nucleic acids having sequence correspondence to mRNAs of at least two, at least three, at least four, at least five, at least six, at least seven, at least eight, at least nine, at least ten, at least twenty, at least 30, at least 40, at least 50, at least 60, at least 70, at least 80, or at least 90 genes selected from autism spectrum disorder-associated genes selected from Table 4, 5, 6, 8, 9, 10, or 11.

According to some aspects of the invention, arrays are provided that comprise, or consist essentially of, antibodies that bind specifically to proteins encoded by at least two, at least three, at least four, at least five, at least six, at least seven, at least eight, at least nine, at least ten, at least twenty, at least 30, at least 40, at least 50, at least 60, at least 70, at least 80, or at least 90 genes selected from autism spectrum disorder-associated genes selected from Table 4, 5, 6, 8, 9, 10, or 11.

According to some aspects of the invention, methods are provided for monitoring progression of an autism spectrum disorder in an individual in need thereof. In some embodiments, the methods involve (a) obtaining a clinical sample from the individual; (b) determining expression levels of a plurality of autism spectrum disorder-associated genes in the clinical sample using an expression level determining system, (c) comparing each expression level determined in (b) with an appropriate reference level, in which the results of the comparison are indicative of the extent of progression of the autism spectrum disorder in the individual.

In some embodiments, the monitoring methods involve (a) obtaining a first clinical sample from the individual, (b) determining expression levels of a plurality of autism spectrum disorder-associated genes in the first clinical sample using an expression level determining system, (c) obtaining a second clinical sample from the individual, (d) determining expression levels of the plurality of autism spectrum disorder-associated genes in the second clinical sample using an expression level determining system, (e) comparing the expression level of each autism spectrum disorder-associated gene determined in (b) with the expression level determined in (d) of the same autism spectrum disorder associated-gene, in which the results of comparing in (e) are indicative of the extent of progression of the autism spectrum disorder in the individual.

In some embodiments, the monitoring methods involve (a) obtaining a first clinical sample from the individual, (b) obtaining a second clinical sample from the individual, (c) determining the expression level of an autism spectrum disorder-associated gene in the first clinical sample using an expression level determining system, (d) determining the expression level of the autism spectrum disorder-associated gene in the second clinical sample using an expression level determining system, (e) comparing the expression level determined in (c) with the expression level determined in (d), (f) performing (c)-(e) for at least one other autism spectrum disorder-associated gene, in which the results of comparing in (e) for the at least two autism spectrum-associated genes are indicative of the extent of progression of the autism spectrum disorder in the individual.

In some embodiments, the monitoring methods involve (a) obtaining a first clinical sample from the individual, (b) obtaining a second clinical sample from the individual, (c) determining a first expression pattern comprising expression levels of at least two autism spectrum disorder-associated genes in the first clinical sample using an expression level determining system, (d) determining a second expression pattern comprising expression levels of at least two autism spectrum disorder-associated genes in the second clinical sample using an expression level determining system, (e) comparing the first expression pattern with the second expression pattern, in which the results of comparing in (e) are indicative of the extent of progression of the autism spectrum disorder in the individual.

In some embodiments of the monitoring methods, the time between obtaining the first clinical sample and obtaining the second clinical sample is a time sufficient for a change in the severity of the autism spectrum disorder to occur in the individual. In some embodiments of the monitoring methods, in the time between obtaining the first clinical sample and obtaining the second clinical sample the individual is treated for the autism spectrum associated disorder. In some embodiments, the time between obtaining the first clinical sample and obtaining the second clinical sample is up to about one week, about one month, about six months, about one year, about two years, about three years, or more. In some embodiments, the time between obtaining the first clinical sample and obtaining the second clinical sample is in a range of one week to one month, one month to six months, one month to one year, six months to one year, six months to two years, one year to three years, or one year to five years.

According to some aspects of the invention, methods are provided for assessing the efficacy of a treatment for an autism spectrum disorder in an individual in need thereof. In some embodiments, the methods involve: (a) obtaining a clinical sample from the individual, (b) administering a treatment to the individual for the autism spectrum disorder, (c) determining an expression pattern comprising expression levels of at least two autism spectrum disorder-associated genes in the clinical sample, (e) comparing the expression pattern with an appropriate reference expression pattern, in which the appropriate reference expression pattern comprises expression levels of the at least two autism spectrum disorder-associated genes in a clinical sample obtained from an individual who does not have the autism spectrum disorder, in which the results of the comparison in (c) are indicative of the efficacy of the treatment.

In some embodiments, the methods for assessing efficacy of a treatment for an autism spectrum disorder involve (a) obtaining a first clinical sample from the individual, (b) administering a treatment to the individual for the autism spectrum disorder, (c) obtaining a second clinical sample from the individual after having administered the treatment to the individual, (d) determining a first expression pattern comprising expression levels of at least two autism spectrum disorder-associated genes in the first clinical sample, (e) comparing the first expression pattern with an appropriate reference expression pattern, in which the appropriate reference expression pattern comprises expression levels of the at least two autism spectrum disorder-associated genes in a clinical sample obtained from an individual who does not have the autism spectrum disorder, (f) determining a second expression pattern comprising expression levels of at least two autism spectrum disorder-associated genes in the second clinical sample, and (g) comparing the second expression pattern with the appropriate reference expression pattern, in which a difference between the second expression pattern and the appropriate reference expression pattern that is less than the difference between the first expression pattern and the appropriate reference pattern is indicative of the treatment being effective.

According to some aspects of the invention, methods are provided for selecting an appropriate dosage of a treatment for an autism spectrum associated disorder in an individual in need thereof. In some embodiments, the methods involve (a) administering a first dosage of a treatment for an autism spectrum associated disorder to the individual, (b) assessing the efficacy of the first dosage of the treatment, in part, by determining at least one expression pattern comprising expression levels of at least two autism spectrum disorder-associated genes in a clinical sample obtained from the individual, (c) administering a second dosage of a treatment for an autism spectrum associated disorder in the individual, (d) assessing the efficacy of the second dosage of the treatment, in part, by determining at least one expression pattern comprising expression levels of at least two autism spectrum disorder-associated genes in a clinical sample obtained from the individual, in which the appropriate dosage is selected as the dosage administered in (a) or (c) that has the greatest efficacy.

According to some aspects of the invention, methods are provided for selecting an appropriate dosage of a treatment for an autism spectrum associated disorder in an individual in need thereof. In some embodiments, the methods involve (a) administering a dosage of a treatment for an autism spectrum associated disorder to the individual; (b) assessing the efficacy of the dosage of the treatment, in part, by determining at least one expression pattern comprising expression levels of at least two autism spectrum disorder-associated genes in a clinical sample obtained from the individual, and (c) selecting the dosage as being appropriate for the treatment for the autism spectrum associated disorder in the individual, if the efficacy determined in (b) is at or above a threshold level, in which the threshold level is an efficacy level at or above which a treatment substantially improves at least one symptom of an autism spectrum disorder.

According to some aspects of the invention, methods are provided for identifying an agent useful for treating an autism spectrum associated disorder in an individual in need thereof. In some embodiments, the methods involve (a) contacting an autism spectrum associated disorder-cell with a test agent, (b) determining at least one expression pattern comprising expression levels of at least two autism spectrum disorder-associated genes in the autism spectrum disorder-associated cell, (c) comparing the at least one expression pattern with a test expression pattern, and (d) identifying the agent as being useful for treating the autism spectrum associated disorder based on the comparison in (c). In some embodiments, the test expression pattern is an expression pattern indicative of an individual who does not have the autism spectrum disorder, and in which a decrease in a difference between the at least one expression pattern and the test expression pattern resulting from contacting the autism spectrum disorder-associated cell with the test agent identifies the test agent as being useful for the treatment of the autism spectrum associated disorder. In some embodiments, the autism spectrum disorder-associated cell is contacted with the test agent in (a) in vivo. In some embodiments, the autism spectrum disorder-associated cell is contacted with the test agent in (a) in vitro.

### BRIEF DESCRIPTION OF DRAWINGS

FIG. 1 depicts a non-limiting example of a procedure for a prediction analysis;

FIG. 2 depicts results of a principal component analysis of 285 blood gene expression profiles;

FIG. 3 depicts a non-limiting example of a method for selecting a minimum number of predictor genes to build a model;

FIG. 4A depicts the performance of an ASD85 prediction model trained with P1 to predict the diagnosis of each sample in P2; and

FIG. 4B depicts the performance of an ASD85 prediction model trained with P2 to predict the diagnosis of each sample in P1.

FIG. 5 depicts results of an analysis of subgroups in dysregulated pathways.

FIG. 6 depicts performance of the ASD55 prediction model. The dotted diagonal line represents random classification accuracy (AUC 0.5).

FIG. 7 depicts a cluster analysis of the 66 genes used in the prediction model (ASD55). The dendrogram and heatmap on top show hierarchical clustering (average linkage) of the 99 samples in the training set (P1) and the 55 genes used in the prediction model.

FIG. 8 depicts selection of predictor genes using repeated cross validation;

FIG. 9 depicts overlap between differentially expressed genes for each diagnostic subgroup in P1.

### DETAILED DESCRIPTION OF INVENTION

Autism Spectrum Disorder (ASD) is a highly heritable neurodevelopmental disorder. Applicants have developed robust profiling methods that classify the ASD status in individuals. In some embodiments, Applicants have developed methods that are useful for classifying the ASD status in males. In other embodiments, Applicants have developed methods that are useful for classifying the ASD status in individuals of particular age groups. In some embodiments, a gene expression based classifier is provided that achieves clinically relevant classification accuracies of ASD status. In other embodiments, gene expression based classifiers are provided that discriminate among autistic disorder (AUT), pervasive developmental disorder—not otherwise specified (PDDNOS), and Asperger's disorder (ASP). In some embodiments, the profiling methods are useful for diagnosing individuals as having ASD. In some embodiments, the profiling methods are also useful for selecting, or aiding in selecting, a treatment for an individual who has ASD or who is suspected of having ASD.

The term “autism spectrum disorder” (which may also be referred to herein by the acronym, “ASD”) refers to a spectrum of neuropsychological conditions that cause severe and pervasive impairment in thinking, feeling, language, and the ability to relate to others. Individuals with autism spectrum disorder may have restricted and/or repetitive behaviors or interests. Autism spectrum disorder may be first suspected or diagnosed in early childhood and may range in severity from a severe form, called autistic disorder, or autism, through pervasive development disorder not otherwise specified (PDD-NOS), to a milder form, Asperger syndrome. Autism spectrum disorder may also include two rare disorders, Rett syndrome and childhood disintegrative disorder. As used herein, the phrase “diagnosing autism spectrum disorder” refers to diagnosing, or aiding in diagnosing, an individual as having autism spectrum disorder.

As described herein, a variety of genes are differentially expressed in individuals having autism spectrum disorder compared with individuals identified as not having autism spectrum disorder. An “autism spectrum disorder-associated gene” is a gene whose expression levels are associated with autism spectrum disorder. Examples of autism spectrum disorder-associated genes include, but are not limited to, the genes listed in Table 4, 5, 6, 8, 9, 10 or 11. In some embodiments, the autism spectrum disorder associated gene is a gene of Table 4. Further examples of autism spectrum disorder genes are provided in Tables 13, 14, 15, 16, 17, 18, 19, 20, 21, 23, and 24.

As used herein, the term “autism spectrum disorder-associated cell” refers to a cell that expresses one or more autism spectrum disorder-associated genes. In some embodiments, an autism spectrum disorder-associated cell expresses at least two autism spectrum disorder associated genes. In some embodiments, an autism spectrum disorder-associated cell is a cell, obtained from an individual, that expresses autism spectrum disorder associated genes, the expression levels of which genes are useful for diagnosing or assessing the status of autism spectrum disorder in the individual. As used herein, the term “autism spectrum disorder-associated tissue” is a tissue comprising an autism spectrum disorder-associated cell.

The term “individual”, as used herein, refers to any mammal, including, humans and non-humans, such as primates. Typically, an individual is a human. An individual may be of any appropriate age for the methods disclosed herein. For example, methods disclosed herein may be used to characterize the autism spectrum disorder status of a child, e.g., a human in a range of about 1 to about 12 years old. An individual may be a non-human that serves as an animal model of autism spectrum disorder. An individual may alternatively be referred to herein synonymously as a subject.

Methods are provided herein for characterizing the autism spectrum disorder status of an individual in need thereof. An individual in need of a characterization of autism spectrum disorder status is any individual at risk of, or suspected of, having autism spectrum disorder. An individual's “autism spectrum disorder status” may be characterized as having autism spectrum disorder or as not having autism spectrum disorder.

An individual in need of diagnosis of autism spectrum disorder is any individual at risk of, or suspected of, having autism spectrum disorder. An individual at risk of having autism spectrum disorder may be an individual having one or more risk factors for autism spectrum disorder. Risk factors for autism spectrum disorder include, but are not limited to, a family history of autism spectrum disorder; elevated age of parents; low birth weight; premature birth; presence of a genetic disease associated with autism; and sex (males are more likely to have autism than females). Other risk factors will be apparent to the skilled artisan. An individual suspected of having autism spectrum disorder may be an individual having one or more clinical symptoms of autism spectrum disorder. A variety of clinical symptoms of Autism Spectrum Disorder are known in the art. Examples of such symptoms include, but are not limited to, no babbling by 12 months; no gesturing (pointing, waving goodbye, etc.) by 12 months; no single words by 16 months; no two-word spontaneous phrases (other than instances of echolalia) by 24 months; any loss of any language or social skills, at any age.

The methods disclosed herein may be used in combination with any one of a number of standard diagnostic approaches, including, but not limited to, clinical or psychological observations and/or ASD-related screening modalities, such as, for example, the Modified Checklist for Autism in Toddlers (M-CHAT), the Early Screening of Autistic Traits Questionnaire, and the First Year Inventory to facilitate or aid in the diagnosis of ASD. In some embodiments, methods disclosed herein are used to identify subgroups of ASD.

The methods disclosed herein typically involve determining expression levels of at least one autism spectrum disorder-associated genes in a clinical sample obtained from an individual. The methods may involve determining expression levels of at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, or more autism spectrum disorder-associated genes in a clinical sample obtained from an individual. The methods may involve determining expression levels of 1 to 10, 10 to 20, 20 to 30, 30 to 40, 40 to 50, 50 to 60, 60 to 70, 70 to 80, 80 to 90, 90 to 100, 100 to 200, 200 to 300, or 300 to 400 autism spectrum disorder-associated genes in a clinical sample obtained from an individual. The methods may involve determining expression levels of about 10, about 20, about 30, about 35, about 40, about 50, about 60, about 70, about 80, about 85, about 90, about 100, or more autism spectrum disorder-associated genes in a clinical sample obtained from an individual.

An expression level determining system may be used in the methods. The term “expression level determining system”, as used herein, refers to a set of components, equipment, and/or reagents, for determining the expression level of a gene in a sample. The expression level of an autism spectrum disorder-associated gene may be determined as the level of an RNA encoded by the gene, in which case, the expression level determining system may comprise components useful for determining levels of nucleic acids. The expression level determining system may comprises, for example, hybridization-based assay components, and related equipment and reagents, for determining the level of the RNA in the clinical sample. Hybridization-based assays are well known in the art and include, but are not limited to, oligonucleotide array assays (e.g., microarray assays), cDNA array assays, oligonucleotide conjugated bead assays (e.g., Multiplex Bead-based Luminex® Assays), molecular inversion probe assay, serial analysis of gene expression (SAGE) assay, RNase Protein Assay, northern blot assay, an in situ hybridization assay, and an RT-PCR assay. Multiplex systems, such as oligonucleotide arrays or bead-based nucleic acid assay systems are particularly useful for evaluating levels of a plurality of nucleic acids in simultaneously. RNA-Seq (mRNA sequencing using Ultra High throughput or Next Generation Sequencing) may also be used to determine expression levels. Other appropriate methods for determining levels of nucleic acids will be apparent to the skilled artisan.

The expression level of an autism spectrum disorder-associated gene may be determined as the level of a protein encoded by the gene, in which case, the expression level determining system may comprise components useful for determining levels of proteins. The expression level determining system may comprises, for example, antibody-based assay components, and related equipment and reagents, for determining the level of the protein in the clinical sample. Antibody-based assays are well known in the art and include, but are not limited to, antibody array assays, antibody conjugated-bead assays, enzyme-linked immuno-sorbent (ELISA) assays, immunofluorescence microscopy assays, and immunoblot assays. Other methods for determining protein levels include mass spectroscopy, spectrophotometry, and enzymatic assays. Still other appropriate methods for determining levels of proteins will be apparent to the skilled artisan.

As used herein, a “level” refers to a value indicative of the amount or occurrence of a molecule, e.g., a protein, a nucleic acid, e.g., RNA. A level may be an absolute value, e.g., a quantity of a molecule in a sample, or a relative value, e.g., a quantity of a molecule in a sample relative to the quantity of the molecule in a reference sample (control sample). The level may also be a binary value indicating the presence or absence of a molecule. For example, a molecule may be identified as being present in a sample when a measurement of the quantity of the molecule in the sample, e.g., a fluorescence measurement from a PCR reaction or microarray, exceeds a background value. Similarly, a molecule may be identified as being absent from a sample (or undetectable in a sample) when a measurement of the quantity of the molecule in the sample is at or below background value.

The methods may involve obtaining a clinical sample from the individual. As used herein, the phrase “obtaining a clinical sample” refers to any process for directly or indirectly acquiring a clinical sample from an individual. For example, a clinical sample may be obtained (e.g., at a point-of-care facility, e.g., a physician's office, a hospital) by procuring a tissue or fluid sample (e.g., blood draw, spinal tap) from an individual. Alternatively, a clinical sample may be obtained by receiving the clinical sample (e.g., at a laboratory facility) from one or more persons who procured the sample directly from the individual.

The term “clinical sample” refers to a sample derived from an individual, e.g., a patient. Clinical samples include, but are not limited to tissue (e.g., brain tissue), cerebrospinal fluid, blood, blood fractions (e.g., serum, plasma), sputum, fine needle biopsy samples, urine, peritoneal fluid, and pleural fluid, or cells therefrom (e.g., blood cells (e.g., white blood cells, red blood cells)). Accordingly, a clinical sample may comprise a tissue, cell or biomolecule (e.g., RNA, protein). In some embodiments, the clinical sample is a sample of peripheral blood, brain tissue, or spinal fluid.

It is to be understood that a clinical sample may be processed in any appropriate manner to facilitate determining expression levels of autism spectrum disorder-associated genes. For example, biochemical, mechanical and/or thermal processing methods may be appropriately used to isolate a biomolecule of interest, e.g., RNA, protein, from a clinical sample. A RNA sample may be isolated from a clinical sample by processing the clinical sample using methods well known in the art and levels of an RNA encoded by an autism spectrum disorder-associated gene may be determined in the RNA sample. A protein sample may be isolated from a clinical sample by processing the clinical sample using methods well known in the art. And levels of a protein encoded by an autism spectrum disorder-associated gene may be determined in the protein sample. The expression levels of autism spectrum disorder-associated genes may also be determined in a clinical sample directly.

The methods disclosed herein also typically comprise comparing expression levels of autism spectrum disorder-associated genes with an appropriate reference level. An “appropriate reference level” is an expression level of a particular autism spectrum disorder gene that is indicative of a known autism spectrum disorder status. An appropriate reference level can be determined or can be a pre-existing reference level. An appropriate reference level may be an expression level indicative of autism spectrum disorder. For example, an appropriate reference level may be representative of the expression level of an autism spectrum disorder-associated gene in a clinical sample obtained from an individual known to have autism spectrum disorder. When an appropriate reference level is indicative of autism spectrum disorder, a lack of a significant difference between an expression level determined from an individual in need of characterization or diagnosis of autism spectrum disorder and the appropriate reference level may be indicative of autism spectrum disorder in the individual. Alternatively, when an appropriate reference level is indicative of autism spectrum disorder, a significant difference between an expression level determined from an individual in need of characterization or diagnosis of autism spectrum disorder and the appropriate reference level may be indicative of the individual being free of autism spectrum disorder.

An appropriate reference level may be a threshold level such that an expression level being above or below the threshold level is indicative of autism spectrum disorder in an individual.

An appropriate reference level may be an expression level indicative of an individual being free of autism spectrum disorder. For example, an appropriate reference level may be representative of the expression level of a particular autism spectrum disorder-associated gene in a clinical sample obtained from an individual who does not have autism spectrum disorder. When an appropriate reference level is indicative of an individual who does not have autism spectrum disorder, a significant difference between an expression level determined from an individual in need of diagnosis of autism spectrum disorder and the appropriate reference level may be indicative of autism spectrum disorder in the individual. Alternatively, when an appropriate reference level is indicative of the individual being free of autism spectrum disorder, a lack of a significant difference between an expression level determined from an individual in need of diagnosis of autism spectrum disorder and the appropriate reference level may be indicative of the individual being free of autism spectrum disorder.

For example, when a higher level, relative to an appropriate reference level that is indicative of an individual who does not have autism spectrum disorder, of at least one autism spectrum disorder-associated gene, which is selected from: ZNF12, RBL2, ZNF292, IVNS1ABP, ZFP36L2, ARFGEF1, UTY, SLA, KIAA0247, HNRNPA2B1, RNF145, PTPRE, SFRS18, ZNF238, TRIP12, PNN, ZDHHC17, MLL3, MTMR10, STK38, SERINC3, NIPBL, TIGD1, DDX42, NUP50, CAB39, ROCK1, SULF2, FABP2, KIDINS220, NCOA6, SIRPA, PCSK5, ADAM10, ZNF33A, ZMAT1, C10orf28, MGAT4A, CEP110, ZZEF1, CREBZF, DOCK11, ATRN, COL4A3BP, FAM133A, TTC14, TMEM30A, MYO5A, KDM2A, ZCCHC14, RNF44, ZBTB44, CLTC, UTRN, ATXN7, PPP1R12A, LBR, TBC1D14, SPATA13, HK2, CREBBP, MED23, ZFYVE16, PAN3, RBBP6, AVL9, ZNF354A, ACTR2, TMBIM1, RPS6KA3, DNMBP, NBEAL2, MYSM1, TMEM2, SNRK, KIAA1109, HECA, DNAJC3, KIF5B, POLR2B, ANTXR2, VPS13C, MANBA, and NIN, is identified, the individual's autism spectrum disorder status may be characterized as having autism spectrum disorder. When a lower level, relative to an appropriate reference level that is indicative of an individual who does not have autism spectrum disorder, of at least one autism spectrum disorder-associated gene, which includes STXBP6, is identified, the individual's autism spectrum disorder status may be characterized as having autism spectrum disorder.

The magnitude of difference between an expression level and an appropriate reference level may vary. For example, a significant difference that indicates an autism spectrum disorder status or diagnosis may be detected when the expression level of an autism spectrum disorder-associated gene in a clinical sample is at least 1%, at least 5%, at least 10%, at least 25%, at least 50%, at least 100%, at least 250%, at least 500%, or at least 1000% higher, or lower, than an appropriate reference level of that gene. Similarly, a significant difference may be detected when the expression level of an autism spectrum disorder-associated gene in a clinical sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference level of that gene. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

It is to be understood that a plurality of expression levels may be compared with plurality of appropriate reference levels, e.g., on a gene-by-gene basis, as a vector difference, in order to assess the autism spectrum disorder status of the individual. In such cases, Multivariate Tests, e.g., Hotelling's T² test, may be used to evaluate the significance of observed differences. Such multivariate tests are well known in the art and are exemplified in Applied Multivariate Statistical Analysis by Richard Arnold Johnson and Dean W. Wichern Prentice Hall; 4^{th} edition (Jul. 13, 1998).

The methods may also involve comparing a set of expression levels (referred to as an expression pattern) of autism spectrum disorder-associated genes in a clinical sample obtained from an individual with a plurality of sets of reference levels (referred to as reference patterns), each reference pattern being associated with a known autism spectrum disorder status; identifying the reference pattern that most closely resembles the expression pattern; and associating the known autism spectrum disorder status of the reference pattern with the expression pattern, thereby classifying (characterizing) the autism spectrum disorder status of the individual.

The methods may also involve building or constructing a prediction model, which may also be referred to as a classifier or predictor, that can be used to classify the disease status of an individual. As used herein, an “autism spectrum disorder-classifier” is a prediction model that characterizes the autism spectrum disorder status of an individual based on expression levels determined in a clinical sample obtained from the individual. Typically the model is built using samples for which the classification (autism spectrum disorder status) has already been ascertained. Once the model is built, it may be applied to expression levels obtained from a clinical sample in order to classify the autism spectrum disorder status of the individual from which the clinical sample was obtained. Thus, the methods may involve applying an autism spectrum disorder-classifier to the expression levels, such that the autism spectrum disorder-classifier characterizes the autism spectrum disorder status of the individual based on the expression levels. The individual may be further diagnosed, e.g., by a health care provider, based on the characterized autism spectrum disorder status.

A variety of prediction models known in the art may be used as an autism spectrum disorder-classifier. For example, an autism spectrum disorder-classifier may be established using logistic regression, partial least squares, linear discriminant analysis, quadratic discriminant analysis, neural network, naïve Bayes, C4.5 decision tree, k-nearest neighbor, random forest, and support vector machine.

The autism spectrum disorder-classifier may be trained on a data set comprising expression levels of the plurality of autism spectrum disorder-associated genes in clinical samples obtained from a plurality of individuals identified as having autism spectrum disorder. For example, the autism spectrum disorder-classifier may be trained on a data set comprising expression levels of a plurality of autism spectrum disorder-associated genes in clinical samples obtained from a plurality of individuals identified as having autism spectrum disorder based on DSM-IV-TR criteria. The training set will typically also comprise control individuals identified as not having autism spectrum disorder, e.g., identified as not satisfying the DSM-IV-TR criteria. As will be appreciated by the skilled artisan, the population of individuals of the training data set may have a variety of characteristics by design, e.g., the characteristics of the population may depend on the characteristics of the individuals for whom diagnostic methods that use the classifier may be useful. For example, the interquartile range of ages of a population in the training data set may be from about 2 years old to about 10 years old, about 1 year old to about 20 years old, about 1 year old to about 30 years old. The median age of a population in the training data set may be about 1 year old, 2 years old, 3 years old, 4 years old, 5 years old, 6 years old, 7 years old, 8 years old, 9 years old, 10 years old, 20 years old, 30 years old, 40 years old, or more. The population may consist of all males, all females or may consist of males and females.

A class prediction strength can also be measured to determine the degree of confidence with which the model classifies a clinical sample. The prediction strength conveys the degree of confidence of the classification of the sample and evaluates when a sample cannot be classified. There may be instances in which a sample is tested, but does not belong, or cannot be reliable assigned to, a particular class. This is done by utilizing a threshold in which a sample which scores above or below the determined threshold is not a sample that can be classified (e.g., a “no call”).

Once a model is developed, the validity of the model can be tested using methods known in the art. One way to test the validity of the model is by cross-validation of the dataset. To perform cross-validation, one, or a subset, of the samples is eliminated and the model is built, as described above, without the eliminated sample, forming a “cross-validation model.” The eliminated sample is then classified according to the model, as described herein. This process is done with all the samples, or subsets, of the initial dataset and an error rate is determined. The accuracy the model is then assessed. This model classifies samples to be tested with high accuracy for classes that are known, or classes have been previously ascertained. Another way to validate the model is to apply the model to an independent data set, such as a new clinical sample having an unknown autism spectrum disorder status. Other appropriate validation methods will be apparent to the skilled artisan.

As will be appreciated by the skilled artisan, the strength of the model may be assessed by a variety of parameters including, but not limited to, the accuracy, sensitivity, specificity and area under the receiver operation characteristic curve. Methods for computing accuracy, sensitivity and specificity are known in the art and described herein (See, e.g., the Examples). The autism spectrum disorder-classifier may have an accuracy of at least 60%, at least 65%, at least 70%, at least 75%, at least 80%, at least 85%, at least 90%, at least 95%, at least 99%, or more. The autism spectrum disorder-classifier may have an accuracy score in a range of about 60% to 70%, 70% to 80%, 80% to 90%, or 90% to 100%. The autism spectrum disorder-classifier may have a sensitivity score of at least 60%, at least 65%, at least 70%, at least 75%, at least 80%, at least 85%, at least 90%, at least 95%, at least 99%, or more. The autism spectrum disorder-classifier may have a sensitivity score in a range of about 60% to 70%, 70% to 80%, 80% to 90%, or 90% to 100%. The autism spectrum disorder-classifier may have a specificity score of at least 60%, at least 65%, at least 70%, at least 75%, at least 80%, at least 85%, at least 90%, at least 95%, at least 99%, or more. The autism spectrum disorder-classifier may have a specificity score in a range of about 60% to 70%, 70% to 80%, 80% to 90%, or 90% to 100%.

Described herein are oligonucleotide (nucleic acid) arrays that are useful in the methods for determining levels of multiple nucleic acids simultaneously. Such arrays may be obtained or produced from commercial sources. Methods for producing nucleic acid arrays are well known in the art. For example, nucleic acid arrays may be constructed by immobilizing to a solid support large numbers of oligonucleotides, polynucleotides, or cDNAs capable of hybridizing to nucleic acids corresponding to mRNAs, or portions thereof. The skilled artisan is also referred to Chapter 22 “Nucleic Acid Arrays” of Current Protocols In Molecular Biology (Eds. Ausubel et al. John Wiley and #38; Sons NY, 2000), International Publication WO00/58516, U.S. Pat. No. 5,677,195 and U.S. Pat. No. 5,445,934 which provide non-limiting examples of methods relating to nucleic acid array construction and use in detection of nucleic acids of interest. In some embodiments, the nucleic acid arrays comprise, or consist essentially of, binding probes for mRNAs of at least 2, at least 5, at least 10, at least 20, at least 50, at least 100, at least 200, at least 300, or more genes selected from Table 6. Kits comprising the oligonucleotide arrays are also provided. Kits may include nucleic acid labeling reagents and instructions for determining expression levels using the arrays.

### EXAMPLES

### Introduction to the Examples

Autism Spectrum Disorder (ASD) relates to a broad spectrum of neurocognitive and social developmental delays including autistic disorder, pervasive developmental disorder—not otherwise specified and Asperger's Disorder as sub classified in the Diagnostic and Statistical Manual of Mental Disorders, 4th edition, Text Revision (DSM-W-TR). Onset of ASD may occur before 3 years of age. Reported prevalence of ASD has been increasing during the last decades, and a current estimation is 1 in 91. There are long waiting lists for evaluation at most centers with expertise. Progress has been made in adopting instruments such as the Autism Diagnostic Interview-Revised (ADI-R) and the Autism Diagnostic Observation Schedule (ADOS). In some cases, the median age at diagnosis is 5.7 years.

Early diagnosis and behavioral intervention may improve outcomes. This example provides diagnostic tests and/or biomarkers that can be used (e.g., in primary pediatric care centers) to reduce the time to accurate diagnosis. This example describes a gene expression study of ASD, and demonstrates the performance of blood expression signatures that classify children with ASD and distinguish ASD from controls. The signature may be useful for making a diagnosis, for example, after an increased index of suspicion is determined based on parent and/or pediatric assessment. Studies on an additional cohort were performed to further validate this signature.

### Example 1

### Materials and Methods

### Blood Gene Expression Profiling

Gene expression profiles of P1 were prepared using Affymetrix HG-U133 Plus 2.0 (U133p2) and those of P2 were profiled using Affymetrix Gene 1.0 ST (GeneST) arrays (Affymetrix, CA). Within the P1 data set, RNAs from 39 ASD and 12 control samples were isolated directly from whole blood using the RiboPure Blood Kit (Ambion). For all other blood samples, total RNA was extracted from 2.5 ml of whole venous blood using the PAX gene Blood RNA System (PreAnalytix). Quality and quantity of these RNAs was assessed using the Nanodrop spectrophotometer (Thermo Scientific) and Bioanalyzer System (Agilent). Fragmented cRNA was hybridized to the appropriate Affymetrix array and scanned on an Affymetrix GeneChip scanner 3000. cRNA from both affected and normal control population groups was prepared in batches consisting of a randomized assortment of the two comparison groups.

### Statistical Analysis

The gene expression levels were calculated using the probe log iterative error algorithm after normalizing the probe intensities using a quantile method. To identify differentially expressed genes in cases compare to controls, we used Welch's t-test for two groups comparison, and one-way analysis of variance with Dunnett's post hoc tests to find significantly changed genes in AUT, PDDNOS, or ASP compare to control group. A general linear model was used to evaluate the significance of diagnosis, gender, age, and the other covariates. P values were corrected for the multiple comparisons by calculating a false discovery rate (FDR). Fisher's exact test was used for categorical data. Spearman's rank correlation coefficients were calculated to evaluate correlation between continuous phenotypic variables such as age at blood drawing and expression level of each gene. The significance of correlation was determined using Fisher's r-to-z transformation. A machine learning method was used to build a prediction model using multi-gene expression profiles. Enriched biological pathways with predictor genes were found using the DAVID functional annotation system. Statistical analyses were performed using the R statistical programming language.

### Prediction Analysis

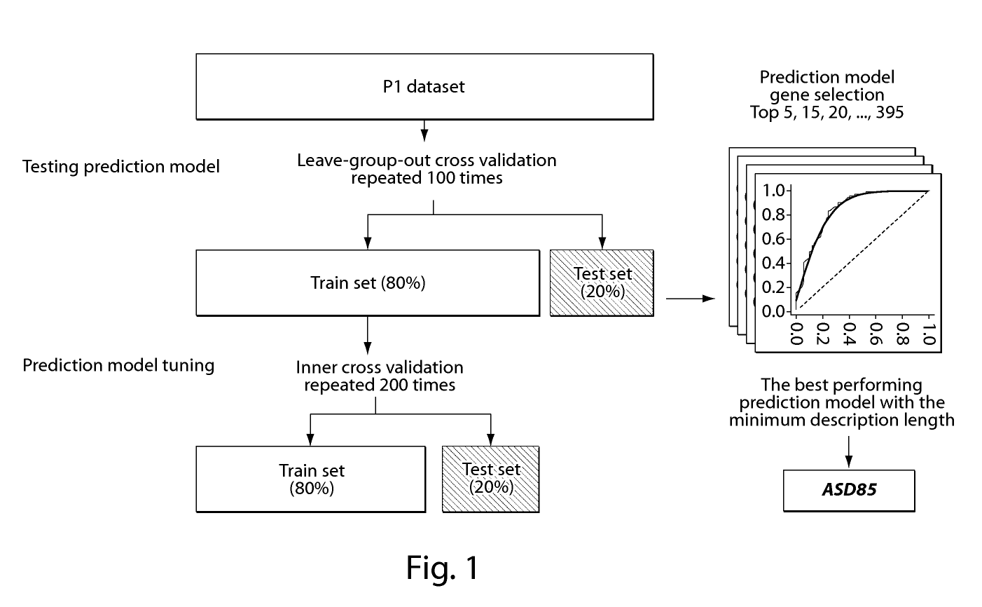

Prediction analyses were performed using the following sequential steps: 1) rank order genes for predictor selection, 2) set up a cross-validation strategy in the training set, 3) select prediction algorithm and build a prediction model, 4) predict a test set, and 5) evaluate prediction performance as illustrated in FIG. 1.

First, all genes were ranked by Welch's t-test p-values between AUT+PDDNOS vs. controls in the P1 dataset. The top N differentially expressed genes from 10 to 395 by 5 were selected and used to build a prediction model with the P1 dataset using a repeated leave-group out cross-validation (LGOCV) strategy. For each prediction model using the top N genes, all P1 samples (N=99) were divided to 80% (a train set) and 20% (a test set), keeping the proportion of ASD and controls the same in each set. This step was repeated 100 times to estimate robust prediction performance. To optimize each prediction model further, an inner cross-validation approach was deployed where 80% of the samples served as an inner train set, and 20% were used as an inner test set. The inner cross-validation procedure was repeated 200 times to find optimal tuning parameters for the specific prediction algorithm used. For each prediction model with top N genes, a total of 20,000 predictions (100 repeated LGOCVs×200 inner cross-validations) had been made. A partial least squares (PLS) method was used to find the best performing model.

For each sample in a test set, the model predicts the probability of being classified as ASD. Thus, the number of false positives among positive predictions changes with the threshold. Overall prediction accuracy was calculated as (the number of true positives+the number of true negatives)/N, where N was the total number of samples in a dataset. Sensitivity, specificity, positive predictive value, and negative predictive value were presented as standard measures of prediction performance with the area under the receiver operation characteristic curve (AUC). Sensitivity was calculated as the number of true positives divided by the sum of the number of true positives and the number of false negatives. Specificity was calculated as the number of true negatives divided by the sum of the number of true negatives and the number of false positives. The receiver operating characteristic (ROC) curve summarizes the result at different thresholds. AUC was calculated from the ROC curve as AUC=∫₀⁰ROC(t)dt. AUC and root mean squared errors (RMSE) were used as performance measurements to decide the number of genes for the final prediction model. RMSE was as defined as Equation 1 where p is a probability of being ASD and a is an integer for each class (1 being ASD and 0 being control) for n^{th} sample.

$$RMSE = \sqrt{\frac{{( {p}_{1} - {a}_{1} )}^{2} + … + {( {p}_{n} - {a}_{n} )}^{2}}{n}} \qquad ( Equation 1 )$$

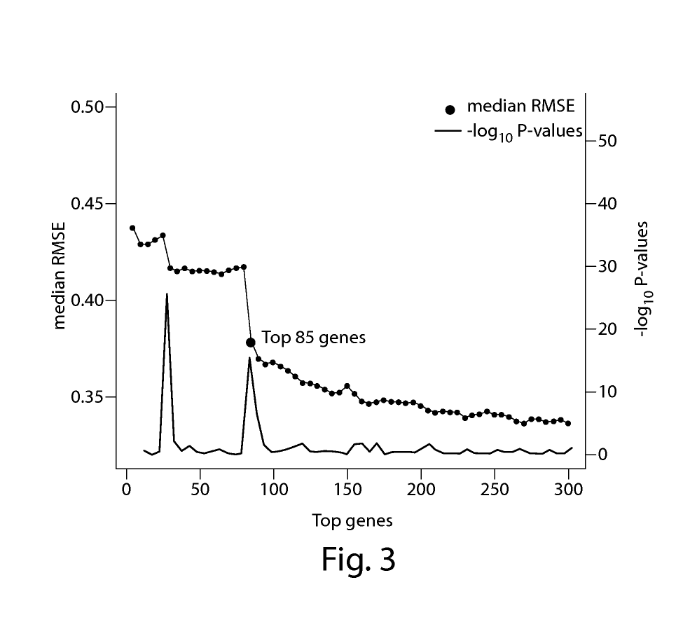

To find a relatively strong performing prediction model with the minimum description length, RMSEs of each prediction model were compared using the top N genes. The mean RMSEs improved gradually with increasing model complexities. As shown in FIG. 3, two significant improvements in prediction performances were found. Although all models produced useful results, the prediction model with top 35 genes (the first 35 genes listed in Table 6) performed significantly better than the 30 genes prediction model (t-test P=2.47×10⁻²⁶), and the 85-genes model performed significantly better than the 80-genes model (t-test P=3.59×10⁻¹⁶) Five additional prediction methods: Logistic regression, Naïve Bayes, k-Nearest Neighbors, Random Forest, and Support Vector Machine using 85 genes with 5 fold LGOCV strategy were tested (Table 7). Statistical prediction analysis was performed using the caret and RWeka R library packages.

### Quantitative RT-PCR Validation

A total of 165 ASD and 103 control samples were run in replicates of four on the Biomark real time PCR system (Fluidigm, CA) using nanoliter reactions and the Taqman system (Applied Biosystems, CA). Following the Biomark protocol, quantitative RT-PCR (qRT-PCR) amplifications were carried out in a 9 nanoliter reaction volume containing 2× Universal Master Mix (Taqman), taqman gene expression assays, and preamplified cDNA. Pre-amplification reactions were done in a PTC-200 thermal cycler from MJ Research, per Biomark protocol. Reactions and analysis were performed using a Biomark system. The cycling program consisted of an initial cycle of 50° C. for 2 minutes and a 10 min incubation at 95° C. followed by 40 cycles of 95° C. for 15 seconds, 70° C. for 5 seconds, and 60° C. for 1 minute. Data was normalized to the housekeeping gene GAPDH, and expressed relative to control.

### Principal Component Analysis

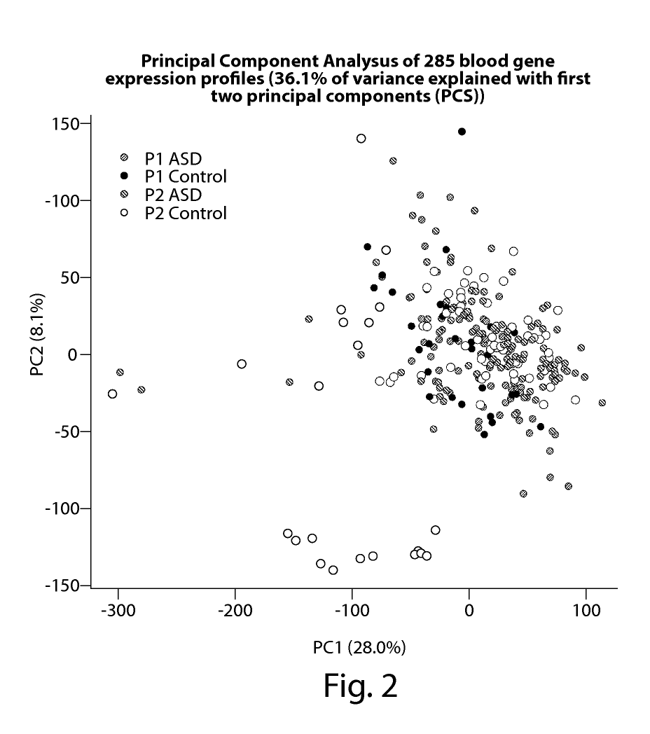

FIG. 2 depicts results of a principal component analysis of 285 blood gene expression profiles. Global gene expression profile of the Training set (P1) and the Validation set (P2) samples. After selecting the best-matching probesets between two Affymetrix microarray platforms, principal component analysis was performed. All samples from P1 and P2 were projected to two-dimensional space of the first (PC1) and the second (PC2) principal components. 36.1% of overall variance was explained by PC1 and PC2. No significant difference was observed between two datasets after normalization.

### Predictor Gene Selection

FIG. 3 depicts a method for selecting a minimum number of predictor genes to build a model. This prediction model selection procedure consisted of three nested loops. The outer-most loop was the selection of the top N genes (10 to 395 by 5) in the ranked gene list by p-values from the comparison between AUT+PDDNOS vs. controls. The second loop was a leave-group out cross validation approach, where 80% of samples were randomly selected as a train set, while maintaining the proportion of each diagnostic class. This step was repeated 100 times for each list of the top N genes. The inner-most loop was used to optimize the parameters that were specific to machine learning methods used for a train set from an outer loop. This parameter tunings were repeated 200 times by randomly selecting 80% of the train set samples. The prediction performance was estimated using the area under the receiver operation characteristic curve and the root mean squared error (RMSE). Mean RMSEs improved gradually when the number of genes was increased to build more complex prediction models; however, the prediction model that used the top 85 genes performed significantly better than the 80 gene model (t-test P=3.59×10-16). Prediction models using 90 or more genes showed minimal improvement.

### Example 2

### Gene Expression Signature Assessment

ASD patients were recruited. Study inclusion criteria comprised a clinical diagnosis of ASD by DSM-IV-TR criteria and an age >24 months. Patients with ASD recruited for this study have underwent diagnostic assessment, using ADOS and ADI-R, as well as clinical testing including cognitive testing, language measures, medical history, height and weight, head circumference, and behavioral questionnaires. Two independently collected data sets (hereafter P1 and P2) consisted of 66 and 104 ASD individuals. Patients with known syndromic disorders such as fragile X mental retardation, tuberous sclerosis, Landau-Kleffner syndrome, and Klinefelter syndrome were not included in this study.

A total of 115 controls were enrolled concurrently. Certain control patients were identified as healthy children with idiopathic short stature, including genetic short stature and constitutional delay of growth, and were having clinical blood draws. Clinical blood draw results were evaluated to confirm they were within normal limits (those that were not were withdrawn from the study). Certain other control patients were offered enrollment during a well-child visit that involved a routine blood draw (for example, to obtain lead levels). A diagnosis of a chronic disease, mental retardation, ASD, or neurological disorder was used as exclusion criteria from our control group. Complete phenotypic information is available with microarray data (Gene Expression Omnibus identifier GSE18123). Each cohort's clinical and demographic information is shown in Table 1.

There was no statistical difference in age between ASD and controls in the P1 (Welch's t-test P=0.29) or P2 cohort (P=0.73). Ages of ASD samples between the P1 and P2 populations were not different (P=0.52). Because of disease incidence discordance in males and females, with males 4 times more likely to develop the disease, and because a preliminary analysis revealed higher heterogeneity in RNA levels in females with ASD than in males, possibly due the smaller number of females or to the sexual dimorphism in the expression of the disorder, only males were included in the P1 cohort (both ASD and controls samples), which was used to build a prediction model for ASD. The performance of the predictive model was tested for both males and females in the P2 cohort (although the number female controls was higher than that of female ASD—Fisher's exact test P=0.01 in P2).

### Blood Gene Expression Changes in ASD

Expression studies were performed by microarray profiling using an earlier version of the Affymetrix array (U133p2) for the P1 data set and a later version (GeneST) for the P2 data set. To match the probeset identifiers from the two different platforms used in this study, a Best Match subset was used. 29,129 out of 54,613 total probesets on U133p2 were best matched to 17,984 unique probesets of GeneST array, and these matched probesets were used for further analysis. After selecting the best matching probesets between two platforms, principal component analysis was performed to project samples onto the first two principal components (FIG. 2). The difference between the two datasets was minimal after normalization.

There were 291 and 4039 genes differentially expressed between ASD and controls in the P1 and P2 datasets, respectively (Welch's t-test P<0.001, corresponding FDRs 0.029 (P1), and 0.0023 (P2)). Of these, 67 genes were significant in both cohorts, as set forth in Table 8. Three genes were randomly selected from the differentially expressed genes in the P1 dataset, and validated changes using quantitative RT-PCR in the P2 and additional samples (total N=165 for ASD and N=103 for controls) (Table 5). All 3 genes, LRRC6, SULF2, and YES1 were significantly up-regulated. When each diagnostic subtype was compared to controls in the P1 dataset, 100, 43, and 9 genes (as set forth in Tables 9, 10, and 11, respectively) were significant for autistic disorder (AUT), pervasive developmental disorder—not otherwise specified (PDDNOS), and Asperger's disorder (ASP) respectively (Welch's t-test P<0.001, corresponding FDRs 0.13 (AUT), 0.31 (PDDNOS), and 1.0 (ASP)). Among the significant genes in ASP, only one gene overlapped with AUT vs. control. None of the significant genes in ASP was differentially expressed in the patients with PDDNOS compared to controls. Interestingly, a larger number of genes were differentially expressed when 9 ASP cases were excluded, and compared ASD with control. A total of 395 genes were significant when the ASP samples were excluded compared to 291 genes when the ASP samples were included at the same statistical threshold (P<0.001, corresponding FDR 0.02 for 395 genes and 0.029 for 291 genes).

To determine which biological processes were implicated by the differentially expressed genes in ASD, an enrichment calculation was performed using a hypergeometric test. This metric allowed a determination of which processes were overrepresented in the 395 top most differentially expressed genes when the ASP samples were excluded (P<0.001, corresponding FDR 0.02) relative to all the processes annotated in the Kyoto Encyclopedia of Genes and Genomes (KEGG). These results are enumerated in Table 3. In this experiments, the Neurotrophin signaling pathway (KEGG pathway identifier: hsa04722) was the most significant (hypergeometric test P=0.0011, FDR 0.012) among 14 overrepresented pathways (hypergeometric test P<0.05, corresponding FDR 0.39). The Neurotrophin signaling pathway includes neurotrophins and their second messenger systems such as the MAPK pathway, PI3K pathway, and PLC pathway, which have been identified by others as important for neural development, learning and memory, and syndromic ASD such as tuberous sclerosis and Smith-Lemli-Opitz syndrome. The second most significant pathway in this experiment was the Long-term potentiation pathway (hypergeometric test P=0.0029, FDR 0.032).

### Prediction of Autism Using Blood Gene Expression Signatures

Peripheral blood gene expression profiles may be used as a molecular diagnostic tool for identifying ASD from controls. A repeated leave-group out cross-validation (LGOCV) strategy was used with P1 to build prediction models. The training set, which consisted of the P1 cohort, was utilized to determine a classification signature (the combination of gene expression measurements) that was used to classify ASD patients in P1 (compared to controls). Genes were ranked according to p-values from AUT+PDDNOS vs. controls comparison in P1 since the differentially expressed genes were more prominent when AUT and PDDNOS samples were compared to controls without the ASP samples. This signature was then tested against the samples in an independent validation cohort (P2). The top N differentially expressed genes (where N ranges from 5 to 395 by 5) were used to build prediction models using a repeated 5-folds LGOCV with a partial least squares (PLS) method, and root mean squared errors (RMSE) were calculated (see Example 1). Mean RMSEs improved gradually when the number of genes was increased to build more complex prediction models; however, the prediction model that used the top 85 genes performed significantly better than the 80 gene model (t-test P=3.59×10⁻¹⁶) (FIG. 3). Prediction models using 90 or more genes showed minimal improvement. The 85-gene prediction model was chosen. The model minimized description length while maintaining good prediction performance, and used it to evaluate the independent dataset, P2 (see Example 1). The 85 significant genes are listed in Table 6. The performance of PLS was comparable to those of other prediction algorithms (Table 7); thus the classification performance was not attributable to a specific prediction algorithm.

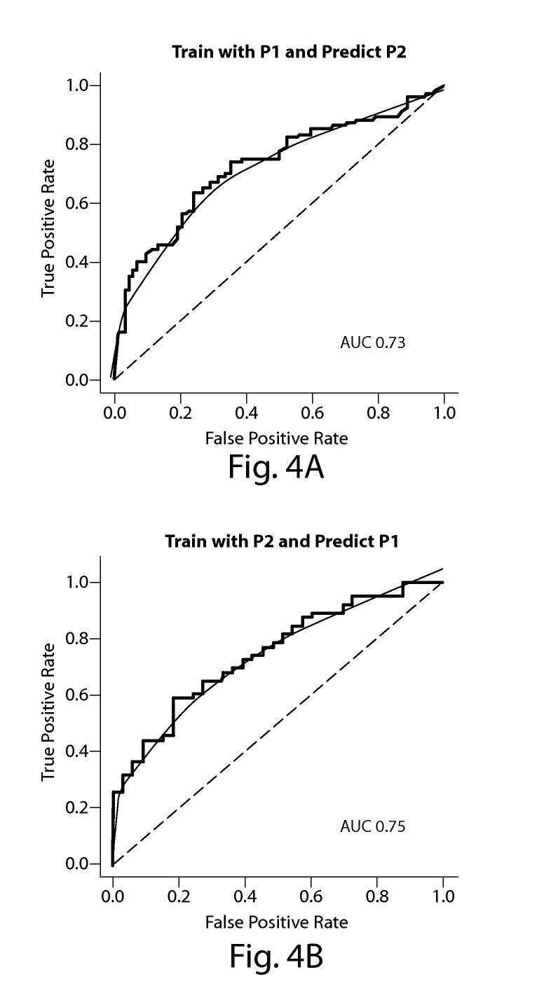

The accuracy of this 85-gene set (hereafter referred to as ASD85) within P1 was relatively high (area under the receiver operating characteristic curve (AUC) 0.96, 95% confidence interval (CI), 0.930-0.996), and also had good performance when applied to the P2 validation population (AUC 0.73, 95% CI 0.654-0.799) (Table 2). When generating a set of genes to classify samples, a tradeoff between specificity and sensitivity may be considered to achieve optimal results as shown by the Receiver Operating Characteristic curves in FIG. 4A. To address whether the ASD85 classifier performed better than expected by chance, 85 genes were randomly sampled 2,000 times and the performances of these random sets were evaluated by AUCs. The ASD85 model outperformed all of the 2,000 trials of randomly chosen sets of 85 genes (permutation P<0.0005). The training set (P1) consisted of males only while the test set (P2) had both genders. The prediction model built with males performed better for males in P2. The AUC for male samples in P2 was 0.74 (95% CI 0.650-0.831) compared to 0.56 (95% CI 0.386-0.734) for female samples. To test the robustness of ASD85, we trained ASD85 with P2 samples to classify P1 samples, switching our training and validation sets. The performance was comparable to the original classification accuracy where P1 was used as the training set (AUC 0.75, 95% CI 0.658-0.858, FIG. 4B).

The receiver operating characteristic (ROC) curve analysis was performed to evaluate the prediction accuracy (FIG. 4). The dotted blue line represents random classification accuracy (AUC 0.5). ASD85 model was trained with P1 to predict the diagnosis of each sample in P2 (FIG. 4A). The performance measured by AUC was 0.73 (95% CI, 0.654-0.799), and male samples were accurately predicted while female samples were not (AUC 0.74 and 0.56 respectively). A non-linear curve fitting is used to smooth the ROC curve and plotted in dark red. The same genes were trained using P2 male samples and tested against P1 samples (FIG. 4B). ASD85 genes showed the same robust performance when training and testing datasets were switched (AUC 0.75, 95% CI 0.658-0.858).

### Effect of Other Clinical and Demographic Factors on Blood Gene Expression

In assessing robustness of the predictor for ASD classification, the expression data for potential confounders was evaluated. Among the demographic and clinical features, age at the time of blood draw may significantly influence gene expression. Within the ASD group, age at blood collection was correlated within the 389 genes at a significance level of P<0.001 (Spearman's rank correlation test, N=66, corresponding FDR 0.018). The one carbon pool by folate pathway (KEGG ID: hsa00670) was significantly enriched with 389 age-correlated genes in the ASD population (hypergeometric test P=6.7×10⁻⁷, FDR 7.7×10⁴). The age-correlated genes in this pathway were MTHFD1, TYMS, SHMT2, ATIC, MTHFD1L, and GART. The ASD85 genes were not significantly correlated with age except for CEP110, CREBZF, C10orf28, and UTY across the patients with ASD. In the P1 control group (N=33), 163 genes correlated significantly with age, but none of the ASD85 genes were among them.

Several other clinical and developmental characteristics were also correlated with gene expression changes as summarized in Table 4. The positive history of developmental delay including a delay in hitting milestones such as sitting, crawling, walking, and speaking was associated with 11 genes including ARX. The aristaless related homeobox (ARX) is a homeodomain transcription factor that plays roles in cerebral development and patterning, and is implicated in X-linked mental retardations. ARX was not differentially expressed in the ASD group of P1 (P=0.64); however, it was significantly down-regulated in the individuals with positive history of developmental delay (P=0.00037, FDR 0.31).

In the P1 cohort, 9 patients with ASD were diagnosed with learning disorders. Sixty-four genes were differentially expressed with regard to learning disorders (Positive History N=9, Negative History N=90, P<0.001, corresponding FDR 0.14). The calcium signaling pathway (KEGG ID: hsa04020) was significant (hypergeometric P=0.023, FDR 0.19) with ADRA1B, CHRM2, PPP3R1, and P2RX3. The Synapsin 2 (SYN2), one of the 64 differentially expressed genes in the patients co-diagnosed with learning disorders, is a synaptic vesicle-associated protein that has been implicated in modulation of neurotransmitter release and in synaptogenesis. A brain gene expression study showed that SYN2 was down-regulated in the prefrontal cortex of schizophrenic patients. The differentially expressed genes that were correlated with other clinical conditions including psychiatric, neurological, gastrointestinal disorders, and seizure disorder are summarized in Table 4.

### Additional Remarks

This example demonstrates, among other things, the usefulness of gene expression profiling to distinguish ASD patients from control samples, with an average accuracy of 72.5% in one population (the P1 cohort) and greater than 72.7% in an independently collected validation population (P2).

The performance of the classification in this example is notable in part because the two groups were relatively heterogeneous and were profiled using two different array-types. The classification of 73% of cases by expression profiling contrasts with the small percentage of ASD cases characterized through genetic mutations or structural variations to date. It also compares favorably to the performance of CMA, which accounts for 7-10% of cases of ASD. Together, these results indicate that gene expression signatures, which comprise multiple perturbed pathways, may serve as signals of genetic change in many patients. Moreover, in some embodiments, peripheral blood cells may be used as a surrogate for gene expression in the developing nervous system.

The biological processes implicated by the differentially expressed genes identified in this example are of interest in part because some of the pathways link to synaptic activity-dependent processes (i.e., Long-Term Potentiation and Neurotrophin signaling pathway in Table 3), for which several ASD mutations have been found. Immune/inflammation pathways were also identified in this analysis (e.g. Chemokine signaling pathway and Fc gamma R-mediated phagocytosis).

CREBBP, RPS6KA3, and NIPBL are associated with mental retardation. Heterozygous mutation of CREBBP is indicated in Rubinstein-Taybi syndrome, of which the core symptom is mental retardation (MIM ID #180849). Coffin-Lowry syndrome (MIM ID #303600) is associated with mutations in RPS6KA3 on chromosome Xp22.12, and is characterized by skeletal malformation, growth retardation, cognitive impairments, hearing deficit, and paroxysmal movement disorders. Mutations in NIPBL result in Cornelia de Lange syndrome (MIM ID #122470), a disorder characterized by dysmorphic facial features, growth delay, limb reduction defects as well as mental retardation.

Moreover, DOCK8 is significantly differentially expressed in ASD (P=3.05×10⁴). Two unrelated patients possessed heterozygous disruptions of the DOCK8 gene, one by deletion and one by a translocation breakpoint; these disruptions are associated with mental retardation and developmental disability (MRD2, MIM ID #614113). In the P2 dataset, 13 differentially expressed genes were associated with mental retardation. These were ATP6AP2, ATRX, CRBN, FXR1, IGF1, INPPSE, KIAA2022, NUFIP2, RPS6KA3, TECT, UBSE2A, and ZDHHC9. The RPS6KA3 was significant in both P1 and the male samples in the P2 datasets. Four out of 66 ASD cases of P1 dataset had mild mental retardation. The comparison of 4 cases with mild mental retardation against 62 ASD cases in P1 found 95 differentially expressed genes (P<0.001, corresponding FDR 0.09).

The differentially expressed genes in the patients with ASP were distinct from the ones in AUT vs. controls or PDDNOS vs. controls. In one embodiment, more genes were differentially expressed without ASP samples compared to with ASP at the same statistical stringency. Since the median age was older for ASP group (9.2, range 4-16) compared to AUT+PDDNOS (6.8, range 3.4-17.5), differential expression was evaluated to determine if it was confounded by age. The expression of PNOC, one of the differentially expressed genes in ASP vs. controls, was correlated with age in the P1 (P=6.42E-05). However, the other significant genes in ASP were not correlated with age in this example.

Expression profiling also identified chromosomal abnormalities. For instance, an affected male that had high expression of the X-inactive-specific transcript (XIST); the expression values were comparable to those of females. Subsequent karyotyping confirmed Klinefelter syndrome in this individual, and the case was excluded in this study for further analysis.

In this example, two data sets were obtained at different times and the methods for RNA acquisition and microarrays used in P1 differed in part from those in P2. Also, the control population in P2 versus P1 differed in the clinics from which they were drawn, and the race and ethnic backgrounds of the patients and control population were not completely matched. Nonetheless, analysis of the independent datasets demonstrates the accuracy of the classifier. Also, the accuracy obtained in this example demonstrates that the geneset used includes predictive biomarkers.

TABLE 1
Characteristics of patients with Autism Spectrum Disorders and
Controls in the training set (P1) and in the validation set (P2).

| | Training Set (P1) | | Validation Set (P2) | |
|---|---|---|---|---|
| Characteristic | ASD | Control | ASD | Control |
| No. | 66 | 33 | 104 | 82 |
| Age-years | | | | |
| Mean | 8.0 | 9.0 | 8.4 | 8.1 |
| Interquartile range | 5.5-9.7 | 4.0-13.1 | 5.0-11.0 | 4.1-12.3 |
| Male, No. (%) | 66 (100) | 33 (100) | 80 (77) | 48 (59) |
| Diagnosis (Male %) | | | | |
| Autistic Disorder | 31 | — | 40 (75) | — |
| PDD, NOS | 26 | — | 49 (76) | — |
| Asperger's Disorder | 9 | — | 15 (87) | — |
| Race-no. | | | | |
| Caucasian | 60 | 13 | 96 | 33 |
| Black | 0 | 5 | 0 | 8 |
| Asian | 1 | 1 | 3 | 2 |
| Mixed | 5 | 1 | 4 | 8 |
| Other | — | 4 | — | 21 |
| Unknown | 1 | 9 | 1 | 10 |
| Ethnicity | | | | |
| Hispanic-no. | 2 | 9 | 8 | 36 |
| Unknown-no. | 1 | — | — | — |
| Developmental delay-no. | 21 | 5 | 51 | 3 |
| Learning Disorder-no | 9 | — | | — |
| Psychiatric Disorder-no. | 14 | 4 | 32 | 1 |
| Neurological Disorder-no. | 8 | — | 18 | — |
| Gastrointestinal Disorder-no. | 24 | — | 20 | — |
| Autoimmune Disorder-no. | — | — | 7 | — |
| Cerebral Palsy-no. | — | — | 1 | — |

TABLE 2
Top 12 Enriched KEGG pathways with the differentially expressed genes in ASD.

| KEGG pathway | Count | % | P-value | FDR | Genes |
|---|---|---|---|---|---|
| Neurotrophin | 10 | 2.6 | 0.0011 | 1.22 | MAPK1, RPS6KA3, YWHAG, |
| signaling pathway | | | | | CRKL, MAP2K1, PIK3CB, |
| | | | | | PIK3CD, SH2B3, MAPK8, |
| | | | | | KIDINS220 |
| Long-term | 7 | 1.8 | 0.0029 | 3.16 | MAPK1, RPS6KA3, GNAQ, |
| potentiation | | | | | MAP2K1, CREBBP, PPP3CB, |
| | | | | | PPP1R12A |
| mTOR signaling | 6 | 1.5 | 0.0044 | 4.87 | MAPK1, RPS6KA3, PIK3CB, |
| pathway | | | | | PIK3CD, CAB39, RICTOR |
| Progesterone-mediated | 7 | 1.8 | 0.0091 | 9.72 | IGF1R, MAPK1, RPS6KA3, |
| oocyte maturation | | | | | MAP2K1, PIK3CB, PIK3CD, |
| | | | | | MAPK8 |
| Regulation of actin | 11 | 2.8 | 0.0144 | 15.02 | GNA13, MAPK1, CRKL, |
| cytoskeleton | | | | | ROCK1, MAP2K1, PIK3CB, |
| | | | | | PIK3CD, SSH2, PPP1R12A, |
| | | | | | IQGAP2, ITGB2 |
| Fc gamma R-mediated | 7 | 1.8 | 0.0144 | 15.03 | MAPK1, PTPRC, DOCK2, |
| phagocytosis | | | | | CRKL, MAP2K1, PIK3CB, |
| | | | | | PIK3CD |
| Renal cell carcinoma | 6 | 1.5 | 0.0154 | 15.95 | MAPK1, CRKL, MAP2K1, |
| | | | | | PIK3CB, PIK3CD, CREBBP |
| Chemokine signaling | 10 | 2.6 | 0.0163 | 16.83 | MAPK1, DOCK2, CRKL, |
| pathway | | | | | ROCK1, MAP2K1, PIK3CB, |
| | | | | | PREX1, PIK3CD, CCR2, |
| | | | | | CCR10 |
| Type II diabetes | 5 | 1.3 | 0.0165 | 17.02 | MAPK1, PIK3CB, PIK3CD, |
| mellitus | | | | | HK2, MAPK8 |
| Non-small cell lung | 5 | 1.3 | 0.0262 | 25.72 | MAPK1, RASSF5, MAP2K1, |
| cancer | | | | | PIK3CB, PIK3CD |
| Colorectal cancer | 6 | 1.5 | 0.0312 | 29.89 | IGF1R, MAPK1, MAP2K1, |
| | | | | | PIK3CB, PIK3CD, MAPK8 |
| ErbB signaling | 6 | 1.5 | 0.0356 | 33.35 | MAPK1, CRKL, MAP2K1, |
| pathway | | | | | PIK3CB, PIK3CD, MAPK8 |
| Prostate cancer | 6 | 1.5 | 0.0387 | 35.71 | IGF1R, MAPK1, MAP2K1, |
| | | | | | PIK3CB, PIK3CD, CREBBP |
| Glioma | 5 | 1.3 | 0.0428 | 38.74 | IGF1R, MAPK1, MAP2K1, |
| | | | | | PIK3CB, PIK3CD |

TABLE 3
Prediction performance of ASD85 trained with P1.

| | | | | | Positive | Negative |
|---|---|---|---|---|---|---|
| | AUC | | | | Predictive | Predictive |
| Validation | (95% Confidence | Accuracy | Sensitivity | Specificity | Value | Value |
| set | Intervals) | (%) | (%) | (%) | (%) | (%) |
| P2 | 0.73 (0.654-0.799) | 69.9 | 74.0 | 64.6 | 72.6 | 66.3 |
| P2 | 0.74 (0.650-0.831) | 72.7 | 85.0 | 52.1 | 74.7 | 67.6 |
| (male) | | | | | | |
| P2 | 0.56 (0.386-0.734) | 63.8 | 58.3 | 67.6 | 56.0 | 69.7 |
| (female) | | | | | | |

Abbreviation:
ASD85, the genes in a classifier developed on P1 with 85 genes listed in Table 6;
AUC, area under the receiver operating characteristic curve.

TABLE 4
Genes significantly correlated with clinical features.

| | Number of | |
|---|---|---|
| Medical and | significant | |
| developmental | genes (p < | |
| history | 0.001) | Significant genes |
| Developmental | 11 | ARX, CCDC18, CDHR3, IBTK, RHBDL2, SGSM1, SPR, |
| delay | | TBX18, TRIM4, ZNF37A, ZNF536 |
| Learning | 64 | ADRA1B, AKNAD1, ANKRD18A, ANKRD30A, APP, BOD1L, C22orf23, |
| disorders | | C3orf34, C6orf114, C6orf195, CA2, CACNG5, CAV2, CHRM2,CLDN5, |
| | | CNTNAP3, CRYGN, DDX11L2, F13A1, FAM184B, FMO3, GGTA1, GIF, GNG11, |
| | | GSC2, HBEGF, HGD, HRCT1, IGSF11, IGSF22, ITPRIPL2, IZUMO1, KCNA1, |
| | | KRT81, LCE1B, LOC126536, LYZL4, MECOM, MSH4, NME5, NPY, NR1H4, |
| | | P2RX3, PACS2, PF4V1, PPFIA2, PPP3R1, RAX2, RNF17, SCGN, SCN9A, SHH, |
| | | SLC16A9, SLCO2B1, SMCR8, SYCE1, SYN2, TCTN2, TEAD1, TMIE, TRH, |
| | | VGLL3, WRB, ZNF652 |
| Neurological | 12 | FAM13A, GSC2, LOC401387, MFAP5, PITX3, PVALB, RAPGEF5, SPRR4, |
| disorders | | TACR2, TP63, WTIP |
| Psychiatric | 5 | CSTT, GPR111, HIP1, MED25, STX19 |
| disorders | | |
| Gastrointestinal | 6 | COL7A1, MARK1, NXPH3, SETMAR, SLC1A6, SLC6A1 |
| disorders | | |
| Seizure | 5 | GPR153, GSC2, LOC401387, MGC39545, PITX3 |
| disorders | | |

TABLE 5
Quantitative RT-PCR validations of 3 differentially expressed genes
across 165 ASD and 103 Controls.

| | | | Microarray results | | | | |
|---|---|---|---|---|---|---|---|
| | | | (P1 excluding ASP) | | | qRTPCR results* | |
| | Gene | | | | Fold | | Fold |
| ProbeID | Symbol | Taqman assay | p-value | FDR | changes | p-value | change |
| 8152962 | LRRC6 | Hs00539072_m1 | 2.53E−04 | 1.06E−02 | 1.4 | 5.57E−05 | 1.8 |
| 8066822 | SULF2 | Hs00378697_m1 | 1.66E−05 | 4.63E−03 | 1.2 | 7.52E−19 | 1.4 |
| 8021984 | YES1 | Hs00736972_m1 | 1.58E−04 | 1.03E−02 | 1.2 | 9.79E−10 | 1.5 |

Fold changes determined by calculating ASD/Control.
*Housekeeping gene used for qRT-PCR normalization was GAPDH (Hs9999905_m1). Values shown are for the entire peripheral blood validation data set (P2) and additional samples that were not prepared with microarrays (41 ASD and 21 Controls).
FDR: False Discovery Rate

TABLE 6
The 85 predictor genes. These are top 85 genes from the ranked list by p-values.
The Affymetrix IDs represent the transcript IDs of Gene ST 1.0 array.
Welch's t-tests were used to calculate the T-statistical scores and p-values.
The false discovery rates (FDR) were calculated using standard methods.

| | | | | | Fold change |
|---|---|---|---|---|---|
| Affymetrix | | | | | (ASD/ |
| ID | Gene | T-statistic | p-value | FDR | Control) |
| 8138116 | ZNF12 | 5.44781703 | 0.00000046 | 0.0021142 | 1.53 |
| 7995631 | RBL2 | 5.409000851 | 0.00000054 | 0.0021142 | 1.30 |
| 8120992 | ZNF292 | 5.250058153 | 0.00000104 | 0.0021142 | 1.53 |
| 7922889 | IVNS1ABP | 5.170733803 | 0.00000145 | 0.0021142 | 1.34 |
| 8051814 | ZFP36L2 | 5.122815439 | 0.00000176 | 0.0021142 | 1.35 |
| 8151149 | ARFGEF1 | 5.095845824 | 0.00000197 | 0.0021142 | 1.32 |
| 8177137 | UTY | 5.08940513 | 0.00000202 | 0.0021142 | 1.53 |
| 8152988 | SLA | 5.078743957 | 0.00000211 | 0.0021142 | 1.35 |
| 7975361 | KIAA0247 | 5.010519336 | 0.00000278 | 0.0024780 | 1.41 |
| 8138670 | HNRNPA2B1 | 4.95479835 | 0.00000348 | 0.0027916 | 1.60 |
| 8115562 | RNF145 | 4.917035545 | 0.00000405 | 0.0029528 | 1.43 |
| 7931353 | PTPRE | 4.879450708 | 0.00000470 | 0.0031453 | 1.32 |
| 8128394 | SFRS18 | 4.840307034 | 0.00000549 | 0.0031573 | 1.42 |
| 7911038 | ZNF238 | 4.839756782 | 0.00000551 | 0.0031573 | 1.32 |
| 8059596 | TRIP12 | 4.750539282 | 0.00000783 | 0.0041926 | 1.30 |
| 7974066 | PNN | 4.715741722 | 0.00000898 | 0.0042751 | 1.70 |
| 7957277 | ZDHHC17 | 4.705490497 | 0.00000935 | 0.0042751 | 1.50 |
| 8143988 | MLL3 | 4.698517233 | 0.00000961 | 0.0042751 | 1.39 |
| 7987048 | MTMR10 | 4.682638474 | 0.00001022 | 0.0042751 | 1.32 |
| 8126018 | STK38 | 4.649679439 | 0.00001162 | 0.0042751 | 1.30 |
| 8066417 | SERINC3 | 4.647603907 | 0.00001172 | 0.0042751 | 1.25 |
| 8104944 | NIPBL | 4.639925524 | 0.00001207 | 0.0042751 | 1.37 |
| 8059770 | TIGD1 | 4.636108174 | 0.00001225 | 0.0042751 | 1.58 |
| 8009205 | DDX42 | 4.62522253 | 0.00001278 | 0.0042751 | 1.28 |
| 8073733 | NUP50 | 4.596891156 | 0.00001426 | 0.0043711 | 1.37 |
| 8048980 | CAB39 | 4.595153056 | 0.00001436 | 0.0043711 | 1.39 |
| 8022441 | ROCK1 | 4.589047446 | 0.00001470 | 0.0043711 | 1.46 |
| 8066822 | SULF2 | 4.557222787 | 0.00001662 | 0.0046258 | 1.46 |
| 8102523 | FABP2 | 4.555816605 | 0.00001671 | 0.0046258 | 1.43 |
| 8050128 | KIDINS220 | 4.524052585 | 0.00001888 | 0.0047373 | 1.43 |
| 8065776 | NCOA6 | 4.52329784 | 0.00001893 | 0.0047373 | 1.35 |
| 8060418 | SIRPA | 4.518802333 | 0.00001926 | 0.0047373 | 1.36 |
| 8155898 | PCSK5 | 4.515942481 | 0.00001947 | 0.0047373 | 1.40 |
| 7989224 | ADAM10 | 4.505495127 | 0.00002027 | 0.0047855 | 1.36 |
| 7927062 | ZNF33A | 4.480641748 | 0.00002229 | 0.0049711 | 1.31 |
| 8174119 | ZMAT1 | 4.480556628 | 0.00002229 | 0.0049711 | 1.79 |
| 7929719 | C10orf28 | 4.462908701 | 0.00002384 | 0.0051030 | 1.26 |
| 8054135 | MGAT4A | 4.453908743 | 0.00002467 | 0.0051030 | 1.46 |
| 8157534 | CEP110 | 4.452645661 | 0.00002479 | 0.0051030 | 1.45 |
| 8011542 | ZZEF1 | 4.441482484 | 0.00002586 | 0.0051262 | 1.30 |
| 7950796 | CREBZF | 4.438271011 | 0.00002618 | 0.0051262 | 1.62 |
| 8169541 | DOCK11 | 4.414199561 | 0.00002868 | 0.0054599 | 1.39 |
| 8060627 | ATRN | 4.409031654 | 0.00002925 | 0.0054599 | 1.49 |
| 8112687 | COL4A3BP | 4.399864402 | 0.00003028 | 0.0055237 | 1.36 |
| 8168678 | FAM133A | 4.387606176 | 0.00003171 | 0.0056018 | 1.37 |
| 8084128 | TTC14 | 4.384351306 | 0.00003210 | 0.0056018 | 1.52 |
| 8127637 | TMEM30A | 4.377503628 | 0.00003294 | 0.0056258 | 1.51 |
| 7988921 | MYO5A | 4.365585155 | 0.00003445 | 0.0057611 | 1.34 |
| 7941769 | KDM2A | 4.347535195 | 0.00003686 | 0.0060391 | 1.33 |
| 8003263 | ZCCHC14 | 4.334383278 | 0.00003872 | 0.0062172 | 1.46 |
| 8115927 | RNF44 | 4.318254064 | 0.00004113 | 0.0064742 | 1.28 |
| 7952739 | ZBTB44 | 4.31004905 | 0.00004241 | 0.0065471 | 1.37 |
| 8008834 | CLTC | 4.30215329 | 0.00004368 | 0.0066155 | 1.28 |
| 8122464 | UTRN | 4.288577406 | 0.00004594 | 0.0066271 | 1.33 |
| 8080878 | ATXN7 | 4.284995793 | 0.00004656 | 0.0066271 | 1.28 |
| 7965123 | PPP1R12A | 4.282208035 | 0.00004704 | 0.0066271 | 1.42 |
| 7924603 | LBR | 4.278968504 | 0.00004761 | 0.0066271 | 1.36 |
| 8093976 | TBC1D14 | 4.276653711 | 0.00004802 | 0.0066271 | 1.29 |
| 7968035 | SPATA13 | 4.265639279 | 0.00005003 | 0.0066271 | 1.43 |
| 8042942 | HK2 | 4.265006057 | 0.00005015 | 0.0066271 | 1.43 |
| 7999044 | CREBBP | 4.261335061 | 0.00005083 | 0.0066271 | 1.38 |
| 8129522 | MED23 | 4.259478286 | 0.00005118 | 0.0066271 | 1.40 |
| 8106602 | ZFYVE16 | 4.239371369 | 0.00005514 | 0.0070257 | 1.40 |
| 7968274 | PAN3 | 4.220489963 | 0.00005912 | 0.0074151 | 1.32 |
| 7994161 | RBBP6 | 4.212035778 | 0.00006099 | 0.0075320 | 1.44 |
| 8132188 | AVL9 | 4.198500819 | 0.00006410 | 0.0076370 | 1.27 |
| 8116247 | ZNF354A | 4.196567741 | 0.00006456 | 0.0076370 | 1.51 |
| 8042337 | ACTR2 | 4.195934461 | 0.00006471 | 0.0076370 | 1.32 |
| 8058927 | TMBIM1 | 4.188657616 | 0.00006646 | 0.0076370 | 1.23 |
| 8171762 | RPS6KA3 | 4.186851199 | 0.00006690 | 0.0076370 | 1.35 |
| 7935660 | DNMBP | 4.184239878 | 0.00006755 | 0.0076370 | 1.22 |
| 8079462 | NBEAL2 | 4.174144466 | 0.00007009 | 0.0078149 | 1.37 |
| 7916592 | MYSM1 | 4.162544299 | 0.00007313 | 0.0079692 | 1.54 |
| 8161701 | TMEM2 | 4.158409899 | 0.00007425 | 0.0079692 | 1.42 |
| 8079140 | SNRK | 4.157642087 | 0.00007445 | 0.0079692 | 1.35 |
| 8097148 | KIAA1109 | 4.14898753 | 0.00007684 | 0.0080544 | 1.45 |
| 8122343 | HECA | 4.146588049 | 0.00007752 | 0.0080544 | 1.31 |
| 7969651 | DNAJC3 | 4.143984924 | 0.00007826 | 0.0080544 | 1.30 |
| 7932911 | KIF5B | 4.131882496 | 0.00008179 | 0.0082738 | 1.36 |
| 8095269 | POLR2B | 4.129660047 | 0.00008245 | 0.0082738 | 1.34 |
| 8101260 | ANTXR2 | 4.124126476 | 0.00008413 | 0.0083379 | 1.35 |
| 7989387 | VPS13C | 4.117850416 | 0.00008607 | 0.0083546 | 1.37 |
| 7978376 | STXBP6 | −4.116866541 | 0.00008638 | 0.0083546 | 0.68 |
| 8102006 | MANBA | 4.110192604 | 0.00008850 | 0.0084007 | 1.38 |
| 7979044 | NIN | 4.108430825 | 0.00008907 | 0.0084007 | 1.31 |

TABLE 7
Prediction performances of ASD85. ASD85 denotes the genes in a
classifier developed on P1 with 85 genes listed in Table 6. The average
prediction the performances from 100-repeated leave-group-out cross
validations using P1 dataset are shown. For each prediction instance,
20% of ASD (N = 13) and 20% of controls (N = 7) were randomly
selected for a testing set, and the other 80% of samples served as a
training set. This procedure was repeated 100 times to calculate
the average performance of ASD85 with 6 machine learning algorithms
listed below. The overall performance of PLS was comparable to
the other 5 methods. The sensitivities were relatively higher than the
specificities for most methods except for the Naïve Bayes classifier.

| | | ACC | SENS | SPEC | PPV | NPV |
|---|---|---|---|---|---|---|
| Machine learning method | AUC | (%) | (%) | (%) | (%) | (%) |
| Partial Least Squares | 0.782 | 76.1 | 81.9 | 65.1 | 82.3 | 67.4 |
| Logistic Regression | 0.687 | 67.3 | 72.3 | 57.2 | 77.2 | 50.9 |
| Naïve Bayes | 0.773 | 70.4 | 68.4 | 74.3 | 84.2 | 54.1 |
| kNN (k = 5) | 0.754 | 72.9 | 87.0 | 44.7 | 75.9 | 63.3 |
| Random Forest | 0.741 | 71.0 | 86.3 | 40.4 | 74.3 | 59.7 |
| Support Vector Machine | 0.742 | 77.4 | 83.7 | 64.8 | 82.6 | 66.6 |

(AUC: Area under the receiver operation characteristics curve, ACC: Accuracy, SENS: Sensitivity, SPEC: Specificity, PPV: Positive Predictive Value, NPV: Negative Predictive Value)

TABLE 8
67 Genes Common
to Both Cohorts

| ProbeID | Gene |
|---|---|
| 7900395 | RLF |
| 7906330 | CD1D |
| 7908931 | OPTC |
| 7922889 | IVNS1ABP |
| 7924603 | LBR |
| 7925201 | ARID4B |
| 7929719 | C10orf28 |
| 7932911 | KIFSB |
| 7933947 | HERC4 |
| 7935320 | TM9SF3 |
| 7938592 | FAR1 |
| 7942839 | PCF11 |
| 7948667 | AHNAK |
| 7950796 | CREBZF |
| 7957277 | ZDHHC17 |
| 7966851 | TAOK3 |
| 7969414 | KLFS |
| 7969651 | DNAJC3 |
| 7969935 | ERCCS |
| 7971422 | ZC3H13 |
| 7974066 | PNN |
| 7975521 | RBM25 |
| 7978739 | TRAPPC6B |
| 7986383 | IGF1R |
| 7986767 | C15orf49 |
| 8009205 | DDX42 |
| 8017634 | DDX5 |
| 8022441 | ROCK1 |
| 8034108 | YIPF2 |
| 8038427 | TSKS |
| 8041713 | PPM1B |
| 8041913 | KLRAQ1 |
| 8045398 | RAB3GAP1 |
| 8050128 | KIDINS220 |
| 8050190 | ADAM17 |
| 8051814 | ZFP36L2 |
| 8053775 | ZNF514 |
| 8055913 | PRPF40A |
| 8056113 | LY75 |
| 8059783 | NGEF |
| 8067113 | ZNF217 |
| 8070629 | C21orf105 |
| 8071597 | LOC96610 |
| 8073733 | NUP50 |
| 8079392 | CCR2 |
| 8084128 | TTC14 |
| 8095269 | POLR2B |
| 8105714 | SREK1 |
| 8112687 | COL4A3BP |
| 8115562 | RNF145 |
| 8120758 | SENP6 |
| 8120992 | ZNF292 |
| 8123644 | TUBB2A |
| 8127637 | TMEM30A |
| 8128394 | SFRS18 |
| 8137715 | MICALL2 |
| 8138116 | ZNF12 |
| 8138670 | HNRNPA2B1 |
| 8138922 | KBTBD2 |
| 8159992 | ERMP1 |
| 8161701 | TMEM2 |
| 8168678 | FAM133A |
| 8168875 | ARMCX3 |
| 8171762 | RPS6KA3 |
| 8172631 | FOXP3 |
| 8176624 | DDX3Y |
| 8177137 | UTY |

TABLE 9
100 Genes Significantly Different between AUT and Controls

| | | AUT vs. control p- |
|---|---|---|
| ProbeID | Genes | value |
| 8138116 | ZNF12 | 9.37E−07 |
| 8120992 | ZNF292 | 9.58E−07 |
| 7995631 | RBL2 | 7.74E−06 |
| 7974066 | PNN | 1.41E−05 |
| 8138670 | HNRNPA2B1 | 1.53E−05 |
| 8051814 | ZFP36L2 | 2.91E−05 |
| 8168678 | FAM133A | 3.78E−05 |
| 8128394 | SFRS18 | 4.34E−05 |
| 8084128 | TTC14 | 4.50E−05 |
| 8009205 | DDX42 | 4.54E−05 |
| 7911038 | ZNF238 | 4.81E−05 |
| 7941769 | KDM2A | 6.62E−05 |
| 8116635 | BPHL | 6.64E−05 |
| 8022441 | ROCK1 | 6.67E−05 |
| 8177137 | UTY | 6.70E−05 |
| 8151149 | ARFGEF1 | 8.37E−05 |
| 8115562 | RNF145 | 8.94E−05 |
| 8126018 | STK38 | 9.82E−05 |
| 8174119 | NA | 0.000120344 |
| 7950796 | CREBZF | 0.000120793 |
| 8080878 | ATXN7 | 0.000127792 |
| 8143988 | MLL3 | 0.000131879 |
| 8104944 | NIPBL | 0.000138753 |
| 8059596 | TRIP12 | 0.000143405 |
| 7922889 | IVNS1ABP | 0.000146016 |
| 8127637 | TMEM30A | 0.000148219 |
| 8135341 | CDHR3 | 0.000151987 |
| 7994161 | RBBP6 | 0.000152147 |
| 8115927 | RNF44 | 0.000155693 |
| 7927062 | ZNF33A | 0.000174898 |
| 8152988 | SLA | 0.000193263 |
| 7970602 | PARP4 | 0.000197885 |
| 7969935 | ERCC5 | 0.000199011 |
| 8169541 | DOCK11 | 0.0001991 |
| 7952739 | ZBTB44 | 0.000202248 |
| 7948667 | AHNAK | 0.000205084 |
| 7975361 | KIAA0247 | 0.000209011 |
| 8104022 | PDLIM3 | 0.000214336 |
| 7972055 | KCTD12 | 0.000216008 |
| 8042942 | HK2 | 0.000227441 |
| 8176624 | DDX3Y | 0.000228357 |
| 8048980 | CAB39 | 0.000236658 |
| 8129522 | MED23 | 0.000239323 |
| 7957277 | ZDHHC17 | 0.000242929 |
| 8102006 | MANBA | 0.000243994 |
| 8107474 | DMXL1 | 0.000247723 |
| 7989387 | VPS13C | 0.000250943 |
| 8087839 | POC1A | 0.000250945 |
| 8155898 | PCSK5 | 0.000285787 |
| 8008834 | CLTC | 0.000303524 |
| 7989224 | ADAM10 | 0.000318093 |
| 8081431 | ALCAM | 0.000327316 |
| 7932911 | KIF5B | 0.000346435 |
| 8157534 | CEP110 | 0.000346788 |
| 7965123 | PPP1R12A | 0.000353201 |
| 8073733 | NUP50 | 0.00036966 |
| 8059770 | TIGD1 | 0.000374778 |
| 7935320 | TM9SF3 | 0.000383103 |
| 8049906 | ING5 | 0.000404127 |
| 8065776 | NCOA6 | 0.000424663 |
| 8050128 | KIDINS220 | 0.000427609 |
| 7968274 | PAN3 | 0.00044294 |
| 8122343 | HECA | 0.000445443 |
| 8042337 | ACTR2 | 0.000455053 |
| 8078187 | PLCL2 | 0.000460868 |
| 8095269 | POLR2B | 0.000462122 |
| 8066417 | SERINC3 | 0.000466596 |
| 8044353 | ACOXL | 0.00046671 |
| 8131614 | AHR | 0.000477053 |
| 8069450 | PRMT2 | 0.000485122 |
| 7981346 | RAGE | 0.000495061 |
| 8065580 | DUSP15 | 0.000571609 |
| 7979044 | NIN | 0.000578188 |
| 8170027 | DDX26B | 0.000579117 |
| 8102523 | FABP2 | 0.000603103 |
| 8105714 | SREK1 | 0.000610858 |
| 8129608 | TAAR3 | 0.000621799 |
| 7989253 | SLTM | 0.000638848 |
| 7995479 | PAPD5 | 0.000650009 |
| 8123644 | TUBB2A | 0.000661275 |
| 7968035 | SPATA13 | 0.000677161 |
| 7927889 | CCAR1 | 0.000684047 |
| 8140398 | YWHAG | 0.000707333 |
| 8116227 | CLK4 | 0.000731085 |
| 8005814 | NLK | 0.000745718 |
| 7999044 | CREBBP | 0.000760111 |
| 8110546 | MAML1 | 0.000783536 |
| 8060418 | SIRPA | 0.000784896 |
| 7916592 | MYSM1 | 0.000810119 |
| 7987048 | MTMR10 | 0.000823132 |
| 8060627 | ATRN | 0.000828175 |
| 8088247 | ARHGEF3 | 0.000834809 |
| 8104506 | TRIO | 0.000862774 |
| 8011542 | ZZEF1 | 0.000872511 |
| 7993478 | ABCC1 | 0.000895265 |
| 7943288 | SRSF8 | 0.000895853 |
| 8017634 | DDX5 | 0.000911494 |
| 8097148 | KIAA1109 | 0.000915623 |
| 8108603 | HARS2 | 0.000933077 |
| 8162236 | SEMA4D | 0.000955441 |

TABLE 10
43 Genes Signficantly Different Between PDDNOS v. Controls

| | | PDDNOS vs. Control p- |
|---|---|---|
| ProbeID | Genes | value |
| 7931353 | PTPRE | 7.73E−05 |
| 8043310 | RMND5A | 8.23E−05 |
| 8066822 | SULF2 | 0.000128734 |
| 7987048 | MTMR10 | 0.000129393 |
| 7975361 | KIAA0247 | 0.000143646 |
| 8151149 | ARFGEF1 | 0.000181757 |
| 8093976 | TBC1D14 | 0.000216158 |
| 8144317 | KBTBD11 | 0.000218663 |
| 8152988 | SLA | 0.000220835 |
| 8059770 | TIGD1 | 0.000221921 |
| 7995631 | RBL2 | 0.00022423 |
| 8003263 | ZCCHC14 | 0.000225181 |
| 7929719 | C10orf28 | 0.000245852 |
| 8144082 | C7orf13 | 0.000246397 |
| 8051814 | ZFP36L2 | 0.000265944 |
| 8066417 | SERINC3 | 0.000293195 |
| 8177137 | UTY | 0.000298957 |
| 8115562 | RNF145 | 0.000305673 |
| 7969651 | DNAJC3 | 0.000324779 |
| 8013965 | SSH2 | 0.000376116 |
| 8054135 | MGAT4A | 0.000416912 |
| 8138116 | ZNF12 | 0.000426425 |
| 8119529 | UBR2 | 0.000441929 |
| 7922889 | IVNS1ABP | 0.000455478 |
| 8119408 | NFYA | 0.000462384 |
| 8059596 | TRIP12 | 0.000463279 |
| 8090893 | MSL2 | 0.000565518 |
| 7939197 | HIPK3 | 0.000571304 |
| 7925622 | AHCTF1 | 0.000591115 |
| 8171762 | RPS6KA3 | 0.000592394 |
| 8073733 | NUP50 | 0.000649031 |
| 7978376 | STXBP6 | 0.000659042 |
| 8117663 | NKAPL | 0.000662362 |
| 8060418 | SIRPA | 0.000666742 |
| 8006123 | CPD | 0.000734829 |
| 7938179 | OR10A4 | 0.000750001 |
| 8068238 | IFNAR2 | 0.000758879 |
| 8065776 | NCOA6 | 0.000828501 |
| 8027439 | ZNF507 | 0.000858712 |
| 7988921 | MYO5A | 0.000895557 |
| 8112687 | COL4A3BP | 0.000935377 |
| 7957277 | ZDHHC17 | 0.000966732 |
| 8155898 | PCSK5 | 0.000970725 |

TABLE 11
9 Genes Significantly Different Between Asperger and Control

| | | Asperger |
|---|---|---|
| | | vs. control |
| ProbeID | Genest | p-value |
| 8102523 | FABP2 | 1.00E−05 |
| 8157751 | LHX2 | 3.77E−05 |
| 8005839 | TMEM97 | 0.0001068 |
| 8126446 | C6orf226 | 0.00037821 |
| 8152355 | SYBU | 0.00053218 |
| 8019910 | NA | 0.00054334 |
| 7999317 | TMEM186 | 0.00062854 |
| 8072413 | SMTN | 0.00076926 |
| 8145603 | PNOC | 0.00090824 |

### Example 3

### Gene Expression Signature Assessment

This example provides the results of a blood transcriptome analysis that aims to identify differences in 170 ASD and 115 age/sex-matched controls and to evaluate the utility of gene expression profiling as a tool to aid in the diagnosis of ASD. Differentially expressed genes were enriched for the neurotrophin signaling, long-term potentiation/depression, and notch signaling pathways, among other pathways. A 55-gene prediction model was developed, using a cross-validation strategy, on a sample cohort of 66 male ASD and 33 age-matched male controls (referred to in Example 3 as P1*). Subsequently, 104 ASD and 82 controls were recruited and used as a validation set (referred to in Example 3 as P2*). This 55-gene expression signature achieved 68% classification accuracy with the validation cohort (area under the receiver operating characteristic curve (AUC): 0.70 [95% confidence interval [CI]: 0.62-0.77]). The prediction model was built and trained with male samples and performed well for males (AUC 0.73, 95% CI 0.65-0.82) The prediction model when applied to female samples had the following performance characteristics: AUC 0.51, 95% CI 0.36-0.67. The 55-gene signature also performed robustly when the prediction model was trained with P2* male samples to classify P1* samples (AUC 0.69, 95% CI 0.58-0.80). The results, which are outlined in Tables 12-24, indicate feasibility of the use of blood expression profiling for ASD detection. Table 18 outlines the differentially expressed genes in P1* data set. Table 19 outlines differentially expressed genes in P2* data set. Table 20 outlines top 6 clusters of Gene Ontology biological process terms enriched for differentially expressed genes in P1* data set. Table 21 outlines the 55 predictor genes. Table 22 outlines the prediction performances of ASD55 using various machine learning algorithms. Table 23 outlines the functional enrichment of genes in ASD55. Table 24 outlines pathways enriched with age-correlated genes.

Expression studies were performed by microarray profiling using an earlier version of the Affymetrix array (U133p2) for the P1* data set and a later version (GeneST) for the P2* data set. After selecting the best matching probesets between the two platforms, principal component analysis was performed to project samples into the first two principal components. P1* and P2* samples did not form two clusters after combining the two datasets, which were centered and scaled independently.

There were 489 and 610 transcripts differentially expressed between ASD and controls in the P1* and P2* datasets, respectively (Welch's t-test P<0.001, corresponding FDRs 0.029 (P1*), and 0.023 (P2*)) (Tables 12 and 13). 23 genes—ARID4B, ARMCX3, C10orf28, CTBP2, DDX3Y, JRKL, MTERFD3, NFYA, NGEF, PNN, RLF, RNF145, TIGD1, TUBB2A, UTY, YES1, ZNF117, ZNF322, ZNF445, ZNF514, ZNF518B, ZNF540, and ZNF763—were significant in both cohorts. To calculate the significance of this overlap, sample labels were shuffled in both data sets 200,000 times and counted the number of permutations with as many or more overlapping genes. Out of 200,000 permutations, only 2 had at least 23 overlapping genes between the two data sets, yielding a permutation P=10⁻⁵. The overlap of 23 genes also showed a significant trend using the hypergeometric distribution (P=0.0721). In the P2* dataset, 352 genes were significant for male patients compared to male controls while 48 genes were significant for female groups (Welch's t-test P<0.001, corresponding FDRs 0.028 (P2* males) and 0.60 (P2* females)). POLR3H was differentially expressed in both males and females.

Twelve of the 489 differentially expressed genes in the P1* dataset were selected for validation by quantitative RT-PCR. The 12 genes had an average fold change between ASD and controls greater than 1.5 and a mean expression level on the array greater than 150. These were CREBZF, HNRNPA2B1, KIDINS220, LBR, MED23, RBBP6, SPATA13, SULF2, TMEM30A, ZDHHC17, ZMAT1, and ZNF12. Eleven genes were validated using qRT-PCR (Table 13).

### Subgroups in Dysregulated Pathways.

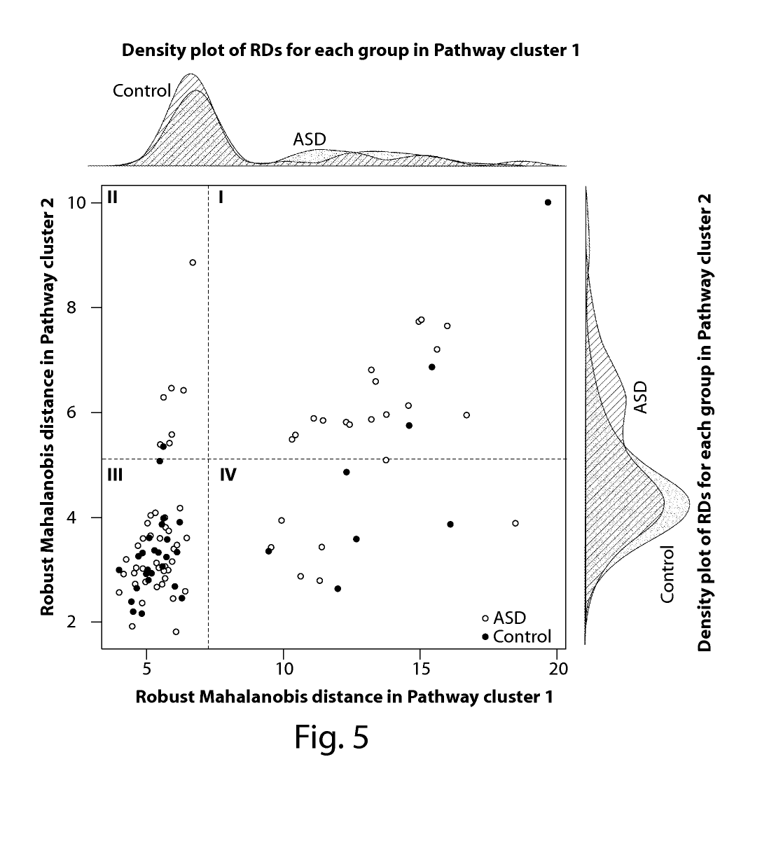

For immune response and synaptic gene sets, robust Mahalanobis distances (RDs) were calculated for all P1* samples. (FIG. 5). The outlier cutoff was set at the 97.5% quantile of the Chi-squared distribution for each gene set (dotted lines). When all samples were plotted in the 2-dimensional plane of pathway cluster 1 (x axis) by RDs in the pathway cluster 2 (y axis) (Table 15), four subgroups of samples were distinct. Both gene sets were perturbed for the samples in quadrant I; however, the samples in quadrants II and IV were significant for one gene set but not the other. A majority of samples were in quadrant III where no significant perturbation was found. The marginal density plots show the RD distributions for each gene set. Twenty-three out of 66 ASD samples (34.8%) were outliers for the synaptic gene set compared to 4 of 33 for controls (12.1%) (Fisher's exact test P=0.017). For the immune response gene set, outliers were not biased towards case or control (Fisher's exact test P=0.36).

### Performance of the ASD55 Prediction Model.

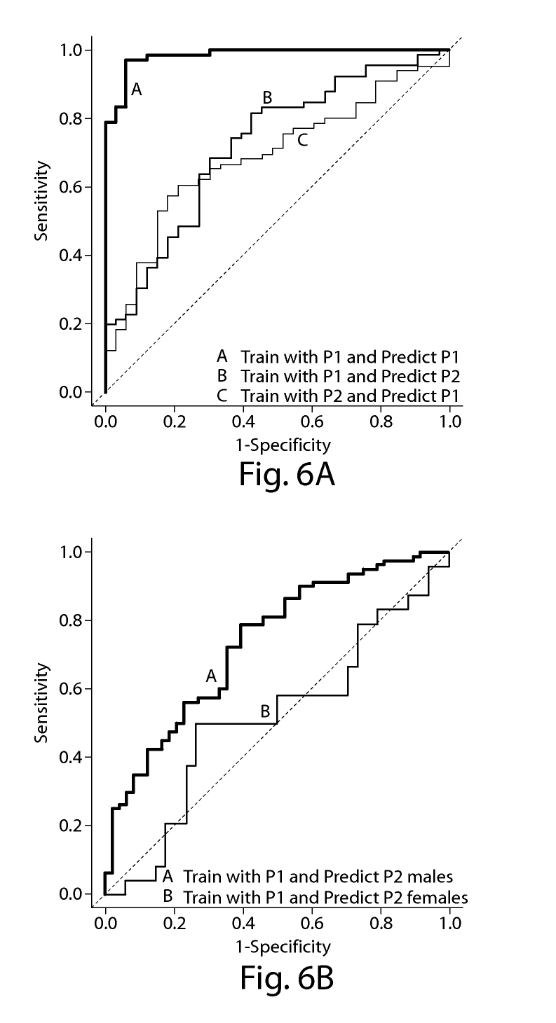

Receiver operating characteristic (ROC) curve analysis was performed to evaluate the prediction accuracy as seen in FIG. 6. The dotted diagonal line represents random classification accuracy (AUC 0.5). As shown in FIG. 6A the accuracy of ASD55 within P1* was relatively high (AUC 0.98, 95% confidence interval (CI), 0.965-1.000, Line A). The ASD55 model was trained with P1* to predict the diagnosis of each sample in an independently collected dataset P2* (Line B). The performance measured by AUC was 0.70 (95% CI, 0.62-0.77). ASD55 genes showed similar performance when the training and testing datasets were switched (AUC 0.69, 95% CI 0.58-0.80, Line C). P2* male samples were predicted (Line A) with relatively high accuracy. Prediction results for female samples (Line B) were also assessed (AUC 0.73 and 0.51 respectively) when the ASD55 model was trained with P1*.

### Cluster Analysis of the 55 Genes Used in the Prediction Model (ASD55).

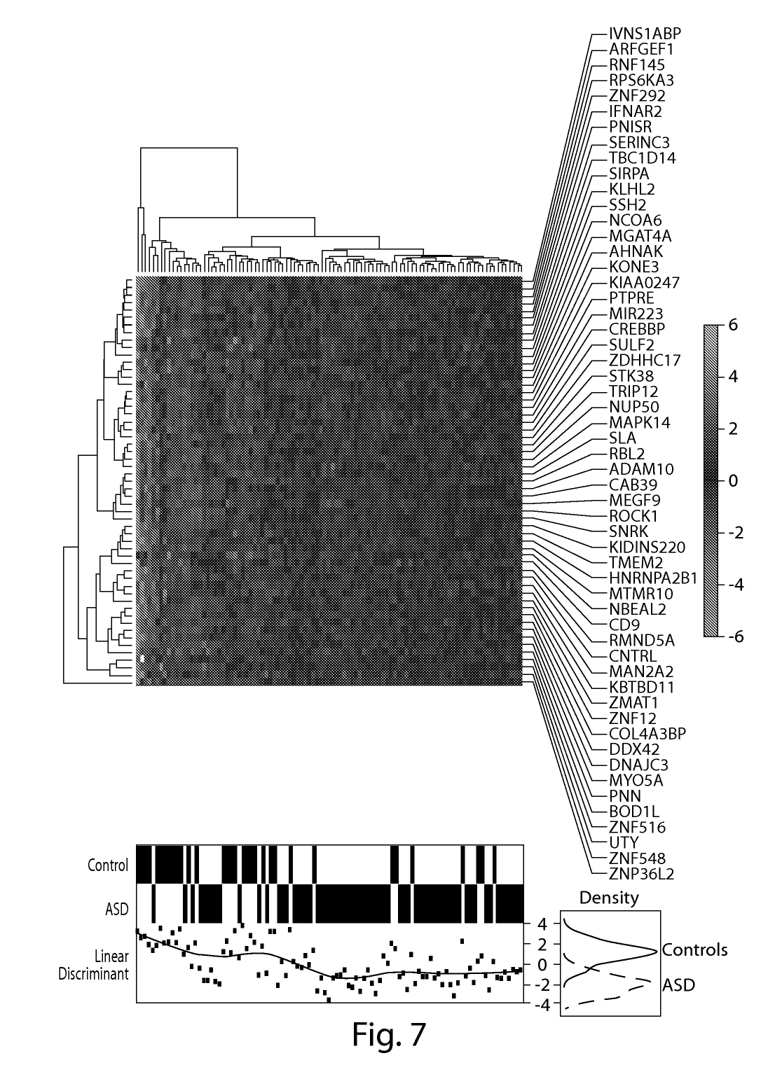

In FIG. 7 a dendrogram and heatmap on top show hierarchical clustering (average linkage) of the 99 samples in the training set (P1*) and the 55 genes used in the prediction model. The first 2 lines in the graph on the bottom indicate whether each sample is from the patient group or the control group. Finally, the bottom line shows the distribution of Fisher's linear discriminant scores (dots) based on ASD55 with moving average (line). The distributions of linear discriminant scores are shown on the right (solid line for controls and broken line for patients). ASD and controls were well separated using linear discriminant analysis on the ASD55 genes.

### Principal Component Analysis of 285 Blood Gene Expression Profiles.

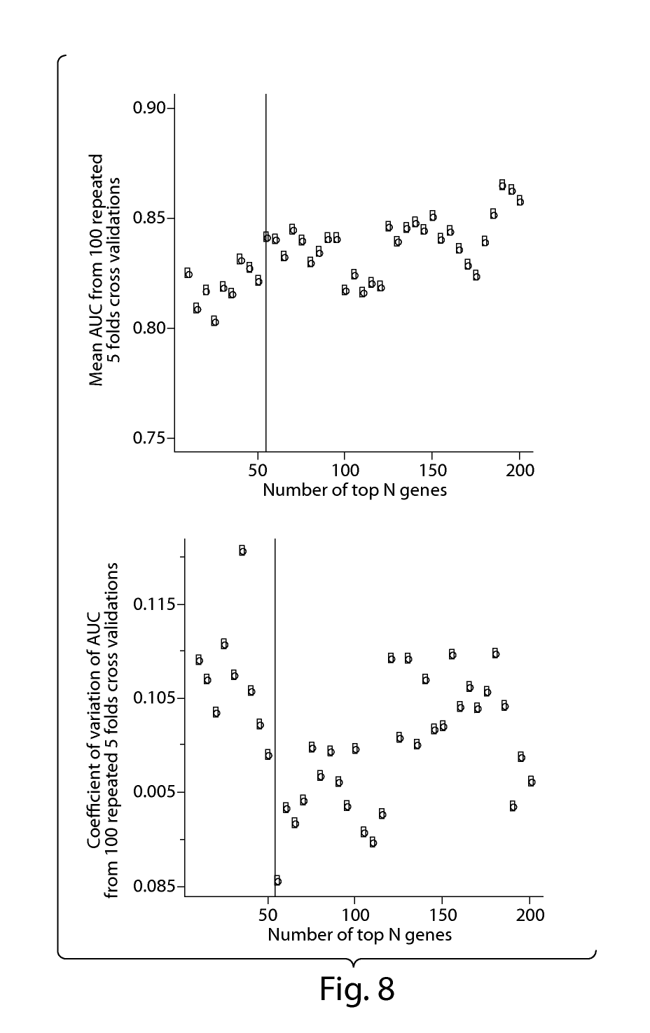

A global gene expression profile of the Training set (P1*) and the Validation set (P2*) samples is depicted in FIG. 8. After selecting the best-matching probesets between two Affymetrix microarray platforms, principal component analysis was performed. The ComBat method was applied to reduce batch effect for each dataset. All samples from P1* and P2* were projected to two-dimensional space of the first (PC1) and the second (PC2) principal components after centering and scaling expression levels in each dataset. 36.5% of overall variance was explained by PC1 and PC2. Global gene expression difference was not observed between ASD and controls.

### Selecting the Predictor Genes Using Repeated Cross Validations

The prediction model selection procedure, shown in FIG. 8, involved three nested loops as illustrated in FIG. 1. The outer-most loop was the selection of the top N genes (from 10 to 395 incremented by 5) from the AUC ranked gene list. The second loop was a leave-group out cross validation approach, where 80% of samples were randomly selected as a train set, while maintaining the proportion of each diagnostic class. This step was repeated 100 times for each list of the top N genes. The inner-most loop was used to optimize the parameters that were specific to machine learning methods used for a train set from an outer loop. This parameter tunings were repeated 100 times by randomly selecting 80% of the train set samples. The prediction performance was estimated using AUC. It was found that the mean AUCs improved gradually when we increased the number of genes to build more complex prediction models (left). In this example, the top 55 genes prediction model performed significantly better than the 50 gene model (t-test P=0.00031) and also presented the smallest coefficient of variation from 100 repeated cross validations (right).

### Overlap Between Differentially Expressed Genes for Each Diagnostic Subgroup (ASP, PDD, AUT) in P1.

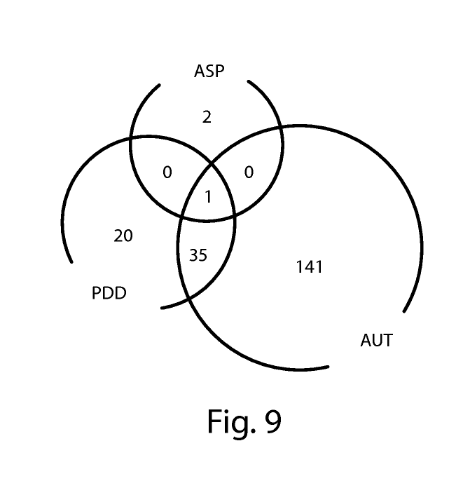

PTPRE was found in common for each diagnostic subgroup vs. control (FIG. 9). And 36 genes were common between AUT vs. control (177 significant genes) and PDDNOS vs. control (56 significant genes).

### Further Analyses

When each diagnostic subtype was compared to controls in the P1* dataset, 178, 56, and 3 genes were significant for autistic disorder (AUT), pervasive developmental disorder—not otherwise specified (PDDNOS), and Asperger's disorder (ASP), respectively (One-way analysis of variance (ANOVA) with Dunnett's post hoc test P<0.001, corresponding FDRs 0.076 (AUT), 0.24 (PDDNOS), and 1.0 (ASP)). Among the genes identified as significant in ASP, PTPRE, overlapped with the AUT vs. control or PDDNOS vs. control comparisons while 36 genes were in common between AUT vs. control and PDDNOS vs. control (FIG. 8).

Four of 66 ASD cases in the P1* dataset had mild mental retardation. When the 4 ASD cases with mild mental retardation were compared to the 62 ASD cases without mental retardation, 70 differentially expressed genes (P<0.001, corresponding FDR 0.12) were found

Expression profiling also identified chromosomal abnormalities. For instance, an affected male that had high expression of the X-inactive-specific transcript (XIST) was identified; the expression values were comparable to those of females. Subsequent karyotyping confirmed Klinefelter syndrome in this individual, and the case was excluded in this study for further analysis.

### Perturbed Biological Pathways and Identification of Heterogeneous Subgroups

A modified Fisher's exact test (i.e., Expression Analysis Systematic Explorer [EASE] score) was used to determine what biological pathways were enriched with the differentially expressed genes in P1* using the DAVID functional annotation system. This metric allowed for the calculation of which processes were overrepresented in the 489 differentially expressed genes in P1* relative to all the processes annotated in the Kyoto Encyclopedia of Genes and Genomes (KEGG). These results are detailed in Table 15. In brief, the neurotrophin signaling pathway (KEGG pathway identifier: hsa04722) was the most significant (EASE score P=0.00023, FDR 0.0026) among 22 overrepresented pathways (EASE score P<0.05, corresponding FDR 0.44). The neurotrophin signaling pathway includes neurotrophins and their second messenger systems such as the MAPK pathway, PI3K pathway, and PLC pathway. Interestingly, long-term potentiation and long-term depression pathways were also significant (EASE score P=0.011, FDR 0.11, and P=0.042, FDR 0.39 respectively). The 22 overrepresented pathways were grouped according to the number of shared genes by calculating Cohen's kappa score. Two enriched clusters of 15 and 3 pathways were significant (Cohen's kappa>0.5) with progesterone-mediated oocyte maturation belonging to both clusters. Five other pathways—notch signaling pathway, lysosome, leukocyte transendothelial migration, endocytosis, and MAPK signaling pathway—were not clustered with the others (Table 15).

Given that multiple pathways were significantly enriched with the differentially expressed genes, the heterogeneity of perturbation was investigated across samples. All the significant genes in the top 14 pathways, from neurotrophin signaling to the VEGF pathway (Table 15), were grouped together as pathway cluster 1. A majority of these genes were associated with immune response. The genes in the long-term potentiation and long-term depression pathways were grouped as pathway cluster 2. In this cluster, synaptic genes were enriched. When the samples were plotted in a multidimensional space corresponding to the two pathway clusters (FIG. 5), four subgroups were distinct. The samples in quadrant I of FIG. 5 were perturbed in both pathway cluster 1 and pathway cluster 2, while the majority of samples in quadrant III were not significantly perturbed for either gene set. A subgroup of ASD samples was perturbed for pathway cluster 2 (quadrant II in FIG. 5), and some were significant for pathway cluster 1 (quadrant IV in FIG. 5). Also found were 6 significant clusters of Gene Ontology biological process terms grouped by the same approach as KEGG pathways (Cohen's kappa>0.5) from 428 overrepresented terms (Table 20), but the heterogeneity in these terms was not as clear as in KEGG pathways.

### Prediction of Autism Using Blood Gene Expression Signatures Using 55-Gene Prediction Model

To test whether peripheral blood gene expression profiles could be used as a molecular diagnostic tool for identifying ASD, a repeated leave-group out cross-validation (LGOCV) strategy was used with P1* to build a prediction model. First, the training set (P1*) was utilized to determine a classification signature (i.e. a combination of gene expression measurements) that was used to classify ASD patients in P1* (compared to controls). Next, the 489 differentially expressed genes were ranked according to their area under the receiver operating characteristic (ROC) curve (AUC). Next, those genes with low expression were excluded, requiring the minimum expression level across all samples to be at least 150. A total of 391 differentially expressed genes were then utilized in building the prediction models, which were subsequently tested against the samples in the independent validation cohort (P2*). The top N genes (where N ranges from 10 to 390 incremented by 5) were used to build prediction models using a repeated 5-folds LGOCV with a partial least squares (PLS) method, and AUCs were calculated for each cross-validation instance (see Methods). The prediction model using the top 55 genes was the most stable from 100-repeated LGOCV, having the smallest coefficient of variation in AUCs from 100 trials. The top 55 genes performed significantly better than the 50-gene model (one sided t test P=0.00031). The 55-gene prediction model was chosen because it minimized description length—i.e., the number of predictor genes—while maintaining good prediction performance, and used it to evaluate the independent dataset, P2*. The 55 significant genes are listed in Table 21. The performance of PLS was comparable to that of other prediction algorithms (Table 22); thus the classification performance was not attributable to a specific prediction algorithm.

The accuracy of this 55-gene set (also referred to as ASD55) within P1* was relatively high which is consistent with P1* being the training set (AUC 0.98, 95% confidence interval (CI), 0.965-1.000), but ASD55 also had good performance when applied to the P2* validation population (AUC 0.70, 95% CI 0.623-0.773) (Table 16). When generating a set of genes to classify samples, a tradeoff between specificity and sensitivity must be considered to achieve optimal results as shown by the ROC curves in FIG. 6A. To determine whether the ASD55 classifier performed better than expected by chance, 55 genes were randomly sampled 2,000 times and the performances of these random sets were evaluated by AUCs. The ASD55 model outperformed all of the 2,000 trials of randomly chosen sets of 55 genes (permutation P<0.0005). Since the majority of the training set (P1*) consisted of ASD patients, the performance of ASD55 was checked for inflation from such imbalances by calculating the ‘balanced accuracy’. The balanced accuracy is defined as the average of the accuracies obtained in either class (patients and control), or, equivalently, the arithmetic mean of specificity and sensitivity. It is essentially equal to conventional accuracy if the classifier performs equally well on both classes, but if the classifier's accuracy is entirely due to imbalance in the data the balanced accuracy will drop to random chance (0.5). The average balanced accuracy of ASD55 within P1* was 0.72, which is higher than random chance (0.5) implying that ASD55 was not entirely affected by imbalanced data. The training set (P1*) consisted of males only while the test set (P2*) had both genders. The prediction model built with males performed better for males in P2*. The AUC for male samples in P2* was 0.73 (95% CI 0.645-0.824) compared to 0.51 (95% CI 0.357-0.672) for female samples. To test the robustness of ASD55, ASD55 was trained with P2* samples to classify P1* samples, switching the training and validation sets. The performance was comparable to the original classification accuracy where P1* was used as the training set (AUC 0.69, 95% CI 0.583-0.797, FIG. 6B). All male patients identified as having mental retardation were accurately classified in both training and validation datasets while two female cases were predicted as non-cases.

Overall, the ASD55 predictor genes were enriched with 2 KEGG pathways (TGF-beta signaling pathway and Neurotrophin signaling pathway) and 8 Gene Ontology biological process terms (Table 23). 29 out of 55 predictor genes were associated with expression in the brain according to enrichment analysis using DAVID on UniProt tissue expression categories (UP_TISSUE, EASE score P=0.071, FDR 53.88). Also, hierarchical clustering of samples in P1* by the ASD55 predictor genes showed a clear distinction between patients and controls (FIG. 7).

### Effect of Other Clinical and Demographic Factors on Blood Gene Expression

In order to ensure that the predictor was robust for ASD classification, the expression data for potential confounders was reviewed. Among the demographic and clinical features, age at time of blood draw significantly influenced gene expression. Within the ASD group, age at blood collection was correlated within 382 genes at a significance level of P<0.001 (Spearman's rank correlation test, N=66, corresponding FDR 0.018). Six KEGG pathways were significantly enriched with the 382 age-correlated genes in the P1* ASD population (Table 24). The carbon pool by folate pathway (KEGG ID: hsa00670) was the most significantly enriched with age-correlated genes (EASE score P=4.6×10⁻⁷, FDR 5.2×10⁴). The age-correlated genes in this pathway were MTHFD1, TYMS, SHMT2, ATIC, DHFR, MTHFD1L, and GART. The ASD55 genes were not significantly correlated with age except for CNTRL and UTY, which were correlated with age in patients but not controls. UTY was one of the 23 genes that were differentially expressed in both datasets (P1* and P2*). In the P1* control group (N=33), 163 genes correlated significantly with age, but none of the ASD55 genes were among them.

Several other clinical and developmental characteristics were also correlated with gene expression changes as summarized in Table 17. A positive personal history of developmental delay including a delay in hitting milestones such as sitting, crawling, walking, and speaking was associated with 12 genes including the aristaless related homeobox gene (ARX). ARX is a homeodomain transcription factor that plays crucial roles in cerebral development and patterning, and is implicated in X-linked mental retardations. ARX was not identified as being differentially expressed in the ASD group of P1 (P=0.74); however, it was significantly down-regulated in the individuals with positive history of developmental delay (P=0.00037, FDR 0.30).

In the P1* cohort, 9 patients with ASD were diagnosed with leaning disorders. Sixty-four genes were differentially expressed with regard to learning disorders (Positive History N=9, Negative History N=90, P<0.001, corresponding FDR 0.14). The calcium signaling pathway (KEGG ID: hsa04020) was significant (hypergeometric P=0.023, FDR 0.19) due to ADRA1B, CHRM2, PPP3R1, and P2RX3. Another gene differentially expressed in patients with learning disorders, Synapsin 2 (SYN2), is a synaptic vesicle-associated protein. The differentially expressed genes that were correlated with other clinical conditions including psychiatric, neurological, gastrointestinal disorders, and seizure disorder are summarized in Table 17.

### Further Description of Materials and Methods

### Processing of Microarray Data

Gene expression levels were calculated using Affymetrix Power Tools version 1.10 (Affymetrix, CA). The Probe Log Iterative ERror (PLIER) algorithm was used that includes a probe-level quantile normalization method for each microarray platform separately. To match the probeset identifiers from the two different platforms used in this study, a Best Match subset was used between the two. 29,129 out of 54,613 total probesets on U133p2 were best-matched to 17,984 unique probesets of the GeneST array, and these matched probesets were used for the cross-platform prediction analysis. For the genes represented by more than two U133p2 probesets, the genes for which all probesets changed to the same direction were included.

To identify hidden confounders such as batch effect, surrogate variable analysis (SVA) was performed with null model for batch effect. For the P1* dataset, SVA found 6 surrogate variables in residuals after fitting with the primary variable of interest, i.e., clinical diagnosis. The first surrogate variable significantly correlated with the year when the microarray profiling was performed. In the P2* dataset, a batch with 12 samples was grouped separately from the other 172 samples from a principal component analysis although none of the surrogate variables was correlated with the 12 outlier samples. The ComBat algorithm was used to reduce the batch effects in P1* and P2* independently as the two array platforms are different in the design of probe sequences such that U133p2 array uses both perfect match (PM) and mismatch (MM) probes while GeneST array only has PM probes. All statistical analyses were performed with the ComBat corrected expression data.

### Statistical Analysis for Differentially Expressed Genes and Enriched Pathways

To identify differentially expressed genes in cases compared to controls, several tests were used, the Welch's t-test for two group comparison, and a one-way analysis of variance with Dunnett's post hoc tests to find significantly changed genes in AUT, PDDNOS, or ASP compared to the control group. To identify differentially expressed genes in the P2* dataset, the significance of diagnosis and gender was determined by two-way analysis of variance and follow-up Welch's t-test for each gender and Dunnett's post hoc tests for subtypes. The threshold for differential expression was set at nominal p-value <0.001. A general linear model was used to evaluate the significance of diagnosis, gender, age, and the other covariates. p-values were corrected for multiple comparisons by calculating a false discovery rate (FDR). Fisher's exact test was used for categorical data. Spearman's rank correlation coefficients were calculated to evaluate correlation between continuous phenotypic variables such as age at blood drawing and the expression level of each gene. The significance of correlation was determined using Fisher's r-to-z transformation. Enriched biological pathways with predictor genes were found using the DAVID functional annotation system. For significant KEGG pathways, the robust Mahalanobis distance of each individual was calculated from the common centroid of all cases and controls to find outliers using the minimum covariance determinant estimator. A quantile of the Chi-squared distribution (e.g., the 97.5% quantile) was used as a cut-off to define outliers, because for multivariate normally distributed data the Mahalanobis distance values are approximately chi-squared distributed. These outliers can be interpreted as biologically distinct subgroups for each pathway. Statistical analyses were performed using the R statistical programming language, and robust multivariate outlier analysis was performed using the chemometrics R library package.

### Statistical Prediction Analysis

Prediction analysis was performed in the following sequential steps; 1) ranking genes for predictor selection, 2) setting up a cross-validation strategy in the training set, 3) tuning parameters and building prediction models, and 4) predicting a test set, and evaluating prediction performances (FIG. 9). First, all genes were ranked by AUC. Next, the top 10 genes were selected from the ranked list to build a prediction model with a partial least square (PLS) method in the P1* dataset using a repeated leave-group out cross-validation (LGOCV) strategy, then repeated the same procedure with the top N genes incremented by 5 up to 390. For each prediction model using the top N genes, all P1* samples (N=99) were divided to 80% (a train set) and 20% (a test set), keeping the proportion of ASD and controls the same in each set. This step was repeated 100 times to estimate robust prediction performance (i.e., outer cross validation). To optimize each prediction model further, an inner cross-validation approach was deployed where 80% of the samples served as an inner train set, and 20% were used as an inner test set. The inner cross-validation procedure was repeated 100 times to find optimal tuning parameters for the specific prediction algorithm used. For each prediction model with the top N genes, a total of 10,000 predictions (i.e., 100 repeated LGOCVs×100 inner cross-validations) were made.

For each sample in a test set, the model predicts the probability of being classified as ASD. Thus, the number of false positives among positive predictions changes with the threshold. Overall prediction accuracy was calculated as (the number of true positives+the number of true negatives)/N, where N was the total number of samples in a dataset. Sensitivity, specificity, positive predictive value, and negative predictive value were presented as standard measures of prediction performance with AUC. The ROC curve summarizes the result at different thresholds.

To find a high performing prediction model with a minimum description length, AUCs between prediction models were compared using the top N genes. The mean AUCs improved gradually with increasing model complexities. However, it was also possible to identify the most stable prediction model by calculating the coefficient of variation of AUCs with 100 trials of outer cross validations. 5 additional prediction methods were tested: Logistic regression, Naïve Bayes, k-Nearest Neighbors, Random Forest, and Support Vector Machine using 55 genes with 5 fold LGOCV strategy. Statistical prediction analysis was performed using the caret and RWeka R library packages.

### Quantitative RT-PCR Validation

A total of 12 genes using 30 ASD and 30 control samples from the P1 population were run in replicates of four on the Biomark real time PCR system (Fluidigm, CA) using nanoliter reactions and the Taqman system (Applied Biosystems, CA). 60 samples were used. Following the Biomark protocol, quantitative RT-PCR (qRT-PCR) amplifications were carried out in a 9 nanoliter reaction volume containing 2× Universal Master Mix (Taqman), taqman gene expression assays, and preamplified cDNA. Pre-amplification reactions were done in a PTC-200 thermal cycler from MJ Research, per Biomark protocol. Reactions and analysis were performed using a Biomark system. The cycling program consisted of an initial cycle of 50° C. for 2 minutes and a 10 min incubation at 95° C. followed by 40 cycles of 95° C. for 15 seconds, 70° C. for 5 seconds, and 60° C. for 1 minute. Data was normalized to the housekeeping gene GAPDH, and expressed relative to control. All primers used for the 12 genes are listed in Table 13.

TABLE 12
Characteristics of patients with Autism Spectrum Disorders and
Controls in the training set (P1*) and in the validation set (P2*).

| | Training | | Validation | |
|---|---|---|---|---|
| | Set (P1*) | | Set (P2*) | |
| Characteristic | ASD | Control | ASD | Control |
| No. | 66 | 33 | 104 | 82 |
| Age-years | | | | |
| Mean | 8.0 | 9.0 | 8.4 | 8.1 |
| Interquartile range | 5.5 ± 9.7 | 4.0-13.1 | 5.0-11.0 | 4.1- 12.3 |
| Male-no. (%) | 66 (100) | 33 (100) | 80 (77) | 48 (59) |
| Diagnosis (Male %) | | | | |
| Autistic Disorder | 31 | — | 40 (75) | — |
| PDD, NOS | 26 | — | 49 (76) | — |
| Asperger's | 9 | — | 15 (87) | — |
| Disorder | | | | |
| Race-no. | | | | |
| Caucasian | 60 | 13 | 96 | 33 |
| Black | 0 | 5 | 0 | 8 |
| Asian | 1 | 1 | 3 | 2 |
| Mixed | 5 | 1 | 4 | 8 |
| Other | — | 4 | — | 21 |
| Unknown | 1 | 9 | 1 | 10 |
| Ethnicity | | | | |
| Hispanic-no. | 2 | 9 | 8 | 36 |
| Unknown-no. | 1 | — | — | — |
| Developmental delay- | 21 | 5 | 51 | 3 |
| no. | | | | |
| Learning Disorder-no. | 9 | — | | — |
| Psychiatric Disorder- | 14 | 4 | 32 | 1 |
| no. | | | | |
| Neurological Disorder- | 8 | — | 18 | — |
| no. | | | | |
| Gastrointestinal | 24 | — | 20 | — |
| Disorder-no. | | | | |
| Autoimmune Disorder- | — | — | 7 | — |
| no. | | | | |
| Cerebral Palsy-no. | — | — | 1 | — |

TABLE 13
Quantitative RT-PCR validations of 12 differentially expressed genes. 12
significantly differentially expressed genes were selected that had average fold change greater
than 1.5 and mean expression levels greater than 150 in the P1* dataset, and validated changes
using quantitative RT-PCR. A total of 30 ASD and 30 control samples from the P1* population
were run in replicates of four on the Biomark real time PCR system (Fluidigm, CA) using
nanoliter reactions and the Taqman system (Applied Biosystems, CA). 60 of the samples were
analyzed. The housekeeping gene used for qRT-PCR normalization was GAPDH
(Hs9999905_m1). The values shown are for 30 ASD and 30 controls from the P1* population,
and fold changes refer to ASD/Control. P-values were calculated using Welch's t-test. For
microarray data, p-values and fold changes were recalculated using the available samples.
Eleven of 12 genes (all except ZMAT1) were successfully validated.

| | | qRT-PCR | | Microarray | |
|---|---|---|---|---|---|
| | TaqMan Primer | Fold | | Fold | |
| Gene | ID | change | p-value | change | p-value |
| CREBZF | Hs02742201_s1 | 1.73 | 0.000127974 | 1.60 | 8.8516E−05 |
| HNRNPA2B1 | Hs00955384_m1 | 1.35 | 0.00119253 | 1.53 | 4.2587E−06 |
| KIDINS220 | Hs01057000_m1 | 2.16 | 8.44446E−10 | 1.57 | 2.674E−05 |
| LBR | Hs01032700_m1 | 2.50 | 7.55278E−10 | 1.63 | 5.85338E−05 |
| MED23 | Hs00606608_m1 | 2.24 | 1.95917E−09 | 1.51 | 0.000259037 |
| RBBP6 | Hs00544663_m1 | 1.98 | 0.000388767 | 1.58 | 0.000156489 |
| SPATA13 | Hs01128069_m1 | 1.61 | 0.000236786 | 1.56 | 6.07308E−05 |
| SULF2 | Hs01016476_m1 | 1.89 | 5.58742E−08 | 1.72 | 7.35118E−06 |
| TMEM30A | Hs01092148_m1 | 3.19 | 4.27915E−10 | 1.84 | 7.26489E−05 |
| ZDHHC17 | Hs00604479_m1 | 3.82 | 7.3983E−12 | 1.61 | 1.22144E−05 |
| ZMAT1 | Hs00736844_m1 | 0.60 | 0.413889282 | 1.86 | 8.81564E−05 |
| ZNF12 | Hs00212385_m1 | 2.35 | 9.12987E−09 | 1.54 | 1.86789E−06 |

TABLE 14
Differentially expressed genes in copy number variation (CNV) regions linked to ASD.

| Copy number variation | Differentially expressed genes in P1* dataset |
|---|---|
| Gain | ADAM10, AP1G1, CCNL1, CLIP1, DDX55, DOCKS, GRIPAP1, HIPK3, |
| | JMJD1C, KLHL2, MAPKB, MTMR10, PCGF3, RNF111, SACS, SNX27, |
| | SPATA13, TAOK3, WDR7, ZNF268, ZZEF1 |
| Loss | ANTXR2, ATRN, FRMD4B, HECA, ING5, LIFR, OR10A4, SIN3A, |
| | UTRN, VAV3, ZC3H13, ZNF548, ZNF592 |
| Gain and loss | AHR, CRKL, DMXL1, KBTBD11, KIAA0947, KIAA1468, MAPK1, |
| | TRIO, ZBED4, ZNF516 |

TABLE 15
Top 22 KEGG pathways enriched for differentially expressed genes in ASD (P1*).

| | | EASE | FDR | |
|---|---|---|---|---|
| KEGG pathways | Count | score P | (%) | Genes |
| Pathway Cluster 1 | | | | |
| Neurotrophin signaling pathway | 13 | 0.00023 | 0.26 | MAP2K1, PIK3CB, PIK3CD, KIDINS220, |
| | | | | MAPK1, YWHAG, MAP3K5, RPS6KA3, |
| | | | | CRKL, MAPK14, SH2B3, MAPK8, CRK |
| Fc gamma R-mediated | 9 | 0.00303 | 3.41 | MAPK1, PTPRC, DOCK2, CRKL, VAV3, |
| phagocytosis | | | | MAP2K1, PIK3CB, PIK3CD, CRK |
| Renal cell carcinoma | 8 | 0.00307 | 3.45 | MAPK1, CRKL, MAP2K1, PIK3CB, |
| | | | | PIK3CD, CREBBP, EGLN1, CRK |
| Chemokine signaling pathway | 12 | 0.01094 | 11.82 | MAPK1, DOCK2, CRKL, VAV3, ROCK1, |
| | | | | MAP2K1, GNAI1, PIK3CB, PREX1, |
| | | | | PIK3CD, CCR2, CRK |
| Regulation of actin | 14 | 0.01174 | 12.62 | GNA13, VAV3, MAP2K1, ROCK1, PIK3CB, |
| cytoskeleton | | | | PIK3CD, SSH2, IQGAP2, ITGB2, MAPK1, |
| | | | | CRKL, ITGAV, PPP1R12A, CRK |
| mTOR signaling pathway | 6 | 0.01358 | 14.47 | MAPK1, RPS6KA3, PIK3CB, PIK3CD, |
| | | | | CAB39, RICTOR |
| Chronic myeloid leukemia | 7 | 0.01413 | 15.01 | MAPK1, CRKL, CTBP2, MAP2K1, PIK3CB, |
| | | | | PIK3CD, CRK |
| Fc epsilon RI signaling pathway | 7 | 0.02189 | 22.35 | MAPK1, VAV3, MAP2K1, PIK3CB, |
| | | | | MAPK14, PIK3CD, MAPK8 |
| B cell receptor signaling | 6 | 0.02773 | 27.48 | MAPK1, VAV3, MAP2K1, PIK3CB, PIK3CD, |
| pathway | | | | PPP3CB |
| T cell receptor signaling | 8 | 0.02797 | 27.69 | MAPK1, PTPRC, VAV3, MAP2K1, PIK3CB, |
| pathway | | | | MAPK14, PIK3CD, PPP3CB |
| Focal adhesion | 12 | 0.02878 | 28.38 | IGF1R, MAPK1, CRKL, VAV3, ROCK1, |
| | | | | MAP2K1, PIK3CB, ITGAV, PIK3CD, |
| | | | | PPP1R12A, MAPK8, CRK |
| ErbB signaling pathway | 7 | 0.02987 | 29.29 | MAPK1, CRKL, MAP2K1, PIK3CB, |
| | | | | PIK3CD, MAPK8, CRK |
| Natural killer cell mediated | 8 | 0.04051 | 37.66 | IFNAR2, MAPK1, VAV3, MAP2K1, PIK3CB, |
| cytotoxicity | | | | PIK3CD, PPP3CB, ITGB2 |
| VEGF signaling pathway | 6 | 0.04888 | 43.6 | MAPK1, MAP2K1, PIK3CB, MAPK14, |
| | | | | PIK3CD, PPP3CB |
| Pathway Cluster 1 and 2 | | | | |
| Progesterone-mediated oocyte | 9 | 0.00408 | 4.57 | IGF1R, MAPK1, RPS6KA3, MAP2K1, |
| maturation | | | | GNAI1, PIK3CB, MAPK14, PIK3CD, |
| | | | | MAPK8 |
| Pathway Cluster 2 | | | | |
| Long-term potentiation | 7 | 0.01054 | 11.4 | MAPK1, RPS6KA3, GNAQ, MAP2K1, |
| | | | | CREBBP, PPP3CB, PPP1R12A |
| Long-term depression | 6 | 0.04209 | 38.82 | GNA13, IGF1R, MAPK1, GNAQ, MAP2K1, |
| | | | | GNAI1 |
| Not clustered | | | | |
| Notch signaling pathway | 6 | 0.00536 | 5.96 | CTBP2, KAT2B, MAML1, CREBBP, |
| | | | | ADAM17, MAML3 |
| Lysosome | 9 | 0.01136 | 12.24 | LAMP1, NPC1, AP1G1, HEXB, GAA, CTSD, |
| | | | | PPT1, CLTC, MANBA |
| Leukocyte transendothelial | 9 | 0.0174 | 18.18 | RASSF5, VAV3, ROCK1, GNAI1, PIK3CB, |
| migration | | | | MAPK14, PIK3CD, PECAM1, ITGB2 |
| Endocytosis | 11 | 0.02135 | 21.85 | EPS15, IGF1R, RNF103, RAB22A, RAB5A, |
| | | | | GIT2, SH3KBP1, PDCD6IP, CLTC, ARAP2, |
| | | | | ARAP1 |
| MAPK signaling pathway | 14 | 0.04635 | 41.86 | MAP2K1, NLK, TAOK3, PPM1B, MAP4K4, |
| | | | | MAPK1, MAP3K5, RPS6KA3, CRKL, |
| | | | | MAPK14, PPP3CB, MAPK8, CRK, RASA1 |

TABLE 16
Prediction performance of ASD55 trained with P1*.

| | | | | | Positive | Negative |
|---|---|---|---|---|---|---|
| | AUC | | | | Predictive | Predictive |
| Validation | (95% Confidence | Accuracy | Sensitivity | Specificity | Value | Value |
| set | Intervals) | (%) | (%) | (%) | (%) | (%) |
| P2 | 0.70 (0.623-0.773) | 67.7 | 69.2 | 65.9 | 72.0 | 62.8 |
| P2 | 0.73 (0.645-0.824) | 72.7 | 90.0 | 43.8 | 72.7 | 72.4 |
| (male) | | | | | | |
| P2 | 0.51 (0.357-0.672) | 63.8 | 50.0 | 73.5 | 57.1 | 67.6 |
| (female) | | | | | | |

Abbreviations:
ASD55, the genes in a classifier developed on P1* with 55 genes listed in Table 21;
AUC, area under the receiver operating characteristic curve.

TABLE 17
Exemplary genes that are significantly correlated with clinical features.

| | Number of | |
|---|---|---|
| Medical and | significant | |
| developmental | genes (p < | |
| history | 0.001) | Significant genes |
| Developmental | 12 | ARX, BMS1P1, C20orf196, CCDC18, IBTK, PNRC1, RHBDL2, TIGD1, TRIM4, |
| delay | | ZNF37A, ZNF415, ZNF536 |
| Learning | 68 | ADRA1B, AKNAD1, ANKRD18A, ANKRD30A, APP, BOD1L, C20orf166-A, |
| disorders | | C6orf195, CA2, CACNG5, CAV2, CEP19, CHRM2, CLDN5, CNTNAP3, CRYGN, |
| | | CXCL5, DDX11L2, ENSG00000217702, EPHA10, F13A1, FAM184B, FMO3, |
| | | GFOD1, GGTA1P, GIF, GNG11, GSC2, HBEGF, HGD, HRCT1, IGSF11, IGSF22, |
| | | ITPRIPL2, IZUMO1, KCNA1, KRT81, LCE1B, LOC126536, LYZL4, MECOM, |
| | | MSH4, NME5, NPY, NR1H4, P2RX3, PACS2, PF4V1, PPFIA2, PPP3R1, |
| | | RAX2, RNF17, RPL21P68, SCGN, SCN9A, SHH, SLC16A9, SLCO2B1, SMCR8, |
| | | SYN2, TCTN2, TEAD1, TMIE, TRH, TXNRD2, VGLL3, WRB, ZNF652 |
| Neurological | 7 | FAM13A, LRRD1, PITX3, SH3PXD2B, SPRR4, SPZ1, TACR2, |
| disorders | | |
| Psychiatric | 5 | CSTT, GPR111, HIP1, MED25, STX19 |
| disorders | | |
| Gastrointestinal | 5 | COL7A1, MARK1, PLA2G4C, SETMAR, TTR |
| disorders | | |
| Seizure | 4 | GPR153, GSC2, MGC39545, PITX3 |
| disorders | | |

TABLE 18
Differentially expressed genes in P1*. Welch' s t-test was used for two groups
comparison, and one-way analysis of variance with Dunnett's post hoc tests were used to find
significantly changed genes in autistic disorder (AUT), PDD-NOS (PDDNOS), or Asperger's
disorder (ASP) compare to control group. p values were corrected for the multiple comparisons
by calculating a false discovery rate (FDR).

| | | | | p-value | FDR | p-value | FDR | p-value | FDR |
|---|---|---|---|---|---|---|---|---|---|
| Affymetrix | | | | (AUT vs. | (AUT vs. | (PDDNOS | (PDDNOS | (ASP vs. | (ASP vs. |
| ID | Gene | p-value | FDR | Control) | Control) | vs. Control) | vs. Control) | Control) | Control) |
| 8177137 | UTY | 4.417E−07 | 1.091E−03 | 5.673E−06 | 1.935E−02 | 1.630E−04 | 1.627E−01 | 5.572E−02 | 1.00 |
| 7931353 | PTPRE | 5.502E−07 | 1.091E−03 | 3.422E−04 | 5.595E−02 | 6.404E−05 | 1.627E−01 | 4.151E−04 | 1.00 |
| 8152988 | SLA | 6.500E−07 | 1.091E−03 | 1.011E−04 | 4.183E−02 | 1.776E−04 | 1.627E−01 | 1.810E−03 | 1.00 |
| 7975361 | KIAA0247 | 8.956E−07 | 1.091E−03 | 5.694E−05 | 3.600E−02 | 1.157E−04 | 1.627E−01 | 2.974E−02 | 1.00 |
| 8066822 | SULF2 | 9.252E−07 | 1.091E−03 | 1.092E−04 | 4.183E−02 | 8.555E−05 | 1.627E−01 | 1.431E−02 | 1.00 |
| 8138116 | ZNF12 | 1.003E−06 | 1.091E−03 | 7.493E−07 | 9.780E−03 | 3.228E−04 | 1.758E−01 | 4.296E−01 | 1.00 |
| 8051814 | ZFP36L2 | 1.129E−06 | 1.091E−03 | 1.074E−05 | 2.441E−02 | 2.233E−04 | 1.627E−01 | 1.764E−01 | 1.00 |
| 8151149 | ARFGEF1 | 1.157E−06 | 1.091E−03 | 9.840E−06 | 2.441E−02 | 1.378E−04 | 1.627E−01 | 2.269E−01 | 1.00 |
| 7995631 | RBL2 | 1.692E−06 | 1.269E−03 | 1.434E−06 | 9.780E−03 | 1.846E−04 | 1.627E−01 | 6.884E−01 | 1.00 |
| 8059596 | TRIP12 | 1.759E−06 | 1.269E−03 | 5.151E−05 | 3.600E−02 | 3.796E−04 | 1.768E−01 | 3.280E−02 | 1.00 |
| 7987048 | MTMR10 | 1.850E−06 | 1.269E−03 | 1.126E−04 | 4.183E−02 | 9.313E−05 | 1.627E−01 | 6.821E−02 | 1.00 |
| 7929719 | C10orf28 | 2.905E−06 | 1.828E−03 | 4.622E−05 | 3.600E−02 | 1.598E−04 | 1.627E−01 | 2.260E−01 | 1.00 |
| 8115562 | RNF145 | 3.517E−06 | 1.836E−03 | 1.538E−05 | 2.493E−02 | 2.551E−04 | 1.627E−01 | 4.539E−01 | 1.00 |
| 8093976 | TBCID14 | 3.882E−06 | 1.836E−03 | 1.021E−03 | 7.694E−02 | 1.744E−04 | 1.627E−01 | 4.688E−03 | 1.00 |
| 7922889 | IVNSIABP | 4.035E−06 | 1.836E−03 | 3.114E−05 | 3.540E−02 | 3.889E−04 | 1.768E−01 | 2.558E−01 | 1.00 |
| 8138670 | HNRNPA2BI | 4.089E−06 | 1.836E−03 | 1.948E−05 | 2.498E−02 | 1.718E−03 | 2.383E−01 | 9.820E−02 | 1.00 |
| 8059770 | TIGD1 | 4.134E−06 | 1.836E−03 | 6.862E−05 | 3.600E−02 | 1.918E−04 | 1.627E−01 | 2.616E−01 | 1.00 |
| 8120992 | ZNF292 | 4.536E−06 | 1.902E−03 | 3.717E−06 | 1.690E−02 | 1.927E−03 | 2.384E−01 | 3.219E−01 | 1.00 |
| 8054135 | MGAT4A | 5.378E−06 | 2.137E−03 | 5.577E−05 | 3.600E−02 | 2.454E−04 | 1.627E−01 | 3.327E−01 | 1.00 |
| 7957277 | ZDHHC17 | 5.866E−06 | 2.214E−03 | 9.908E−05 | 4.183E−02 | 8.332E−04 | 2.229E−01 | 8.174E−02 | 1.00 |
| 8043310 | RMND5A | 6.235E−06 | 2.241E−03 | 1.423E−03 | 8.466E−02 | 3.588E−05 | 1.627E−01 | 7.715E−02 | 1.00 |
| 8066417 | SERINC3 | 6.565E−06 | 2.253E−03 | 6.775E−05 | 3.600E−02 | 2.249E−04 | 1.627E−01 | 4.116E−01 | 1.00 |
| 8003263 | ZCCHC14 | 6.927E−06 | 2.273E−03 | 8.767E−05 | 3.987E−02 | 1.281E−04 | 1.627E−01 | 4.914E−01 | 1.00 |
| 8073733 | NUP50 | 8.267E−06 | 2.463E−03 | 7.331E−05 | 3.654E−02 | 5.210E−04 | 2.031E−01 | 2.903E−01 | 1.00 |
| 8128394 | PNISR | 8.889E−06 | 2.463E−03 | 2.014E−05 | 2.498E−02 | 1.033E−03 | 2.383E−01 | 4.726E−01 | 1.00 |
| 8065776 | NCOA6 | 8.941E−06 | 2.463E−03 | 7.500E−05 | 3.654E−02 | 7.022E−04 | 2.212E−01 | 2.724E−01 | 1.00 |
| 8060418 | SIRPA | 9.081E−06 | 2.463E−03 | 3.902E−04 | 5.696E−02 | 5.632E−04 | 2.120E−01 | 6.010E−02 | 1.00 |
| 8126018 | STK38 | 9.137E−06 | 2.463E−03 | 5.748E−05 | 3.600E−02 | 1.446E−03 | 2.383E−01 | 1.994E−01 | 1.00 |
| 7969651 | DNAJC3 | 9.525E−06 | 2.479E−03 | 1.576E−03 | 8.812E−02 | 2.682E−04 | 1.627E−01 | 1.538E−02 | 1.00 |
| 7989224 | ADAMI0 | 1.028E−05 | 2.523E−03 | 1.135E−04 | 4.183E−02 | 1.433E−03 | 2.383E−01 | 1.246E−01 | 1.00 |
| 8068238 | IFNAR2 | 1.059E−05 | 2.523E−03 | 3.661E−04 | 5.595E−02 | 4.412E−04 | 1.824E−01 | 1.161E−01 | 1.00 |
| 8155898 | PCSK5 | 1.101E−05 | 2.523E−03 | 4.529E−05 | 3.600E−02 | 7.142E−04 | 2.212E−01 | 4.958E−01 | 1.00 |
| 8144082 | C7orf13 | 1.103E−05 | 2.523E−03 | 4.556E−04 | 5.809E−02 | 1.348E−04 | 1.627E−01 | 2.818E−01 | 1.00 |
| 8112687 | COL4A3BP | 1.227E−05 | 2.725E−03 | 3.665E−04 | 5.595E−02 | 7.460E−04 | 2.212E−01 | 8.820E−02 | 1.00 |
| 8009205 | DDX42 | 1.432E−05 | 2.856E−03 | 1.644E−05 | 2.493E−02 | 2.239E−03 | 2.410E−01 | 5.261E−01 | 1.00 |
| 8171762 | RPS6KA3 | 1.432E−05 | 2.856E−03 | 3.773E−04 | 5.595E−02 | 4.168E−04 | 1.800E−01 | 1.991E−01 | 1.00 |
| 8143988 | MLL3 | 1.433E−05 | 2.856E−03 | 1.973E−04 | 5.196E−02 | 1.817E−03 | 2.383E−01 | 9.309E−02 | 1.00 |
| 7988921 | MYO5A | 1.438E−05 | 2.856E−03 | 2.864E−04 | 5.495E−02 | 6.789E−04 | 2.212E−01 | 1.707E−01 | 1.00 |
| 8022441 | ROCK1 | 1.478E−05 | 2.860E−03 | 6.595E−05 | 3.600E−02 | 3.804E−03 | 2.706E−01 | 1.206E−01 | 1.00 |
| 8048980 | CAB39 | 1.656E−05 | 3.069E−03 | 8.724E−05 | 3.987E−02 | 1.560E−03 | 2.383E−01 | 3.342E−01 | 1.00 |
| 8090893 | MSL2 | 1.667E−05 | 3.069E−03 | 1.508E−03 | 8.673E−02 | 3.691E−04 | 1.768E−01 | 5.291E−02 | 1.00 |
| 7974066 | PNN | 1.815E−05 | 3.112E−03 | 1.626E−05 | 2.493E−02 | 3.167E−03 | 2.558E−01 | 5.266E−01 | 1.00 |
| 8157534 | CNTRL | 1.872E−05 | 3.112E−03 | 1.072E−04 | 4.183E−02 | 1.217E−03 | 2.383E−01 | 3.610E−01 | 1.00 |
| 8079140 | SNRK | 1.898E−05 | 3.112E−03 | 4.342E−04 | 5.735E−02 | 9.348E−04 | 2.383E−01 | 1.356E−01 | 1.00 |
| 8144317 | KBTBD11 | 1.927E−05 | 3.112E−03 | 2.463E−04 | 5.313E−02 | 1.279E−04 | 1.627E−01 | 7.561E−01 | 1.00 |
| 8079462 | NBEAL2 | 1.967E−05 | 3.112E−03 | 3.075E−03 | 1.107E−01 | 1.477E−03 | 2.383E−01 | 1.159E−03 | 1.00 |
| 8050128 | KIDINS220 | 1.988E−05 | 3.112E−03 | 3.281E−04 | 5.595E−02 | 2.784E−03 | 2.518E−01 | 6.124E−02 | 1.00 |
| 7911038 | ZNF238 | 1.999E−05 | 3.112E−03 | 4.167E−05 | 3.600E−02 | 1.564E−03 | 2.383E−01 | 6.198E−01 | 1.00 |
| 8119408 | NFYA | 2.064E−05 | 3.112E−03 | 9.081E−04 | 7.359E−02 | 2.487E−04 | 1.627E−01 | 2.321E−01 | 1.00 |
| 8119529 | UBR2 | 2.101E−05 | 3.112E−03 | 8.660E−04 | 7.324E−02 | 2.674E−04 | 1.627E−01 | 2.376E−01 | 1.00 |
| 8013965 | SSH2 | 2.132E−05 | 3.112E−03 | 5.606E−03 | 1.403E−01 | 2.742E−04 | 1.627E−01 | 9.314E−03 | 1.00 |
| 7950409 | KCNE3 | 2.144E−05 | 3.112E−03 | 8.796E−04 | 7.324E−02 | 1.544E−03 | 2.383E−01 | 3.592E−02 | 1.00 |
| 7924603 | LBR | 2.269E−05 | 3.232E−03 | 3.632E−04 | 5.595E−02 | 1.623E−03 | 2.383E−01 | 1.342E−01 | 1.00 |
| 8174119 | ZMAT1 | 2.377E−05 | 3.324E−03 | 3.832E−05 | 3.600E−02 | 2.385E−03 | 2.410E−01 | 5.808E−01 | 1.00 |
| 8060627 | ATRN | 2.435E−05 | 3.341E−03 | 1.530E−04 | 4.519E−02 | 9.548E−04 | 2.383E−01 | 4.788E−01 | 1.00 |
| 8104944 | NIPBL | 2.548E−05 | 3.435E−03 | 1.299E−04 | 4.350E−02 | 2.580E−03 | 2.448E−01 | 2.779E−01 | 1.00 |
| 7925622 | AHCTF1 | 2.769E−05 | 3.654E−03 | 1.784E−03 | 9.197E−02 | 3.371E−04 | 1.758E−01 | 1.451E−01 | 1.00 |
| 7973298 | TRAV8−3 | 2.808E−05 | 3.654E−03 | 3.744E−04 | 5.595E−02 | 1.702E−03 | 2.383E−01 | 1.797E−01 | 1.00 |
| 7953291 | CD9 | 2.865E−05 | 3.666E−03 | 5.348E−04 | 6.235E−02 | 9.636E−04 | 2.383E−01 | 2.236E−01 | 1.00 |
| 7939197 | HIPK3 | 2.970E−05 | 3.737E−03 | 2.021E−03 | 9.508E−02 | 3.479E−04 | 1.758E−01 | 1.362E−01 | 1.00 |
| 8169541 | DOCK11 | 3.204E−05 | 3.965E−03 | 6.086E−05 | 3.600E−02 | 2.367E−03 | 2.410E−01 | 6.122E−01 | 1.00 |
| 8132188 | AVL9 | 3.387E−05 | 4.047E−03 | 2.311E−04 | 5.313E−02 | 1.119E−03 | 2.383E−01 | 4.837E−01 | 1.00 |
| 8042337 | ACTR2 | 3.470E−05 | 4.047E−03 | 5.056E−04 | 6.235E−02 | 4.588E−03 | 2.782E−01 | 5.213E−02 | 1.00 |
| 7999044 | CREBBP | 3.472E−05 | 4.047E−03 | 6.236E−04 | 6.594E−02 | 2.037E−03 | 2.406E−01 | 1.325E−01 | 1.00 |
| 8042942 | HK2 | 3.485E−05 | 4.047E−03 | 4.227E−04 | 5.735E−02 | 6.774E−03 | 3.020E−01 | 3.975E−02 | 1.00 |
| 7968035 | SPATA13 | 3.572E−05 | 4.073E−03 | 3.566E−04 | 5.595E−02 | 5.894E−03 | 3.013E−01 | 6.520E−02 | 1.00 |
| 8106602 | ZFYVE16 | 3.615E−05 | 4.073E−03 | 6.618E−04 | 6.617E−02 | 3.508E−03 | 2.688E−01 | 6.420E−02 | 1.00 |
| 7950796 | CREBZF | 3.746E−05 | 4.158E−03 | 4.964E−05 | 3.600E−02 | 3.724E−03 | 2.688E−01 | 6.082E−01 | 1.00 |
| 7954711 | C12orf35 | 3.847E−05 | 4.168E−03 | 2.185E−03 | 9.773E−02 | 3.083E−03 | 2.556E−01 | 9.418E−03 | 1.00 |
| 7963244 | TFCP2 | 3.865E−05 | 4.168E−03 | 1.599E−03 | 8.867E−02 | 6.656E−04 | 2.212E−01 | 1.666E−01 | 1.00 |
| 8011542 | ZZEF1 | 3.932E−05 | 4.181E−03 | 2.337E−04 | 5.313E−02 | 1.167E−03 | 2.383E−01 | 5.894E−01 | 1.00 |
| 8111698 | RICTOR | 4.065E−05 | 4.262E−03 | 1.862E−03 | 9.213E−02 | 1.958E−03 | 2.385E−01 | 3.851E−02 | 1.00 |
| 7938179 | OR10A4 | 4.257E−05 | 4.402E−03 | 7.336E−03 | 1.565E−01 | 5.749E−04 | 2.120E−01 | 1.600E−02 | 1.00 |
| 8101260 | ANTXR2 | 4.555E−05 | 4.565E−03 | 7.085E−04 | 6.712E−02 | 2.069E−03 | 2.410E−01 | 1.779E−01 | 1.00 |
| 8109843 | DOCK2 | 4.579E−05 | 4.565E−03 | 1.148E−03 | 8.112E−02 | 3.140E−03 | 2.558E−01 | 6.035E−02 | 1.00 |
| 8163775 | MEGF9 | 4.637E−05 | 4.565E−03 | 8.905E−03 | 1.740E−01 | 1.629E−03 | 2.383E−01 | 1.446E−03 | 1.00 |
| 8023882 | ZNF516 | 4.656E−05 | 4.565E−03 | 1.576E−03 | 8.812E−02 | 1.267E−03 | 2.383E−01 | 1.228E−01 | 1.00 |
| 8058927 | TMBIM1 | 4.836E−05 | 4.680E−03 | 5.951E−04 | 6.443E−02 | 1.162E−03 | 2.383E−01 | 4.167E−01 | 1.00 |
| 8031737 | ZNF548 | 4.914E−05 | 4.696E−03 | 3.560E−04 | 5.595E−02 | 2.141E−03 | 2.410E−01 | 3.939E−01 | 1.00 |
| 8116247 | ZNF354A | 5.019E−05 | 4.736E−03 | 2.900E−04 | 5.495E−02 | 3.006E−03 | 2.540E−01 | 3.559E−01 | 1.00 |
| 8117663 | NKAPL | 5.224E−05 | 4.839E−03 | 8.708E−03 | 1.722E−01 | 2.977E−04 | 1.692E−01 | 5.358E−02 | 1.00 |
| 8045398 | RAB3GAP1 | 5.281E−05 | 4.839E−03 | 2.747E−03 | 1.066E−01 | 4.193E−03 | 2.733E−01 | 9.278E−03 | 1.00 |
| 8122464 | UTRN | 5.323E−05 | 4.839E−03 | 9.699E−04 | 7.560E−02 | 5.972E−03 | 3.013E−01 | 4.178E−02 | 1.00 |
| 8161701 | TMEM2 | 5.385E−05 | 4.839E−03 | 1.609E−03 | 8.886E−02 | 2.632E−03 | 2.459E−01 | 6.625E−02 | 1.00 |
| 8152148 | UBR5 | 5.716E−05 | 5.003E−03 | 5.697E−04 | 6.443E−02 | 1.464E−03 | 2.383E−01 | 4.441E−01 | 1.00 |
| 7970301 | TMC03 | 5.818E−05 | 5.003E−03 | 4.448E−04 | 5.735E−02 | 2.851E−03 | 2.521E−01 | 3.266E−01 | 1.00 |
| 7927389 | MAPK8 | 5.964E−05 | 5.003E−03 | 1.629E−04 | 4.629E−02 | 1.940E−03 | 2.384E−01 | 7.483E−01 | 1.00 |
| 8167971 | MIR223 | 5.968E−05 | 5.003E−03 | 3.739E−03 | 1.201E−01 | 5.027E−03 | 2.894E−01 | 4.056E−03 | 1.00 |
| 7965123 | PPP1R12A | 5.990E−05 | 5.003E−03 | 2.515E−04 | 5.313E−02 | 6.397E−03 | 3.013E−01 | 2.637E−01 | 1.00 |
| 8127637 | TMEM30A | 6.017E−05 | 5.003E−03 | 1.339E−04 | 4.350E−02 | 7.255E−03 | 3.064E−01 | 3.966E−01 | 1.00 |
| 8097148 | KIAA1109 | 6.031E−05 | 5.003E−03 | 6.421E−04 | 6.617E−02 | 7.063E−03 | 3.048E−01 | 8.549E−02 | 1.00 |
| 7916592 | MYSM1 | 6.305E−05 | 5.173E−03 | 3.500E−04 | 5.595E−02 | 3.485E−03 | 2.688E−01 | 3.880E−01 | 1.00 |
| 8117106 | RNF144B | 6.419E−05 | 5.210E−03 | 6.130E−03 | 1.462E−01 | 1.913E−03 | 2.384E−01 | 1.220E−02 | 1.00 |
| 8025978 | ZNF763 | 6.532E−05 | 5.245E−03 | 1.877E−03 | 9.213E−02 | 1.901E−03 | 2.384E−01 | 1.362E−01 | 1.00 |
| 8040695 | KCNK3 | 6.738E−05 | 5.272E−03 | 5.165E−03 | 1.355E−01 | 2.185E−03 | 2.410E−01 | 1.785E−02 | 1.00 |
| 7930682 | FAMI60BI | 6.777E−05 | 5.272E−03 | 1.383E−03 | 8.459E−02 | 1.799E−03 | 2.383E−01 | 2.100E−01 | 1.00 |
| 8077528 | SETD5 | 6.906E−05 | 5.272E−03 | 1.833E−03 | 9.213E−02 | 2.089E−03 | 2.410E−01 | 1.344E−01 | 1.00 |
| 8053775 | ZNF5I4 | 6.916E−05 | 5.272E−03 | 3.352E−03 | 1.143E−01 | 2.453E−03 | 2.443E−01 | 3.865E−02 | 1.00 |
| 8059783 | NGEF | 7.074E−05 | 5.272E−03 | 1.435E−03 | 8.466E−02 | 1.048E−03 | 2.383E−01 | 3.492E−01 | 1.00 |
| 7986132 | MAN2A2 | 7.207E−05 | 5.272E−03 | 1.358E−03 | 8.459E−02 | 7.960E−04 | 2.229E−01 | 4.665E−01 | 1.00 |
| 7933877 | JMJD1C | 7.259E−05 | 5.272E−03 | 9.215E−04 | 7.359E−02 | 3.531E−03 | 2.688E−01 | 2.089E−01 | 1.00 |
| 7927062 | ZNF33A | 7.331E−05 | 5.272E−03 | 1.037E−04 | 4.183E−02 | 1.886E−03 | 2.384E−01 | 9.717E−01 | 1.00 |
| 8070826 | ITGB2 | 7.384E−05 | 5.272E−03 | 2.866E−03 | 1.070E−01 | 4.939E−03 | 2.894E−01 | 2.155E−02 | 1.00 |
| 8007023 | MSLI | 7.509E−05 | 5.272E−03 | 8.398E−03 | 1.702E−01 | 1.383E−03 | 2.383E−01 | 1.894E−02 | 1.00 |
| 7905789 | IL6R | 7.510E−05 | 5.272E−03 | 2.258E−03 | 9.935E−02 | 4.815E−03 | 2.894E−01 | 3.583E−02 | 1.00 |
| 7957806 | SCYL2 | 7.526E−05 | 5.272E−03 | 1.221E−03 | 8.193E−02 | 3.110E−03 | 2.556E−01 | 1.722E−01 | 1.00 |
| 8021496 | KIAA1468 | 7.527E−05 | 5.272E−03 | 3.053E−04 | 5.595E−02 | 4.304E−03 | 2.733E−01 | 4.422E−01 | 1.00 |
| 8091009 | PIK3CB | 7.543E−05 | 5.272E−03 | 1.237E−03 | 8.193E−02 | 1.746E−03 | 2.383E−01 | 3.102E−01 | 1.00 |
| 7968274 | PAN3 | 7.890E−05 | 5.432E−03 | 2.230E−04 | 5.313E−02 | 4.136E−03 | 2.733E−01 | 5.832E−01 | 1.00 |
| 8008834 | CLTC | 8.057E−05 | 5.432E−03 | 1.816E−04 | 4.956E−02 | 4.088E−03 | 2.733E−01 | 6.522E−01 | 1.00 |
| 7948667 | AHNAK | 8.217E−05 | 5.432E−03 | 3.769E−04 | 5.595E−02 | 1.254E−02 | 3.653E−01 | 1.309E−01 | 1.00 |
| 8122343 | HECA | 8.309E−05 | 5.432E−03 | 1.472E−04 | 4.463E−02 | 4.186E−03 | 2.733E−01 | 7.267E−01 | 1.00 |
| 8132531 | SPDYE1 | 8.318E−05 | 5.432E−03 | 4.123E−03 | 1.241E−01 | 1.450E−03 | 2.383E−01 | 9.949E−02 | 1.00 |
| 7969796 | TM9SF2 | 8.351E−05 | 5.432E−03 | 1.018E−02 | 1.830E−01 | 1.487E−03 | 2.383E−01 | 1.412E−02 | 1.00 |
| 8067113 | ZNF217 | 8.392E−05 | 5.432E−03 | 2.844E−03 | 1.070E−01 | 2.524E−03 | 2.448E−01 | 9.011E−02 | 1.00 |
| 7983953 | RNE111 | 8.483E−05 | 5.432E−03 | 3.954E−04 | 5.696E−02 | 1.576E−03 | 2.383E−01 | 7.611E−01 | 1.00 |
| 8164398 | GOLGA2 | 8.487E−05 | 5.432E−03 | 1.797E−03 | 9.197E−02 | 6.951E−04 | 2.212E−01 | 4.913E−01 | 1.00 |
| 8124059 | NUP153 | 8.491E−05 | 5.432E−03 | 6.792E−04 | 6.617E−02 | 2.373E−03 | 2.410E−01 | 4.700E−01 | 1.00 |
| 8171248 | KAL1 | 8.791E−05 | 5.577E−03 | 3.557E−04 | 5.595E−02 | 1.016E−02 | 3.402E−01 | 2.127E−01 | 1.00 |
| 8152962 | LRRC6 | 8.866E−05 | 5.577E−03 | 1.848E−03 | 9.213E−02 | 1.708E−03 | 2.383E−01 | 2.662E−01 | 1.00 |
| 8115927 | RNF44 | 9.048E−05 | 5.642E−03 | 5.910E−05 | 3.600E−02 | 3.646E−03 | 2.688E−01 | 9.750E−01 | 1.00 |
| 8110546 | MAML1 | 9.117E−05 | 5.642E−03 | 2.123E−04 | 5.313E−02 | 3.931E−03 | 2.709E−01 | 6.939E−01 | 1.00 |
| 8074791 | MAPK1 | 9.710E−05 | 5.920E−03 | 1.281E−03 | 8.285E−02 | 2.212E−03 | 2.410E−01 | 3.531E−01 | 1.00 |
| 7971422 | ZC3H13 | 9.725E−05 | 5.920E−03 | 1.211E−03 | 8.193E−02 | 1.735E−03 | 2.383E−01 | 4.355E−01 | 1.00 |
| 8158597 | GPR107 | 9.903E−05 | 5.980E−03 | 1.680E−03 | 9.035E−02 | 8.137E−04 | 2.229E−01 | 5.604E−01 | 1.00 |
| 8011945 | KIAA0753 | 1.021E−04 | 6.117E−03 | 3.652E−04 | 5.595E−02 | 1.318E−03 | 2.383E−01 | 9.054E−01 | 1.00 |
| 8041713 | PPM1B | 1.033E−04 | 6.138E−03 | 4.171E−04 | 5.735E−02 | 6.687E−03 | 3.018E−01 | 3.850E−01 | 1.00 |
| 7897482 | PIK3CD | 1.046E−04 | 6.170E−03 | 1.641E−03 | 8.988E−02 | 2.480E−03 | 2.448E−01 | 2.947E−01 | 1.00 |
| 8107356 | DCP2 | 1.077E−04 | 6.305E−03 | 8.118E−04 | 7.191E−02 | 6.800E−03 | 3.021E−01 | 2.212E−01 | 1.00 |
| 8082688 | DNAJC13 | 1.089E−04 | 6.306E−03 | 9.224E−04 | 7.359E−02 | 1.058E−02 | 3.429E−01 | 1.140E−01 | 1.00 |
| 8165077 | NACC2 | 1.113E−04 | 6.306E−03 | 1.435E−03 | 8.466E−02 | 3.174E−03 | 2.558E−01 | 2.947E−01 | 1.00 |
| 7897441 | H6PD | 1.115E−04 | 6.306E−03 | 2.172E−03 | 9.773E−02 | 1.624E−03 | 2.383E−01 | 3.410E−01 | 1.00 |
| 7994161 | RBBP6 | 1.119E−04 | 6.306E−03 | 1.318E−04 | 4.350E−02 | 9.558E−03 | 3.400E−01 | 6.694E−01 | 1.00 |
| 7954436 | LRMP | 1.125E−04 | 6.306E−03 | 1.983E−03 | 9.410E−02 | 2.554E−03 | 2.448E−01 | 2.554E−01 | 1.00 |
| 8027439 | ZNVF507 | 1.128E−04 | 6.306E−03 | 4.019E−04 | 5.711E−02 | 4.222E−04 | 1.800E−01 | 9.994E−01 | 1.00 |
| 8080878 | ATXN7 | 1.141E−04 | 6.335E−03 | 1.299E−03 | 8.360E−02 | 2.657E−02 | 4.521E−01 | 1.017E−02 | 1.00 |
| 7952739 | ZBTB44 | 1.155E−04 | 6.344E−03 | 1.470E−04 | 4.463E−02 | 6.843E−03 | 3.021E−01 | 7.556E−01 | 1.00 |
| 7932911 | KIF5B | 1.163E−04 | 6.344E−03 | 4.456E−04 | 5.735E−02 | 1.339E−02 | 3.697E−01 | 2.244E−01 | 1.00 |
| 8006123 | CPD | 1.181E−04 | 6.344E−03 | 2.166E−02 | 2.694E−01 | 4.680E−04 | 1.878E−01 | 3.936E−02 | 1.00 |
| 8095269 | POLR2B | 1.183E−04 | 6.344E−03 | 3.178E−04 | 5.595E−02 | 6.372E−03 | 3.013E−01 | 5.808E−01 | 1.00 |
| 8102024 | UBE2D3 | 1.185E−04 | 6.344E−03 | 7.556E−04 | 6.914E−02 | 6.357E−03 | 3.013E−01 | 3.119E−01 | 1.00 |
| 8105714 | SREK1 | 1.224E−04 | 6.493E−03 | 2.483E−04 | 5.313E−02 | 7.140E−03 | 3.054E−01 | 6.199E−01 | 1.00 |
| 7979044 | NIN | 1.235E−04 | 6.493E−03 | 3.350E−04 | 5.595E−02 | 6.104E−03 | 3.013E−01 | 5.875E−01 | 1.00 |
| 8106252 | HEXB | 1.239E−04 | 6.493E−03 | 6.731E−04 | 6.617E−02 | 5.563E−03 | 2.958E−01 | 4.063E−01 | 1.00 |
| 8081740 | ATP6V1A | 1.256E−04 | 6.537E−03 | 9.379E−03 | 1.772E−01 | 9.886E−04 | 2.383E−01 | 1.034E−01 | 1.00 |
| 8129522 | MED23 | 1.279E−04 | 6.612E−03 | 1.238E−04 | 4.330E−02 | 8.130E−03 | 3.187E−01 | 8.073E−01 | 1.00 |
| 8161906 | GNAQ | 1.314E−04 | 6.697E−03 | 3.585E−03 | 1.176E−01 | 5.901E−03 | 3.013E−01 | 5.777E−02 | 1.00 |
| 8021984 | YES1 | 1.317E−04 | 6.697E−03 | 4.084E−04 | 5.728E−02 | 2.733E−03 | 2.518E−01 | 8.157E−01 | 1.00 |
| 8098177 | KLHL2 | 1.322E−04 | 6.697E−03 | 1.876E−03 | 9.213E−02 | 7.986E−03 | 3.187E−01 | 9.800E−02 | 1.00 |
| 8046726 | SSFA2 | 1.353E−04 | 6.809E−03 | 2.286E−03 | 9.963E−02 | 1.380E−02 | 3.713E−01 | 2.787E−02 | 1.00 |
| 8020068 | ANKRD12 | 1.384E−04 | 6.884E−03 | 1.056E−03 | 7.844E−02 | 5.383E−03 | 2.957E−01 | 3.408E−01 | 1.00 |
| 8138922 | KBTBD2 | 1.386E−04 | 6.884E−03 | 5.833E−04 | 6.443E−02 | 3.709E−03 | 2.688E−01 | 6.654E−01 | 1.00 |
| 8108873 | ARHGAP26 | 1.399E−04 | 6.887E−03 | 4.115E−03 | 1.241E−01 | 7.514E−03 | 3.115E−01 | 3.240E−02 | 1.00 |
| 8052654 | PELI1 | 1.413E−04 | 6.887E−03 | 2.097E−03 | 9.675E−02 | 6.831E−03 | 3.021E−01 | 1.245E−01 | 1.00 |
| 8122909 | SCAF8 | 1.414E−04 | 6.887E−03 | 6.605E−04 | 6.617E−02 | 1.545E−03 | 2.383E−01 | 8.718E−01 | 1.00 |
| 8168678 | FAM133A | 1.426E−04 | 6.898E−03 | 1.981E−04 | 5.196E−02 | 1.869E−02 | 4.110E−01 | 4.117E−01 | 1.00 |
| 7964413 | R3HDM2 | 1.453E−04 | 6.945E−03 | 3.799E−03 | 1.211E−01 | 4.103E−03 | 2.733E−01 | 1.121E−01 | 1.00 |
| 8040552 | NCOA1 | 1.457E−04 | 6.945E−03 | 5.588E−03 | 1.403E−01 | 1.559E−03 | 2.383E−01 | 1.997E−01 | 1.00 |
| 7916045 | EPS15 | 1.463E−04 | 6.945E−03 | 6.195E−04 | 6.594E−02 | 8.212E−03 | 3.187E−01 | 4.063E−01 | 1.00 |
| 7935660 | DNMBP | 1.478E−04 | 6.972E−03 | 3.454E−04 | 5.595E−02 | 2.271E−03 | 2.410E−01 | 9.513E−01 | 1.00 |
| 8020129 | VAPA | 1.487E−04 | 6.972E−03 | 3.378E−03 | 1.149E−01 | 2.355E−03 | 2.410E−01 | 2.592E−01 | 1.00 |
| 8137404 | CHPF2 | 1.499E−04 | 6.986E−03 | 1.226E−03 | 8.193E−02 | 2.920E−03 | 2.521E−01 | 5.361E−01 | 1.00 |
| 8064790 | RASSF2 | 1.511E−04 | 6.987E−03 | 6.747E−03 | 1.525E−01 | 1.787E−03 | 2.383E−01 | 1.441E−01 | 1.00 |
| 7970287 | LAMPI | 1.518E−04 | 6.987E−03 | 1.124E−02 | 1.941E−01 | 1.150E−03 | 2.383E−01 | 9.946E−02 | 1.00 |
| 8021275 | POLI | 1.552E−04 | 7.099E−03 | 3.967E−04 | 5.696E−02 | 5.012E−03 | 2.894E−01 | 7.431E−01 | 1.00 |
| 8063755 | C20orf177 | 1.584E−04 | 7.135E−03 | 1.178E−03 | 8.180E−02 | 3.004E−03 | 2.540E−01 | 5.930E−01 | 1.00 |
| 8143341 | JHDM1D | 1.586E−04 | 7.135E−03 | 8.150E−03 | 1.673E−01 | 1.701E−03 | 2.383E−01 | 1.252E−01 | 1.00 |
| 7950838 | PICALM | 1.588E−04 | 7.135E−03 | 7.547E−03 | 1.587E−01 | 4.306E−03 | 2.733E−01 | 4.032E−02 | 1.00 |
| 7904086 | LRIG2 | 1.607E−04 | 7.151E−03 | 5.863E−03 | 1.439E−01 | 2.257E−03 | 2.410E−01 | 1.571E−01 | 1.00 |
| 7941769 | KDM2A | 1.610E−04 | 7.151E−03 | 1.179E−04 | 4.232E−02 | 1.609E−02 | 3.872E−01 | 7.187E−01 | 1.00 |
| 8071434 | CRKL | 1.623E−04 | 7.161E−03 | 3.536E−04 | 5.595E−02 | 4.370E−03 | 2.734E−01 | 8.534E−01 | 1.00 |
| 8041048 | FOSL2 | 1.632E−04 | 7.161E−03 | 1.922E−02 | 2.563E−01 | 3.655E−03 | 2.688E−01 | 4.539E−03 | 1.00 |
| 8102523 | FABP2 | 1.661E−04 | 7.247E−03 | 4.798E−03 | 1.320E−01 | 4.669E−02 | 5.250E−01 | 9.486E−05 | 1.00 |
| 7967255 | CLIP1 | 1.679E−04 | 7.285E−03 | 1.800E−03 | 9.197E−02 | 1.083E−02 | 3.459E−01 | 1.221E−01 | 1.00 |
| 7942839 | PCF11 | 1.711E−04 | 7.328E−03 | 7.861E−04 | 7.055E−02 | 8.455E−03 | 3.206E−01 | 4.000E−01 | 1.00 |
| 7970569 | SACS | 1.718E−04 | 7.328E−03 | 2.588E−04 | 5.313E−02 | 5.397E−03 | 2.957E−01 | 8.913E−01 | 1.00 |
| 7978376 | STXBP6 | 1.718E−04 | 7.328E−03 | 7.229E−03 | 1.555E−01 | 6.148E−04 | 2.207E−01 | 4.120E−01 | 1.00 |
| 8140398 | YWHAG | 1.738E−04 | 7.372E−03 | 5.830E−04 | 6.443E−02 | 1.040E−02 | 3.428E−01 | 4.325E−01 | 1.00 |
| 7909214 | RASSF5 | 1.751E−04 | 7.384E−03 | 4.005E−03 | 1.239E−01 | 2.584E−03 | 2.448E−01 | 2.634E−01 | 1.00 |
| 7990487 | SIN3A | 1.797E−04 | 7.490E−03 | 8.659E−03 | 1.719E−01 | 6.864E−04 | 2.212E−01 | 3.519E−01 | 1.00 |
| 8084128 | TTC14 | 1.798E−04 | 7.490E−03 | 1.394E−04 | 4.422E−02 | 1.505E−02 | 3.753E−01 | 7.792E−01 | 1.00 |
| 7909529 | RCOR3 | 1.806E−04 | 7.490E−03 | 4.382E−04 | 5.735E−02 | 6.522E−03 | 3.018E−01 | 7.201E−01 | 1.00 |
| 8097417 | PHF17 | 1.821E−04 | 7.511E−03 | 4.115E−04 | 5.728E−02 | 1.737E−03 | 2.383E−01 | 9.918E−01 | 1.00 |
| 7926565 | MLLT10 | 1.838E−04 | 7.511E−03 | 6.097E−04 | 6.549E−02 | 5.298E−03 | 2.944E−01 | 6.987E−01 | 1.00 |
| 7936614 | EIF3A | 1.850E−04 | 7.511E−03 | 1.739E−03 | 9.125E−02 | 5.375E−03 | 2.957E−01 | 3.459E−01 | 1.00 |
| 8072757 | CSF2RB | 1.875E−04 | 7.511E−03 | 7.051E−03 | 1.539E−01 | 7.093E−03 | 3.048E−01 | 3.242E−02 | 1.00 |
| 8159992 | ERMP1 | 1.882E−04 | 7.511E−03 | 6.569E−04 | 6.617E−02 | 6.658E−03 | 3.018E−01 | 5.982E−01 | 1.00 |
| 8083183 | U2SURP | 1.882E−04 | 7.511E−03 | 1.161E−03 | 8.166E−02 | 7.177E−03 | 3.060E−01 | 3.841E−01 | 1.00 |
| 8070010 | SYNJ1 | 1.895E−04 | 7.511E−03 | 1.403E−03 | 8.466E−02 | 6.994E−03 | 3.038E−01 | 3.494E−01 | 1.00 |
| 7969414 | KLF5 | 1.898E−04 | 7.511E−03 | 1.928E−03 | 9.294E−02 | 8.508E−03 | 3.206E−01 | 2.075E−01 | 1.00 |
| 7987325 | AQR | 1.905E−04 | 7.511E−03 | 1.428E−03 | 8.466E−02 | 4.288E−03 | 2.733E−01 | 5.099E−01 | 1.00 |
| 8070046 | GCFC1 | 1.910E−04 | 7.511E−03 | 1.069E−03 | 7.844E−02 | 3.645E−03 | 2.688E−01 | 6.775E−01 | 1.00 |
| 7989387 | VPS13C | 1.922E−04 | 7.516E−03 | 5.253E−04 | 6.235E−02 | 2.347E−02 | 4.428E−01 | 2.286E−01 | 1.00 |
| 8056113 | LY75 | 1.935E−04 | 7.529E−03 | 9.859E−04 | 7.598E−02 | 1.199E−02 | 3.586E−01 | 2.826E−01 | 1.00 |
| 7975521 | RBM25 | 1.956E−04 | 7.574E−03 | 7.602E−04 | 6.914E−02 | 1.449E−02 | 3.734E−01 | 3.088E−01 | 1.00 |
| 8108603 | HARS2 | 1.980E−04 | 7.598E−03 | 1.709E−04 | 4.758E−02 | 4.440E−03 | 2.740E−01 | 9.927E−01 | 1.00 |
| 7900395 | RLF | 1.992E−04 | 7.598E−03 | 1.918E−03 | 9.278E−02 | 6.269E−03 | 3.013E−01 | 3.079E−01 | 1.00 |
| 8038427 | TSKS | 1.993E−04 | 7.598E−03 | 1.871E−03 | 9.213E−02 | 9.744E−03 | 3.402E−01 | 2.016E−01 | 1.00 |
| 8010354 | GAA | 2.047E−04 | 7.764E−03 | 6.240E−03 | 1.478E−01 | 2.777E−03 | 2.518E−01 | 1.862E−01 | 1.00 |
| 7933947 | HERC4 | 2.075E−04 | 7.798E−03 | 7.674E−04 | 6.933E−02 | 8.614E−03 | 3.216E−01 | 5.529E−01 | 1.00 |
| 8094948 | SLAIN2 | 2.084E−04 | 7.798E−03 | 1.823E−03 | 9.213E−02 | 5.250E−03 | 2.944E−01 | 4.175E−01 | 1.00 |
| 7908841 | PPPIRI2B | 2.095E−04 | 7.798E−03 | 3.727E−02 | 3.432E−01 | 7.835E−04 | 2.229E−01 | 2.963E−02 | 1.00 |
| 8136662 | MGAM | 2.104E−04 | 7.798E−03 | 4.790E−03 | 1.320E−01 | 7.783E−03 | 3.150E−01 | 7.760E−02 | 1.00 |
| 8088745 | FRMD4B | 2.109E−04 | 7.798E−03 | 8.880E−03 | 1.740E−01 | 7.029E−03 | 3.044E−01 | 2.668E−02 | 1.00 |
| 8156199 | DAPK1 | 2.124E−04 | 7.798E−03 | 1.670E−03 | 9.035E−02 | 2.392E−02 | 4.442E−01 | 7.182E−02 | 1.00 |
| 8002592 | AP1G1 | 2.137E−04 | 7.798E−03 | 3.840E−03 | 1.215E−01 | 1.908E−03 | 2.384E−01 | 4.718E−01 | 1.00 |
| 8017599 | PECAM1 | 2.144E−04 | 7.798E−03 | 5.527E−03 | 1.396E−01 | 3.187E−03 | 2.558E−01 | 2.131E−01 | 1.00 |
| 7942174 | PPFIA1 | 2.174E−04 | 7.798E−03 | 2.300E−03 | 9.991E−02 | 3.857E−03 | 2.706E−01 | 4.565E−01 | 1.00 |
| 8106556 | CMYA5 | 2.185E−04 | 7.798E−03 | 3.341E−03 | 1.143E−01 | 4.914E−03 | 2.894E−01 | 2.629E−01 | 1.00 |
| 8170027 | DDX26B | 2.191E−04 | 7.798E−03 | 2.076E−04 | 5.313E−02 | 9.722E−03 | 3.402E−01 | 9.061E−01 | 1.00 |
| 7993478 | ABCC1 | 2.198E−04 | 7.798E−03 | 1.557E−04 | 4.519E−02 | 4.496E−03 | 2.750E−01 | 9.992E−01 | 1.00 |
| 8078479 | PDCD6IP | 2.199E−04 | 7.798E−03 | 2.158E−03 | 9.773E−02 | 7.653E−03 | 3.126E−01 | 2.697E−01 | 1.00 |
| 8011884 | NLRPI | 2.200E−04 | 7.798E−03 | 2.181E−03 | 9.773E−02 | 6.404E−03 | 3.013E−01 | 3.267E−01 | 1.00 |
| 8049906 | ING5 | 2.219E−04 | 7.827E−03 | 2.305E−04 | 5.313E−02 | 1.136E−02 | 3.530E−01 | 8.298E−01 | 1.00 |
| 7934393 | PPP3CB | 2.255E−04 | 7.876E−03 | 3.507E−04 | 5.595E−02 | 4.876E−03 | 2.894E−01 | 9.495E−01 | 1.00 |
| 7958749 | SH2B3 | 2.263E−04 | 7.876E−03 | 3.768E−03 | 1.207E−01 | 1.638E−02 | 3.893E−01 | 3.973E−02 | 1.00 |
| 8027304 | ZNF493 | 2.264E−04 | 7.876E−03 | 4.434E−03 | 1.268E−01 | 2.333E−03 | 2.410E−01 | 3.931E−01 | 1.00 |
| 8124553 | ZKSCAN4 | 2.301E−04 | 7.967E−03 | 7.169E−04 | 6.733E−02 | 5.879E−03 | 3.013E−01 | 7.508E−01 | 1.00 |
| 8056102 | CD302 | 2.317E−04 | 7.987E−03 | 8.655E−03 | 1.719E−01 | 3.721E−03 | 2.688E−01 | 1.074E−01 | 1.00 |
| 7933413 | BMS1P1 | 2.357E−04 | 8.073E−03 | 2.269E−02 | 2.738E−01 | 1.270E−03 | 2.383E−01 | 7.119E−02 | 1.00 |
| 8178727 | ATF6B | 2.363E−04 | 8.073E−03 | 2.520E−02 | 2.886E−01 | 8.270E−04 | 2.229E−01 | 9.856E−02 | 1.00 |
| 7926851 | WAC | 2.394E−04 | 8.140E−03 | 7.206E−04 | 6.733E−02 | 5.529E−03 | 2.958E−01 | 7.974E−01 | 1.00 |
| 7924526 | TP53BP2 | 2.478E−04 | 8.364E−03 | 1.945E−03 | 9.342E−02 | 1.101E−02 | 3.478E−01 | 2.570E−01 | 1.00 |
| 7969060 | FNDC3A | 2.483E−04 | 8.364E−03 | 1.377E−03 | 8.459E−02 | 1.006E−02 | 3.402E−01 | 3.824E−01 | 1.00 |
| 8019463 | CSNK1D | 2.493E−04 | 8.364E−03 | 6.887E−04 | 6.617E−02 | 6.276E−03 | 3.013E−01 | 7.871E−01 | 1.00 |
| 7980051 | C14orf43 | 2.577E−04 | 8.569E−03 | 9.687E−03 | 1.797E−01 | 3.026E−03 | 2.540E−01 | 1.533E−01 | 1.00 |
| 8120758 | SENP6 | 2.586E−04 | 8.569E−03 | 2.454E−03 | 1.039E−01 | 9.990E−03 | 3.402E−01 | 2.335E−01 | 1.00 |
| 7978997 | MAP4K5 | 2.591E−04 | 8.569E−03 | 8.343E−04 | 7.263E−02 | 1.293E−02 | 3.684E−01 | 4.945E−01 | 1.00 |
| 7974533 | PELI2 | 2.599E−04 | 8.569E−03 | 9.834E−03 | 1.809E−01 | 4.932E−03 | 2.894E−01 | 7.760E−02 | 1.00 |
| 8153959 | DOCK8 | 2.630E−04 | 8.631E−03 | 5.406E−03 | 1.387E−01 | 2.024E−02 | 4.222E−01 | 1.931E−02 | 1.00 |
| 8115783 | STK10 | 2.706E−04 | 8.814E−03 | 1.105E−02 | 1.922E−01 | 6.703E−03 | 3.018E−01 | 4.189E−02 | 1.00 |
| 8063814 | LSM14B | 2.714E−04 | 8.814E−03 | 8.399E−04 | 7.263E−02 | 1.063E−02 | 3.434E−01 | 5.912E−01 | 1.00 |
| 8096675 | TET2 | 2.721E−04 | 8.814E−03 | 5.081E−03 | 1.351E−01 | 1.153E−02 | 3.559E−01 | 7.651E−02 | 1.00 |
| 7938592 | FAR1 | 2.742E−04 | 8.847E−03 | 2.958E−03 | 1.076E−01 | 2.248E−02 | 4.330E−01 | 5.940E−02 | 1.00 |
| 8071069 | ILI7RA | 2.799E−04 | 8.929E−03 | 1.012E−02 | 1.826E−01 | 1.416E−02 | 3.734E−01 | 1.221E−02 | 1.00 |
| 8165674 | SH3KBP1 | 2.819E−04 | 8.929E−03 | 9.261E−03 | 1.762E−01 | 4.465E−03 | 2.744E−01 | 1.230E−01 | 1.00 |
| 8164701 | SETX | 2.820E−04 | 8.929E−03 | 1.568E−02 | 2.321E−01 | 2.343E−03 | 2.410E−01 | 1.101E−01 | 1.00 |
| 8119000 | MAPK14 | 2.825E−04 | 8.929E−03 | 6.792E−03 | 1.525E−01 | 1.013E−02 | 3.402E−01 | 6.157E−02 | 1.00 |
| 8079392 | CCR2 | 2.843E−04 | 8.929E−03 | 8.313E−03 | 1.693E−01 | 1.397E−02 | 3.734E−01 | 2.170E−02 | 1.00 |
| 8106354 | IQGAP2 | 2.851E−04 | 8.929E−03 | 1.405E−03 | 8.466E−02 | 1.853E−02 | 4.090E−01 | 2.481E−01 | 1.00 |
| 8090351 | ZXDC | 2.854E−04 | 8.929E−03 | 2.844E−03 | 1.070E−01 | 1.008E−02 | 3.402E−01 | 2.394E−01 | 1.00 |
| 8114050 | SEPT8 | 2.862E−04 | 8.929E−03 | 4.661E−03 | 1.303E−01 | 7.106E−03 | 3.048E−01 | 1.982E−01 | 1.00 |
| 8089000 | CGGBPI | 2.902E−04 | 9.014E−03 | 1.335E−03 | 8.431E−02 | 1.070E−02 | 3.442E−01 | 4.771E−01 | 1.00 |
| 8041913 | KLRAQ1 | 2.941E−04 | 9.098E−03 | 6.696E−04 | 6.617E−02 | 8.903E−03 | 3.283E−01 | 7.897E−01 | 1.00 |
| 8151890 | TP53INPI | 2.969E−04 | 9.147E−03 | 2.011E−02 | 2.605E−01 | 8.998E−04 | 2.361E−01 | 2.371E−01 | 1.00 |
| 8099410 | BOD1L | 2.988E−04 | 9.151E−03 | 8.895E−03 | 1.740E−01 | 5.836E−03 | 3.013E−01 | 1.062E−01 | 1.00 |
| 7986767 | C15orf49 | 3.001E−04 | 9.151E−03 | 2.598E−03 | 1.058E−01 | 1.808E−03 | 2.383E−01 | 8.488E−01 | 1.00 |
| 8067011 | ADNP | 3.009E−04 | 9.151E−03 | 5.115E−04 | 6.235E−02 | 4.650E−03 | 2.807E−01 | 9.754E−01 | 1.00 |
| 8053576 | RNF103 | 3.018E−04 | 9.151E−03 | 1.130E−03 | 8.029E−02 | 3.864E−03 | 2.706E−01 | 8.774E−01 | 1.00 |
| 7980338 | IRF2BPL | 3.071E−04 | 9.228E−03 | 4.188E−03 | 1.242E−01 | 6.976E−03 | 3.038E−01 | 2.666E−01 | 1.00 |
| 8126402 | TRERF1 | 3.078E−04 | 9.228E−03 | 3.381E−04 | 5.595E−02 | 1.002E−02 | 3.402E−01 | 9.306E−01 | 1.00 |
| 8143088 | CNOT4 | 3.080E−04 | 9.228E−03 | 2.184E−03 | 9.773E−02 | 7.625E−03 | 3.124E−01 | 4.731E−01 | 1.00 |
| 8166876 | DDX3X | 3.118E−04 | 9.303E−03 | 3.729E−03 | 1.201E−01 | 1.176E−02 | 3.582E−01 | 1.689E−01 | 1.00 |
| 8052554 | FAM161A | 3.199E−04 | 9.509E−03 | 2.205E−03 | 9.799E−02 | 1.706E−03 | 2.383E−01 | 9.251E−01 | 1.00 |
| 8045514 | SPOPL | 3.247E−04 | 9.532E−03 | 6.045E−03 | 1.457E−01 | 8.818E−03 | 3.269E−01 | 1.406E−01 | 1.00 |
| 7986350 | ARRDC4 | 3.268E−04 | 9.532E−03 | 1.006E−02 | 1.823E−01 | 4.278E−03 | 2.733E−01 | 1.597E−01 | 1.00 |
| 8133788 | PTPN12 | 3.270E−04 | 9.532E−03 | 5.135E−03 | 1.352E−01 | 9.776E−03 | 3.402E−01 | 1.618E−01 | 1.00 |
| 8139430 | PURB | 3.276E−04 | 9.532E−03 | 1.101E−03 | 7.985E−02 | 2.918E−03 | 2.521E−01 | 9.703E−01 | 1.00 |
| 8099760 | ARAP2 | 3.288E−04 | 9.532E−03 | 1.258E−03 | 8.210E−02 | 1.460E−02 | 3.734E−01 | 4.428E−01 | 1.00 |
| 7929511 | ENTPD1 | 3.302E−04 | 9.532E−03 | 6.751E−03 | 1.525E−01 | 9.903E−03 | 3.402E−01 | 1.013E−01 | 1.00 |
| 8063607 | RAB22A | 3.307E−04 | 9.532E−03 | 6.355E−03 | 1.483E−01 | 2.500E−03 | 2.448E−01 | 4.752E−01 | 1.00 |
| 8019885 | SMCHD1 | 3.308E−04 | 9.532E−03 | 1.968E−02 | 2.593E−01 | 1.058E−02 | 3.429E−01 | 6.974E−03 | 1.00 |
| 8123644 | TUBB2A | 3.379E−04 | 9.698E−03 | 4.851E−03 | 1.324E−01 | 1.215E−02 | 3.588E−01 | 1.339E−01 | 1.00 |
| 7952707 | PRDM10 | 3.403E−04 | 9.718E−03 | 2.521E−03 | 1.045E−01 | 5.142E−03 | 2.929E−01 | 6.327E−01 | 1.00 |
| 8154151 | PPAPDC2 | 3.423E−04 | 9.718E−03 | 1.027E−03 | 7.699E−02 | 5.195E−03 | 2.929E−01 | 9.014E−01 | 1.00 |
| 8140782 | ABCB1 | 3.434E−04 | 9.718E−03 | 6.797E−03 | 1.525E−01 | 2.037E−03 | 2.406E−01 | 5.366E−01 | 1.00 |
| 8102862 | MAML3 | 3.463E−04 | 9.718E−03 | 2.459E−03 | 1.039E−01 | 1.339E−02 | 3.697E−01 | 2.974E−01 | 1.00 |
| 8131614 | AHR | 3.468E−04 | 9.718E−03 | 6.692E−04 | 6.617E−02 | 2.488E−02 | 4.495E−01 | 4.757E−01 | 1.00 |
| 8037433 | ZNF45 | 3.468E−04 | 9.718E−03 | 8.435E−04 | 7.263E−02 | 8.402E−03 | 3.206E−01 | 8.356E−01 | 1.00 |
| 7966003 | APPL2 | 3.487E−04 | 9.718E−03 | 3.575E−03 | 1.176E−01 | 6.071E−03 | 3.013E−01 | 4.421E−01 | 1.00 |
| 7986383 | IGF1R | 3.489E−04 | 9.718E−03 | 1.098E−03 | 7.985E−02 | 2.083E−02 | 4.234E−01 | 3.860E−01 | 1.00 |
| 8052149 | PSME4 | 3.526E−04 | 9.786E−03 | 9.469E−04 | 7.467E−02 | 3.222E−03 | 2.570E−01 | 9.818E−01 | 1.00 |
| 7966851 | TAOK3 | 3.593E−04 | 9.936E−03 | 1.376E−03 | 8.459E−02 | 5.504E−03 | 2.958E−01 | 8.438E−01 | 1.00 |
| 8021312 | WDR7 | 3.632E−04 | 1.001E−02 | 1.124E−03 | 8.029E−02 | 8.553E−03 | 3.214E−01 | 7.798E−01 | 1.00 |
| 8047248 | PLCLI | 3.650E−04 | 1.002E−02 | 5.315E−04 | 6.235E−02 | 9.887E−03 | 3.402E−01 | 9.169E−01 | 1.00 |
| 8105191 | PARP8 | 3.673E−04 | 1.002E−02 | 4.378E−03 | 1.261E−01 | 6.748E−03 | 3.018E−01 | 3.599E−01 | 1.00 |
| 8014841 | MED1 | 3.717E−04 | 1.002E−02 | 1.586E−03 | 8.831E−02 | 1.065E−02 | 3.434E−01 | 5.644E−01 | 1.00 |
| 7961767 | KIAA0528 | 3.717E−04 | 1.002E−02 | 8.906E−04 | 7.324E−02 | 1.633E−02 | 3.888E−01 | 6.295E−01 | 1.00 |
| 8026365 | ZNF333 | 3.758E−04 | 1.002E−02 | 1.566E−02 | 2.320E−01 | 4.987E−03 | 2.894E−01 | 8.796E−02 | 1.00 |
| 7970602 | PARP4 | 3.766E−04 | 1.002E−02 | 5.435E−04 | 6.283E−02 | 2.830E−02 | 4.603E−01 | 5.536E−01 | 1.00 |
| 7957260 | GLIPR1 | 3.781E−04 | 1.002E−02 | 2.714E−03 | 1.066E−01 | 1.428E−02 | 3.734E−01 | 3.000E−01 | 1.00 |
| 7925565 | HNRNPU | 3.786E−04 | 1.002E−02 | 1.232E−03 | 8.193E−02 | 8.503E−03 | 3.206E−01 | 7.721E−01 | 1.00 |
| 7907773 | TOR1AIP1 | 3.787E−04 | 1.002E−02 | 8.907E−03 | 1.740E−01 | 4.326E−03 | 2.733E−01 | 2.570E−01 | 1.00 |
| 7905444 | SNX27 | 3.793E−04 | 1.002E−02 | 2.108E−02 | 2.668E−01 | 4.995E−03 | 2.894E−01 | 4.684E−02 | 1.00 |
| 8015152 | KRT40 | 3.793E−04 | 1.002E−02 | 3.450E−03 | 1.160E−01 | 8.871E−03 | 3.280E−01 | 3.733E−01 | 1.00 |
| 8093961 | KIAA0232 | 3.803E−04 | 1.002E−02 | 3.053E−03 | 1.102E−01 | 7.834E−03 | 3.159E−01 | 4.603E−01 | 1.00 |
| 8056545 | STK39 | 3.810E−04 | 1.002E−02 | 5.309E−04 | 6.235E−02 | 6.550E−03 | 3.018E−01 | 9.831E−01 | 1.00 |
| 8041236 | SPAST | 3.826E−04 | 1.003E−02 | 1.270E−03 | 8.253E−02 | 1.539E−02 | 3.808E−01 | 5.209E−01 | 1.00 |
| 8168345 | ACRC | 3.859E−04 | 1.008E−02 | 2.759E−04 | 5.377E−02 | 1.347E−02 | 3.705E−01 | 9.688E−01 | 1.00 |
| 8063785 | C20orf197 | 3.883E−04 | 1.011E−02 | 1.214E−03 | 8.193E−02 | 1.748E−02 | 3.994E−01 | 5.091E−01 | 1.00 |
| 8106784 | RASA1 | 3.928E−04 | 1.016E−02 | 8.465E−04 | 7.263E−02 | 8.054E−03 | 3.187E−01 | 9.046E−01 | 1.00 |
| 7902822 | PKN2 | 3.931E−04 | 1.016E−02 | 7.343E−03 | 1.565E−01 | 1.019E−02 | 3.402E−01 | 1.327E−01 | 1.00 |
| 8168875 | ARMCX3 | 3.942E−04 | 1.016E−02 | 8.285E−04 | 7.263E−02 | 2.159E−02 | 4.262E−01 | 5.704E−01 | 1.00 |
| 8078738 | OXSRI | 3.960E−04 | 1.017E−02 | 6.769E−04 | 6.617E−02 | 1.360E−02 | 3.708E−01 | 8.227E−01 | 1.00 |
| 8052233 | C2orf63 | 3.995E−04 | 1.022E−02 | 1.988E−03 | 9.410E−02 | 2.756E−03 | 2.518E−01 | 9.455E−01 | 1.00 |
| 8025964 | ZNF439 | 4.034E−04 | 1.028E−02 | 2.688E−04 | 5.313E−02 | 1.288E−02 | 3.683E−01 | 9.845E−01 | 1.00 |
| 7943314 | JRKL | 4.043E−04 | 1.028E−02 | 2.028E−03 | 9.508E−02 | 7.548E−03 | 3.115E−01 | 6.808E−01 | 1.00 |
| 8041179 | CLIP4 | 4.057E−04 | 1.028E−02 | 1.790E−03 | 9.197E−02 | 1.485E−02 | 3.734E−01 | 4.543E−01 | 1.00 |
| 7954511 | STK38L | 4.082E−04 | 1.031E−02 | 4.511E−03 | 1.279E−01 | 5.594E−03 | 2.958E−01 | 4.828E−01 | 1.00 |
| 7933115 | SEPT7L | 4.101E−04 | 1.032E−02 | 3.336E−02 | 3.274E−01 | 1.473E−03 | 2.383E−01 | 1.243E−01 | 1.00 |
| 7935320 | TM9SF3 | 4.118E−04 | 1.033E−02 | 2.271E−04 | 5.313E−02 | 2.018E−02 | 4.222E−01 | 9.459E−01 | 1.00 |
| 7965652 | CDK17 | 4.156E−04 | 1.038E−02 | 2.850E−03 | 1.070E−01 | 1.170E−02 | 3.582E−01 | 4.026E−01 | 1.00 |
| 7935403 | ARHGAP19 | 4.165E−04 | 1.038E−02 | 2.718E−03 | 1.066E−01 | 1.360E−03 | 2.383E−01 | 9.875E−01 | 1.00 |
| 7944667 | SORL1 | 4.187E−04 | 1.040E−02 | 1.258E−02 | 2.080E−01 | 7.367E−03 | 3.092E−01 | 9.988E−02 | 1.00 |
| 8107474 | DMXL1 | 4.252E−04 | 1.052E−02 | 2.639E−04 | 5.313E−02 | 2.290E−02 | 4.356E−01 | 9.079E−01 | 1.00 |
| 8064868 | GPCPD1 | 4.276E−04 | 1.054E−02 | 3.147E−03 | 1.117E−01 | 2.438E−03 | 2.443E−01 | 9.015E−01 | 1.00 |
| 8132013 | CHN2 | 4.288E−04 | 1.054E−02 | 2.744E−03 | 1.066E−01 | 1.421E−02 | 3.734E−01 | 3.693E−01 | 1.00 |
| 8102006 | MANBA | 4.309E−04 | 1.056E−02 | 2.681E−04 | 5.313E−02 | 1.446E−02 | 3.734E−01 | 9.868E−01 | 1.00 |
| 8050190 | ADAMI7 | 4.333E−04 | 1.058E−02 | 1.015E−03 | 7.693E−02 | 8.720E−03 | 3.241E−01 | 8.841E−01 | 1.00 |
| 7993433 | PDXDC1 | 4.360E−04 | 1.058E−02 | 1.331E−03 | 8.431E−02 | 6.648E−03 | 3.018E−01 | 8.847E−01 | 1.00 |
| 7958532 | UBE3B | 4.360E−04 | 1.058E−02 | 5.315E−04 | 6.235E−02 | 3.828E−03 | 2.706E−01 | 9.999E−01 | 1.00 |
| 8025973 | ZNF700 | 4.423E−04 | 1.065E−02 | 8.912E−04 | 7.324E−02 | 1.627E−02 | 3.888E−01 | 7.359E−01 | 1.00 |
| 8091863 | SLITRK3 | 4.437E−04 | 1.065E−02 | 2.178E−02 | 2.701E−01 | 1.462E−03 | 2.383E−01 | 3.019E−01 | 1.00 |
| 8045381 | CCNT2 | 4.443E−04 | 1.065E−02 | 2.269E−03 | 9.954E−02 | 1.484E−02 | 3.734E−01 | 4.331E−01 | 1.00 |
| 7942603 | MOGAT2 | 4.446E−04 | 1.065E−02 | 5.901E−02 | 4.206E−01 | 1.583E−03 | 2.383E−01 | 3.587E−02 | 1.00 |
| 7948455 | MS4A6A | 4.482E−04 | 1.070E−02 | 4.250E−03 | 1.244E−01 | 2.330E−02 | 4.410E−01 | 1.234E−01 | 1.00 |
| 7995479 | PAPD5 | 4.492E−04 | 1.070E−02 | 4.423E−04 | 5.735E−02 | 2.213E−02 | 4.296E−01 | 8.328E−01 | 1.00 |
| 8022531 | NPC1 | 4.525E−04 | 1.074E−02 | 4.746E−03 | 1.318E−01 | 7.427E−03 | 3.108E−01 | 4.314E−01 | 1.00 |
| 8059413 | DOCK10 | 4.551E−04 | 1.075E−02 | 9.563E−04 | 7.498E−02 | 1.707E−02 | 3.942E−01 | 7.241E−01 | 1.00 |
| 7966046 | MTERFD3 | 4.557E−04 | 1.075E−02 | 1.975E−03 | 9.410E−02 | 7.764E−03 | 3.150E−01 | 7.580E−01 | 1.00 |
| 8036820 | ZNF780A | 4.626E−04 | 1.085E−02 | 2.955E−03 | 1.076E−01 | 1.431E−02 | 3.734E−01 | 3.913E−01 | 1.00 |
| 7958216 | KIAA1033 | 4.646E−04 | 1.085E−02 | 1.372E−03 | 8.459E−02 | 1.553E−02 | 3.818E−01 | 6.398E−01 | 1.00 |
| 8174893 | THOC2 | 4.652E−04 | 1.085E−02 | 3.869E−03 | 1.217E−01 | 9.611E−03 | 3.400E−01 | 4.348E−01 | 1.00 |
| 8166826 | USP9X | 4.656E−04 | 1.085E−02 | 3.538E−03 | 1.171E−01 | 3.528E−03 | 2.688E−01 | 8.259E−01 | 1.00 |
| 8070269 | DSCR3 | 4.681E−04 | 1.085E−02 | 4.776E−03 | 1.320E−01 | 3.621E−03 | 2.688E−01 | 7.109E−01 | 1.00 |
| 8113733 | CEP120 | 4.684E−04 | 1.085E−02 | 9.467E−03 | 1.784E−01 | 7.531E−03 | 3.115E−01 | 2.084E−01 | 1.00 |
| 8176624 | DDX3Y | 4.725E−04 | 1.088E−02 | 2.393E−04 | 5.313E−02 | 3.954E−02 | 5.038E−01 | 7.991E−01 | 1.00 |
| 8169750 | STAG2 | 4.725E−04 | 1.088E−02 | 9.794E−04 | 7.592E−02 | 1.455E−02 | 3.734E−01 | 7.783E−01 | 1.00 |
| 7968761 | NAAI6 | 4.740E−04 | 1.088E−02 | 1.251E−03 | 8.203E−02 | 1.280E−02 | 3.675E−01 | 7.625E−01 | 1.00 |
| 7924874 | TRIM17 | 4.769E−04 | 1.090E−02 | 2.453E−03 | 1.039E−01 | 5.488E−03 | 2.958E−01 | 8.275E−01 | 1.00 |
| 8115524 | CLINT1 | 4.781E−04 | 1.090E−02 | 1.401E−03 | 8.466E−02 | 1.476E−02 | 3.734E−01 | 6.838E−01 | 1.00 |
| 7915286 | PPT1 | 4.794E−04 | 1.090E−02 | 5.336E−03 | 1.381E−01 | 1.269E−02 | 3.667E−01 | 2.469E−01 | 1.00 |
| 8058161 | ORC2 | 4.818E−04 | 1.091E−02 | 2.817E−03 | 1.070E−01 | 4.094E−03 | 2.733E−01 | 8.761E−01 | 1.00 |
| 7936904 | CTBP2 | 4.828E−04 | 1.091E−02 | 5.507E−03 | 1.396E−01 | 1.933E−02 | 4.172E−01 | 1.351E−01 | 1.00 |
| 7925048 | EGLN1 | 4.884E−04 | 1.100E−02 | 9.626E−03 | 1.794E−01 | 8.236E−03 | 3.187E−01 | 1.997E−01 | 1.00 |
| 7984319 | MAP2K1 | 4.899E−04 | 1.100E−02 | 1.007E−03 | 7.693E−02 | 8.270E−03 | 3.187E−01 | 9.435E−01 | 1.00 |
| 8116227 | CLK4 | 4.912E−04 | 1.100E−02 | 8.893E−04 | 7.324E−02 | 2.411E−02 | 4.442E−01 | 6.391E−01 | 1.00 |
| 7925201 | ARID4B | 4.978E−04 | 1.110E−02 | 2.159E−02 | 2.694E−01 | 6.038E−03 | 3.013E−01 | 7.715E−02 | 1.00 |
| 7960165 | ZNF268 | 4.984E−04 | 1.110E−02 | 8.564E−04 | 7.302E−02 | 5.847E−03 | 3.013E−01 | 9.944E−01 | 1.00 |
| 7936683 | TIAL1 | 4.998E−04 | 1.110E−02 | 3.882E−03 | 1.217E−01 | 3.712E−03 | 2.688E−01 | 8.174E−01 | 1.00 |
| 7974473 | FBXO34 | 5.039E−04 | 1.116E−02 | 1.560E−03 | 8.805E−02 | 7.705E−03 | 3.138E−01 | 8.905E−01 | 1.00 |
| 8110486 | ZNFS79 | 5.095E−04 | 1.125E−02 | 1.892E−03 | 9.251E−02 | 8.041E−03 | 3.187E−01 | 8.227E−01 | 1.00 |
| 8017634 | DDX5 | 5.123E−04 | 1.127E−02 | 1.377E−03 | 8.459E−02 | 3.024E−02 | 4.709E−01 | 4.235E−01 | 1.00 |
| 8137437 | GALNTL5 | 5.152E−04 | 1.130E−02 | 1.867E−01 | 7.239E−01 | 7.339E−04 | 2.212E−01 | 2.157E−03 | 1.00 |
| 8011018 | CRK | 5.162E−04 | 1.130E−02 | 6.319E−03 | 1.483E−01 | 1.165E−02 | 3.582E−01 | 2.594E−01 | 1.00 |
| 8073943 | ZBED4 | 5.229E−04 | 1.140E−02 | 4.784E−03 | 1.320E−01 | 6.057E−03 | 3.013E−01 | 6.002E−01 | 1.00 |
| 8111339 | MTMR12 | 5.238E−04 | 1.140E−02 | 2.854E−03 | 1.070E−01 | 4.989E−03 | 2.894E−01 | 8.589E−01 | 1.00 |
| 7985605 | ZNF592 | 5.277E−04 | 1.145E−02 | 4.366E−03 | 1.261E−01 | 8.628E−03 | 3.216E−01 | 5.185E−01 | 1.00 |
| 8022488 | ABHD3 | 5.319E−04 | 1.147E−02 | 2.738E−03 | 1.066E−01 | 3.309E−02 | 4.802E−01 | 1.976E−01 | 1.00 |
| 7975416 | PCNX | 5.320E−04 | 1.147E−02 | 4.963E−02 | 3.913E−01 | 2.243E−03 | 2.410E−01 | 6.000E−02 | 1.00 |
| 8078187 | PLCL2 | 5.361E−04 | 1.151E−02 | 2.534E−04 | 5.313E−02 | 1.700E−02 | 3.942E−01 | 9.985E−01 | 1.00 |
| 8152222 | AZIN1 | 5.369E−04 | 1.151E−02 | 4.390E−03 | 1.261E−01 | 1.147E−02 | 3.549E−01 | 4.222E−01 | 1.00 |
| 7965606 | HAL | 5.396E−04 | 1.154E−02 | 2.860E−02 | 3.036E−01 | 3.692E−03 | 2.688E−01 | 1.123E−01 | 1.00 |
| 7928558 | ZMIZ1 | 5.420E−04 | 1.156E−02 | 2.767E−03 | 1.066E−01 | 1.829E−02 | 4.066E−01 | 4.176E−01 | 1.00 |
| 8081431 | ALCAM | 5.447E−04 | 1.158E−02 | 6.744E−04 | 6.617E−02 | 3.952E−02 | 5.038E−01 | 5.889E−01 | 1.00 |
| 8148049 | NOV | 5.480E−04 | 1.158E−02 | 8.370E−02 | 4.971E−01 | 1.369E−03 | 2.383E−01 | 3.032E−02 | 1.00 |
| 8106141 | FCHO2 | 5.485E−04 | 1.158E−02 | 1.051E−02 | 1.870E−01 | 5.513E−03 | 2.958E−01 | 3.361E−01 | 1.00 |
| 8028991 | CYP2S1 | 5.492E−04 | 1.158E−02 | 3.681E−02 | 3.426E−01 | 2.886E−03 | 2.521E−01 | 9.412E−02 | 1.00 |
| 8161988 | UBQLN1 | 5.629E−04 | 1.179E−02 | 5.185E−04 | 6.235E−02 | 1.580E−02 | 3.842E−01 | 9.733E−01 | 1.00 |
| 8081256 | TBC1D23 | 5.652E−04 | 1.179E−02 | 6.400E−03 | 1.487E−01 | 9.976E−03 | 3.402E−01 | 3.614E−01 | 1.00 |
| 8056977 | NFE2L2 | 5.655E−04 | 1.179E−02 | 6.896E−03 | 1.530E−01 | 2.152E−02 | 4.261E−01 | 1.214E−01 | 1.00 |
| 8104506 | TRIO | 5.656E−04 | 1.179E−02 | 5.901E−04 | 6.443E−02 | 1.266E−02 | 3.667E−01 | 9.880E−01 | 1.00 |
| 7956910 | CAND1 | 5.672E−04 | 1.180E−02 | 1.673E−03 | 9.035E−02 | 1.004E−02 | 3.402E−01 | 8.600E−01 | 1.00 |
| 8028266 | ZNF540 | 5.698E−04 | 1.182E−02 | 6.391E−04 | 6.617E−02 | 7.316E−03 | 3.080E−01 | 9.998E−01 | 1.00 |
| 7915160 | RRAGC | 5.753E−04 | 1.184E−02 | 3.846E−03 | 1.215E−01 | 4.108E−03 | 2.733E−01 | 8.706E−01 | 1.00 |
| 8091658 | CCNL1 | 5.757E−04 | 1.184E−02 | 5.968E−03 | 1.446E−01 | 1.973E−02 | 4.198E−01 | 1.760E−01 | 1.00 |
| 8129804 | MAP3K5 | 5.757E−04 | 1.184E−02 | 8.292E−03 | 1.693E−01 | 1.938E−02 | 4.173E−01 | 1.131E−01 | 1.00 |
| 8139632 | FIGNL1 | 5.776E−04 | 1.185E−02 | 2.900E−03 | 1.075E−01 | 1.314E−02 | 3.697E−01 | 5.794E−01 | 1.00 |
| 8094599 | KLF3 | 5.810E−04 | 1.189E−02 | 3.821E−03 | 1.212E−01 | 9.454E−03 | 3.385E−01 | 6.023E−01 | 1.00 |
| 8150714 | PCMTD1 | 5.866E−04 | 1.189E−02 | 3.299E−03 | 1.143E−01 | 2.710E−02 | 4.521E−01 | 2.586E−01 | 1.00 |
| 8088247 | ARHGEF3 | 5.876E−04 | 1.189E−02 | 5.914E−04 | 6.443E−02 | 3.669E−02 | 4.946E−01 | 7.130E−01 | 1.00 |
| 8037856 | ZC3H4 | 5.890E−04 | 1.189E−02 | 1.180E−03 | 8.180E−02 | 5.188E−03 | 2.929E−01 | 9.980E−01 | 1.00 |
| 8172504 | GRIPAP1 | 5.894E−04 | 1.189E−02 | 5.469E−02 | 4.087E−01 | 2.552E−03 | 2.448E−01 | 5.434E−02 | 1.00 |
| 8108217 | TGFBI | 5.919E−04 | 1.189E−02 | 2.815E−03 | 1.070E−01 | 3.313E−02 | 4.802E−01 | 2.465E−01 | 1.00 |
| 8096781 | SEC24B | 5.935E−04 | 1.189E−02 | 1.799E−03 | 9.197E−02 | 1.364E−02 | 3.708E−01 | 7.588E−01 | 1.00 |
| 8012961 | NCOR1 | 5.940E−04 | 1.189E−02 | 6.775E−03 | 1.525E−01 | 3.677E−03 | 2.688E−01 | 7.236E−01 | 1.00 |
| 8162236 | SEMA4D | 5.957E−04 | 1.189E−02 | 7.442E−04 | 6.860E−02 | 1.523E−02 | 3.777E−01 | 9.446E−01 | 1.00 |
| 7985166 | IREB2 | 5.969E−04 | 1.189E−02 | 3.685E−02 | 3.426E−01 | 3.869E−03 | 2.706E−01 | 7.876E−02 | 1.00 |
| 7980680 | FOXN3 | 5.972E−04 | 1.189E−02 | 2.378E−03 | 1.020E−01 | 6.302E−03 | 3.013E−01 | 9.077E−01 | 1.00 |
| 8111677 | LIFR | 6.016E−04 | 1.195E−02 | 4.052E−02 | 3.568E−01 | 1.610E−03 | 2.383E−01 | 1.954E−01 | 1.00 |
| 7942520 | LOC100287896 | 6.056E−04 | 1.199E−02 | 4.868E−03 | 1.326E−01 | 3.520E−03 | 2.688E−01 | 8.716E−01 | 1.00 |
| 8063739 | PHACTR3 | 6.068E−04 | 1.199E−02 | 1.719E−02 | 2.415E−01 | 2.317E−03 | 2.410E−01 | 4.633E−01 | 1.00 |
| 7908553 | PTPRC | 6.088E−04 | 1.200E−02 | 2.908E−03 | 1.075E−01 | 5.063E−02 | 5.372E−01 | 1.243E−01 | 1.00 |
| 8034334 | ZNF20 | 6.104E−04 | 1.200E−02 | 6.295E−03 | 1.482E−01 | 6.228E−03 | 3.013E−01 | 5.892E−01 | 1.00 |
| 8107942 | RAD50 | 6.135E−04 | 1.201E−02 | 1.648E−02 | 2.384E−01 | 4.555E−03 | 2.774E−01 | 2.891E−01 | 1.00 |
| 8046333 | CYBRD1 | 6.141E−04 | 1.201E−02 | 2.577E−03 | 1.056E−01 | 3.227E−02 | 4.790E−01 | 3.030E−01 | 1.00 |
| 7978739 | TRAPPC6B | 6.173E−04 | 1.204E−02 | 3.246E−03 | 1.138E−01 | 2.132E−02 | 4.261E−01 | 3.868E−01 | 1.00 |
| 8045090 | UGGT1 | 6.225E−04 | 1.211E−02 | 6.120E−02 | 4.271E−01 | 3.091E−03 | 2.556E−01 | 3.485E−02 | 1.00 |
| 7964555 | AVIL | 6.361E−04 | 1.234E−02 | 2.206E−02 | 2.714E−01 | 2.814E−03 | 2.518E−01 | 3.263E−01 | 1.00 |
| 8014551 | SYNRG | 6.425E−04 | 1.237E−02 | 2.006E−02 | 2.605E−01 | 5.291E−03 | 2.944E−01 | 2.073E−01 | 1.00 |
| 7956670 | USP15 | 6.443E−04 | 1.237E−02 | 5.187E−02 | 3.970E−01 | 6.029E−03 | 3.013E−01 | 1.927E−02 | 1.00 |
| 8062492 | RALGAPB | 6458E−04 | 1.237E−02 | 1.417E−02 | 2.196E−01 | 4.345E−03 | 2.733E−01 | 3.914E−01 | 1.00 |
| 8011324 | ORIGI | 6.459E−04 | 1.237E−02 | 7.240E−03 | 1.555E−01 | 1.336E−02 | 3.697E−01 | 2.899E−01 | 1.00 |
| 8162352 | NOL8 | 6.479E−04 | 1.237E−02 | 8.758E−04 | 7.324E−02 | 2.333E−02 | 4.410E−01 | 8.418E−01 | 1.00 |
| 8057377 | CCDCI4I | 6.501E−04 | 1.237E−02 | 7.048E−04 | 6.712E−02 | 1.016E−02 | 3.402E−01 | 9.978E−01 | 1.00 |
| 7988286 | SPG11 | 6.504E−04 | 1.237E−02 | 2.278E−03 | 9.962E−02 | 1.593E−02 | 3.859E−01 | 6.730E−01 | 1.00 |
| 8120043 | RUNX2 | 6.504E−04 | 1.237E−02 | 8.929E−03 | 1.740E−01 | 2.876E−03 | 2.521E−01 | 7.536E−01 | 1.00 |
| 7943231 | ANKRD49 | 6.532E−04 | 1.239E−02 | 2.104E−03 | 9.675E−02 | 1.744E−02 | 3.993E−01 | 6.807E−01 | 1.00 |
| 7917604 | ZNF644 | 6.552E−04 | 1.240E−02 | 1.706E−03 | 9.090E−02 | 1.498E−02 | 3.748E−01 | 8.012E−01 | 1.00 |
| 7906330 | CDID | 6.646E−04 | 1.252E−02 | 4.025E−03 | 1.240E−01 | 4.006E−02 | 5.040E−01 | 1.483E−01 | 1.00 |
| 8043945 | MAP4K4 | 6.672E−04 | 1.252E−02 | 9.757E−03 | 1.801E−01 | 1.512E−02 | 3.760E−01 | 1.798E−01 | 1.00 |
| 7925257 | LYST | 6.674E−04 | 1.252E−02 | 4.576E−03 | 1.290E−01 | 2.201E−02 | 4.296E−01 | 3.042E−01 | 1.00 |
| 8095760 | THAP6 | 6.685E−04 | 1.252E−02 | 5.090E−03 | 1.351E−01 | 3.932E−03 | 2.709E−01 | 8.737E−01 | 1.00 |
| 7952022 | AMICA1 | 6.803E−04 | 1.270E−02 | 1.727E−02 | 2.420E−01 | 2.413E−02 | 4.442E−01 | 2.698E−02 | 1.00 |
| 7939341 | CD44 | 6.821E−04 | 1.270E−02 | 1.783E−03 | 9.197E−02 | 4.594E−02 | 5.247E−01 | 3.413E−01 | 1.00 |
| 7946610 | EIF4G2 | 6.833E−04 | 1.270E−02 | 2.750E−03 | 1.066E−01 | 2.657E−02 | 4.521E−01 | 4.233E−01 | 1.00 |
| 8099364 | ZNF5I8B | 6.853E−04 | 1.271E−02 | 3.876E−03 | 1.217E−01 | 3.654E−03 | 2.688E−01 | 9.552E−01 | 1.00 |
| 8083092 | ZBTB38 | 6.874E−04 | 1.272E−02 | 6.433E−03 | 1.489E−01 | 1.353E−02 | 3.708E−01 | 3.646E−01 | 1.00 |
| 7931951 | SFMBT2 | 6.890E−04 | 1.272E−02 | 5.491E−04 | 6.295E−02 | 3.040E−02 | 4.717E−01 | 8.948E−01 | 1.00 |
| 8035905 | ANKRD27 | 6.912E−04 | 1.273E−02 | 1.665E−03 | 9.035E−02 | 9.156E−03 | 3.349E−01 | 9.596E−01 | 1.00 |
| 7923659 | PPP1R15B | 6.955E−04 | 1.277E−02 | 5.162E−03 | 1.355E−01 | 1.835E−02 | 4.066E−01 | 3.503E−01 | 1.00 |
| 8019018 | CBX4 | 7.002E−04 | 1.283E−02 | 6.927E−03 | 1.532E−01 | 1.047E−02 | 3.428E−01 | 4.466E−01 | 1.00 |
| 8056798 | SP3 | 7.061E−04 | 1.289E−02 | 2.871E−03 | 1.070E−01 | 3.055E−02 | 4.725E−01 | 3.780E−01 | 1.00 |
| 7903049 | CCDC18 | 7.071E−04 | 1.289E−02 | 6.784E−03 | 1.525E−01 | 6.175E−03 | 3.013E−01 | 6.676E−01 | 1.00 |
| 8066848 | PREX1 | 7.165E−04 | 1.303E−02 | 4.371E−03 | 1.261E−01 | 9.918E−03 | 3.402E−01 | 6.808E−01 | 1.00 |
| 7959314 | SETD1B | 7.216E−04 | 1.309E−02 | 1.562E−03 | 8.805E−02 | 1.358E−02 | 3.708E−01 | 9.041E−01 | 1.00 |
| 7966268 | GIT2 | 7.258E−04 | 1.314E−02 | 4.086E−03 | 1.241E−01 | 6.252E−03 | 3.013E−01 | 8.679E−01 | 1.00 |
| 8068974 | TRAPPC10 | 7.332E−04 | 1.324E−02 | 7.930E−04 | 7.071E−02 | 3.187E−02 | 4.783E−01 | 8.181E−01 | 1.00 |
| 8065580 | DUSP15 | 7.360E−04 | 1.325E−02 | 5.419E−03 | 1.387E−01 | 3.133E−02 | 4.725E−01 | 1.896E−01 | 1.00 |
| 8147221 | OSGIN2 | 7.369E−04 | 1.325E−02 | 3.836E−02 | 3.482E−01 | 3.019E−03 | 2.540E−01 | 1.822E−01 | 1.00 |
| 8129590 | STX7 | 7.393E−04 | 1.326E−02 | 4.232E−03 | 1.244E−01 | 1.020E−02 | 3.402E−01 | 7.089E−01 | 1.00 |
| 8139832 | ZNF117 | 7.422E−04 | 1.328E−02 | 3.838E−02 | 3.482E−01 | 1.544E−02 | 3.808E−01 | 1.095E−02 | 1.00 |
| 8005814 | NLK | 7.468E−04 | 1.333E−02 | 4.850E−04 | 6.070E−02 | 1.800E−02 | 4.045E−01 | 9.985E−01 | 1.00 |
| 7943288 | SRSF8 | 7.576E−04 | 1.348E−02 | 4.754E−04 | 6.005E−02 | 2.426E−02 | 4.443E−01 | 9.842E−01 | 1.00 |
| 8111552 | NADKD1 | 7.608E−04 | 1.348E−02 | 1.624E−02 | 2.370E−01 | 3.985E−03 | 2.718E−01 | 4.780E−01 | 1.00 |
| 8150599 | PRKDC | 7.620E−04 | 1.348E−02 | 1.168E−02 | 1.992E−01 | 8.505E−03 | 3.206E−01 | 3.610E−01 | 1.00 |
| 8078214 | RAB5A | 7.622E−04 | 1.348E−02 | 8.549E−03 | 1.710E−01 | 2.098E−02 | 4.241E−01 | 1.929E−01 | 1.00 |
| 7945666 | CTSD | 7.652E−04 | 1.350E−02 | 3.083E−02 | 3.146E−01 | 9.186E−03 | 3.350E−01 | 6.607E−02 | 1.00 |
| 8100714 | YTHDC1 | 7.686E−04 | 1.351E−02 | 1.059E−03 | 7.844E−02 | 2.964E−02 | 4.681E−01 | 7.910E−01 | 1.00 |
| 8175492 | ATP11C | 7.704E−04 | 1.351E−02 | 1.332E−03 | 8.431E−02 | 1.652E−02 | 3.914E−01 | 9.195E−01 | 1.00 |
| 7932160 | FAM107B | 7.725E−04 | 1.351E−02 | 1.209E−03 | 8.193E−02 | 1.205E−02 | 3.588E−01 | 9.828E−01 | 1.00 |
| 8136293 | EXOC4 | 7.730E−04 | 1.351E−02 | 2.945E−03 | 1.076E−01 | 4.165E−03 | 2.733E−01 | 9.921E−01 | 1.00 |
| 8051998 | MCFD2 | 7.756E−04 | 1.352E−02 | 1.181E−03 | 8.180E−02 | 1.754E−02 | 3.995E−01 | 9.317E−01 | 1.00 |
| 7971208 | KBTBD6 | 7.790E−04 | 1.353E−02 | 4.475E−03 | 1.275E−01 | 3.831E−03 | 2.706E−01 | 9.651E−01 | 1.00 |
| 8017711 | GNA13 | 7.797E−04 | 1.353E−02 | 2.509E−03 | 1.045E−01 | 2.110E−02 | 4.246E−01 | 6.574E−01 | 1.00 |
| 7957177 | RAB21 | 7.823E−04 | 1.355E−02 | 7.501E−03 | 1.584E−01 | 1.245E−02 | 3.641E−01 | 4.281E−01 | 1.00 |
| 7950197 | ARAP1 | 7.867E−04 | 1.359E−02 | 7.209E−03 | 1.554E−01 | 1.335E−02 | 3.697E−01 | 4.272E−01 | 1.00 |
| 7925763 | SH3BP5L | 7.930E−04 | 1.367E−02 | 1.513E−03 | 8.673E−02 | 2.823E−03 | 2.518E−01 | 9.884E−01 | 1.00 |
| 8107129 | SLCO4C1 | 7.959E−04 | 1.369E−02 | 4.126E−02 | 3.587E−01 | 1.329E−02 | 3.697E−01 | 1.718E−02 | 1.00 |
| 8046861 | ITGAV | 8.008E−04 | 1.374E−02 | 2.598E−03 | 1.058E−01 | 2.275E−02 | 4.352E−01 | 6.256E−01 | 1.00 |
| 8104350 | KIAA0947 | 8.180E−04 | 1.400E−02 | 1.010E−03 | 7.693E−02 | 1.295E−02 | 3.684E−01 | 9.941E−01 | 1.00 |
| 8055913 | PRPF40A | 8.214E−04 | 1.403E−02 | 6.348E−03 | 1.483E−01 | 4.101E−02 | 5.065E−01 | 1.281E−01 | 1.00 |
| 7973377 | BCL2L2 | 8.337E−04 | 1.418E−02 | 5.595E−02 | 4.126E−01 | 6.363E−03 | 3.013E−01 | 3.675E−02 | 1.00 |
| 7966135 | CORO1C | 8.346E−04 | 1.418E−02 | 3.763E−02 | 3.441E−01 | 6.968E−03 | 3.038E−01 | 8.536E−02 | 1.00 |
| 7918157 | VAV3 | 8.361E−04 | 1.418E−02 | 1.440E−03 | 8.466E−02 | 2.106E−02 | 4.244E−01 | 8.724E−01 | 1.00 |
| 7926410 | MRC1 | 8.428E−04 | 1.426E−02 | 9.114E−03 | 1.749E−01 | 9.268E−03 | 3.354E−01 | 5.073E−01 | 1.00 |
| 7916747 | JAK1 | 8.478E−04 | 1.432E−02 | 8.019E−03 | 1.657E−01 | 1.426E−02 | 3.734E−01 | 4.017E−01 | 1.00 |
| 8093398 | PCGF3 | 8.538E−04 | 1.439E−02 | 1.876E−03 | 9.213E−02 | 2.579E−02 | 4.521E−01 | 7.374E−01 | 1.00 |
| 8162086 | AGTPBP1 | 8.562E−04 | 1.439E−02 | 8.789E−03 | 1.728E−01 | 1.109E−02 | 3.495E−01 | 4.642E−01 | 1.00 |
| 8127145 | ELOVL5 | 8.592E−04 | 1.441E−02 | 2.639E−03 | 1.066E−01 | 2.740E−02 | 4.548E−01 | 5.938E−01 | 1.00 |
| 7969935 | ERCC5 | 8.637E−04 | 1.446E−02 | 2.264E−04 | 5.313E−02 | 3.709E−02 | 4.960E−01 | 9.993E−01 | 1.00 |
| 8169519 | WDR44 | 8.707E−04 | 1.453E−02 | 2.717E−03 | 1.066E−01 | 3.247E−02 | 4.800E−01 | 5.204E−01 | 1.00 |
| 7974214 | KLHDC1 | 8.730E−04 | 1.453E−02 | 2.489E−03 | 1.045E−01 | 1.700E−02 | 3.942E−01 | 8.151E−01 | 1.00 |
| 8042291 | AFTPH | 8.746E−04 | 1.453E−02 | 2.764E−03 | 1.066E−01 | 2.591E−02 | 4.521E−01 | 6.097E−01 | 1.00 |
| 8078227 | KAT2B | 8.765E−04 | 1.453E−02 | 6.293E−03 | 1.482E−01 | 2.633E−02 | 4.521E−01 | 2.906E−01 | 1.00 |
| 7959604 | DDX55 | 8.777E−04 | 1.453E−02 | 2.375E−03 | 1.020E−01 | 1.833E−02 | 4.066E−01 | 8.060E−01 | 1.00 |
| 8123006 | SYNJ2 | 8.801E−04 | 1.454E−02 | 3.515E−03 | 1.169E−01 | 1.100E−02 | 3.478E−01 | 8.578E−01 | 1.00 |
| 7904158 | OLFML3 | 8.838E−04 | 1.456E−02 | 2.123E−02 | 2.682E−01 | 2.046E−03 | 2.406E−01 | 6.764E−01 | 1.00 |
| 7922162 | SLCI9A2 | 8.853E−04 | 1.456E−02 | 4.501E−03 | 1.279E−01 | 1.053E−02 | 3.428E−01 | 7.885E−01 | 1.00 |
| 7965064 | OSBPL8 | 8.873E−04 | 1.456E−02 | 8.667E−03 | 1.719E−01 | 2.398E−02 | 4.442E−01 | 2.212E−01 | 1.00 |
| 7987869 | TMEM87A | 8.898E−04 | 1.457E−02 | 1.730E−03 | 9.125E−02 | 2.374E−02 | 4.436E−01 | 8.220E−01 | 1.00 |
| 7924092 | SLC30A1 | 8.939E−04 | 1.460E−02 | 2.942E−03 | 1.076E−01 | 3.103E−02 | 4.725E−01 | 5.289E−01 | 1.00 |
| 8124459 | ZNF322 | 8.965E−04 | 1.460E−02 | 5.561E−03 | 1.400E−01 | 9.621E−03 | 3.400E−01 | 7.527E−01 | 1.00 |
| 8146000 | ADAM9 | 8.990E−04 | 1.460E−02 | 1.839E−02 | 2.499E−01 | 1.836E−02 | 4.066E−01 | 1.020E−01 | 1.00 |
| 8090469 | GATA2 | 8.995E−04 | 1.460E−02 | 4.334E−03 | 1.261E−01 | 1.607E−02 | 3.872E−01 | 6.525E−01 | 1.00 |
| 8056860 | WIPFI | 9.018E−04 | 1.461E−02 | 1.011E−02 | 1.826E−01 | 2.840E−02 | 4.606E−01 | 1.453E−01 | 1.00 |
| 8083075 | ACPL2 | 9.057E−04 | 1.464E−02 | 8.978E−04 | 7.334E−02 | 1.191E−02 | 3.582E−01 | 1.000E−00 | 1.00 |
| 7903188 | PTBP2 | 9.183E−04 | 1.481E−02 | 3.128E−03 | 1.117E−01 | 2.037E−02 | 4.230E−01 | 7.024E−01 | 1.00 |
| 8161288 | CNTNAP3 | 9.233E−04 | 1.486E−02 | 6.556E−02 | 4.430E−01 | 4.389E−03 | 2.734E−01 | 6.183E−02 | 1.00 |
| 7932938 | EPC1 | 9.250E−04 | 1.486E−02 | 6.081E−03 | 1.461E−01 | 4.867E−03 | 2.894E−01 | 9.274E−01 | 1.00 |
| 7934812 | WAPAL | 9.288E−04 | 1.489E−02 | 5.005E−03 | 1.339E−01 | 2.034E−02 | 4.230E−01 | 5.279E−01 | 1.00 |
| 7976876 | DYNC1H1 | 9.358E−04 | 1.497E−02 | 2.504E−03 | 1.045E−01 | 3.465E−02 | 4.902E−01 | 5.601E−01 | 1.00 |
| 8064939 | TMX4 | 9.380E−04 | 1.497E−02 | 4.164E−03 | 1.241E−01 | 9.327E−03 | 3.366E−01 | 8.830E−01 | 1.00 |
| 8006336 | LRRC37B | 9.402E−04 | 1.497E−02 | 4.212E−03 | 1.244E−01 | 2.067E−02 | 4.234E−01 | 5.954E−01 | 1.00 |
| 8096753 | HADH | 9.436E−04 | 1.500E−02 | 1.619E−03 | 8.904E−02 | 2.600E−02 | 4.521E−01 | 8.496E−01 | 1.00 |
| 7902883 | LRRC8D | 9.491E−04 | 1.505E−02 | 2.641E−03 | 1.066E−01 | 1.299E−02 | 3.684E−01 | 9.168E−01 | 1.00 |
| 7930956 | SEC23IP | 9.517E−04 | 1.506E−02 | 1.003E−02 | 1.823E−01 | 1.889E−02 | 4.127E−01 | 2.873E−01 | 1.00 |
| 7966052 | CRY1 | 9.577E−04 | 1.512E−02 | 1.756E−03 | 9.178E−02 | 1.485E−02 | 3.734E−01 | 9.651E−01 | 1.00 |
| 8160238 | PSIP1 | 9.704E−04 | 1.523E−02 | 1.069E−03 | 7.844E−02 | 1.958E−02 | 4.180E−01 | 9.884E−01 | 1.00 |
| 7969576 | MIR17HG | 9.709E−04 | 1.523E−02 | 1.513E−03 | 8.673E−02 | 2.124E−02 | 4.257E−01 | 9.312E−01 | 1.00 |
| 8059739 | NPPC | 9.714E−04 | 1.523E−02 | 1.649E−01 | 6.848E−01 | 2.631E−03 | 2.459E−01 | 6.874E−03 | 1.00 |
| 8085815 | TOP2B | 9.727E−04 | 1.523E−02 | 1.728E−03 | 9.125E−02 | 2.366E−02 | 4.434E−01 | 8.747E−01 | 1.00 |
| 8133860 | GNAI1 | 9.750E−04 | 1.524E−02 | 4.141E−03 | 1.241E−01 | 3.888E−03 | 2.706E−01 | 9.981E−01 | 1.00 |
| 7916282 | LRPS | 9.824E−04 | 1.532E−02 | 2.153E−03 | 9.773E−02 | 6.881E−03 | 3.028E−01 | 9.997E−01 | 1.00 |
| 8164002 | ZBTB26 | 9.852E−04 | 1.533E−02 | 2.567E−03 | 1.055E−01 | 1.040E−02 | 3.428E−01 | 9.694E−01 | 1.00 |
| 8003116 | HSDL1 | 9.915E−04 | 1.540E−02 | 9.260E−03 | 1.762E−01 | 1.101E−02 | 3.478E−01 | 5.614E−01 | 1.00 |
| 8170364 | AFF2 | 9.946E−04 | 1.541E−02 | 2.950E−02 | 3.091E−01 | 1.302E−02 | 3.684E−01 | 9.171E−02 | 1.00 |
| 8086482 | ZNF445 | 9.960E−04 | 1.541E−02 | 3.280E−03 | 1.143E−01 | 1.753E−02 | 3.995E−01 | 7.965E−01 | 1.00 |
| 7978428 | STRN3 | 9.982E−04 | 1.541E−02 | 7.445E−03 | 1.582E−01 | 1.949E−02 | 4.176E−01 | 4.263E−01 | 1.00 |

TABLE 19
Differentially expressed genes in P2*. Welch's t-test was used for the comparison between ASD and controls.
To identify differentially expressed genes in P2* dataset, significance of diagnosis (p(Dx)) and gender
(p(Gender)) was determined by two-way analysis of variance (ANOVA) and follow-up Welch's t-test for each
gender. p(Dx*Gender) denotes the interaction between diagnosis and gender effects for significance. A total of 469
unique genes were differentially expressed (P < 0.001, corresponding FDR 0.023) as there were transcripts
without official gene symbols (i.e., — in Gene field) and several genes have multiple Affymetrix IDs.

| | | | | ASD vs. Controls | | ASD vs. Controls | | | | |
|---|---|---|---|---|---|---|---|---|---|---|
| Affymetrix | | ASD vs. Controls | | (males) | | (females) | | Two-way ANOVA | | |
| ID | Gene | p-value | FDR | p-value | FDR | p-value | FDR | p(Dx) | p(Gender) | p(Dx*Gender) |
| 8021181 | SCARNA17 | 1.61E−07 | 1.48E−03 | 1.34E−04 | 2.75E−02 | 1.39E−03 | 5.98E−01 | 5.05E−07 | 2.64E−01 | 4.65E−01 |
| 8076344 | POLR3H | 3.30E−07 | 1.48E−03 | 2.08E−04 | 2.75E−02 | 9.70E−04 | 5.98E−01 | 7.57E−07 | 7.57E−01 | 3.71E−01 |
| 7913644 | E2F2 | 4.92E−07 | 1.48E−03 | 2.88E−04 | 2.75E−02 | 3.10E−03 | 6.06E−01 | 1.51E−06 | 1.20E−01 | 3.16E−01 |
| 7970999 | SPG20 | 6.00E−07 | 1.48E−03 | 1.08E−04 | 2.75E−02 | 6.97E−03 | 6.35E−01 | 2.77E−06 | 2.46E−01 | 6.69E−01 |
| 7894952 | — | 6.33E−07 | 1.48E−03 | 3.44E−05 | 2.75E−02 | 4.64E−02 | 6.67E−01 | 1.36E−06 | 3.98E−02 | 4.60E−01 |
| 8005204 | CCDC144A | 6.81E−07 | 1.48E−03 | 2.10E−04 | 2.75E−02 | 1.10E−02 | 6.35E−01 | 9.07E−07 | 8.63E−03 | 7.42E−01 |
| 8013272 | CCDC144A | 7.35E−07 | 1.48E−03 | 2.87E−04 | 2.75E−02 | 7.42E−03 | 6.35E−01 | 1.00E−06 | 1.07E−02 | 8.45E−01 |
| 7998952 | TIGD7 | 8.34E−07 | 1.48E−03 | 3.91E−04 | 2.75E−02 | 1.21E−03 | 5.98E−01 | 2.24E−06 | 7.19E−01 | 3.83E−01 |
| 8180286 | RBM8A | 1.46E−06 | 2.30E−03 | 1.31E−03 | 3.00E−02 | 5.78E−04 | 5.79E−01 | 4.00E−06 | 5.60E−01 | 9.60E−02 |
| 7893512 | — | 2.99E−06 | 3.72E−03 | 1.05E−04 | 2.75E−02 | 1.06E−01 | 7.01E−01 | 4.32E−06 | 1.08E−03 | 4.86E−01 |
| 7913593 | TCEA3 | 3.02E−06 | 3.72E−03 | 4.12E−04 | 2.77E−02 | 5.49E−03 | 6.33E−01 | 2.16E−06 | 7.98E−01 | 7.43E−01 |
| 8098758 | ZNF721 | 4.14E−06 | 3.72E−03 | 1.08E−03 | 3.00E−02 | 1.73E−03 | 5.98E−01 | 1.23E−05 | 9.29E−01 | 2.40E−01 |
| 8005547 | SNORD3A | 4.46E−06 | 3.72E−03 | 6.09E−03 | 3.15E−02 | 3.89E−05 | 1.15E−01 | 6.69E−06 | 3.87E−01 | 2.49E−02 |
| 8005553 | SNORD3A | 4.46E−06 | 3.72E−03 | 6.09E−03 | 3.15E−02 | 3.89E−05 | 1.15E−01 | 6.69E−06 | 3.87E−01 | 2.49E−02 |
| 8013323 | SNORD3A | 4.46E−06 | 3.72E−03 | 6.09E−03 | 3.15E−02 | 3.89E−05 | 1.15E−01 | 6.69E−06 | 3.87E−01 | 2.49E−02 |
| 8013325 | SNORD3A | 4.46E−06 | 3.72E−03 | 6.09E−03 | 3.15E−02 | 3.89E−05 | 1.15E−01 | 6.69E−06 | 3.87E−01 | 2.49E−02 |
| 8013329 | SNORD3A | 4.46E−06 | 3.72E−03 | 6.09E−03 | 3.15E−02 | 3.89E−05 | 1.15E−01 | 6.69E−06 | 3.87E−01 | 2.49E−02 |
| 8176624 | DDX3Y | 7.49E−06 | 5.03E−03 | 1.99E−07 | 2.22E−03 | 4.85E−01 | 8.08E−01 | 2.21E−17 | 5.60E−58 | 4.12E−04 |
| 8040503 | UBXN2A | 7.85E−06 | 5.03E−03 | 4.75E−05 | 2.75E−02 | 3.62E−02 | 6.59E−01 | 2.04E−05 | 5.16E−01 | 8.26E−01 |
| 7941272 | MALAT1 | 8.00E−06 | 5.03E−03 | 2.84E−03 | 3.12E−02 | 2.35E−03 | 6.06E−01 | 2.56E−05 | 3.00E−01 | 2.08E−01 |
| 7972921 | — | 8.11E−06 | 5.03E−03 | 2.35E−03 | 3.11E−02 | 1.93E−03 | 5.98E−01 | 2.05E−05 | 7.13E−01 | 1.82E−01 |
| 7934215 | SPOCK2 | 8.61E−06 | 5.03E−03 | 1.30E−04 | 2.75E−02 | 2.25E−02 | 6.45E−01 | 3.49E−05 | 7.17E−01 | 8.50E−01 |
| 8011823 | ZNF594 | 8.96E−06 | 5.03E−03 | 5.85E−03 | 3.15E−02 | 1.60E−03 | 5.98E−01 | 9.09E−06 | 1.92E−01 | 1.78E−01 |
| 8026390 | CCDC105 | 9.14E−06 | 5.03E−03 | 1.16E−04 | 2.75E−02 | 2.83E−02 | 6.45E−01 | 5.89E−05 | 9.07E−01 | 8.52E−01 |
| 8105612 | CWC27 | 9.23E−06 | 5.03E−03 | 9.08E−04 | 3.00E−02 | 4.47E−03 | 6.11E−01 | 3.61E−05 | 8.81E−01 | 4.06E−01 |
| 8055978 | FAM133B | 9.23E−06 | 5.03E−03 | 1.00E−03 | 3.00E−02 | 3.72E−03 | 6.06E−01 | 2.82E−05 | 9.18E−01 | 3.89E−01 |
| 8171024 | — | 1.03E−05 | 5.37E−03 | 1.46E−03 | 3.01E−02 | 4.82E−03 | 6.20E−01 | 2.32E−05 | 6.19E−01 | 4.63E−01 |
| 7895954 | — | 1.06E−05 | 5.37E−03 | 1.77E−03 | 3.09E−02 | 1.65E−03 | 5.98E−01 | 3.08E−05 | 7.01E−01 | 1.47E−01 |
| 7943158 | SCARNA9 | 1.11E−05 | 5.40E−03 | 9.82E−04 | 3.00E−02 | 7.42E−03 | 6.35E−01 | 2.30E−05 | 6.79E−01 | 5.01E−01 |
| 8036395 | ZNF569 | 1.38E−05 | 6.42E−03 | 5.25E−04 | 2.98E−02 | 1.40E−02 | 6.41E−01 | 3.29E−05 | 9.70E−01 | 6.69E−01 |
| 8065032 | ESF1 | 1.40E−05 | 6.42E−03 | 8.76E−03 | 3.34E−02 | 4.64E−04 | 5.79E−01 | 2.19E−05 | 9.52E−01 | 4.67E−02 |
| 8005679 | CCDC144C | 1.46E−05 | 6.46E−03 | 7.40E−03 | 3.28E−02 | 3.97E−03 | 6.08E−01 | 1.13E−06 | 7.94E−03 | 6.72E−01 |
| 7965357 | GALNT4 | 1.67E−05 | 6.91E−03 | 8.08E−04 | 3.00E−02 | 1.49E−02 | 6.41E−01 | 5.66E−05 | 6.08E−01 | 6.03E−01 |
| 8105504 | FAM133B | 1.71E−05 | 6.91E−03 | 9.09E−04 | 3.00E−02 | 8.57E−03 | 6.35E−01 | 4.84E−05 | 9.29E−01 | 5.56E−01 |
| 7964642 | C12orf61 | 1.79E−05 | 6.91E−03 | 1.84E−03 | 3.09E−02 | 1.04E−02 | 6.35E−01 | 5.62E−05 | 2.95E−01 | 4.57E−01 |
| 7997940 | SNORD68 | 1.80E−05 | 6.91E−03 | 1.53E−03 | 3.01E−02 | 2.64E−03 | 6.06E−01 | 3.05E−05 | 5.74E−01 | 4.11E−01 |
| 7955721 | ZNF740 | 1.81E−05 | 6.91E−03 | 3.86E−04 | 2.75E−02 | 5.34E−02 | 6.73E−01 | 2.51E−05 | 2.65E−01 | 8.34E−01 |
| 7895072 | — | 2.16E−05 | 8.05E−03 | 9.69E−04 | 3.00E−02 | 3.01E−02 | 6.45E−01 | 8.32E−05 | 1.56E−01 | 7.57E−01 |
| 7903519 | PRPF38B | 2.23E−05 | 8.09E−03 | 2.32E−03 | 3.11E−02 | 9.72E−03 | 6.35E−01 | 8.39E−05 | 2.29E−01 | 3.94E−01 |
| 8060196 | MTERFD2 | 2.29E−05 | 8.09E−03 | 1.50E−03 | 3.01E−02 | 9.46E−03 | 6.35E−01 | 5.13E−05 | 7.72E−01 | 5.08E−01 |
| 7941795 | — | 2.34E−05 | 8.09E−03 | 5.44E−04 | 2.99E−02 | 1.52E−02 | 6.41E−01 | 2.72E−04 | 7.24E−01 | 8.14E−01 |
| 7894657 | — | 2.48E−05 | 8.09E−03 | 3.74E−04 | 2.75E−02 | 2.24E−02 | 6.45E−01 | 1.02E−04 | 7.46E−01 | 7.15E−01 |
| 8065569 | BCL2L1 | 2.53E−05 | 8.09E−03 | 4.78E−02 | 5.17E−02 | 7.60E−04 | 5.98E−01 | 1.67E−05 | 8.59E−03 | 2.17E−02 |
| 8151788 | RBM12B | 2.57E−05 | 8.09E−03 | 5.43E−04 | 2.99E−02 | 3.00E−02 | 6.45E−01 | 1.05E−04 | 6.77E−01 | 8.56E−01 |
| 8171760 | SCARNA9L | 2.64E−05 | 8.09E−03 | 3.05E−03 | 3.13E−02 | 1.93E−03 | 5.98E−01 | 5.29E−05 | 6.08E−01 | 1.94E−01 |
| 8137693 | COX19 | 2.83E−05 | 8.09E−03 | 4.43E−03 | 3.13E−02 | 9.36E−03 | 6.35E−01 | 8.27E−05 | 9.01E−02 | 3.12E−01 |
| 7945058 | FAM118B | 2.87E−05 | 8.09E−03 | 3.70E−03 | 3.13E−02 | 1.32E−02 | 6.40E−01 | 6.94E−05 | 5.19E−02 | 5.39E−01 |
| 8025998 | ZNF136 | 2.88E−05 | 8.09E−03 | 9.79E−04 | 3.00E−02 | 2.07E−02 | 6.45E−01 | 9.51E−05 | 6.13E−01 | 7.32E−01 |
| 8176384 | ZFY | 2.92E−05 | 8.09E−03 | 1.76E−05 | 2.75E−02 | 2.96E−01 | 7.64E−01 | 2.05E−13 | 2.82E−50 | 1.48E−03 |
| 7893519 | — | 2.93E−05 | 8.09E−03 | 4.02E−03 | 3.13E−02 | 1.06E−02 | 6.35E−01 | 5.02E−05 | 1.51E−01 | 3.33E−01 |
| 8137232 | GIMAP8 | 3.00E−05 | 8.09E−03 | 4.02E−04 | 2.75E−02 | 3.77E−02 | 6.59E−01 | 9.48E−05 | 8.43E−01 | 8.49E−01 |
| 7908614 | CAMSAP2 | 3.01E−05 | 8.09E−03 | 3.67E−04 | 2.75E−02 | 1.19E−02 | 6.35E−01 | 1.55E−05 | 1.17E−01 | 6.33E−01 |
| 8099235 | MRFAP1L1 | 3.08E−05 | 8.09E−03 | 6.27E−03 | 3.18E−02 | 5.60E−03 | 6.33E−01 | 7.75E−05 | 1.15E−01 | 2.43E−01 |
| 7894905 | — | 3.08E−05 | 8.09E−03 | 1.03E−03 | 3.00E−02 | 8.78E−02 | 6.94E−01 | 3.56E−05 | 9.90E−03 | 7.73E−01 |
| 8021183 | SCARNA17 | 3.72E−05 | 9.42E−03 | 3.58E−03 | 3.13E−02 | 6.93E−03 | 6.35E−01 | 9.74E−05 | 5.18E−01 | 3.44E−01 |
| 7893547 | — | 3.73E−05 | 9.42E−03 | 1.32E−04 | 2.75E−02 | 8.67E−02 | 6.94E−01 | 1.31E−04 | 9.90E−01 | 8.17E−01 |
| 8150877 | SNORD54 | 3.92E−05 | 9.42E−03 | 9.57E−05 | 2.75E−02 | 1.36E−01 | 7.20E−01 | 8.43E−05 | 9.62E−01 | 4.15E−01 |
| 8022045 | MYOM1 | 3.94E−05 | 9.42E−03 | 3.83E−03 | 3.13E−02 | 1.32E−02 | 6.40E−01 | 1.63E−05 | 3.60E−01 | 4.95E−01 |
| 8072382 | OSBP2 | 3.95E−05 | 9.42E−03 | 1.61E−02 | 3.68E−02 | 1.01E−02 | 6.35E−01 | 2.45E−05 | 2.14E−03 | 1.29E−01 |
| 8006477 | ZNF830 | 3.99E−05 | 9.42E−03 | 3.00E−03 | 3.13E−02 | 1.93E−02 | 6.45E−01 | 1.03E−04 | 1.22E−01 | 4.39E−01 |
| 7960654 | ING4 | 4.23E−05 | 9.76E−03 | 5.40E−04 | 2.99E−02 | 6.26E−02 | 6.79E−01 | 1.09E−04 | 4.27E−01 | 9.89E−01 |
| 8167815 | MAGED2 | 4.27E−05 | 9.76E−03 | 9.96E−04 | 3.00E−02 | 2.33E−02 | 6.45E−01 | 1.81E−04 | 8.11E−01 | 8.71E−01 |
| 8093130 | RNF168 | 4.37E−05 | 9.83E−03 | 8.87E−04 | 3.00E−02 | 4.84E−02 | 6.67E−01 | 1.68E−04 | 3.20E−01 | 9.92E−01 |
| 7926283 | PRPF18 | 4.52E−05 | 9.92E−03 | 3.85E−03 | 3.13E−02 | 7.54E−03 | 6.35E−01 | 1.46E−04 | 4.96E−01 | 3.19E−01 |
| 8119408 | NFYA | 4.63E−05 | 9.92E−03 | 4.03E−04 | 2.75E−02 | 7.49E−02 | 6.93E−01 | 1.77E−04 | 5.76E−01 | 7.91E−01 |
| 7982574 | FAM98B | 4.68E−05 | 9.92E−03 | 1.69E−03 | 3.04E−02 | 3.22E−02 | 6.45E−01 | 1.67E−04 | 2.21E−01 | 7.59E−01 |
| 7959251 | P2RX7 | 4.69E−05 | 9.92E−03 | 1.14E−02 | 3.48E−02 | 6.90E−03 | 6.35E−01 | 6.85E−05 | 9.21E−02 | 1.26E−01 |
| 8176698 | TXLNG2P | 4.92E−05 | 9.92E−03 | 5.76E−05 | 2.75E−02 | 3.04E−01 | 7.66E−01 | 3.52E−13 | 2.20E−51 | 2.69E−03 |
| 7899377 | PPP1P8 | 5.05E−05 | 9.92E−03 | 5.83E−03 | 3.15E−02 | 1.34E−02 | 6.40E−01 | 1.15E−04 | 7.82E−02 | 3.81E−01 |
| 8016433 | HOXB1 | 5.07E−05 | 9.92E−03 | 2.78E−04 | 2.75E−02 | 8.24E−02 | 6.94E−01 | 1.97E−04 | 9.99E−01 | 5.84E−01 |
| 7970681 | RNF6 | 5.08E−05 | 9.92E−03 | 2.67E−03 | 3.12E−02 | 1.55E−02 | 6.41E−01 | 1.56E−04 | 4.34E−01 | 3.93E−01 |
| 7919394 | LOC728855 | 5.12E−05 | 9.92E−03 | 2.60E−03 | 3.12E−02 | 1.13E−02 | 6.35E−01 | 1.63E−04 | 7.61E−01 | 4.06E−01 |
| 7898910 | PNRC2 | 5.18E−05 | 9.92E−03 | 9.99E−03 | 3.40E−02 | 2.61E−03 | 6.06E−01 | 1.31E−04 | 3.31E−01 | 1.82E−01 |
| 8039933 | PNRC2 | 5.18E−05 | 9.92E−03 | 9.99E−03 | 3.40E−02 | 2.61E−03 | 6.06E−01 | 1.31E−04 | 3.31E−01 | 1.82E−01 |
| 8082504 | C3orf37 | 5.27E−05 | 9.92E−03 | 1.25E−03 | 3.00E−02 | 4.84E−02 | 6.67E−01 | 1.09E−04 | 2.77E−01 | 9.70E−01 |
| 8164907 | REXO4 | 5.37E−05 | 9.92E−03 | 5.84E−02 | 5.64E−02 | 6.07E−04 | 5.79E−01 | 2.54E−05 | 2.09E−02 | 4.34E−02 |
| 8176709 | CYorf15B | 5.53E−05 | 9.92E−03 | 1.25E−04 | 2.75E−02 | 3.54E−01 | 7.78E−01 | 2.91E−11 | 3.04E−44 | 4.74E−03 |
| 8003621 | RNMTL1 | 5.56E−05 | 9.92E−03 | 6.63E−02 | 6.00E−02 | 5.39E−04 | 5.79E−01 | 2.49E−05 | 7.01E−02 | 2.49E−02 |
| 8021984 | YES1 | 5.62E−05 | 9.92E−03 | 2.35E−03 | 3.11E−02 | 5.38E−03 | 6.33E−01 | 1.22E−04 | 3.99E−01 | 3.27E−01 |
| 8084146 | FXR1 | 5.64E−05 | 9.92E−03 | 1.97E−03 | 3.11E−02 | 1.64E−02 | 6.41E−01 | 1.79E−04 | 7.61E−01 | 4.47E−01 |
| 8177137 | UTY | 5.75E−05 | 9.92E−03 | 1.13E−05 | 2.75E−02 | 3.43E−01 | 7.73E−01 | 2.00E−14 | 5.41E−57 | 1.47E−03 |
| 8079346 | SACM1L | 5.76E−05 | 9.92E−03 | 5.52E−04 | 3.00E−02 | 3.01E−02 | 6.45E−01 | 2.91E−04 | 6.74E−01 | 7.50E−01 |
| 7893397 | — | 6.03E−05 | 9.92E−03 | 1.56E−03 | 3.02E−02 | 6.27E−02 | 6.79E−01 | 3.28E−05 | 1.91E−01 | 6.79E−01 |
| 8024255 | MUM1 | 6.14E−05 | 9.92E−03 | 3.47E−04 | 2.75E−02 | 4.72E−02 | 6.67E−01 | 1.04E−04 | 5.37E−01 | 9.47E−01 |
| 8077931 | MKRN2 | 6.24E−05 | 9.92E−03 | 3.44E−04 | 2.75E−02 | 9.32E−02 | 6.97E−01 | 2.16E−04 | 6.34E−01 | 8.61E−01 |
| 8020806 | RNF125 | 6.25E−05 | 9.92E−03 | 7.48E−04 | 3.00E−02 | 2.16E−02 | 6.45E−01 | 2.61E−04 | 5.51E−01 | 7.98E−01 |
| 7892909 | — | 6.26E−05 | 9.92E−03 | 2.40E−02 | 4.06E−02 | 4.04E−03 | 6.08E−01 | 8.98E−05 | 1.92E−02 | 1.52E−01 |
| 8063636 | STX16 | 6.34E−05 | 9.92E−03 | 1.66E−03 | 3.04E−02 | 4.98E−02 | 6.67E−01 | 2.68E−04 | 1.44E−01 | 8.74E−01 |
| 8141133 | SHFM1 | 6.35E−05 | 9.92E−03 | 1.71E−02 | 3.73E−02 | 2.69E−03 | 6.06E−01 | 9.93E−05 | 1.92E−01 | 1.66E−01 |
| 8167638 | — | 6.41E−05 | 9.92E−03 | 2.25E−03 | 3.11E−02 | 3.23E−02 | 6.45E−01 | 1.19E−04 | 3.14E−01 | 7.28E−01 |
| 8176578 | USP9Y | 6.42E−05 | 9.92E−03 | 8.18E−05 | 2.75E−02 | 3.86E−01 | 7.87E−01 | 2.73E−12 | 2.15E−49 | 4.34E−03 |
| 8176276 | ATRX | 6.46E−05 | 9.92E−03 | 5.04E−04 | 2.96E−02 | 4.16E−02 | 6.61E−01 | 2.92E−04 | 7.92E−01 | 8.26E−01 |
| 8110392 | TMED9 | 6.51E−05 | 9.92E−03 | 4.65E−03 | 3.13E−02 | 7.48E−03 | 6.35E−01 | 3.33E−04 | 3.35E−01 | 3.98E−01 |
| 8115166 | — | 6.72E−05 | 1.00E−02 | 4.33E−03 | 3.13E−02 | 2.77E−03 | 6.06E−01 | 6.68E−05 | 8.71E−01 | 7.97E−01 |
| 8059770 | TIGD1 | 6.85E−05 | 1.00E−02 | 1.58E−02 | 3.66E−02 | 2.82E−03 | 6.06E−01 | 1.03E−04 | 2.82E−01 | 1.99E−01 |
| 8176719 | EIF1AY | 6.90E−05 | 1.00E−02 | 4.02E−04 | 2.75E−02 | 3.40E−01 | 7.72E−01 | 2.32E−09 | 1.12E−36 | 8.91E−03 |
| 8110112 | — | 6.94E−05 | 1.00E−02 | 2.06E−03 | 3.11E−02 | 2.61E−02 | 6.45E−01 | 1.83E−04 | 5.32E−01 | 9.11E−01 |
| 7895774 | — | 6.94E−05 | 1.00E−02 | 1.94E−02 | 3.85E−02 | 2.48E−03 | 6.06E−01 | 2.37E−05 | 2.44E−01 | 2.89E−01 |
| 8067585 | BHLHE23 | 7.07E−05 | 1.00E−02 | 3.09E−03 | 3.13E−02 | 1.82E−02 | 6.43E−01 | 2.76E−04 | 5.01E−01 | 4.46E−01 |
| 8005110 | ZNF286A | 7.08E−05 | 1.00E−02 | 7.00E−03 | 3.24E−02 | 2.64E−03 | 6.06E−01 | 1.59E−04 | 5.78E−01 | 1.01E−01 |
| 8137715 | MICALL2 | 7.22E−05 | 1.01E−02 | 3.82E−04 | 2.75E−02 | 6.09E−02 | 6.79E−01 | 4.61E−04 | 8.78E−01 | 6.24E−01 |
| 8156826 | TGFBR1 | 7.46E−05 | 1.04E−02 | 1.90E−04 | 2.75E−02 | 1.09E−01 | 7.01E−01 | 2.91E−04 | 7.41E−01 | 6.10E−01 |
| 7895369 | — | 7.57E−05 | 1.04E−02 | 1.93E−05 | 2.75E−02 | 2.87E−01 | 7.62E−01 | 4.98E−05 | 1.75E−01 | 1.01E−01 |
| 7895955 | — | 7.64E−05 | 1.04E−02 | 2.84E−03 | 3.12E−02 | 3.73E−02 | 6.59E−01 | 1.42E−04 | 1.76E−01 | 8.95E−01 |
| 7978754 | C11orf58 | 7.73E−05 | 1.04E−02 | 3.11E−03 | 3.13E−02 | 2.27E−02 | 6.45E−01 | 2.67E−04 | 3.01E−01 | 5.87E−01 |
| 7933659 | CSTF2T | 7.95E−05 | 1.05E−02 | 5.97E−04 | 3.00E−02 | 1.10E−01 | 7.01E−01 | 2.28E−04 | 2.88E−01 | 8.24E−01 |
| 8034199 | TSPAN16 | 8.02E−05 | 1.05E−02 | 5.66E−03 | 3.15E−02 | 1.40E−02 | 6.41E−01 | 1.33E−04 | 1.99E−01 | 7.98E−01 |
| 7944401 | HMBS | 8.06E−05 | 1.05E−02 | 8.45E−03 | 3.34E−02 | 2.91E−03 | 6.06E−01 | 1.29E−04 | 9.76E−01 | 2.72E−01 |
| 8020717 | CHST9-AS1 | 8.09E−05 | 1.05E−02 | 2.07E−04 | 2.75E−02 | 5.56E−02 | 6.77E−01 | 3.49E−04 | 2.00E−01 | 5.22E−01 |
| 8099364 | ZNF518B | 8.18E−05 | 1.05E−02 | 9.71E−05 | 2.75E−02 | 1.47E−01 | 7.23E−01 | 3.23E−04 | 7.94E−01 | 6.15E−01 |
| 8119016 | MAPK13 | 8.52E−05 | 1.06E−02 | 2.17E−03 | 3.11E−02 | 2.42E−02 | 6.45E−01 | 2.16E−04 | 7.68E−01 | 8.13E−01 |
| 7896007 | — | 8.68E−05 | 1.06E−02 | 3.46E−04 | 2.75E−02 | 1.57E−01 | 7.26E−01 | 4.54E−04 | 3.45E−01 | 6.74E−01 |
| 8117020 | MYLIP | 8.69E−05 | 1.06E−02 | 1.06E−03 | 3.00E−02 | 2.58E−02 | 6.45E−01 | 3.51E−04 | 7.79E−01 | 9.10E−01 |
| 7971373 | — | 8.70E−05 | 1.06E−02 | 2.90E−02 | 4.31E−02 | 3.14E−04 | 5.15E−01 | 1.05E−04 | 4.08E−01 | 1.65E−02 |
| 8030946 | ZNF808 | 8.75E−05 | 1.06E−02 | 2.14E−03 | 3.11E−02 | 2.16E−02 | 6.45E−01 | 3.51E−04 | 8.10E−01 | 6.80E−01 |
| 7981439 | BAG5 | 8.78E−05 | 1.06E−02 | 3.64E−03 | 3.13E−02 | 1.53E−02 | 6.41E−01 | 2.27E−04 | 7.14E−01 | 4.64E−01 |
| 7979691 | LOC100128233 | 8.79E−05 | 1.06E−02 | 9.44E−05 | 2.75E−02 | 2.43E−01 | 7.47E−01 | 3.13E−04 | 8.98E−01 | 3.32E−01 |
| 7943314 | JRKL | 8.83E−05 | 1.06E−02 | 5.52E−03 | 3.15E−02 | 1.45E−02 | 6.41E−01 | 1.88E−04 | 3.96E−01 | 4.14E−01 |
| 8116649 | TUBB2A | 8.96E−05 | 1.06E−02 | 2.07E−03 | 3.11E−02 | 2.74E−02 | 6.45E−01 | 2.28E−04 | 6.96E−01 | 4.57E−01 |
| 8116653 | TUBB2A | 8.96E−05 | 1.06E−02 | 2.07E−03 | 3.11E−02 | 2.74E−02 | 6.45E−01 | 2.28E−04 | 6.96E−01 | 4.57E−01 |
| 8093336 | ZNF141 | 9.08E−05 | 1.06E−02 | 4.05E−03 | 3.13E−02 | 8.54E−03 | 6.35E−01 | 2.05E−04 | 7.91E−01 | 3.36E−01 |
| 8177232 | KDMSD | 9.21E−05 | 1.06E−02 | 8.85E−05 | 2.75E−02 | 4.99E−01 | 8.10E−01 | 1.64E−14 | 5.00E−58 | 5.19E−03 |
| 7893619 | — | 9.23E−05 | 1.06E−02 | 6.51E−03 | 3.19E−02 | 9.27E−03 | 6.35E−01 | 2.74E−04 | 4.87E−01 | 1.80E−01 |
| 7933228 | MARCH8 | 9.41E−05 | 1.08E−02 | 6.33E−02 | 5.87E−02 | 1.22E−03 | 5.98E−01 | 9.71E−05 | 5.99E−02 | 2.44E−02 |
| 7960052 | SNORA49 | 9.63E−05 | 1.09E−02 | 1.47E−03 | 3.01E−02 | 7.80E−03 | 6.35E−01 | 1.89E−04 | 1.13E−01 | 3.73E−01 |
| 8173364 | — | 9.75E−05 | 1.09E−02 | 8.39E−03 | 3.34E−02 | 1.04E−02 | 6.35E−01 | 1.24E−04 | 5.08E−01 | 2.42E−01 |
| 8085537 | ZFYVE20 | 9.80E−05 | 1.09E−02 | 1.12E−03 | 3.00E−02 | 6.26E−02 | 6.79E−01 | 2.81E−04 | 6.21E−01 | 9.14E−01 |
| 8073875 | TRMU | 9.89E−05 | 1.10E−02 | 8.41E−03 | 3.34E−02 | 1.25E−02 | 6.35E−01 | 9.86E−05 | 2.80E−01 | 3.87E−01 |
| 8028266 | ZNF540 | 1.01E−04 | 1.10E−02 | 2.03E−03 | 3.11E−02 | 9.64E−03 | 6.35E−01 | 2.46E−04 | 3.36E−01 | 5.86E−01 |
| 8137252 | GIMAP1 | 1.01E−04 | 1.10E−02 | 7.78E−04 | 3.00E−02 | 5.93E−02 | 6.79E−01 | 3.84E−04 | 9.01E−01 | 8.81E−01 |
| 7896169 | — | 1.06E−04 | 1.13E−02 | 2.49E−03 | 3.12E−02 | 1.48E−02 | 6.41E−01 | 4.04E−04 | 9.88E−01 | 7.27E−01 |
| 8116651 | — | 1.06E−04 | 1.13E−02 | 4.23E−03 | 3.13E−02 | 2.01E−02 | 6.45E−01 | 2.12E−04 | 5.65E−01 | 4.24E−01 |
| 7896139 | — | 1.07E−04 | 1.13E−02 | 1.09E−03 | 3.00E−02 | 9.62E−02 | 6.99E−01 | 3.13E−04 | 2.52E−01 | 9.03E−01 |
| 8066009 | RBM39 | 1.08E−04 | 1.13E−02 | 9.90E−04 | 3.00E−02 | 4.95E−02 | 6.67E−01 | 4.61E−04 | 8.81E−01 | 7.25E−01 |
| 8063345 | SNORD12C | 1.10E−04 | 1.13E−02 | 2.94E−03 | 3.13E−02 | 5.18E−02 | 6.71E−01 | 3.85E−04 | 1.21E−01 | 7.92E−01 |
| 8088151 | ACTR8 | 1.11E−04 | 1.13E−02 | 8.90E−04 | 3.00E−02 | 1.18E−01 | 7.04E−01 | 3.80E−04 | 2.20E−01 | 8.66E−01 |
| 8091778 | SCARNA7 | 1.11E−04 | 1.13E−02 | 4.95E−03 | 3.13E−02 | 1.20E−02 | 6.35E−01 | 2.01E−04 | 6.51E−01 | 6.85E−01 |
| 7961339 | LRP6 | 1.12E−04 | 1.13E−02 | 2.00E−02 | 3.88E−02 | 2.72E−03 | 6.06E−01 | 1.74E−05 | 5.04E−01 | 4.03E−01 |
| 7894700 | — | 1.14E−04 | 1.13E−02 | 3.26E−03 | 3.13E−02 | 8.70E−03 | 6.35E−01 | 2.74E−04 | 4.48E−01 | 3.06E−01 |
| 8119993 | HSP90AB1 | 1.14E−04 | 1.13E−02 | 3.45E−04 | 2.75E−02 | 1.17E−01 | 7.03E−01 | 3.62E−04 | 9.03E−01 | 7.94E−01 |
| 8038981 | ZNF611 | 1.16E−04 | 1.13E−02 | 2.24E−03 | 3.11E−02 | 2.77E−02 | 6.45E−01 | 4.45E−04 | 8.31E−01 | 6.25E−01 |
| 7980983 | MOAP1 | 1.16E−04 | 1.13E−02 | 1.73E−03 | 3.06E−02 | 3.72E−02 | 6.59E−01 | 2.80E−04 | 8.51E−01 | 7.58E−01 |
| 8134730 | CNPY4 | 1.16E−04 | 1.13E−02 | 3.35E−02 | 4.52E−02 | 7.23E−03 | 6.35E−01 | 1.00E−04 | 1.99E−02 | 1.18E−01 |
| 7896634 | — | 1.17E−04 | 1.13E−02 | 5.87E−05 | 2.75E−02 | 5.29E−01 | 8.17E−01 | 2.62E−04 | 7.16E−02 | 1.47E−01 |
| 7926299 | HSPA14 | 1.18E−04 | 1.13E−02 | 5.58E−04 | 3.00E−02 | 7.78E−02 | 6.94E−01 | 3.79E−04 | 8.10E−01 | 8.00E−01 |
| 8014755 | SNORA21 | 1.18E−04 | 1.13E−02 | 2.43E−02 | 4.07E−02 | 4.79E−04 | 5.79E−01 | 6.30E−05 | 2.62E−01 | 4.45E−02 |
| 8107208 | FER | 1.19E−04 | 1.13E−02 | 3.59E−04 | 2.75E−02 | 1.08E−01 | 7.01E−01 | 4.16E−04 | 8.41E−01 | 6.79E−01 |
| 7918911 | — | 1.20E−04 | 1.13E−02 | 3.09E−03 | 3.13E−02 | 5.86E−02 | 6.79E−01 | 3.62E−04 | 9.24E−02 | 7.58E−01 |
| 7948904 | SNORD28 | 1.20E−04 | 1.13E−02 | 2.82E−03 | 3.12E−02 | 2.82E−02 | 6.45E−01 | 2.69E−04 | 6.47E−01 | 8.56E−01 |
| 8053496 | POLR1A | 1.21E−04 | 1.13E−02 | 1.62E−03 | 3.02E−02 | 6.32E−02 | 6.79E−01 | 2.37E−04 | 4.00E−01 | 8.11E−01 |
| 7967685 | STX2 | 1.21E−04 | 1.13E−02 | 3.65E−04 | 2.75E−02 | 1.36E−01 | 7.20E−01 | 3.82E−04 | 6.58E−01 | 7.59E−01 |
| 7895350 | — | 1.24E−04 | 1.13E−02 | 2.71E−03 | 3.12E−02 | 2.58E−02 | 6.45E−01 | 5.06E−04 | 6.89E−01 | 6.87E−01 |
| 8140942 | FAM133B | 1.26E−04 | 1.13E−02 | 1.47E−03 | 3.01E−02 | 2.84E−02 | 6.45E−01 | 4.05E−04 | 6.50E−01 | 7.53E−01 |
| 7893442 | — | 1.26E−04 | 1.13E−02 | 1.65E−03 | 3.03E−02 | 6.18E−02 | 6.79E−01 | 4.05E−04 | 4.79E−01 | 9.55E−01 |
| 8140859 | MTERF | 1.27E−04 | 1.13E−02 | 5.85E−03 | 3.15E−02 | 9.97E−03 | 6.35E−01 | 3.12E−04 | 8.75E−01 | 4.30E−01 |
| 7924760 | ITPKB | 1.27E−04 | 1.13E−02 | 5.22E−04 | 2.98E−02 | 1.83E−01 | 7.31E−01 | 3.57E−04 | 4.34E−01 | 4.55E−01 |
| 7903032 | MTF2 | 1.27E−04 | 1.13E−02 | 2.70E−03 | 3.12E−02 | 3.06E−02 | 6.45E−01 | 4.26E−04 | 6.26E−01 | 5.12E−01 |
| 7894439 | — | 1.28E−04 | 1.13E−02 | 1.65E−04 | 2.75E−02 | 2.54E−01 | 7.53E−01 | 6.24E−04 | 3.83E−01 | 5.55E−01 |
| 7895634 | — | 1.28E−04 | 1.13E−02 | 7.98E−03 | 3.32E−02 | 8.37E−03 | 6.35E−01 | 3.07E−04 | 8.12E−01 | 2.86E−01 |
| 8025978 | ZNF763 | 1.28E−04 | 1.13E−02 | 2.16E−03 | 3.11E−02 | 4.95E−02 | 6.67E−01 | 1.45E−04 | 5.72E−01 | 8.32E−01 |
| 8056359 | SNORA70F | 1.29E−04 | 1.13E−02 | 1.24E−03 | 3.00E−02 | 5.22E−02 | 6.72E−01 | 3.91E−04 | 9.82E−01 | 9.30E−01 |
| 8028219 | ZNF420 | 1.31E−04 | 1.14E−02 | 5.53E−03 | 3.15E−02 | 6.02E−03 | 6.35E−01 | 2.74E−04 | 5.40E−01 | 2.75E−01 |
| 8096314 | PKD2 | 1.32E−04 | 1.15E−02 | 5.20E−04 | 2.98E−02 | 1.85E−01 | 7.32E−01 | 2.75E−04 | 5.18E−01 | 3.94E−01 |
| 8015827 | SOST | 1.33E−04 | 1.15E−02 | 4.27E−05 | 2.75E−02 | 3.57E−01 | 7.79E−01 | 5.21E−04 | 9.12E−01 | 2.79E−01 |
| 7894110 | — | 1.34E−04 | 1.15E−02 | 3.69E−04 | 2.75E−02 | 1.89E−01 | 7.34E−01 | 5.85E−04 | 4.49E−01 | 6.02E−01 |
| 8016980 | MIR142 | 1.34E−04 | 1.15E−02 | 3.61E−03 | 3.13E−02 | 5.76E−02 | 6.79E−01 | 3.07E−04 | 1.01E−01 | 9.81E−01 |
| 7961483 | HIST4H4 | 1.36E−04 | 1.15E−02 | 9.73E−04 | 3.00E−02 | 1.33E−01 | 7.16E−01 | 3.73E−04 | 2.75E−01 | 6.96E−01 |
| 8175835 | BCAP31 | 1.36E−04 | 1.15E−02 | 1.14E−03 | 3.00E−02 | 1.14E−01 | 7.03E−01 | 5.74E−04 | 3.01E−01 | 6.41E−01 |
| 7913776 | IL28RA | 1.39E−04 | 1.16E−02 | 5.39E−05 | 2.75E−02 | 5.43E−01 | 8.19E−01 | 6.17E−04 | 7.75E−01 | 1.11E−01 |
| 8030366 | SNORD35A | 1.39E−04 | 1.16E−02 | 3.52E−02 | 4.60E−02 | 5.95E−04 | 5.79E−01 | 1.85E−04 | 3.52E−01 | 1.21E−01 |
| 8038919 | ZNF350 | 1.41E−04 | 1.16E−02 | 1.11E−02 | 3.47E−02 | 1.06E−02 | 6.35E−01 | 3.94E−04 | 2.38E−01 | 2.98E−01 |
| 8115562 | RNF145 | 1.41E−04 | 1.16E−02 | 1.39E−03 | 3.00E−02 | 8.00E−02 | 6.94E−01 | 7.69E−04 | 4.23E−01 | 8.06E−01 |
| 7940857 | STIP1 | 1.44E−04 | 1.17E−02 | 2.78E−04 | 2.75E−02 | 1.98E−01 | 7.37E−01 | 3.89E−04 | 7.46E−01 | 5.02E−01 |
| 8005225 | LOC162632 | 1.46E−04 | 1.17E−02 | 1.73E−03 | 3.06E−02 | 1.00E−01 | 7.01E−01 | 2.20E−04 | 2.50E−01 | 7.59E−01 |
| 8168875 | ARMCX3 | 1.46E−04 | 1.17E−02 | 1.11E−03 | 3.00E−02 | 8.52E−02 | 6.94E−01 | 6.24E−04 | 4.65E−01 | 9.12E−01 |
| 7942592 | SNORD15A | 1.47E−04 | 1.17E−02 | 2.92E−02 | 4.32E−02 | 1.99E−03 | 6.06E−01 | 1.50E−04 | 9.47E−01 | 5.02E−02 |
| 8162472 | BARX1 | 1.47E−04 | 1.17E−02 | 2.91E−04 | 2.75E−02 | 2.16E−01 | 7.43E−01 | 2.94E−04 | 3.88E−01 | 5.34E−01 |
| 7982663 | BUB1B | 1.47E−04 | 1.17E−02 | 6.03E−04 | 3.00E−02 | 2.08E−01 | 7.42E−01 | 4.30E−04 | 3.22E−01 | 4.11E−01 |
| 8094772 | — | 1.49E−04 | 1.18E−02 | 6.20E−03 | 3.17E−02 | 2.34E−02 | 6.45E−01 | 2.88E−04 | 2.33E−01 | 6.01E−01 |
| 8122684 | SUMO4 | 1.51E−04 | 1.18E−02 | 7.53E−05 | 2.75E−02 | 3.31E−01 | 7.69E−01 | 5.13E−04 | 8.81E−01 | 2.67E−01 |
| 8096030 | — | 1.51E−04 | 1.18E−02 | 2.60E−04 | 2.75E−02 | 3.64E−01 | 7.82E−01 | 3.07E−04 | 2.67E−01 | 2.53E−01 |
| 8006715 | TADA2A | 1.53E−04 | 1.19E−02 | 1.15E−02 | 3.49E−02 | 6.57E−03 | 6.35E−01 | 2.56E−04 | 8.81E−01 | 1.50E−01 |
| 7980115 | ABCD4 | 1.53E−04 | 1.19E−02 | 2.04E−02 | 3.89E−02 | 5.26E−03 | 6.33E−01 | 1.47E−04 | 1.64E−01 | 4.38E−01 |
| 7894884 | — | 1.54E−04 | 1.19E−02 | 8.97E−03 | 3.35E−02 | 1.44E−02 | 6.41E−01 | 2.83E−04 | 3.65E−01 | 4.01E−01 |
| 8139832 | ZNF117 | 1.58E−04 | 1.21E−02 | 1.07E−03 | 3.00E−02 | 1.23E−01 | 7.08E−01 | 4.26E−04 | 4.27E−01 | 6.44E−01 |
| 8004144 | M1S12 | 1.59E−04 | 1.21E−02 | 6.98E−03 | 3.24E−02 | 1.28E−02 | 6.40E−01 | 3.40E−04 | 6.89E−01 | 4.26E−01 |
| 8053775 | ZNF514 | 1.63E−04 | 1.23E−02 | 4.06E−03 | 3.13E−02 | 5.31E−02 | 6.73E−01 | 2.67E−04 | 2.02E−01 | 8.52E−01 |
| 8013479 | CCDC144NL | 1.63E−04 | 1.23E−02 | 3.19E−02 | 4.45E−02 | 7.77E−03 | 6.35E−01 | 4.23E−06 | 5.86E−03 | 7.46E−01 |
| 8096251 | NUDT9 | 1.65E−04 | 1.24E−02 | 2.11E−02 | 3.92E−02 | 8.38E−03 | 6.35E−01 | 2.00E−04 | 1.98E−01 | 1.99E−01 |
| 7892951 | — | 1.67E−04 | 1.25E−02 | 2.65E−04 | 2.75E−02 | 2.63E−01 | 7.56E−01 | 4.90E−04 | 5.42E−01 | 3.80E−01 |
| 8039655 | ZNF550 | 1.68E−04 | 1.25E−02 | 7.31E−03 | 3.27E−02 | 1.15E−02 | 6.35E−01 | 1.73E−04 | 9.01E−01 | 3.49E−01 |
| 8004699 | CHD3 | 1.69E−04 | 1.25E−02 | 6.32E−04 | 3.00E−02 | 1.74E−01 | 7.28E−01 | 3.25E−04 | 5.37E−01 | 5.21E−01 |
| 8004508 | SNORA67 | 1.72E−04 | 1.26E−02 | 5.36E−03 | 3.14E−02 | 1.51E−02 | 6.41E−01 | 2.62E−04 | 9.91E−01 | 5.66E−01 |
| 7929768 | CUTC | 1.72E−04 | 1.26E−02 | 7.61E−03 | 3.29E−02 | 2.48E−02 | 6.45E−01 | 4.62E−04 | 1.85E−01 | 4.54E−01 |
| 8036813 | ZNF780B | 1.73E−04 | 1.26E−02 | 1.50E−03 | 3.01E−02 | 4.94E−02 | 6.67E−01 | 5.31E−04 | 8.91E−01 | 9.28E−01 |
| 8144078 | SHH | 1.75E−04 | 1.26E−02 | 5.85E−04 | 3.00E−02 | 7.32E−02 | 6.93E−01 | 6.46E−04 | 4.46E−01 | 6.16E−01 |
| 7929945 | — | 1.76E−04 | 1.26E−02 | 2.27E−04 | 2.75E−02 | 2.15E−01 | 7.43E−01 | 1.48E−03 | 7.91E−01 | 3.44E−01 |
| 7945979 | TRIM68 | 1.76E−04 | 1.26E−02 | 1.04E−02 | 3.42E−02 | 4.43E−02 | 6.66E−01 | 1.80E−04 | 1.32E−02 | 4.32E−01 |
| 8160016 | RANBP6 | 1.77E−04 | 1.26E−02 | 7.27E−04 | 3.00E−02 | 7.59E−02 | 6.93E−01 | 5.84E−04 | 6.79E−01 | 8.82E−01 |
| 8023259 | SNORD58A | 1.78E−04 | 1.26E−02 | 1.21E−03 | 3.00E−02 | 7.79E−02 | 6.94E−01 | 3.25E−04 | 9.51E−01 | 8.16E−01 |
| 8029399 | ZNF226 | 1.81E−04 | 1.27E−02 | 3.97E−03 | 3.13E−02 | 3.95E−02 | 6.60E−01 | 4.41E−04 | 4.01E−01 | 9.33E−01 |
| 8089714 | LSAMP | 1.81E−04 | 1.27E−02 | 2.78E−05 | 2.75E−02 | 4.22E−01 | 7.97E−01 | 3.50E−04 | 1.98E−01 | 8.94E−02 |
| 8081465 | BBX | 1.83E−04 | 1.27E−02 | 2.46E−04 | 2.75E−02 | 1.40E−01 | 7.23E−01 | 6.40E−04 | 4.60E−01 | 5.74E−01 |
| 8160581 | TOPORS | 1.83E−04 | 1.27E−02 | 2.88E−03 | 3.13E−02 | 5.80E−02 | 6.79E−01 | 6.07E−04 | 3.50E−01 | 7.90E−01 |
| 8156610 | HABP4 | 1.85E−04 | 1.27E−02 | 6.62E−03 | 3.20E−02 | 3.31E−02 | 6.45E−01 | 1.84E−04 | 2.97E−01 | 6.76E−01 |
| 7979743 | RDH11 | 1.85E−04 | 1.27E−02 | 2.84E−03 | 3.12E−02 | 3.31E−02 | 6.45E−01 | 8.42E−04 | 7.64E−01 | 7.07E−01 |
| 8034401 | ZNF564 | 1.89E−04 | 1.28E−02 | 1.98E−03 | 3.11E−02 | 7.29E−02 | 6.93E−01 | 6.93E−04 | 4.64E−01 | 8.82E−01 |
| 8180310 | DNAJB6 | 1.89E−04 | 1.28E−02 | 1.04E−03 | 3.00E−02 | 1.78E−01 | 7.29E−01 | 7.79E−04 | 1.33E−01 | 7.31E−01 |
| 8028186 | ZNF146 | 1.89E−04 | 1.28E−02 | 5.70E−03 | 3.15E−02 | 1.55E−02 | 6.41E−01 | 5.74E−04 | 8.94E−01 | 4.39E−01 |
| 8116494 | ZFP62 | 1.91E−04 | 1.29E−02 | 1.90E−03 | 3.11E−02 | 5.83E−02 | 6.79E−01 | 4.03E−04 | 8.34E−01 | 9.10E−01 |
| 7952335 | SNORD14E | 1.92E−04 | 1.29E−02 | 1.05E−03 | 3.00E−02 | 7.54E−02 | 6.93E−01 | 4.56E−04 | 9.37E−01 | 9.90E−01 |
| 8180218 | — | 1.93E−04 | 1.29E−02 | 2.48E−03 | 3.12E−02 | 8.40E−02 | 6.94E−01 | 5.54E−04 | 2.61E−01 | 9.08E−01 |
| 7947189 | CCDC34 | 1.94E−04 | 1.29E−02 | 4.34E−05 | 2.75E−02 | 6.33E−01 | 8.36E−01 | 1.66E−03 | 9.17E−01 | 9.38E−02 |
| 8132118 | AQP1 | 1.95E−04 | 1.29E−02 | 8.00E−05 | 2.75E−02 | 4.65E−01 | 8.06E−01 | 7.87E−04 | 7.61E−01 | 1.51E−01 |
| 7996837 | CDH1 | 1.98E−04 | 1.31E−02 | 9.75E−03 | 3.38E−02 | 1.11E−02 | 6.35E−01 | 9.93E−04 | 4.48E−01 | 2.80E−01 |
| 8139085 | — | 2.01E−04 | 1.31E−02 | 3.42E−05 | 2.75E−02 | 9.06E−01 | 8.78E−01 | 1.12E−03 | 6.75E−01 | 3.88E−02 |
| 8110408 | THOC3 | 2.01E−04 | 1.31E−02 | 1.74E−02 | 3.75E−02 | 3.21E−03 | 6.06E−01 | 2.72E−04 | 4.64E−01 | 2.82E−01 |
| 8049959 | FLJ41327 | 2.03E−04 | 1.31E−02 | 6.52E−04 | 3.00E−02 | 1.89E−01 | 7.34E−01 | 9.36E−04 | 3.93E−01 | 6.77E−01 |
| 8115476 | MED7 | 2.04E−04 | 1.31E−02 | 3.06E−03 | 3.13E−02 | 2.70E−02 | 6.45E−01 | 6.76E−04 | 8.92E−01 | 7.06E−01 |
| 7968265 | PDX1 | 2.04E−04 | 1.31E−02 | 2.87E−04 | 2.75E−02 | 1.93E−01 | 7.35E−01 | 6.02E−04 | 7.64E−01 | 5.28E−01 |
| 8122701 | — | 2.05E−04 | 1.31E−02 | 1.47E−03 | 3.01E−02 | 9.03E−02 | 6.96E−01 | 6.48E−04 | 6.19E−01 | 9.29E−01 |
| 8089993 | WDR5B | 2.06E−04 | 1.31E−02 | 2.34E−03 | 3.11E−02 | 6.12E−02 | 6.79E−01 | 6.01E−04 | 6.10E−01 | 9.25E−01 |
| 8038962 | ZNF836 | 2.08E−04 | 1.31E−02 | 8.55E−03 | 3.34E−02 | 2.11E−02 | 6.45E−01 | 2.65E−04 | 4.67E−01 | 6.08E−01 |
| 7927071 | ZNF37A | 2.08E−04 | 1.31E−02 | 1.02E−03 | 3.00E−02 | 8.49E−02 | 6.94E−01 | 4.93E−04 | 9.03E−01 | 8.69E−01 |
| 8152759 | TATDN1 | 2.08E−04 | 1.31E−02 | 2.01E−02 | 3.88E−02 | 1.32E−03 | 5.98E−01 | 2.21E−04 | 2.56E−01 | 7.38E−02 |
| 7980463 | SNW1 | 2.09E−04 | 1.31E−02 | 9.64E−03 | 3.37E−02 | 2.88E−02 | 6.45E−01 | 4.30E−04 | 1.36E−01 | 3.45E−01 |
| 7894426 | — | 2.10E−04 | 1.31E−02 | 6.60E−02 | 5.99E−02 | 1.80E−03 | 5.98E−01 | 7.59E−05 | 1.34E−01 | 1.34E−01 |
| 7893523 | — | 2.11E−04 | 1.31E−02 | 5.94E−03 | 3.15E−02 | 1.28E−01 | 7.12E−01 | 2.53E−04 | 2.47E−03 | 7.19E−01 |
| 7895833 | — | 2.13E−04 | 1.31E−02 | 9.57E−03 | 3.37E−02 | 2.90E−02 | 6.45E−01 | 4.88E−04 | 1.44E−01 | 4.88E−01 |
| 8092905 | LSG1 | 2.16E−04 | 1.31E−02 | 5.38E−03 | 3.14E−02 | 3.70E−02 | 6.59E−01 | 6.82E−04 | 3.16E−01 | 6.71E−01 |
| 8067798 | SOX18 | 2.17E−04 | 1.31E−02 | 1.58E−02 | 3.66E−02 | 1.66E−03 | 5.98E−01 | 3.38E−04 | 4.35E−01 | 1.75E−01 |
| 7893821 | — | 2.18E−04 | 1.31E−02 | 1.00E−03 | 3.00E−02 | 9.82E−02 | 7.00E−01 | 6.79E−04 | 9.17E−01 | 8.56E−01 |
| 8180356 | RPL7L1 | 2.18E−04 | 1.31E−02 | 1.51E−02 | 3.63E−02 | 1.98E−02 | 6.45E−01 | 2.32E−04 | 1.59E−01 | 3.64E−01 |
| 8084812 | — | 2.19E−04 | 1.31E−02 | 2.03E−04 | 2.75E−02 | 2.08E−01 | 7.42E−01 | 9.61E−04 | 4.87E−01 | 3.43E−01 |
| 8005141 | TTC19 | 2.19E−04 | 1.31E−02 | 8.84E−03 | 3.34E−02 | 1.55E−02 | 6.41E−01 | 5.94E−04 | 5.58E−01 | 4.41E−01 |
| 8007745 | HEX1M1 | 2.20E−04 | 1.31E−02 | 4.56E−03 | 3.13E−02 | 8.71E−02 | 6.94E−01 | 5.92E−04 | 5.17E−02 | 9.77E−01 |
| 8118613 | SLC39A7 | 2.22E−04 | 1.31E−02 | 2.20E−03 | 3.11E−02 | 1.73E−01 | 7.28E−01 | 4.88E−04 | 2.42E−02 | 8.24E−01 |
| 8178225 | SLC39A7 | 2.22E−04 | 1.31E−02 | 2.20E−03 | 3.11E−02 | 1.73E−01 | 7.28E−01 | 4.88E−04 | 2.42E−02 | 8.24E−01 |
| 8179525 | SLC39A7 | 2.22E−04 | 1.31E−02 | 2.20E−03 | 3.11E−02 | 1.73E−01 | 7.28E−01 | 4.88E−04 | 2.42E−02 | 8.24E−01 |
| 8176460 | PRKY | 2.22E−04 | 1.31E−02 | 3.37E−04 | 2.75E−02 | 3.54E−01 | 7.78E−01 | 2.99E−12 | 3.64E−54 | 6.09E−03 |
| 8072573 | — | 2.25E−04 | 1.32E−02 | 5.79E−02 | 5.62E−02 | 5.58E−04 | 5.79E−01 | 9.62E−05 | 7.54E−01 | 1.02E−01 |
| 8033780 | ZNF426 | 2.27E−04 | 1.32E−02 | 3.95E−03 | 3.13E−02 | 3.65E−02 | 6.59E−01 | 6.26E−04 | 6.96E−01 | 5.86E−01 |
| 7918792 | DENND2C | 2.27E−04 | 1.32E−02 | 3.41E−04 | 2.75E−02 | 1.52E−01 | 7.25E−01 | 8.94E−04 | 4.46E−01 | 4.40E−01 |
| 8066776 | TP53RK | 2.31E−04 | 1.34E−02 | 3.49E−03 | 3.13E−02 | 3.89E−02 | 6.59E−01 | 7.51E−04 | 7.20E−01 | 7.72E−01 |
| 7955110 | — | 2.32E−04 | 1.34E−02 | 8.57E−03 | 3.34E−02 | 1.75E−02 | 6.43E−01 | 4.49E−04 | 5.36E−01 | 6.07E−01 |
| 8121319 | SOBP | 2.33E−04 | 1.34E−02 | 5.84E−05 | 2.75E−02 | 8.99E−01 | 8.76E−01 | 1.41E−03 | 5.13E−01 | 4.62E−02 |
| 7914141 | RPA2 | 2.36E−04 | 1.35E−02 | 2.01E−02 | 3.88E−02 | 1.94E−03 | 5.98E−01 | 6.19E−04 | 6.43E−01 | 6.10E−02 |
| 8176476 | — | 2.37E−04 | 1.35E−02 | 2.12E−03 | 3.11E−02 | 2.04E−02 | 6.45E−01 | 2.27E−04 | 1.68E−01 | 9.11E−01 |
| 7910217 | WNT3A | 2.38E−04 | 1.35E−02 | 2.97E−04 | 2.75E−02 | 1.64E−01 | 7.26E−01 | 1.62E−03 | 4.29E−01 | 4.02E−01 |
| 8034512 | SNORD41 | 2.39E−04 | 1.36E−02 | 7.59E−04 | 3.00E−02 | 1.05E−01 | 7.01E−01 | 4.96E−04 | 6.20E−01 | 5.85E−01 |
| 8020889 | ZNF397 | 2.41E−04 | 1.36E−02 | 2.05E−03 | 3.11E−02 | 1.10E−01 | 7.01E−01 | 6.94E−04 | 2.77E−01 | 9.70E−01 |
| 7897089 | PLCH2 | 2.43E−04 | 1.36E−02 | 3.48E−04 | 2.75E−02 | 7.46E−02 | 6.93E−01 | 1.30E−03 | 1.48E−01 | 9.33E−01 |
| 8145793 | SNORD13 | 2.43E−04 | 1.36E−02 | 4.39E−03 | 3.13E−02 | 2.36E−02 | 6.45E−01 | 2.95E−04 | 7.95E−01 | 7.16E−01 |
| 7970732 | PRHOXNB | 2.45E−04 | 1.36E−02 | 4.39E−05 | 2.75E−02 | 5.23E−01 | 8.16E−01 | 4.77E−04 | 7.26E−01 | 9.77E−02 |
| 8176375 | RPS4Y1 | 2.45E−04 | 1.36E−02 | 1.98E−03 | 3.11E−02 | 4.41E−01 | 8.00E−01 | 9.21E−10 | 2.42E−44 | 2.44E−02 |
| 8010078 | SNORD1C | 2.48E−04 | 1.38E−02 | 9.78E−04 | 3.00E−02 | 7.92E−02 | 6.94E−01 | 6.10E−04 | 5.67E−01 | 6.20E−01 |
| 7917597 | — | 2.50E−04 | 1.38E−02 | 2.78E−04 | 2.75E−02 | 2.88E−01 | 7.62E−01 | 1.15E−03 | 9.92E−01 | 3.16E−01 |
| 7896546 | — | 2.53E−04 | 1.39E−02 | 2.40E−03 | 3.12E−02 | 2.26E−02 | 6.45E−01 | 1.11E−03 | 4.18E−01 | 6.94E−01 |
| 7962811 | C12orf41 | 2.55E−04 | 1.39E−02 | 2.56E−03 | 3.12E−02 | 1.10E−01 | 7.01E−01 | 6.83E−04 | 1.81E−01 | 9.88E−01 |
| 8098752 | ABCA11P | 2.56E−04 | 1.39E−02 | 2.55E−02 | 4.13E−02 | 1.12E−02 | 6.35E−01 | 1.71E−04 | 2.52E−01 | 2.76E−01 |
| 8174047 | TIMM8A | 2.60E−04 | 1.41E−02 | 7.99E−03 | 3.32E−02 | 1.46E−02 | 6.41E−01 | 6.40E−04 | 9.49E−01 | 3.80E−01 |
| 8134880 | MOSPD3 | 2.61E−04 | 1.41E−02 | 2.71E−04 | 2.75E−02 | 2.14E−01 | 7.43E−01 | 7.97E−04 | 3.93E−01 | 2.49E−01 |
| 7894330 | — | 2.63E−04 | 1.41E−02 | 2.28E−03 | 3.11E−02 | 1.22E−01 | 7.08E−01 | 4.21E−04 | 2.72E−01 | 7.83E−01 |
| 8026122 | RAD23A | 2.64E−04 | 1.41E−02 | 1.01E−01 | 7.46E−02 | 1.84E−04 | 4.02E−01 | 1.68E−04 | 7.81E−01 | 2.70E−02 |
| 8115886 | THOC3 | 2.64E−04 | 1.41E−02 | 2.00E−02 | 3.87E−02 | 3.13E−03 | 6.06E−01 | 3.68E−04 | 5.32E−01 | 2.96E−01 |
| 8034578 | KLF1 | 2.65E−04 | 1.41E−02 | 1.85E−03 | 3.10E−02 | 1.12E−01 | 7.01E−01 | 1.42E−03 | 3.75E−01 | 8.71E−01 |
| 7908758 | SHISA4 | 2.67E−04 | 1.41E−02 | 8.60E−05 | 2.75E−02 | 2.75E−01 | 7.58E−01 | 1.02E−03 | 1.85E−01 | 2.89E−01 |
| 8116532 | SNORD95 | 2.68E−04 | 1.41E−02 | 2.68E−03 | 3.12E−02 | 2.37E−02 | 6.45E−01 | 3.17E−04 | 3.08E−01 | 8.08E−01 |
| 8114030 | KIF3A | 2.69E−04 | 1.41E−02 | 2.95E−03 | 3.13E−02 | 5.14E−02 | 6.71E−01 | 5.98E−04 | 8.90E−01 | 8.37E−01 |
| 7922414 | SNORD76 | 2.70E−04 | 1.41E−02 | 2.07E−03 | 3.11E−02 | 5.85E−02 | 6.79E−01 | 4.50E−04 | 8.51E−01 | 9.43E−01 |
| 7998931 | ZNF200 | 2.71E−04 | 1.41E−02 | 1.39E−02 | 3.56E−02 | 6.76E−03 | 6.35E−01 | 8.47E−04 | 8.11E−01 | 2.82E−01 |
| 8149733 | TNFRSF10B | 2.71E−04 | 1.41E−02 | 4.75E−03 | 3.13E−02 | 2.18E−01 | 7.43E−01 | 6.00E−04 | 3.59E−04 | 7.18E−01 |
| 7895846 | — | 2.71E−04 | 1.41E−02 | 1.04E−02 | 3.42E−02 | 2.32E−02 | 6.45E−01 | 5.50E−04 | 3.44E−01 | 4.91E−01 |
| 8026007 | ZNF791 | 2.73E−04 | 1.41E−02 | 4.88E−03 | 3.13E−02 | 3.63E−02 | 6.59E−01 | 7.24E−04 | 6.66E−01 | 7.61E−01 |
| 8143065 | C7orf49 | 2.75E−04 | 1.42E−02 | 3.29E−03 | 3.13E−02 | 7.30E−02 | 6.93E−01 | 6.45E−04 | 4.37E−01 | 9.09E−01 |
| 7960143 | ZNF84 | 2.77E−04 | 1.42E−02 | 5.30E−03 | 3.13E−02 | 2.60E−02 | 6.45E−01 | 6.51E−04 | 9.71E−01 | 6.04E−01 |
| 8112649 | FAM169A | 2.78E−04 | 1.42E−02 | 6.52E−03 | 3.19E−02 | 3.57E−02 | 6.54E−01 | 6.08E−04 | 4.85E−01 | 6.38E−01 |
| 8059783 | NGEF | 2.79E−04 | 1.42E−02 | 1.22E−04 | 2.75E−02 | 2.50E−01 | 7.51E−01 | 2.38E−03 | 3.02E−01 | 4.40E−01 |
| 7919560 | — | 2.82E−04 | 1.42E−02 | 1.19E−02 | 3.51E−02 | 1.40E−02 | 6.41E−01 | 3.90E−04 | 6.36E−01 | 5.31E−01 |
| 8059014 | FEV | 2.84E−04 | 1.42E−02 | 6.02E−03 | 3.15E−02 | 1.56E−02 | 6.41E−01 | 4.90E−04 | 7.72E−01 | 7.38E−01 |
| 7893779 | — | 2.85E−04 | 1.42E−02 | 2.42E−03 | 3.12E−02 | 1.26E−01 | 7.10E−01 | 1.07E−03 | 1.87E−01 | 9.08E−01 |
| 8004325 | EIF5A | 2.85E−04 | 1.42E−02 | 1.34E−02 | 3.54E−02 | 4.14E−03 | 6.08E−01 | 3.31E−04 | 4.02E−01 | 2.54E−01 |
| 7976158 | — | 2.85E−04 | 1.42E−02 | 1.97E−04 | 2.75E−02 | 3.07E−01 | 7.66E−01 | 1.52E−03 | 8.03E−01 | 4.50E−01 |
| 8002087 | RANBP10 | 2.86E−04 | 1.42E−02 | 9.07E−02 | 7.02E−02 | 1.66E−03 | 5.98E−01 | 3.59E−04 | 1.90E−01 | 2.60E−02 |
| 8052731 | PPP3R1 | 2.86E−04 | 1.42E−02 | 8.53E−04 | 3.00E−02 | 7.72E−02 | 6.94E−01 | 8.25E−04 | 4.04E−01 | 8.98E−01 |
| 7969228 | ALG11 | 2.88E−04 | 1.42E−02 | 2.53E−03 | 3.12E−02 | 5.47E−02 | 6.75E−01 | 9.16E−04 | 9.22E−01 | 6.78E−01 |
| 8073949 | CRELD2 | 2.91E−04 | 1.42E−02 | 9.54E−05 | 2.75E−02 | 4.65E−01 | 8.06E−01 | 7.65E−04 | 8.38E−01 | 1.96E−01 |
| 8162880 | MRPL50 | 2.91E−04 | 1.42E−02 | 1.38E−02 | 3.56E−02 | 8.64E−03 | 6.35E−01 | 5.60E−04 | 9.42E−01 | 3.02E−01 |
| 8058373 | WDR12 | 2.92E−04 | 1.42E−02 | 2.04E−02 | 3.89E−02 | 8.12E−03 | 6.35E−01 | 5.36E−04 | 6.64E−01 | 1.77E−01 |
| 8086752 | SNORD13 | 2.92E−04 | 1.42E−02 | 8.08E−03 | 3.32E−02 | 1.58E−02 | 6.41E−01 | 3.12E−04 | 9.52E−01 | 6.17E−01 |
| 8046560 | HOXD3 | 2.92E−04 | 1.42E−02 | 7.51E−04 | 3.00E−02 | 6.92E−02 | 6.90E−01 | 1.65E−03 | 3.10E−01 | 7.32E−01 |
| 7919556 | — | 2.92E−04 | 1.42E−02 | 1.29E−02 | 3.53E−02 | 1.23E−02 | 6.35E−01 | 3.97E−04 | 6.77E−01 | 4.99E−01 |
| 7925174 | TOMM20 | 2.94E−04 | 1.42E−02 | 4.62E−03 | 3.13E−02 | 3.13E−02 | 6.45E−01 | 9.12E−04 | 9.15E−01 | 6.59E−01 |
| 8099362 | — | 2.94E−04 | 1.42E−02 | 1.76E−03 | 3.08E−02 | 2.04E−01 | 7.39E−01 | 1.20E−03 | 1.02E−01 | 6.38E−01 |
| 8011027 | MYO1C | 2.95E−04 | 1.42E−02 | 5.66E−04 | 3.00E−02 | 8.80E−02 | 6.94E−01 | 2.33E−03 | 2.85E−01 | 5.95E−01 |
| 7993298 | ERCC4 | 2.98E−04 | 1.42E−02 | 2.79E−03 | 3.12E−02 | 5.16E−02 | 6.71E−01 | 1.01E−03 | 9.54E−01 | 7.36E−01 |
| 8115164 | — | 2.99E−04 | 1.43E−02 | 1.21E−02 | 3.51E−02 | 4.26E−02 | 6.62E−01 | 5.43E−04 | 6.67E−02 | 5.14E−01 |
| 8154765 | DNAJA1 | 3.01E−04 | 1.43E−02 | 1.11E−02 | 3.47E−02 | 2.05E−02 | 6.45E−01 | 6.93E−04 | 4.68E−01 | 3.35E−01 |
| 8062766 | MYBL2 | 3.01E−04 | 1.43E−02 | 1.79E−03 | 3.09E−02 | 7.15E−02 | 6.93E−01 | 1.05E−03 | 9.95E−01 | 8.80E−01 |
| 8026272 | IL27RA | 3.02E−04 | 1.43E−02 | 3.67E−04 | 2.75E−02 | 1.62E−01 | 7.26E−01 | 8.94E−04 | 3.85E−01 | 4.93E−01 |
| 8176730 | RPS4Y2 | 3.04E−04 | 1.43E−02 | 2.13E−03 | 3.11E−02 | 4.27E−01 | 7.97E−01 | 1.99E−10 | 1.60E−48 | 2.31E−02 |
| 8155268 | POLR1E | 3.05E−04 | 1.43E−02 | 1.46E−02 | 3.59E−02 | 1.77E−02 | 6.43E−01 | 4.47E−04 | 3.46E−01 | 3.71E−01 |
| 8034589 | FARSA | 3.05E−04 | 1.43E−02 | 6.09E−03 | 3.15E−02 | 4.12E−02 | 6.61E−01 | 5.54E−04 | 4.75E−01 | 8.74E−01 |
| 8027566 | CEBPG | 3.07E−04 | 1.43E−02 | 2.48E−03 | 3.12E−02 | 1.33E−01 | 7.16E−01 | 1.07E−03 | 2.02E−01 | 8.35E−01 |
| 8162669 | ZNF322 | 3.07E−04 | 1.43E−02 | 7.28E−04 | 3.00E−02 | 1.86E−01 | 7.32E−01 | 8.31E−04 | 7.41E−01 | 6.04E−01 |
| 8096081 | ENOPH1 | 3.09E−04 | 1.43E−02 | 1.24E−02 | 3.52E−02 | 1.63E−02 | 6.41E−01 | 7.60E−04 | 4.23E−01 | 4.53E−01 |
| 7931926 | — | 3.10E−04 | 1.43E−02 | 4.25E−03 | 3.13E−02 | 6.67E−02 | 6.88E−01 | 5.78E−04 | 4.50E−01 | 9.46E−01 |
| 8066214 | TGM2 | 3.11E−04 | 1.43E−02 | 1.54E−04 | 2.75E−02 | 4.04E−01 | 7.92E−01 | 1.59E−03 | 7.49E−01 | 2.28E−01 |
| 8086482 | ZNF445 | 3.12E−04 | 1.43E−02 | 3.38E−03 | 3.13E−02 | 1.24E−01 | 7.08E−01 | 5.94E−04 | 1.06E−01 | 9.80E−01 |
| 8093826 | ADRA2C | 3.12E−04 | 1.43E−02 | 1.30E−02 | 3.53E−02 | 9.37E−03 | 6.35E−01 | 8.79E−04 | 7.36E−01 | 3.38E−01 |
| 8052940 | PAIP2B | 3.16E−04 | 1.44E−02 | 6.16E−03 | 3.16E−02 | 3.03E−02 | 6.45E−01 | 3.03E−04 | 9.64E−01 | 4.98E−01 |
| 8029321 | ZNF283 | 3.19E−04 | 1.44E−02 | 6.93E−03 | 3.24E−02 | 2.63E−02 | 6.45E−01 | 5.12E−04 | 9.36E−01 | 4.78E−01 |
| 7979565 | WDR89 | 3.21E−04 | 1.44E−02 | 1.66E−02 | 3.70E−02 | 4.50E−03 | 6.11E−01 | 6.21E−04 | 7.34E−01 | 2.36E−01 |
| 7896464 | — | 3.21E−04 | 1.44E−02 | 8.53E−03 | 3.34E−02 | 2.72E−02 | 6.45E−01 | 9.44E−04 | 5.58E−01 | 4.45E−01 |
| 8060745 | SMOX | 3.22E−04 | 1.44E−02 | 4.34E−02 | 4.97E−02 | 9.08E−03 | 6.35E−01 | 3.26E−04 | 3.79E−02 | 2.60E−01 |
| 8151909 | UQCRB | 3.23E−04 | 1.44E−02 | 6.94E−03 | 3.24E−02 | 4.02E−02 | 6.61E−01 | 9.53E−04 | 4.02E−01 | 5.84E−01 |
| 7969792 | — | 3.23E−04 | 1.44E−02 | 1.43E−03 | 3.01E−02 | 2.09E−02 | 6.45E−01 | 1.01E−03 | 6.81E−02 | 7.13E−01 |
| 7971561 | — | 3.24E−04 | 1.44E−02 | 4.07E−04 | 2.76E−02 | 1.59E−01 | 7.26E−01 | 1.33E−03 | 5.14E−01 | 6.79E−01 |
| 8137627 | DNAJ86 | 3.27E−04 | 1.44E−02 | 1.08E−03 | 3.00E−02 | 2.18E−01 | 7.43E−01 | 1.59E−03 | 3.21E−01 | 5.63E−01 |
| 7901046 | SNORD55 | 3.28E−04 | 1.44E−02 | 4.55E−03 | 3.13E−02 | 4.19E−02 | 6.61E−01 | 5.53E−04 | 8.26E−01 | 9.15E−01 |
| 8065018 | TASP1 | 3.28E−04 | 1.44E−02 | 1.27E−03 | 3.00E−02 | 1.57E−01 | 7.26E−01 | 6.18E−04 | 4.01E−01 | 8.54E−01 |
| 8072488 | DRG1 | 3.29E−04 | 1.44E−02 | 1.73E−02 | 3.74E−02 | 2.74E−02 | 6.45E−01 | 7.24E−04 | 6.80E−02 | 4.08E−01 |
| 8027385 | VSTM2B | 3.30E−04 | 1.44E−02 | 3.23E−04 | 2.75E−02 | 2.92E−01 | 7.63E−01 | 1.07E−03 | 9.00E−01 | 3.73E−01 |
| 8180376 | AKR1C1 | 3.30E−04 | 1.44E−02 | 4.88E−02 | 5.21E−02 | 9.37E−04 | 5.98E−01 | 2.36E−04 | 2.37E−01 | 3.12E−01 |
| 8024013 | C19orf21 | 3.30E−04 | 1.44E−02 | 1.93E−03 | 3.11E−02 | 4.31E−02 | 6.65E−01 | 1.11E−03 | 4.24E−01 | 9.75E−01 |
| 7966321 | GPN3 | 3.33E−04 | 1.45E−02 | 3.26E−02 | 4.48E−02 | 2.62E−03 | 6.06E−01 | 8.39E−04 | 4.22E−01 | 1.71E−01 |
| 7970413 | PSPC1 | 3.35E−04 | 1.45E−02 | 1.42E−03 | 3.01E−02 | 1.49E−01 | 7.24E−01 | 1.56E−03 | 4.60E−01 | 8.45E−01 |
| 7894914 | — | 3.35E−04 | 1.45E−02 | 4.52E−04 | 2.88E−02 | 2.59E−01 | 7.55E−01 | 1.61E−03 | 6.70E−01 | 5.71E−01 |
| 8176469 | — | 3.42E−04 | 1.47E−02 | 5.29E−03 | 3.13E−02 | 7.18E−01 | 8.53E−01 | 3.11E−10 | 1.70E−47 | 5.84E−02 |
| 8113491 | STARD4 | 3.46E−04 | 1.48E−02 | 3.91E−03 | 3.13E−02 | 2.67E−02 | 6.45E−01 | 1.23E−03 | 6.96E−01 | 7.12E−01 |
| 7938331 | ZNF143 | 3.46E−04 | 1.48E−02 | 6.10E−03 | 3.15E−02 | 8.53E−02 | 6.94E−01 | 9.74E−04 | 8.09E−02 | 7.73E−01 |
| 7911371 | C1orf170 | 3.47E−04 | 1.48E−02 | 2.38E−03 | 3.11E−02 | 4.71E−02 | 6.67E−01 | 1.33E−03 | 8.18E−01 | 8.67E−01 |
| 8129363 | HDDC2 | 3.48E−04 | 1.48E−02 | 1.41E−02 | 3.57E−02 | 7.93E−03 | 6.35E−01 | 6.99E−04 | 8.67E−01 | 2.24E−01 |
| 7927854 | HNRNPH3 | 3.50E−04 | 1.48E−02 | 4.29E−03 | 3.13E−02 | 8.40E−02 | 6.94E−01 | 1.17E−03 | 2.31E−01 | 8.34E−01 |
| 8140151 | RFC2 | 3.51E−04 | 1.48E−02 | 1.80E−02 | 3.78E−02 | 2.15E−02 | 6.45E−01 | 9.45E−04 | 1.42E−01 | 3.09E−01 |
| 7983616 | GALK2 | 3.52E−04 | 1.48E−02 | 1.58E−03 | 3.02E−02 | 1.08E−01 | 7.01E−01 | 1.38E−03 | 7.98E−01 | 9.68E−01 |
| 7953409 | PTMS | 3.53E−04 | 1.48E−02 | 1.61E−02 | 3.68E−02 | 2.17E−02 | 6.45E−01 | 8.17E−04 | 2.95E−01 | 1.80E−01 |
| 7893629 | — | 3.54E−04 | 1.48E−02 | 7.31E−04 | 3.00E−02 | 2.88E−01 | 7.62E−01 | 3.03E−04 | 7.93E−01 | 2.21E−01 |
| 8135064 | TRIM56 | 3.57E−04 | 1.49E−02 | 7.16E−03 | 3.26E−02 | 3.38E−02 | 6.46E−01 | 1.01E−03 | 5.38E−01 | 7.06E−01 |
| 8010295 | ENGASE | 3.58E−04 | 1.49E−02 | 4.12E−02 | 4.87E−02 | 1.35E−02 | 6.40E−01 | 8.36E−05 | 3.20E−02 | 5.53E−01 |
| 8107321 | EPB41L4A- | 3.62E−04 | 1.50E−02 | 1.26E−02 | 3.52E−02 | 1.11E−02 | 6.35E−01 | 7.40E−04 | 8.22E−01 | 4.78E−01 |
| | AS1 | | | | | | | | | |
| 7925201 | ARID4B | 3.64E−04 | 1.51E−02 | 4.26E−03 | 3.13E−02 | 2.92E−02 | 6.45E−01 | 1.54E−03 | 7.61E−01 | 5.62E−01 |
| 8062623 | PLCG1 | 3.68E−04 | 1.52E−02 | 2.65E−03 | 3.12E−02 | 7.24E−02 | 6.93E−01 | 7.55E−04 | 9.65E−01 | 9.41E−01 |
| 7950307 | UCP2 | 3.73E−04 | 1.54E−02 | 4.18E−02 | 4.89E−02 | 5.99E−03 | 6.35E−01 | 3.63E−04 | 1.15E−01 | 3.10E−01 |
| 7958331 | RIC8B | 3.76E−04 | 1.55E−02 | 1.28E−03 | 3.00E−02 | 1.15E−01 | 7.03E−01 | 1.14E−03 | 8.52E−01 | 7.69E−01 |
| 7995421 | LONP2 | 3.77E−04 | 1.55E−02 | 4.63E−03 | 3.13E−02 | 6.04E−02 | 6.79E−01 | 1.20E−03 | 5.15E−01 | 7.49E−01 |
| 7897953 | SNORA59A | 3.81E−04 | 1.55E−02 | 4.46E−03 | 3.13E−02 | 1.82E−02 | 6.43E−01 | 5.93E−04 | 2.64E−01 | 5.56E−01 |
| 8005626 | SNORA59A | 3.81E−04 | 1.55E−02 | 4.46E−03 | 3.13E−02 | 1.82E−02 | 6.43E−01 | 5.93E−04 | 2.64E−01 | 5.56E−01 |
| 8020898 | ZNF271 | 3.88E−04 | 1.57E−02 | 4.05E−03 | 3.13E−02 | 7.55E−02 | 6.93E−01 | 9.95E−04 | 5.04E−01 | 8.90E−01 |
| 8113059 | MBLAC2 | 3.88E−04 | 1.57E−02 | 4.05E−03 | 3.13E−02 | 5.04E−02 | 6.69E−01 | 5.32E−04 | 9.19E−01 | 9.46E−01 |
| 8122317 | HEBP2 | 3.90E−04 | 1.57E−02 | 1.01E−02 | 3.40E−02 | 3.48E−02 | 6.51E−01 | 1.24E−03 | 3.00E−01 | 5.80E−01 |
| 7992987 | HMOX2 | 3.92E−04 | 1.58E−02 | 1.25E−02 | 3.52E−02 | 2.72E−02 | 6.45E−01 | 9.12E−04 | 3.27E−01 | 3.27E−01 |
| 7927033 | ANKRD30A | 3.96E−04 | 1.59E−02 | 1.19E−03 | 3.00E−02 | 1.74E−01 | 7.28E−01 | 1.15E−03 | 7.74E−01 | 6.20E−01 |
| 8036304 | ZFP14 | 3.97E−04 | 1.59E−02 | 2.26E−03 | 3.11E−02 | 8.79E−02 | 6.94E−01 | 9.03E−04 | 9.51E−01 | 9.65E−01 |
| 7920839 | RIT1 | 4.03E−04 | 1.61E−02 | 1.28E−03 | 3.00E−02 | 1.96E−01 | 7.36E−01 | 1.64E−03 | 3.87E−01 | 7.41E−01 |
| 8031815 | ZNF776 | 4.04E−04 | 1.61E−02 | 3.51E−03 | 3.13E−02 | 9.21E−02 | 6.96E−01 | 1.33E−03 | 4.42E−01 | 9.98E−01 |
| 8015445 | NT5C3L | 4.13E−04 | 1.64E−02 | 1.84E−02 | 3.79E−02 | 1.25E−02 | 6.35E−01 | 2.94E−04 | 7.64E−01 | 5.14E−01 |
| 7984215 | — | 4.18E−04 | 1.65E−02 | 3.24E−02 | 4.47E−02 | 9.21E−03 | 6.35E−01 | 1.59E−04 | 3.90E−01 | 4.39E−01 |
| 8098707 | HSP90AA4P | 4.22E−04 | 1.66E−02 | 2.24E−02 | 3.98E−02 | 7.88E−03 | 6.35E−01 | 9.00E−04 | 8.83E−01 | 1.45E−01 |
| 8014037 | CRLF3 | 4.22E−04 | 1.66E−02 | 3.23E−03 | 3.13E−02 | 8.07E−02 | 6.94E−01 | 1.61E−03 | 5.94E−01 | 7.87E−01 |
| 8034390 | ZNF799 | 4.24E−04 | 1.67E−02 | 1.30E−03 | 3.00E−02 | 1.66E−01 | 7.26E−01 | 8.88E−04 | 8.31E−01 | 6.11E−01 |
| 7955425 | ATF1 | 4.29E−04 | 1.68E−02 | 3.22E−03 | 3.13E−02 | 1.08E−01 | 7.01E−01 | 1.50E−03 | 4.22E−01 | 9.21E−01 |
| 8036737 | RPS16 | 4.30E−04 | 1.68E−02 | 8.21E−03 | 3.32E−02 | 2.85E−02 | 6.45E−01 | 1.04E−03 | 8.04E−01 | 5.80E−01 |
| 7895953 | — | 4.31E−04 | 1.68E−02 | 1.18E−03 | 3.00E−02 | 2.05E−01 | 7.40E−01 | 9.93E−04 | 3.84E−01 | 7.40E−01 |
| 8085571 | METTL6 | 4.33E−04 | 1.68E−02 | 3.34E−03 | 3.13E−02 | 1.23E−01 | 7.08E−01 | 9.45E−04 | 3.36E−01 | 8.76E−01 |
| 7977879 | PSMB5 | 4.38E−04 | 1.70E−02 | 2.49E−02 | 4.10E−02 | 4.52E−03 | 6.11E−01 | 1.54E−03 | 8.56E−01 | 1.83E−01 |
| 8029377 | ZNF224 | 4.41E−04 | 1.70E−02 | 2.79E−03 | 3.12E−02 | 1.01E−01 | 7.01E−01 | 1.44E−03 | 5.91E−01 | 9.25E−01 |
| 8146268 | FNTA | 4.42E−04 | 1.70E−02 | 4.38E−03 | 3.13E−02 | 5.64E−02 | 6.78E−01 | 1.56E−03 | 7.57E−01 | 8.05E−01 |
| 8076393 | CENPM | 4.44E−04 | 1.70E−02 | 2.44E−03 | 3.12E−02 | 5.11E−02 | 6.70E−01 | 1.27E−03 | 4.90E−01 | 8.94E−01 |
| 8099897 | UGDH | 4.45E−04 | 1.70E−02 | 4.05E−03 | 3.13E−02 | 6.12E−02 | 6.79E−01 | 1.23E−03 | 8.44E−01 | 8.08E−01 |
| 8030362 | SNORD33 | 4.48E−04 | 1.71E−02 | 2.72E−03 | 3.12E−02 | 3.47E−02 | 6.50E−01 | 6.66E−04 | 2.40E−01 | 8.09E−01 |
| 8041015 | SLC4A1AP | 4.50E−04 | 1.72E−02 | 1.56E−02 | 3.65E−02 | 2.39E−02 | 6.45E−01 | 1.35E−03 | 3.07E−01 | 3.23E−01 |
| 8084986 | FYTTD1 | 4.52E−04 | 1.72E−02 | 5.30E−03 | 3.13E−02 | 4.96E−02 | 6.67E−01 | 1.51E−03 | 7.34E−01 | 5.37E−01 |
| 8133961 | RUNDC3B | 4.56E−04 | 1.73E−02 | 6.13E−03 | 3.16E−02 | 1.70E−02 | 6.41E−01 | 2.28E−03 | 9.48E−01 | 6.14E−01 |
| 7987361 | ZNF770 | 4.57E−04 | 1.73E−02 | 8.36E−04 | 3.00E−02 | 1.53E−01 | 7.25E−01 | 1.85E−03 | 7.55E−01 | 7.31E−01 |
| 7893067 | — | 4.60E−04 | 1.73E−02 | 4.22E−03 | 3.13E−02 | 2.11E−02 | 6.45E−01 | 1.51E−03 | 3.22E−01 | 6.13E−01 |
| 8128133 | LYRM2 | 4.64E−04 | 1.74E−02 | 5.33E−02 | 5.42E−02 | 5.58E−03 | 6.33E−01 | 8.05E−04 | 3.58E−01 | 7.79E−02 |
| 8169709 | GLRX5 | 4.70E−04 | 1.76E−02 | 2.43E−01 | 1.27E−01 | 2.75E−03 | 6.06E−01 | 3.09E−04 | 8.45E−03 | 5.92E−03 |
| 8086515 | — | 4.71E−04 | 1.76E−02 | 1.45E−03 | 3.01E−02 | 1.80E−01 | 7.29E−01 | 1.41E−03 | 5.52E−01 | 8.34E−01 |
| 8127662 | — | 4.73E−04 | 1.76E−02 | 9.57E−03 | 3.37E−02 | 4.21E−02 | 6.61E−01 | 8.63E−04 | 4.73E−01 | 6.47E−01 |
| 7976515 | GLRX5 | 4.75E−04 | 1.77E−02 | 2.21E−01 | 1.19E−01 | 2.45E−03 | 6.06E−01 | 4.35E−04 | 1.89E−02 | 5.10E−03 |
| 8002381 | COG4 | 4.80E−04 | 1.78E−02 | 2.57E−03 | 3.12E−02 | 1.46E−01 | 7.23E−01 | 9.94E−04 | 4.38E−01 | 8.09E−01 |
| 8096411 | TIGD2 | 4.82E−04 | 1.78E−02 | 2.22E−02 | 3.97E−02 | 5.26E−03 | 6.33E−01 | 7.11E−04 | 6.40E−01 | 2.17E−01 |
| 7957242 | ATXN7L3B | 4.86E−04 | 1.79E−02 | 1.66E−03 | 3.04E−02 | 1.65E−01 | 7.26E−01 | 1.64E−03 | 6.05E−01 | 8.07E−01 |
| 8036902 | SERTAD1 | 4.86E−04 | 1.79E−02 | 3.23E−03 | 3.13E−02 | 4.64E−02 | 6.67E−01 | 9.20E−04 | 6.20E−01 | 8.94E−01 |
| 7915543 | SLC6A9 | 4.90E−04 | 1.79E−02 | 1.47E−03 | 3.01E−02 | 2.22E−01 | 7.44E−01 | 2.22E−03 | 4.52E−01 | 6.53E−01 |
| 7940160 | DTX4 | 4.91E−04 | 1.79E−02 | 6.06E−04 | 3.00E−02 | 6.27E−02 | 6.79E−01 | 2.74E−03 | 5.10E−02 | 9.29E−01 |
| 8137240 | GIMAP7 | 4.93E−04 | 1.79E−02 | 7.48E−02 | 6.38E−02 | 9.26E−04 | 5.98E−01 | 7.93E−04 | 9.71E−01 | 2.56E−02 |
| 8017829 | — | 4.94E−04 | 1.79E−02 | 4.60E−03 | 3.13E−02 | 1.83E−02 | 6.44E−01 | 2.77E−03 | 4.65E−01 | 5.74E−01 |
| 7896483 | — | 4.94E−04 | 1.79E−02 | 3.07E−02 | 4.39E−02 | 2.36E−02 | 6.45E−01 | 8.49E−04 | 5.83E−02 | 2.60E−01 |
| 7895490 | — | 4.95E−04 | 1.79E−02 | 2.38E−04 | 2.75E−02 | 7.36E−01 | 8.56E−01 | 1.07E−03 | 9.16E−02 | 1.17E−01 |
| 7893816 | — | 4.96E−04 | 1.79E−02 | 3.19E−02 | 4.44E−02 | 1.36E−02 | 641E−01 | 1.22E−03 | 1.57E−01 | 1.74E−01 |
| 8039010 | ZNF765 | 4.98E−04 | 1.79E−02 | 4.24E−02 | 4.92E−02 | 1.67E−02 | 6.41E−01 | 4.90E−04 | 9.24E−02 | 2.08E−01 |
| 8132465 | HECW1 | 4.99E−04 | 1.79E−02 | 1.37E−03 | 3.00E−02 | 9.41E−03 | 6.35E−01 | 3.27E−03 | 2.12E−02 | 7.67E−01 |
| 8169009 | BEX4 | 5.00E−04 | 1.79E−02 | 7.46E−03 | 3.28E−02 | 3.74E−02 | 6.59E−01 | 1.36E−03 | 7.45E−01 | 7.29E−01 |
| 7951447 | CWF19L2 | 5.01E−04 | 1.79E−02 | 2.44E−02 | 4.08E−02 | 7.70E−03 | 6.35E−01 | 1.06E−03 | 9.45E−01 | 1.10E−01 |
| 8101828 | TSPAN5 | 5.08E−04 | 1.81E−02 | 3.33E−01 | 1.57E−01 | 1.73E−03 | 5.98E−01 | 2.22E−04 | 5.47E−03 | 4.13E−03 |
| 8075585 | C22orf28 | 5.10E−04 | 1.81E−02 | 1.52E−02 | 3.63E−02 | 2.26E−02 | 6.45E−01 | 1.36E−03 | 4.80E−01 | 2.86E−01 |
| 8003249 | FBXO31 | 5.14E−04 | 1.83E−02 | 8.72E−04 | 3.00E−02 | 4.71E−01 | 8.07E−01 | 7.26E−04 | 1.08E−01 | 2.39E−01 |
| 7969096 | CDADC1 | 5.17E−04 | 1.83E−02 | 8.46E−03 | 3.34E−02 | 3.34E−02 | 6.46E−01 | 1.21E−03 | 8.19E−01 | 6.02E−01 |
| 7961755 | ST8SIA1 | 5.22E−04 | 1.84E−02 | 3.02E−03 | 3.13E−02 | 1.80E−01 | 7.30E−01 | 1.17E−03 | 1.31E−01 | 9.13E−01 |
| 8060379 | PSMF1 | 5.26E−04 | 1.85E−02 | 1.29E−01 | 8.54E−02 | 1.19E−03 | 5.98E−01 | 3.33E−04 | 3.23E−01 | 3.94E−02 |
| 8175572 | SPANXN3 | 5.27E−04 | 1.85E−02 | 4.32E−05 | 2.75E−02 | 9.16E−01 | 8.79E−01 | 9.99E−04 | 2.25E−01 | 2.85E−02 |
| 8139163 | FAM183B | 5.28E−04 | 1.85E−02 | 1.31E−02 | 3.53E−02 | 1.16E−02 | 6.35E−01 | 5.46E−04 | 5.33E−01 | 4.30E−01 |
| 7896157 | — | 5.28E−04 | 1.85E−02 | 9.30E−03 | 3.36E−02 | 3.36E−02 | 6.46E−01 | 1.75E−03 | 5.63E−01 | 6.09E−01 |
| 8027292 | ZNF431 | 5.29E−04 | 1.85E−02 | 6.12E−03 | 3.15E−02 | 2.53E−02 | 6.45E−01 | 1.65E−03 | 6.81E−01 | 6.74E−01 |
| 7988342 | — | 5.31E−04 | 1.85E−02 | 3.98E−02 | 4.82E−02 | 3.62E−03 | 6.06E−01 | 6.03E−04 | 8.82E−01 | 1.96E−01 |
| 7894574 | — | 5.34E−04 | 1.85E−02 | 1.37E−03 | 3.00E−02 | 2.05E−01 | 7.40E−01 | 2.05E−03 | 7.24E−01 | 5.62E−01 |
| 7920707 | FAM189B | 5.35E−04 | 1.85E−02 | 2.11E−04 | 2.75E−02 | 2.40E−01 | 7.47E−01 | 2.41E−03 | 1.50E−01 | 4.12E−01 |
| 8005638 | ALDH3A2 | 5.37E−04 | 1.85E−02 | 2.25E−03 | 3.11E−02 | 1.99E−01 | 7.38E−01 | 1.24E−03 | 2.26E−01 | 7.93E−01 |
| 8045171 | IMP4 | 5.37E−04 | 1.85E−02 | 6.01E−03 | 3.15E−02 | 7.38E−02 | 6.93E−01 | 9.60E−04 | 5.41E−01 | 9.44E−01 |
| 8167790 | TSR2 | 5.38E−04 | 1.85E−02 | 1.04E−02 | 3.42E−02 | 4.42E−02 | 6.66E−01 | 1.24E−03 | 4.35E−01 | 4.72E−01 |
| 7927669 | TFAM | 5.40E−04 | 1.85E−02 | 1.81E−03 | 3.09E−02 | 1.23E−01 | 7.08E−01 | 2.03E−03 | 9.42E−01 | 9.57E−01 |
| 8038989 | ZNF600 | 5.41E−04 | 1.85E−02 | 1.91E−02 | 3.83E−02 | 8.14E−03 | 6.35E−01 | 1.34E−03 | 9.38E−01 | 3.41E−01 |
| 7968670 | UFM1 | 5.42E−04 | 1.85E−02 | 1.92E−03 | 3.11E−02 | 1.07E−01 | 7.01E−01 | 1.66E−03 | 7.80E−01 | 8.23E−01 |
| 8167786 | — | 5.42E−04 | 1.85E−02 | 9.50E−03 | 3.36E−02 | 6.15E−02 | 6.79E−01 | 1.56E−03 | 2.23E−01 | 5.06E−01 |
| 8014749 | RPL23 | 5.47E−04 | 1.86E−02 | 1.50E−02 | 3.62E−02 | 1.53E−02 | 6.41E−01 | 1.02E−03 | 7.69E−01 | 5.69E−01 |
| 8084694 | EIF4A2 | 5.53E−04 | 1.86E−02 | 1.34E−02 | 3.54E−02 | 2.54E−02 | 6.45E−01 | 1.31E−03 | 6.85E−01 | 3.22E−01 |
| 7895320 | — | 5.54E−04 | 1.86E−02 | 1.29E−02 | 3.53E−02 | 2.47E−02 | 6.45E−01 | 1.24E−03 | 5.53E−01 | 5.92E−01 |
| 8127987 | SNORD50A | 5.55E−04 | 1.86E−02 | 7.43E−03 | 3.28E−02 | 3.96E−02 | 6.60E−01 | 4.73E−04 | 9.76E−01 | 9.44E−01 |
| 7934295 | — | 5.56E−04 | 1.86E−02 | 4.22E−04 | 2.77E−02 | 5.19E−01 | 8.15E−01 | 7.27E−04 | 5.19E−01 | 1.62E−01 |
| 8008627 | NOG | 5.56E−04 | 1.86E−02 | 1.41E−02 | 3.57E−02 | 6.31E−03 | 6.35E−01 | 1.09E−03 | 2.27E−01 | 1.45E−01 |
| 8109901 | FOX11 | 5.58E−04 | 1.86E−02 | 3.21E−04 | 2.75E−02 | 4.22E−01 | 7.97E−01 | 2.48E−03 | 8.76E−01 | 2.41E−01 |
| 7892509 | — | 5.59E−04 | 1.86E−02 | 5.79E−04 | 3.00E−02 | 2.53E−01 | 7.52E−01 | 1.18E−03 | 6.61E−01 | 6.74E−01 |
| 7974066 | PNN | 5.59E−04 | 1.86E−02 | 1.01E−02 | 3.40E−02 | 4.98E−02 | 6.67E−01 | 9.49E−04 | 4.03E−01 | 4.87E−01 |
| 8047401 | CFLAR | 5.65E−04 | 1.88E−02 | 3.44E−03 | 3.13E−02 | 3.52E−02 | 6.53E−01 | 1.84E−03 | 3.18E−01 | 7.20E−01 |
| 7956009 | METTL7B | 5.66E−04 | 1.88E−02 | 2.67E−04 | 2.75E−02 | 4.54E−01 | 8.02E−01 | 2.78E−03 | 9.12E−01 | 2.56E−01 |
| 8026139 | NFIX | 5.68E−04 | 1.88E−02 | 3.27E−02 | 4.48E−02 | 2.63E−02 | 6.45E−01 | 1.56E−03 | 4.16E−02 | 2.24E−01 |
| 8062695 | SRSF6 | 5.69E−04 | 1.88E−02 | 6.18E−04 | 3.00E−02 | 2.02E−01 | 7.38E−01 | 2.22E−03 | 7.00E−01 | 6.66E−01 |
| 8180207 | — | 5.76E−04 | 1.90E−02 | 3.22E−03 | 3.13E−02 | 9.62E−02 | 6.99E−01 | 1.65E−03 | 8.79E−01 | 9.40E−01 |
| 8103745 | HAND2 | 5.82E−04 | 1.91E−02 | 8.87E−03 | 3.35E−02 | 8.91E−02 | 6.94E−01 | 5.85E−04 | 1.69E−01 | 8.04E−01 |
| 7929719 | C10orf28 | 5.85E−04 | 1.91E−02 | 5.12E−03 | 3.13E−02 | 7.48E−02 | 6.93E−01 | 2.20E−03 | 6.01E−01 | 8.38E−01 |
| 8120937 | RIPPLY2 | 5.86E−04 | 1.91E−02 | 1.06E−02 | 3.43E−02 | 2.32E−02 | 6.45E−01 | 1.29E−03 | 9.42E−01 | 3.44E−01 |
| 8156521 | MIRLET7F1 | 5.86E−04 | 1.91E−02 | 3.69E−03 | 3.13E−02 | 1.15E−01 | 7.03E−01 | 1.69E−03 | 5.24E−01 | 9.42E−01 |
| 8094030 | AFAP1 | 5.88E−04 | 1.91E−02 | 9.86E−03 | 3.39E−02 | 3.21E−02 | 6.45E−01 | 2.42E−03 | 4.13E−01 | 6.15E−01 |
| 7989245 | HSP90AB4P | 5.89E−04 | 1.91E−02 | 9.01E−03 | 3.35E−02 | 3.61E−02 | 6.59E−01 | 9.06E−04 | 9.00E−01 | 8.18E−01 |
| 7895663 | — | 5.90E−04 | 1.91E−02 | 1.14E−02 | 3.48E−02 | 3.19E−02 | 6.45E−01 | 9.41E−04 | 8.58E−01 | 4.37E−01 |
| 8027760 | FXYD1 | 5.91E−04 | 1.91E−02 | 1.99E−03 | 3.11E−02 | 1.18E−01 | 7.04E−01 | 1.60E−03 | 7.63E−01 | 7.41E−01 |
| 7943160 | SCARNA9 | 5.91E−04 | 1.91E−02 | 2.31E−02 | 4.02E−02 | 3.79E−02 | 6.59E−01 | 7.95E−04 | 8.39E−02 | 4.99E−01 |
| 7914334 | SNRNP40 | 5.93E−04 | 1.91E−02 | 2.31E−02 | 4.02E−02 | 2.45E−02 | 6.45E−01 | 1.62E−03 | 2.05E−01 | 2.94E−01 |
| 8046804 | NUP35 | 5.95E−04 | 1.91E−02 | 4.35E−03 | 3.13E−02 | 3.46E−02 | 6.50E−01 | 1.83E−03 | 3.99E−01 | 5.69E−01 |
| 8150149 | — | 5.98E−04 | 1.91E−02 | 5.91E−03 | 3.15E−02 | 4.86E−02 | 6.67E−01 | 2.13E−03 | 9.38E−01 | 6.85E−01 |
| 8134680 | ZKSCAN1 | 5.98E−04 | 1.91E−02 | 4.27E−04 | 2.78E−02 | 2.89E−01 | 7.62E−01 | 2.11E−03 | 9.53E−01 | 4.91E−01 |
| 7989885 | DNAJB14 | 6.01E−04 | 1.91E−02 | 1.70E−03 | 3.05E−02 | 1.17E−01 | 7.03E−01 | 1.99E−03 | 7.43E−01 | 8.89E−01 |
| 7896319 | — | 6.01E−04 | 1.91E−02 | 3.69E−03 | 3.13E−02 | 1.37E−01 | 7.20E−01 | 1.53E−03 | 3.90E−01 | 9.13E−01 |
| 7898602 | OTUD3 | 6.04E−04 | 1.91E−02 | 1.08E−02 | 3.44E−02 | 5.15E−02 | 6.71E−01 | 8.73E−04 | 4.51E−01 | 5.31E−01 |
| 8035765 | ZNF14 | 6.04E−04 | 1.91E−02 | 1.29E−02 | 3.53E−02 | 2.39E−02 | 6.45E−01 | 1.32E−03 | 8.93E−01 | 4.21E−01 |
| 7896368 | — | 6.05E−04 | 1.91E−02 | 3.52E−02 | 4.60E−02 | 1.70E−02 | 6.41E−01 | 7.11E−04 | 1.21E−01 | 4.38E−01 |
| 8052087 | — | 6.07E−04 | 1.91E−02 | 9.06E−05 | 2.75E−02 | 8.53E−01 | 8.71E−01 | 2.88E−03 | 7.04E−01 | 6.56E−02 |
| 8129181 | GOPC | 6.07E−04 | 1.91E−02 | 7.36E−04 | 3.00E−02 | 2.91E−01 | 7.62E−01 | 2.09E−03 | 6.75E−01 | 5.46E−01 |
| 8130087 | PPIL4 | 6.11E−04 | 1.92E−02 | 7.59E−03 | 3.29E−02 | 4.96E−02 | 6.67E−01 | 1.81E−03 | 6.94E−01 | 5.56E−01 |
| 8030049 | CYTH2 | 6.14E−04 | 1.92E−02 | 8.59E−03 | 3.34E−02 | 1.08E−01 | 7.01E−01 | 1.58E−03 | 8.40E−02 | 9.43E−01 |
| 7999532 | GSPT1 | 6.15E−04 | 1.92E−02 | 1.35E−01 | 8.76E−02 | 4.26E−03 | 6.11E−01 | 4.56E−04 | 9.71E−02 | 2.79E−02 |
| 7895847 | — | 6.16E−04 | 1.92E−02 | 4.07E−03 | 3.13E−02 | 5.66E−02 | 6.79E−01 | 1.61E−03 | 7.39E−01 | 9.91E−01 |
| 8128075 | — | 6.24E−04 | 1.94E−02 | 2.65E−04 | 2.75E−02 | 5.81E−01 | 8.27E−01 | 2.20E−03 | 5.84E−01 | 1.74E−01 |
| 8077858 | ATG7 | 6.25E−04 | 1.94E−02 | 4.72E−03 | 3.13E−02 | 1.07E−01 | 7.01E−01 | 1.93E−03 | 3.51E−01 | 7.98E−01 |
| 8083457 | RAP2B | 6.28E−04 | 1.94E−02 | 5.30E−03 | 3.13E−02 | 1.13E−01 | 7.01E−01 | 2.73E−03 | 2.48E−01 | 9.53E−01 |
| 8016868 | — | 6.28E−04 | 1.94E−02 | 1.16E−03 | 3.00E−02 | 5.41E−02 | 6.74E−01 | 4.81E−03 | 8.56E−02 | 8.61E−01 |
| 7963139 | BCDIN3D | 6.30E−04 | 1.95E−02 | 1.09E−02 | 3.45E−02 | 8.63E−02 | 6.94E−01 | 7.36E−04 | 1.31E−01 | 8.20E−01 |
| 7896440 | — | 6.34E−04 | 1.95E−02 | 1.60E−03 | 3.02E−02 | 2.99E−01 | 7.66E−01 | 5.59E−04 | 2.58E−01 | 4.19E−01 |
| 7908988 | SNRPE | 6.38E−04 | 1.96E−02 | 6.57E−02 | 5.97E−02 | 5.01E−03 | 6.25E−01 | 3.98E−04 | 4.20E−01 | 1.49E−01 |
| 8048717 | SGPP2 | 6.45E−04 | 1.98E−02 | 5.65E−03 | 3.15E−02 | 7.58E−03 | 6.35E−01 | 2.24E−03 | 8.25E−02 | 4.59E−01 |
| 7894168 | — | 6.48E−04 | 1.98E−02 | 3.07E−02 | 4.39E−02 | 8.49E−03 | 6.35E−01 | 7.39E−04 | 8.01E−01 | 3.16E−01 |
| 8116534 | TRIM52 | 6.52E−04 | 1.99E−02 | 6.04E−03 | 3.15E−02 | 1.02E−01 | 7.01E−01 | 1.67E−03 | 3.18E−01 | 9.46E−01 |
| 8059854 | ARL4C | 6.54E−04 | 1.99E−02 | 7.02E−03 | 3.24E−02 | 5.35E−02 | 6.73E−01 | 2.16E−03 | 7.71E−01 | 6.97E−01 |
| 8036420 | ZFP30 | 6.57E−04 | 2.00E−02 | 5.18E−03 | 3.13E−02 | 9.04E−02 | 6.96E−01 | 1.49E−03 | 6.35E−01 | 9.83E−01 |
| 7935865 | POLL | 6.62E−04 | 2.01E−02 | 5.53E−02 | 5.51E−02 | 5.46E−03 | 6.33E−01 | 7.97E−04 | 8.85E−01 | 5.41E−02 |
| 7946680 | BTBD10 | 6.64E−04 | 2.01E−02 | 1.30E−03 | 3.00E−02 | 3.02E−01 | 7.66E−01 | 2.31E−03 | 4.89E−01 | 4.45E−01 |
| 8111925 | C5orf39 | 6.65E−04 | 2.01E−02 | 7.86E−03 | 3.31E−02 | 3.78E−02 | 6.59E−01 | 1.73E−03 | 9.66E−01 | 6.27E−01 |
| 8080991 | HNRNPA3 | 6.68E−04 | 2.01E−02 | 2.55E−02 | 4.12E−02 | 1.92E−02 | 6.45E−01 | 1.13E−03 | 4.26E−01 | 3.52E−01 |
| 8051133 | FTH1P3 | 6.68E−04 | 2.01E−02 | 6.46E−03 | 3.19E−02 | 5.96E−03 | 6.35E−01 | 1.33E−03 | 3.99E−02 | 4.39E−01 |
| 8171848 | PCYT1B | 6.69E−04 | 2.01E−02 | 4.65E−04 | 2.91E−02 | 4.05E−01 | 7.92E−01 | 8.05E−04 | 2.07E−01 | 3.84E−01 |
| 8122409 | PEX3 | 6.70E−04 | 2.01E−02 | 7.67E−03 | 3.29E−02 | 4.44E−02 | 6.66E−01 | 2.01E−03 | 8.87E−01 | 6.64E−01 |
| 8169291 | — | 6.73E−04 | 2.01E−02 | 2.20E−02 | 3.96E−02 | 1.19E−02 | 6.35E−01 | 7.76E−04 | 7.98E−01 | 2.90E−01 |
| 7893571 | — | 6.75E−04 | 2.01E−02 | 8.48E−03 | 3.34E−02 | 3.77E−02 | 6.59E−01 | 8.33E−04 | 8.30E−01 | 5.31E−01 |
| 7958844 | — | 6.78E−04 | 2.02E−02 | 2.59E−03 | 3.12E−02 | 4.63E−02 | 6.67E−01 | 1.98E−03 | 2.20E−01 | 7.40E−01 |
| 8133442 | LAT2 | 6.80E−04 | 2.02E−02 | 1.69E−02 | 3.72E−02 | 4.82E−02 | 6.67E−01 | 1.68E−03 | 1.25E−01 | 6.78E−01 |
| 8104166 | SDHA | 6.82E−04 | 2.02E−02 | 1.16E−03 | 3.00E−02 | 3.84E−01 | 7.87E−01 | 2.23E−03 | 3.35E−01 | 3.64E−01 |
| 7926541 | — | 6.82E−04 | 2.02E−02 | 4.77E−03 | 3.13E−02 | 9.41E−02 | 6.97E−01 | 8.01E−04 | 8.55E−01 | 7.90E−01 |
| 8041867 | MSH2 | 6.83E−04 | 2.02E−02 | 1.38E−03 | 3.00E−02 | 1.20E−01 | 7.06E−01 | 1.90E−03 | 5.78E−01 | 9.79E−01 |
| 7912537 | DHRS3 | 6.87E−04 | 2.02E−02 | 2.11E−02 | 3.92E−02 | 1.00E−02 | 6.35E−01 | 1.36E−03 | 8.80E−01 | 3.07E−01 |
| 8139790 | — | 6.88E−04 | 2.02E−02 | 8.38E−05 | 2.75E−02 | 5.22E−01 | 8.16E−01 | 1.70E−03 | 6.23E−01 | 1.89E−01 |
| 7900395 | RLF | 6.89E−04 | 2.02E−02 | 6.52E−04 | 3.00E−02 | 3.46E−01 | 7.75E−01 | 2.62E−03 | 7.49E−01 | 4.12E−01 |
| 8147019 | FAM164A | 6.92E−04 | 2.02E−02 | 2.11E−02 | 3.92E−02 | 2.05E−02 | 6.45E−01 | 1.32E−03 | 6.26E−01 | 3.59E−01 |
| 8114320 | HNRNPA0 | 6.94E−04 | 2.02E−02 | 8.24E−04 | 3.00E−02 | 6.43E−01 | 8.38E−01 | 1.63E−03 | 1.33E−01 | 1.62E−01 |
| 7990582 | SCAPER | 6.94E−04 | 2.02E−02 | 2.02E−02 | 3.88E−02 | 2.18E−02 | 6.45E−01 | 1.54E−03 | 5.22E−01 | 3.68E−01 |
| 7996934 | NIP7 | 6.95E−04 | 2.02E−02 | 3.56E−02 | 4.61E−02 | 8.78E−03 | 6.35E−01 | 1.45E−03 | 5.05E−01 | 2.31E−01 |
| 7950606 | RSF1 | 6.96E−04 | 2.02E−02 | 2.54E−03 | 3.12E−02 | 1.28E−01 | 7.12E−01 | 1.95E−03 | 7.58E−01 | 9.17E−01 |
| 8034393 | ZNF443 | 6.97E−04 | 2.02E−02 | 1.94E−02 | 3.85E−02 | 2.08E−02 | 6.45E−01 | 1.32E−03 | 7.79E−01 | 3.60E−01 |
| 7991126 | WDR73 | 6.98E−04 | 2.02E−02 | 3.78E−03 | 3.13E−02 | 1.69E−01 | 7.26E−01 | 1.95E−03 | 2.06E−01 | 9.85E−01 |
| 8168852 | HNRNPH2 | 6.99E−04 | 2.02E−02 | 8.11E−03 | 3.32E−02 | 8.01E−02 | 6.94E−01 | 2.05E−03 | 3.08E−01 | 7.58E−01 |
| 8111136 | FAM134B | 7.02E−04 | 2.02E−02 | 5.98E−03 | 3.15E−02 | 2.49E−02 | 6.45E−01 | 1.32E−03 | 2.72E−01 | 5.53E−01 |
| 8123825 | SLC35B3 | 7.03E−04 | 2.02E−02 | 1.69E−02 | 3.72E−02 | 1.65E−02 | 6.41E−01 | 2.36E−03 | 8.88E−01 | 3.46E−01 |
| 7975203 | MPP5 | 7.07E−04 | 2.03E−02 | 2.27E−03 | 3.11E−02 | 9.22E−02 | 6.96E−01 | 1.56E−03 | 6.16E−01 | 8.92E−01 |
| 7896644 | — | 7.10E−04 | 2.03E−02 | 1.83E−01 | 1.06E−01 | 7.24E−04 | 5.98E−01 | 6.11E−04 | 2.56E−01 | 2.14E−02 |
| 7973458 | DHRS4L2 | 7.10E−04 | 2.03E−02 | 5.45E−02 | 5.47E−02 | 2.54E−02 | 6.45E−01 | 6.72E−04 | 2.53E−02 | 2.61E−01 |
| 8050302 | ROCK2 | 7.11E−04 | 2.03E−02 | 7.74E−04 | 3.00E−02 | 2.35E−01 | 7.47E−01 | 2.67E−03 | 8.59E−01 | 5.96E−01 |
| 7945864 | ZNF195 | 7.14E−04 | 2.03E−02 | 4.36E−03 | 3.13E−02 | 1.18E−01 | 7.04E−01 | 1.18E−03 | 4.55E−01 | 8.61E−01 |
| 7975976 | ANSA1 | 7.20E−04 | 2.04E−02 | 6.32E−03 | 3.18E−02 | 1.02E−01 | 7.01E−01 | 1.21E−03 | 3.63E−01 | 8.07E−01 |
| 8138045 | EIF2AK1 | 7.22E−04 | 2.04E−02 | 1.25E−01 | 8.38E−02 | 2.65E−03 | 6.06E−01 | 6.55E−04 | 2.81E−01 | 3.97E−02 |
| 8175933 | RENBP | 7.26E−04 | 2.05E−02 | 1.70E−02 | 3.72E−02 | 3.87E−02 | 6.59E−01 | 1.36E−03 | 3.52E−01 | 5.69E−01 |
| 8001841 | DYNC1LI2 | 7.34E−04 | 2.06E−02 | 2.00E−03 | 3.11E−02 | 4.02E−01 | 7.91E−01 | 2.16E−03 | 4.26E−02 | 4.68E−01 |
| 7960134 | ZNF26 | 7.35E−04 | 2.06E−02 | 1.25E−02 | 3.52E−02 | 4.08E−02 | 6.61E−01 | 1.96E−03 | 5.70E−01 | 5.22E−01 |
| 7893172 | — | 7.36E−04 | 2.06E−02 | 1.55E−02 | 3.65E−02 | 8.46E−03 | 6.35E−01 | 8.47E−04 | 3.75E−01 | 4.56E−01 |
| 8158930 | C9orf9 | 7.38E−04 | 2.06E−02 | 1.65E−04 | 2.75E−02 | 7.33E−01 | 8.56E−01 | 2.02E−03 | 9.77E−01 | 8.87E−02 |
| 7977482 | TTC5 | 7.39E−04 | 2.06E−02 | 1.77E−02 | 3.76E−02 | 5.75E−02 | 6.79E−01 | 1.41E−03 | 1.20E−01 | 6.21E−01 |
| 7995017 | STX4 | 7.39E−04 | 2.06E−02 | 2.06E−02 | 3.90E−02 | 2.31E−02 | 6.45E−01 | 1.68E−03 | 4.25E−01 | 5.11E−01 |
| 8007921 | MYL4 | 7.40E−04 | 2.06E−02 | 2.92E−01 | 1.44E−01 | 8.28E−04 | 5.98E−01 | 2.76E−04 | 1.11E−01 | 9.21E−03 |
| 7942586 | RPS3 | 7.42E−04 | 2.07E−02 | 9.42E−03 | 3.36E−02 | 4.76E−02 | 6.67E−01 | 1.85E−03 | 7.42E−01 | 6.02E−01 |
| 7946354 | LMO1 | 7.44E−04 | 2.07E−02 | 1.16E−02 | 3.50E−02 | 1.91E−02 | 6.45E−01 | 2.42E−03 | 7.66E−01 | 4.47E−01 |
| 8124459 | ZNF322 | 7.47E−04 | 2.07E−02 | 2.89E−03 | 3.13E−02 | 1.57E−01 | 7.26E−01 | 2.07E−03 | 6.15E−01 | 8.46E−01 |
| 7921228 | ETV3 | 7.49E−04 | 2.07E−02 | 4.52E−03 | 3.13E−02 | 1.89E−01 | 7.34E−01 | 1.79E−03 | 1.03E−01 | 9.34E−01 |
| 8008493 | LUC7L3 | 7.51E−04 | 2.07E−02 | 4.66E−03 | 3.13E−02 | 9.85E−02 | 7.00E−01 | 1.83E−03 | 6.85E−01 | 8.11E−01 |
| 8112302 | C5orf43 | 7.54E−04 | 2.07E−02 | 1.49E−02 | 3.61E−02 | 3.35E−02 | 6.46E−01 | 1.98E−03 | 5.98E−01 | 4.20E−01 |
| 7937667 | BRSK2 | 7.55E−04 | 2.07E−02 | 3.58E−04 | 2.75E−02 | 3.55E−01 | 7.78E−01 | 3.75E−03 | 7.20E−01 | 4.10E−01 |
| 8158022 | ZNF79 | 7.57E−04 | 2.07E−02 | 7.23E−02 | 6.27E−02 | 2.94E−02 | 6.45E−01 | 9.73E−05 | 4.78E−03 | 4.09E−01 |
| 8052956 | EXOC6B | 7.58E−04 | 2.07E−02 | 4.10E−03 | 3.13E−02 | 9.50E−02 | 6.97E−01 | 1.96E−03 | 8.97E−01 | 8.94E−01 |
| 8009176 | TACO1 | 7.60E−04 | 2.07E−02 | 3.53E−02 | 4.61E−02 | 2.36E−02 | 6.45E−01 | 4.87E−04 | 2.95E−01 | 3.20E−01 |
| 7935002 | SRP9 | 7.61E−04 | 2.07E−02 | 2.00E−02 | 3.87E−02 | 1.68E−02 | 6.41E−01 | 2.15E−03 | 7.59E−01 | 3.40E−01 |
| 8072610 | FBXO7 | 7.61E−04 | 2.07E−02 | 1.49E−01 | 9.32E−02 | 2.97E−03 | 6.06E−01 | 6.90E−04 | 8.02E−02 | 4.39E−02 |
| 7967060 | SRSF9 | 7.64E−04 | 2.08E−02 | 1.11E−02 | 3.47E−02 | 9.59E−02 | 6.98E−01 | 2.84E−03 | 5.06E−02 | 8.39E−01 |
| 7985920 | MESP2 | 7.65E−04 | 2.08E−02 | 1.29E−03 | 3.00E−02 | 2.80E−01 | 7.60E−01 | 5.74E−03 | 6.46E−01 | 5.38E−01 |
| 8104570 | FAM105A | 7.69E−04 | 2.08E−02 | 1.06E−02 | 3.44E−02 | 7.38E−02 | 6.93E−01 | 2.30E−03 | 2.37E−01 | 5.85E−01 |
| 8169920 | RBMX2 | 7.72E−04 | 2.09E−02 | 1.07E−01 | 7.68E−02 | 2.78E−03 | 6.06E−01 | 1.43E−03 | 2.36E−01 | 3.45E−02 |
| 8038993 | ZNF28 | 7.76E−04 | 2.09E−02 | 6.89E−03 | 3.24E−02 | 4.53E−02 | 6.66E−01 | 2.02E−03 | 8.03E−01 | 6.91E−01 |
| 8131292 | RBAK | 7.77E−04 | 2.09E−02 | 5.41E−03 | 3.15E−02 | 5.09E−02 | 6.70E−01 | 1.47E−03 | 6.14E−01 | 6.21E−01 |
| 8135488 | LRRN3 | 7.86E−04 | 2.11E−02 | 4.00E−03 | 3.13E−02 | 6.62E−02 | 6.87E−01 | 1.77E−03 | 6.22E−01 | 7.51E−01 |
| 8030950 | ZNF701 | 7.91E−04 | 2.12E−02 | 1.03E−02 | 3.42E−02 | 3.78E−02 | 6.59E−01 | 2.63E−03 | 8.72E−01 | 5.38E−01 |
| 7921677 | CD244 | 8.02E−04 | 2.15E−02 | 1.04E−02 | 3.42E−02 | 7.28E−02 | 6.93E−01 | 1.83E−03 | 3.35E−01 | 8.55E−01 |
| 8175311 | CXorf48 | 8.08E−04 | 2.16E−02 | 6.41E−02 | 5.90E−02 | 2.76E−03 | 6.06E−01 | 7.68E−04 | 9.54E−01 | 1.28E−01 |
| 8123644 | TUBB2A | 8.10E−04 | 2.16E−02 | 8.87E−03 | 3.35E−02 | 3.72E−02 | 6.59E−01 | 1.01E−03 | 6.26E−01 | 2.07E−01 |
| 8013305 | ZNF286B | 8.10E−04 | 2.16E−02 | 1.68E−02 | 3.71E−02 | 1.03E−02 | 6.35E−01 | 1.57E−03 | 4.44E−01 | 3.28E−01 |
| 8137244 | GIMAP4 | 8.12E−04 | 2.16E−02 | 3.05E−02 | 4.39E−02 | 1.39E−02 | 6.41E−01 | 2.26E−03 | 5.11E−01 | 1.90E−01 |
| 8122142 | SNORD101 | 8.13E−04 | 2.16E−02 | 6.33E−04 | 3.00E−02 | 4.24E−01 | 7.97E−01 | 2.38E−03 | 4.97E−01 | 3.77E−01 |
| 8017102 | — | 8.19E−04 | 2.17E−02 | 2.55E−03 | 3.12E−02 | 2.01E−01 | 7.38E−01 | 2.37E−03 | 5.36E−01 | 7.36E−01 |
| 7966046 | MTERFD3 | 8.24E−04 | 2.17E−02 | 2.02E−02 | 3.88E−02 | 5.25E−02 | 6.73E−01 | 9.10E−04 | 2.05E−01 | 5.68E−01 |
| 8039025 | ZNF702P | 8.24E−04 | 2.17E−02 | 1.76E−02 | 3.76E−02 | 2.25E−02 | 6.45E−01 | 1.97E−03 | 9.16E−01 | 3.42E−01 |
| 8171491 | — | 8.27E−04 | 2.18E−02 | 9.31E−04 | 3.00E−02 | 2.93E−01 | 7.63E−01 | 3.60E−03 | 6.75E−01 | 3.29E−01 |
| 8094271 | MED28 | 8.28E−04 | 2.18E−02 | 6.82E−03 | 3.24E−02 | 1.65E−01 | 7.26E−01 | 2.26E−03 | 6.76E−02 | 9.48E−01 |
| 7969271 | SUGT1 | 8.30E−04 | 2.18E−02 | 6.57E−03 | 3.20E−02 | 1.06E−01 | 7.01E−01 | 2.30E−03 | 3.64E−01 | 8.06E−01 |
| 8131067 | GPR146 | 8.37E−04 | 2.19E−02 | 1.82E−01 | 1.05E−01 | 4.59E−03 | 6.11E−01 | 6.89E−04 | 3.90E−02 | 2.92E−02 |
| 7971550 | MED4 | 8.43E−04 | 2.20E−02 | 2.31E−03 | 3.11E−02 | 2.23E−01 | 7.44E−01 | 2.81E−03 | 3.85E−01 | 7.87E−01 |
| 8031744 | ZNF17 | 8.44E−04 | 2.20E−02 | 1.08E−02 | 3.45E−02 | 3.54E−02 | 6.54E−01 | 1.99E−03 | 9.97E−01 | 6.13E−01 |
| 8166498 | — | 8.46E−04 | 2.20E−02 | 5.92E−03 | 3.15E−02 | 1.89E−01 | 7.34E−01 | 1.19E−03 | 1.18E−01 | 8.06E−01 |
| 8023868 | LOC400657 | 8.52E−04 | 2.21E−02 | 6.02E−03 | 3.15E−02 | 3.75E−02 | 6.59E−01 | 2.21E−03 | 4.24E−01 | 6.57E−01 |
| 7893690 | — | 8.52E−04 | 2.21E−02 | 6.80E−03 | 3.23E−02 | 1.24E−01 | 7.08E−01 | 1.52E−03 | 3.46E−01 | 7.41E−01 |
| 8173135 | ALAS2 | 8.54E−04 | 2.21E−02 | 2.27E−01 | 1.21E−01 | 7.12E−03 | 6.35E−01 | 4.54E−04 | 6.52E−04 | 4.24E−02 |
| 7922410 | SNORD44 | 8.55E−04 | 2.21E−02 | 1.29E−03 | 3.00E−02 | 1.32E−01 | 7.16E−01 | 1.60E−03 | 4.22E−01 | 8.97E−01 |
| 8009476 | MAP2K6 | 8.58E−04 | 2.21E−02 | 3.80E−03 | 3.13E−02 | 5.89E−02 | 6.79E−01 | 2.74E−03 | 5.43E−01 | 5.11E−01 |
| 8048272 | C2orf62 | 8.61E−04 | 2.21E−02 | 2.95E−03 | 3.13E−02 | 8.77E−02 | 6.94E−01 | 1.61E−03 | 4.58E−01 | 9.26E−01 |
| 7972365 | — | 8.62E−04 | 2.21E−02 | 9.70E−03 | 3.38E−02 | 4.51E−02 | 6.66E−01 | 3.03E−04 | 6.84E−01 | 9.50E−01 |
| 7984405 | C15orf61 | 8.62E−04 | 2.21E−02 | 7.89E−02 | 6.55E−02 | 1.02E−02 | 6.35E−01 | 1.09E−03 | 7.64E−02 | 1.76E−01 |
| 7965436 | EEA1 | 8.62E−04 | 2.21E−02 | 2.84E−03 | 3.12E−02 | 1.11E−01 | 7.01E−01 | 2.22E−03 | 7.97E−01 | 9.90E−01 |
| 8157933 | ZBTB43 | 8.66E−04 | 2.21E−02 | 1.66E−02 | 3.70E−02 | 6.06E−02 | 6.79E−01 | 1.29E−03 | 2.35E−01 | 6.50E−01 |
| 8070141 | CRYZL1 | 8.76E−04 | 2.23E−02 | 2.24E−02 | 3.98E−02 | 1.98E−02 | 6.45E−01 | 2.08E−03 | 8.56E−01 | 1.75E−01 |
| 7951422 | KIAA1826 | 8.77E−04 | 2.23E−02 | 1.47E−02 | 3.60E−02 | 3.62E−02 | 6.59E−01 | 1.89E−03 | 7.85E−01 | 3.45E−01 |
| 7968915 | GTF2F2 | 8.80E−04 | 2.23E−02 | 2.50E−03 | 3.12E−02 | 1.03E−01 | 7.01E−01 | 2.58E−03 | 6.42E−01 | 8.80E−01 |
| 7893266 | — | 8.81E−04 | 2.23E−02 | 2.02E−02 | 3.88E−02 | 3.63E−02 | 6.59E−01 | 2.60E−03 | 2.90E−01 | 3.86E−01 |
| 7894258 | — | 8.82E−04 | 2.23E−02 | 1.05E−03 | 3.00E−02 | 3.91E−01 | 7.89E−01 | 2.45E−03 | 7.80E−01 | 2.94E−01 |
| 8024170 | HMHA1 | 8.84E−04 | 2.23E−02 | 6.98E−04 | 3.00E−02 | 5.36E−01 | 8.18E−01 | 2.62E−03 | 8.83E−01 | 1.54E−01 |
| 8173673 | ATRX | 8.85E−04 | 2.23E−02 | 1.54E−03 | 3.01E−02 | 1.28E−01 | 7.12E−01 | 3.40E−03 | 4.64E−01 | 9.24E−01 |
| 7896632 | — | 8.89E−04 | 2.24E−02 | 1.69E−03 | 3.05E−02 | 2.99E−01 | 7.66E−01 | 3.04E−03 | 3.60E−01 | 6.09E−01 |
| 8066697 | SLC35C2 | 9.00E−04 | 2.26E−02 | 5.96E−03 | 3.15E−02 | 1.96E−01 | 7.36E−01 | 2.15E−03 | 9.78E−02 | 8.54E−01 |
| 8022473 | ESCO1 | 9.05E−04 | 2.26E−02 | 4.02E−03 | 3.13E−02 | 7.47E−02 | 6.93E−01 | 2.76E−03 | 6.34E−01 | 8.58E−01 |
| 8168968 | GPRASP1 | 9.10E−04 | 2.26E−02 | 4.56E−03 | 3.13E−02 | 4.56E−02 | 6.66E−01 | 2.90E−03 | 3.76E−01 | 6.98E−01 |
| 7894056 | — | 9.12E−04 | 2.26E−02 | 9.27E−03 | 3.36E−02 | 6.64E−02 | 6.87E−01 | 3.23E−03 | 5.73E−01 | 8.42E−01 |
| 8102352 | PITX2 | 9.14E−04 | 2.26E−02 | 3.09E−03 | 3.13E−02 | 4.95E−02 | 6.67E−01 | 4.01E−03 | 2.64E−01 | 9.23E−01 |
| 8174197 | — | 9.14E−04 | 2.26E−02 | 7.99E−04 | 3.00E−02 | 3.75E−01 | 7.85E−01 | 1.58E−03 | 5.04E−01 | 4.54E−01 |
| 7916590 | AK2 | 9.16E−04 | 2.26E−02 | 1.45E−02 | 3.59E−02 | 1.75E−01 | 7.28E−01 | 1.46E−03 | 2.22E−03 | 8.56E−01 |
| 7932637 | ANKRD26 | 9.17E−04 | 2.26E−02 | 5.16E−03 | 3.13E−02 | 4.24E−02 | 6.62E−01 | 2.07E−03 | 3.18E−01 | 7.06E−01 |
| 7899534 | EPB41 | 9.18E−04 | 2.26E−02 | 1.25E−01 | 8.36E−02 | 4.13E−03 | 6.08E−01 | 8.61E−04 | 2.23E−01 | 5.41E−02 |
| 8162562 | LINC00476 | 9.19E−04 | 2.26E−02 | 2.22E−03 | 3.11E−02 | 1.79E−01 | 7.29E−01 | 2.46E−03 | 9.24E−01 | 7.79E−01 |
| 7936134 | OBFC1 | 9.19E−04 | 2.26E−02 | 6.05E−03 | 3.15E−02 | 1.83E−01 | 7.32E−01 | 1.74E−03 | 1.84E−01 | 7.53E−01 |
| 7961798 | SOX5 | 9.21E−04 | 2.26E−02 | 1.58E−03 | 3.02E−02 | 3.65E−01 | 7.82E−01 | 7.39E−03 | 4.86E−01 | 3.39E−01 |
| 7894202 | — | 9.21E−04 | 2.26E−02 | 2.15E−02 | 3.94E−02 | 1.24E−02 | 6.35E−01 | 3.55E−04 | 3.27E−01 | 3.59E−01 |
| 8086494 | ZNF852 | 9.23E−04 | 2.26E−02 | 1.99E−03 | 3.11E−02 | 3.73E−01 | 7.85E−01 | 1.84E−03 | 1.96E−01 | 4.18E−01 |
| 7904448 | — | 9.23E−04 | 2.26E−02 | 1.50E−04 | 2.75E−02 | 4.52E−01 | 8.02E−01 | 2.18E−03 | 7.65E−02 | 1.28E−01 |
| 7994675 | ASPHD1 | 9.23E−04 | 2.26E−02 | 6.95E−05 | 2.75E−02 | 8.94E−01 | 8.76E−01 | 3.40E−03 | 3.66E−01 | 5.21E−02 |
| 8076909 | TUBGCP6 | 9.31E−04 | 2.28E−02 | 6.17E−03 | 3.16E−02 | 1.41E−01 | 7.23E−01 | 9.05E−04 | 4.49E−01 | 7.75E−01 |
| 7969835 | PCCA | 9.34E−04 | 2.28E−02 | 4.98E−03 | 3.13E−02 | 9.58E−02 | 6.98E−01 | 2.34E−03 | 9.57E−01 | 8.96E−01 |
| 8153935 | ZNF252 | 9.35E−04 | 2.28E−02 | 1.86E−02 | 3.80E−02 | 3.94E−02 | 6.59E−01 | 1.71E−03 | 4.76E−01 | 5.87E−01 |
| 8117622 | OR2B6 | 9.36E−04 | 2.28E−02 | 2.28E−03 | 3.11E−02 | 1.36E−01 | 7.20E−01 | 4.75E−03 | 7.26E−01 | 7.81E−01 |
| 8180351 | CTBP2 | 9.37E−04 | 2.28E−02 | 5.23E−02 | 5.38E−02 | 1.01E−02 | 6.35E−01 | 7.95E−04 | 3.47E−01 | 3.57E−01 |
| 7948898 | SNORD31 | 9.41E−04 | 2.28E−02 | 2.73E−02 | 4.22E−02 | 1.51E−02 | 6.41E−01 | 1.16E−03 | 9.20E−01 | 2.76E−01 |
| 8025766 | CARM1 | 9.41E−04 | 2.28E−02 | 2.51E−01 | 1.30E−01 | 1.76E−03 | 5.98E−01 | 5.29E−04 | 9.76E−02 | 1.73E−02 |
| 8039054 | ZNF347 | 9.43E−04 | 2.28E−02 | 7.68E−03 | 3.29E−02 | 5.63E−02 | 6.78E−01 | 2.42E−03 | 9.32E−01 | 6.94E−01 |
| 7894933 | — | 9.44E−04 | 2.28E−02 | 1.61E−02 | 3.68E−02 | 1.09E−01 | 7.01E−01 | 9.45E−04 | 6.19E−02 | 9.62E−01 |
| 8161024 | RMRP | 9.46E−04 | 2.28E−02 | 4.28E−03 | 3.13E−02 | 5.34E−02 | 6.73E−01 | 2.10E−03 | 3.39E−01 | 9.11E−01 |
| 7986687 | WHAMMP3 | 9.49E−04 | 2.28E−02 | 8.77E−03 | 3.34E−02 | 7.27E−02 | 6.93E−01 | 2.17E−03 | 6.33E−01 | 5.69E−01 |
| 8171170 | — | 9.51E−04 | 2.29E−02 | 3.13E−03 | 3.13E−02 | 2.81E−01 | 7.60E−01 | 4.07E−03 | 2.14E−01 | 6.12E−01 |
| 8017421 | CCDC47 | 9.53E−04 | 2.29E−02 | 1.09E−02 | 3.45E−02 | 9.38E−02 | 6.97E−01 | 2.72E−03 | 2.13E−01 | 7.74E−01 |
| 7963061 | C1QL4 | 9.62E−04 | 2.30E−02 | 1.49E−03 | 3.01E−02 | 3.49E−01 | 7.76E−01 | 1.02E−03 | 4.22E−01 | 3.78E−01 |
| 8142977 | MIR29B1 | 9.63E−04 | 2.30E−02 | 3.33E−03 | 3.13E−02 | 1.32E−01 | 7.16E−01 | 9.46E−04 | 9.03E−01 | 8.63E−01 |
| 7894072 | — | 9.66E−04 | 2.30E−02 | 1.62E−02 | 3.68E−02 | 4.42E−02 | 6.66E−01 | 1.48E−03 | 6.56E−01 | 5.63E−01 |
| 8072143 | HSCB | 9.68E−04 | 2.31E−02 | 5.58E−02 | 5.53E−02 | 1.17E−02 | 6.35E−01 | 1.60E−03 | 2.78E−01 | 2.02E−01 |
| 8073194 | GRAP2 | 9.76E−04 | 2.32E−02 | 5.36E−03 | 3.14E−02 | 7.17E−02 | 6.93E−01 | 3.03E−03 | 7.86E−01 | 9.01E−01 |
| 7896006 | — | 9.77E−04 | 2.32E−02 | 3.22E−03 | 3.13E−02 | 2.25E−01 | 7.44E−01 | 1.67E−03 | 5.11E−01 | 5.74E−01 |
| 7999903 | C16orf88 | 9.78E−04 | 2.32E−02 | 1.52E−02 | 3.63E−02 | 3.85E−02 | 6.59E−01 | 5.75E−04 | 9.91E−01 | 5.24E−01 |
| 8032899 | TICAM1 | 9.85E−04 | 2.32E−02 | 7.15E−04 | 3.00E−02 | 4.13E−01 | 7.94E−01 | 3.33E−03 | 7.09E−01 | 3.83E−01 |
| 8025458 | ZNF317 | 9.87E−04 | 2.32E−02 | 2.49E−02 | 4.10E−02 | 2.10E−02 | 6.45E−01 | 2.99E−03 | 4.66E−01 | 4.36E−01 |
| 8045247 | PLEKHB2 | 9.88E−04 | 2.32E−02 | 2.58E−03 | 3.12E−02 | 2.59E−01 | 7.56E−01 | 4.00E−03 | 3.85E−01 | 6.48E−01 |
| 8139244 | C7orf44 | 9.88E−04 | 2.32E−02 | 2.40E−02 | 4.06E−02 | 3.28E−02 | 6.45E−01 | 2.71E−03 | 3.16E−01 | 4.19E−01 |
| 8005857 | TMEM199 | 9.88E−04 | 2.32E−02 | 7.14E−02 | 6.22E−02 | 3.87E−03 | 6.08E−01 | 2.75E−03 | 1.43E−01 | 1.31E−01 |
| 8150219 | BRF2 | 9.89E−04 | 2.32E−02 | 1.70E−01 | 1.01E−01 | 1.45E−03 | 5.98E−01 | 8.77E−04 | 2.35E−01 | 4.13E−02 |
| 8060503 | SNORD57 | 9.90E−04 | 2.32E−02 | 9.35E−03 | 3.36E−02 | 2.22E−02 | 6.45E−01 | 1.18E−03 | 3.18E−01 | 7.09E−01 |
| 8150439 | ANK1 | 9.94E−04 | 2.32E−02 | 2.14E−01 | 1.17E−01 | 3.60E−03 | 6.06E−01 | 1.20E−03 | 4.17E−02 | 1.56E−02 |
| 7922391 | CENPL | 9.98E−04 | 2.32E−02 | 1.30E−02 | 3.53E−02 | 3.51E−02 | 6.52E−01 | 1.40E−03 | 8.02E−01 | 5.42E−01 |
| 7945283 | ACAD8 | 9.99E−04 | 2.32E−02 | 4.21E−03 | 3.13E−02 | 3.46E−01 | 7.75E−01 | 1.62E−03 | 4.70E−02 | 4.77E−01 |
| 7894699 | — | 9.99E−04 | 2.32E−02 | 7.46E−03 | 3.28E−02 | 8.32E−02 | 6.94E−01 | 2.96E−03 | 7.06E−01 | 8.92E−01 |
| 8073939 | FLJ44385 | 1.00E−03 | 2.32E−02 | 2.99E−04 | 2.75E−02 | 3.49E−01 | 7.76E−01 | 4.36E−03 | 1.88E−01 | 2.88E−01 |

TABLE 20
Top 6 clusters of Gene Ontology biological process terms enriched for differentially
expressed genes in P1* data set.

| | | EASE | FDR | |
|---|---|---|---|---|
| Term | Count | score P | (%) | Genes |
| Cluster 1 | | | | |
| Enrichment Score: 4.47 | | | | |
| GO: 0006793 | 50 | 1.85E−05 | 0.031 | C20ORF57, CDK17, STK38, SYNJ1, HK2P1, |
| phosphorus metabolic process | | | | MAP3K5, COL4A3BP, CLK4, SYNJ2, STK39, ADAM9, |
| | | | | ADAM10, PAN3, ROCK1, PIK3CB, PIK3CD, ND3, |
| | | | | PKN2, DAPK1, MTMR12, MAPK1, ATP6V1A, |
| | | | | MAP4K4, MAP4K5, SCYL2, MTMR10, HIPK3, |
| | | | | MAPK8, STK10, SSH2, HK2, PRKDC, PPM1B, |
| | | | | IGF1R, SNRK, DUSP15, PPP3CB, YES1, STK38L, |
| | | | | PTPRC, PTPRE, MAP2K1, NIN, NLK, TAOK3, TRIO, |
| | | | | OXSR1, PTPN12, RPS6KA3, CSNK1D, ROCK1P1, |
| | | | | MAPK14, JAK1, LOC731751 |
| GO: 0006796 | 50 | 1.85E−05 | 0.031 | C20ORF57, CDK17, STK38, SYNJ1, HK2P1, |
| phosphate metabolic process | | | | MAP3K5, COL4A3BP, CLK4, SYNJ2, STK39, ADAM9, |
| | | | | ADAM10, PAN3, ROCK1, PIK3CB, PIK3CD, ND3, |
| | | | | PKN2, DAPK1, MTMR12, MAPK1, ATP6V1A, |
| | | | | MAP4K4, MAP4K5, SCYL2, MTMR10, HIPK3, |
| | | | | MAPK8, STK10, SSH2, HK2, PRKDC, PPM1B, |
| | | | | IGF1R, SNRK, DUSP15, PPP3CB, YES1, STK38L, |
| | | | | PTPRC, PTPRE, MAP2K1, NIN, NLK, TAOK3, TRIO, |
| | | | | OXSR1, PTPN12, RPS6KA3, CSNK1D, ROCK1P1, |
| | | | | MAPK14, JAK1, LOC731751 |
| GO: 0006468 | 38 | 2.82E−05 | 0.048 | CDK17, STK38, STK10, PRKDC, IGF1R, MAP3K5, |
| protein amino acid | | | | SNRK, COL4A3BP, CLK4, STK39, YES1, STK38L, |
| phosphorylation | | | | ADAM9, PTPRC, ADAM10, PAN3, PTPRE, MAP2K1, |
| | | | | ROCK1, NIN, PIK3CB, NLK, TAOK3, PIK3CD, PKN2, |
| | | | | TRIO, OXSR1, DAPK1, MAPK1, MAP4K4, RPS6KA3, |
| | | | | MAP4K5, CSNK1D, ROCK1P1, SCYL2, MAPK14, |
| | | | | HIPK3, LOC731751, JAK1, MAPK8 |
| GO: 0016310 | 41 | 1.34E−04 | 0.227 | CDK17, STK38, STK10, HK2, PRKDC, HK2P1, |
| phosphorylation | | | | IGF1R, MAP3K5, SNRK, COL4A3BP, CLK4, STK39, |
| | | | | YES1, STK38L, ADAM9, PTPRC, ADAM10, PAN3, |
| | | | | PTPRE, MAP2K1, NIN, ROCK1, PIK3CB, NLK, |
| | | | | TAOK3, PIK3CD, ND3, PKN2, TRIO, OXSR1, DAPK1, |
| | | | | MAPK1, MAP4K4, ATP6V1A, MAP4K5, RPS6KA3, |
| | | | | CSNK1D, ROCK1P1, SCYL2, MAPK14, HIPK3, |
| | | | | LOC731751, JAK1, MAPK8 |
| Cluster 2 | | | | |
| Enrichment Score: 3.74 | | | | |
| GO: 0006350 | 92 | 2.05E−06 | 0.003 | CCNT2, ZNF292, ZNF518B, ARID4B, CBX4, ZXDC, |
| transcription | | | | ZNF12, MED23, PNN, ZBTB38, PMS2L3, ZKSCAN4, |
| | | | | CNOT4, EPC1, GATA2, PCGF3, SIN3A, CGGBP1, |
| | | | | AHCTF1P1, ATF6B, PSIP1, ZNF540, ZNF879, |
| | | | | ZNF445, CRY1, MLL3, MTERFD3, ZNF493, ZNF33A, |
| | | | | ZNF548, ZNF45, ZNF592, ZNF354A, ZNF644, RBL2, |
| | | | | RCOR3, ZNF507, ADNP, CCNL1, ZNF333, ZBTB26, |
| | | | | C14ORF43, TRERF1, AHR, FOXN3, PURB, NCOA1, |
| | | | | ZNF439, KDM2A, ZNF238, HIPK3, ZMIZ1, NCOA6, |
| | | | | CAND1, NFE2L2, JMJD1C, MED1, ING5, ZNF516, |
| | | | | SETD1B, AHCTF1, NFYA, IVNS1ABP, ZNF514, |
| | | | | ZNF780A, POLR2B, RRAGC, PRDM10, MAML3, |
| | | | | ZNF268, RUNX2, ZNF700, MYSM1, KLF5, KAT2B, |
| | | | | CREBZF, MAML1, NLK, CREBBP, TFCP2, ZNF20, |
| | | | | ZBTB44, GCFC1, RLF, PHF17, ZNF217, ZNF322A, |
| | | | | SP3, ATXN7, ZNF763, ZNF117, NCOR1, RBM16, |
| | | | | KLF3 |
| GO: 0045449 | 105 | 1.19E−05 | 0.02 | ZXDC, CBX4, NAA16, MED23, PNN, CNOT4, GATA2, |
| regulation of transcription | | | | EPC1, SIN3A, CGGBP1, ATF6B, PSIP1, ZNF445, |
| | | | | MLL3, CRY1, MTERFD3, ZNF45, ZNF592, TIGD1, |
| | | | | RBL2, ZNF644, RCOR3, ZNF507, STRN3, C14ORF43, |
| | | | | TRERF1, FOXN3, AHR, MAPK1, ZNF439, ZNF238, |
| | | | | KDM2A, TIAL1, NFE2L2, ZNF516, SETD1B, ZNF514, |
| | | | | MAML3, RUNX2, KLF5, MAP2K1, CREBZF, MAML1, |
| | | | | CREBBP, JRKL, TFCP2, DDX5, ZBTB44, SFMBT2, |
| | | | | GCFC1, ZNF217, ZNF117, NCOR1, KLF3, CCNT2, |
| | | | | ZNF292, FOSL2, ZNF518B, ARID4B, ZNF12, |
| | | | | PMS2L3, ZKSCAN4, ZBTB38, PCGF3, ZNF540, |
| | | | | ZNF879, ZNF493, ZNF548, ZNF33A, CTBP2, |
| | | | | ZNF354A, ADNP, CCNL1, ZNF333, ZBTB26, PURB, |
| | | | | IFNAR2, NCOA1, ZMIZ1, HIPK3, NCOA6, CAND1, |
| | | | | JMJD1C, MED1, ING5, PRKDC, EGLN1, NFYA, |
| | | | | ZNF780A, ORC2L, PRDM10, ZNF268, ZNF700, |
| | | | | MYSM1, NACC2, KAT2B, NLK, ZNF20, RLF, PHF17, |
| | | | | ZNF322A, MAPK14, SP3, ATXN7, LOC731751, |
| | | | | ZNF763, CRK |
| GO: 0051252 | 68 | 0.00475 | 7.772 | FOSL2, CBX4, ZNF12, NAA16, MED23, ZKSCAN4, |
| regulation of RNA metabolic | | | | PMS2L3, ZBTB38, GATA2, EPC1, SIN3A, ATF6B, |
| process | | | | ZNF540, ZNF879, ZNF445, MLL3, ZNF493, ZNF33A, |
| | | | | ZNF548, ZNF45, CTBP2, ZNF354A, STRN3, ADNP, |
| | | | | ZNF333, TRERF1, AHR, FOXN3, HNRNPU, PURB, |
| | | | | IFNAR2, NCOA1, ZNF439, ZNF238, TIAL1, ZMIZ1, |
| | | | | NCOA6, CAND1, JMJD1C, NFE2L2, MED1, PRKDC, |
| | | | | NFYA, ZNF514, ZNF780A, ZFP36L2, ORC2L, |
| | | | | MAML3, ZNF268, RUNX2, ZNF700, MYSM1, RASA1, |
| | | | | KLF5, KAT2B, CREBZF, MAP2K1, MAML1, |
| | | | | CREBBP, TFCP2, ZNF20, GCFC1, MAPK14, SP3, |
| | | | | ATXN7, LOC731751, ZNF763, ZNF117, CRK, NCOR1 |
| GO: 0006355 | 65 | 0.0098 | 15.398 | FOSL2, CBX4, ZNF12, NAA16, MED23, ZKSCAN4, |
| regulation of transcription, | | | | PMS2L3, ZBTB38, GATA2, EPC1, SIN3A, ATF6B, |
| DNA-dependent | | | | ZNF540, ZNF879, ZNF445, MLL3, ZNF493, ZNF33A, |
| | | | | ZNF45, ZNF548, CTBP2, ZNF354A, STRN3, ADNP, |
| | | | | ZNF333, TRERF1, AHR, FOXN3, PURB, IFNAR2, |
| | | | | NCOA1, ZNF439, ZNF238, TIAL1, ZMIZ1, NCOA6, |
| | | | | CANDI, JMJD1C, NFE2L2, MED1, PRKDC, NFYA, |
| | | | | ZNF514, ZNF780A, ORC2L, MAML3, ZNF268, |
| | | | | RUNX2, ZNF700, MYSM1, KLF5, KAT2B, CREBZF, |
| | | | | MAP2K1, MAML1, CREBBP, TFCP2, ZNF20, |
| | | | | GCFC1, MAPK14, SP3, ATXN7, LOC731751, |
| | | | | ZNF763, ZNF117, CRK, NCOR1 |
| Cluster 3 | | | | |
| Enrichment Score: 3.21 | | | | |
| GO: 0016044 | 24 | 2.98E−04 | 0.505 | SEC24B, STX7, VAPA, AP1G1, SORL1, SYNJ1, PPT1, |
| membrane organization | | | | CLTC, GATA2, CD9, DOCK2, PICALM, ITGAV, |
| | | | | CLINT1, MRC1, LY75, SYNRG, VAV3, CORO1C, |
| | | | | NPC1, RAB22A, RAB5A, LRMP, LRP8, CD302 |
| GO: 0016192 | 31 | 4.88E−04 | 0.826 | SEC24B, STX7, AP1G1, SORL1, SYNJ1, PPT1, CLTC, |
| vesicle-mediated transport | | | | ARFGEF1, GATA2, PICALM, TRAPPC6B, ZFYVE16, |
| | | | | ITGAV, EXOC4, CLINT1, MRC1, LY75, SYNRG, |
| | | | | VAV3, CORO1C, ANKRD27, NPC1, STXBP6, LYST, |
| | | | | MCFD2, RAB22A, RAB5A, LRMP, LRP8, CD302, |
| | | | | SPAST, TRAPPC10 |
| GO: 0006897 | 16 | 9.92E−04 | 1.671 | LY75, MRC1, SYNRG, AP1G1, SYNJI, SORL1, PPT1, |
| endocytosis | | | | CORO1C, GATA2, NPC1, PICALM, ITGAV, RAB22A, |
| | | | | RAB5A, LRP8, CD302, CLINT1 |
| GO: 0010324 | 16 | 9.92E−04 | 1.671 | LY75, MRC1, SYNRG, AP1G1, SYNJ1, SORL1, PPT1, |
| membrane invagination | | | | CORO1C, GATA2, NPC1, PICALM, ITGAV, RAB22A, |
| | | | | RAB5A, LRP8, CD302, CLINT1 |
| Cluster 4 Enrichment Score: | | | | |
| 1.94 | | | | |
| GO: 0045935 | 31 | 0.0017 | 2.903 | ING5, ZXDC, NAA16, PRKDC, NFYA, ZBTB38, |
| positive regulation of | | | | GATA2, EPC1, IGF1R, MAML3, RUNX2, MYSM1, |
| nucleobase, nucleoside, | | | | KLF5, PTPRC, MAP2K1, STRN3, MAML1, CREBBP, |
| nucleotide and nucleic acid | | | | DDX5, TRERF1, AHR, MAPK1, NCOA1, MAPK14, |
| metabolic process | | | | ZMIZ1, ATXN7, NCOA6, NPPC, LOC731751, CAND1, |
| | | | | NFE2L2, MED1 |
| GO: 0051173positive regulation | 31 | 0.0028 | 4.632 | ING5, ZXDC, NAA16, PRKDC, NFYA, ZBTB38, |
| of nitrogen compound | | | | GATA2, EPC1, IGF1R, MAML3, RUNX2, MYSM1, |
| metabolic process | | | | KLF5, PTPRC, MAP2K1, STRN3, MAML1, CREBBP, |
| | | | | DDX5, TRERF1, AHR, MAPK1, NCOA1, MAPK14, |
| | | | | ZMIZ1, ATXN7, NCOA6, NPPC, LOC731751, CAND1, |
| | | | | NFE2L2, MED1 |
| GO: 0045941 | 27 | 0.006 | 9.693 | ING5, ZXDC, NAA16, PRKDC, NFYA, ZBTB38, |
| positive regulation of | | | | GATA2, EPC1, MAML3, RUNX2, MYSM1, KLF5, |
| transcription | | | | MAML1, STRN3, CREBBP, DDX5, TRERF1, AHR, |
| | | | | MAPK1, NCOA1, MAPK14, ATXN7, ZMIZ1, NCOA6, |
| | | | | LOC731751, CAND1, NFE2L2, MED1 |
| GO: 0010628 | 27 | 0.0086 | 13.697 | ING5, ZXDC, NAA16, PRKDC, NFYA, ZBTB38, |
| positive regulation of gene | | | | GATA2, EPC1, MAML3, RUNX2, MYSM1, KLF5, |
| expression | | | | MAML1, STRN3, CREBBP, DDX5, TRERF1, AHR, |
| | | | | MAPK1, NCOA1, MAPK14, ATXN7, ZMIZ1, NCOA6, |
| | | | | LOC731751, CAND1, NFE2L2, MED1 |
| GO: 0051254 | 23 | 0.012 | 18.519 | MAP2K1, STRN3, MAML1, CREBBP, PRKDC, |
| positive regulation of RNA | | | | NAA16, NFYA, TRERF1, AHR, ZBTB38, EPC1, |
| metabolic process | | | | GATA2, NCOA1, ZMIZ1, MAPK14, ATXN7, NCOA6, |
| | | | | LOC731751, CAND1, MAML3, NFE2L2, RUNX2, |
| | | | | MYSM1, MED1 |
| GO: 0006357 | 31 | 0.015 | 21.979 | FOSL2, CBX4, PRKDC, MED23, NFYA, ZBTB38, |
| regulation of transcription from | | | | GATA2, EPC1, ORC2L, SIN3A, MAML3, RUNX2, |
| RNA polymerase II promoter | | | | ZNF354A, MAP2K1, STRN3, MAML1, CREBBP, |
| | | | | TFCP2, AHR, IFNAR2, NCOA1, ZNF238, MAPK14, |
| | | | | ZMIZ1, ATXN7, TIAL1, NCOA6, LOC731751, CAND1, |
| | | | | CRK, NCOR1, MED1 |
| GO: 0010557 | 28 | 0.02 | 29.025 | ING5, ZXDC, NAA16, PRKDC, NFYA, ZBTB38, |
| positive regulation of | | | | GATA2, EPC1, IGF1R, MAML3, RUNX2, MYSM1, |
| macromolecule biosynthetic | | | | KLF5, MAML1, STRN3, CREBBP, DDX5, TRERF1, |
| process | | | | AHR, MAPK1, NCOA1, MAPK14, ZMIZ1, ATXN7, |
| | | | | NCOA6, LOC731751, CAND1, NFE2L2, MED1 |
| GO: 0045893 | 22 | 0.02 | 29.418 | STRN3, MAML1, CREBBP, PRKDC, NAA16, NFYA, |
| positive regulation of | | | | TRERF1, AHR, ZBTB38, EPC1, GATA2, NCOA1, |
| transcription, DNA-dependent | | | | ZMIZ1, MAPK14, ATXN7, NCOA6, LOC731751, |
| | | | | CAND1, MAML3, NFE2L2, RUNX2, MYSM1, MED1 |
| GO: 0031328 | 29 | 0.02 | 29.611 | ING5, ZXDC, NAA16, PRKDC, NFYA, ZBTB38, |
| positive regulation of cellular | | | | GATA2, EPC1, IGF1R, MAML3, RUNX2, MYSM1, |
| biosynthetic process | | | | KLF5, MAML1, STRN3, CREBBP, DDX5, TRERF1, |
| | | | | AHR, MAPK1, NCOA1, MAPK14, ZMIZI, ATXN7, |
| | | | | NCOA6, NPPC, LOC731751, CAND1, NFE2L2, |
| | | | | MED1 |
| GO: 0009891 | 29 | 0.024 | 33.467 | ING5, ZXDC, NAA16, PRKDC, NFYA, ZBTB38, |
| positive regulation of | | | | GATA2, EPC1, IGF1R, MAML3, RUNX2, MYSM1, |
| biosynthetic process | | | | KLF5, MAML1, STRN3, CREBBP, DDX5, TRERF1, |
| | | | | AHR, MAPK1, NCOA1, MAPK14, ZMIZ1, ATXN7, |
| | | | | NCOA6, NPPC, LOC731751, CAND1, NFE2L2, |
| | | | | MED1 |
| GO: 0045944 | 18 | 0.025 | 34.407 | MAML1, STRN3, CREBBP, PRKDC, NFYA, AHR, |
| positive regulation of | | | | ZBTB38, EPC1, GATA2, NCOA1, ZMIZ1, MAPK14, |
| transcription from RNA | | | | ATXN7, NCOA6, LOC731751, CAND1, MAML3, |
| polymerase II promoter | | | | RUNX2, MED1 |
| GO: 0010604 | 34 | 0.027 | 36.811 | ING5, ZXDC, PRKDC, NAA16, NFYA, ZBTB38, |
| positive regulation of | | | | GATA2, EPC1, IGF1R, MAML3, RUNX2, MYSM1, |
| macromolecule metabolic | | | | ADAM9, KLF5, PTPRC, MAP2K1, STRN3, MAML1, |
| process | | | | CREBBP, IL6R, RICTOR, DDX5, TRERF1, AHR, |
| | | | | MAPK1, NCOA1, ZMIZ1, MAPK14, ATXN7, NCOA6, |
| | | | | LOC731751, ADAM17, CAND1, NFE2L2, MED1 |
| Cluster 5 Enrichment Score: | | | | |
| 1.79 | | | | |
| GO: 0051056 | 19 | 1.81E−04 | 0.307 | ARHGEF3, NGEF, VAV3, MAP2K1, PREX1, |
| regulation of small GTPase | | | | RALGAPB, IQGAP2, TRIO, RICTOR, ARFGEF1, |
| mediated signal transduction | | | | DNMBP, TBC1D23, TBC1D14, GIT2, CRK, ARAP2, |
| | | | | SPATA13, ARAP1, RASA1 |
| GO: 0046578 | 16 | 6.15E−04 | 1.04 | ARHGEF3, NGEF, VAV3, MAP2K1, PREX1, TRIO, |
| regulation of Ras protein signal | | | | RICTOR, ARFGEF1, DNMBP, TBC1D23, TBC1D14, |
| transduction | | | | GIT2, CRK, ARAP2, SPATA13, ARAP1 |
| GO: 0035023 | 9 | 0.0055 | 8.862 | ARHGEF3, NGEF, VAV3, PREX1, TRIO, RICTOR, |
| regulation of Rho protein signal | | | | SPATA13, ARAP1, DNMBP |
| transduction | | | | |
| GO: 0043087 | 9 | 0.019 | 27.581 | TBC1D23, VAV3, MAP2K1, RAB3GAP1, TBC1D14, |
| regulation of GTPase activity | | | | GIT2, RICTOR, ARAP2, ARAP1 |
| GO: 0032318 | 7 | 0.064 | 67.231 | TBC1D23, MAP2K1, TBC1D14, GIT2, RICTOR, |
| regulation of Ras GTPase | | | | ARAP2, ARAP1 |
| activity | | | | |
| GO: 0032012 | 4 | 0.12 | 87.775 | GIT2, ARFGEF1, ARAP2, ARAP1 |
| regulation of ARF protein | | | | |
| signal transduction | | | | |
| GO: 0032312 | 3 | 0.18 | 96.221 | GIT2, ARAP2, ARAP1 |
| regulation of ARF GTPase | | | | |
| activity | | | | |
| GO: 0051336 | 12 | 0.31 | 99.801 | GNA13, TBC1D23, VAV3, GNAQ, MAP2K1, |
| regulation of hydrolase activity | | | | RAB3GAP1, TBC1D14, GIT2, RICTOR, ARAP2, |
| | | | | NLRP1, ARAP1 |
| Cluster 6 Enrichment Score: | | | | |
| 1.52 | | | | |
| GO: 0043543 | 7 | 0.0056 | 9.064 | ING5, EPC1, PHF17, ZDHHC17, KAT2B, CREBBP, |
| protein amino acid acylation | | | | NAA16 |
| GO: 0016568 | 16 | 0.008 | 12.634 | ING5, KAT2B, RBL2, UTY, SETD1B, CREBBP, CBX4, |
| chromatin modification | | | | PHF17, EPC1, KDM2A, ATXN7, MSL1, JMJD1C, |
| | | | | MLL3, NCOR1, MYSM1 |
| GO: 0006473 | 6 | 0.013 | 20.039 | ING5, EPC1, PHF17, KAT2B, CREBBP, NAA16 |
| protein amino acid acetylation | | | | |
| GO: 0006474 | 3 | 0.014 | 21.402 | KAT2B, CREBBP, NAA16 |
| N-terminal protein amino acid | | | | |
| acetylation | | | | |
| GO: 0016573 | 5 | 0.041 | 50.597 | ING5, EPC1, PHF17, KAT2B, CREBBP |
| histone acetylation | | | | |
| GO: 0051276 | 21 | 0.042 | 51.515 | ING5, KAT2B, SMCHD1, RBL2, UTY, SETD1B, |
| chromosome organization | | | | CREBBP, ARID4B, CBX4, PRKDC, RAD50, EPC1, |
| | | | | PHF17, NIPBL, KDM2A, ATXN7, MSL1, LOC731751, |
| | | | | JMJD1C, MLL3, NCOR1, MYSM1 |
| GO: 0006325 | 17 | 0.053 | 60.502 | ING5, KAT2B, RBL2, UTY, SETD1B, CREBBP, |
| chromatin organization | | | | ARID4B, CBX4, PHT17, EPC1, KDM2A, ATXN7, |
| | | | | MSL1, JMJD1C, MLL3, NCOR1, MYSM1 |
| GO: 0031365 | 3 | 0.054 | 60.945 | KAT2B, CREBBP, NAA16 |
| N-terminal protein amino acid | | | | |
| modification | | | | |
| GO: 0016570 | 7 | 0.11 | 87.323 | ING5, EPC1, PHF17, KAT2B, ATXN7, CREBBP, |
| histone modification | | | | MYSM1 |
| GO: 0016569 | 7 | 0.13 | 90.232 | ING5, EPC1, PHF17, KAT2B, ATXN7, CREBBP, |
| covalent chromatin | | | | MYSM1 |
| modification | | | | |

TABLE 21
The predictor genes for final prediction model. Differentially
expressed genes were ranked by AUC, and top 55 genes were
selected to build the final prediction model. Affymetrix
IDs represent the transcript IDs of Gene ST 1.0 array.
Welch's t-tests were used to calculate p-values, and false
discovery rates (FDR) were calculated using standard methods.

| Affymetrix ID | Gene | AUC | Welch's t-test p-value | FDR(%) |
|---|---|---|---|---|
| 8066822 | SULF2 | 0.805 | 0.00000093 | 0.11 |
| 8177137 | UTY | 0.799 | 0.00000044 | 0.11 |
| 8138116 | ZNF12 | 0.792 | 0.00000100 | 0.11 |
| 8152988 | SLA | 0.790 | 0.00000065 | 0.11 |
| 7975361 | KIAA0247 | 0.789 | 0.00000090 | 0.11 |
| 8051814 | ZFP36L2 | 0.781 | 0.00000113 | 0.11 |
| 8043310 | RMND5A | 0.780 | 0.00000624 | 0.22 |
| 7931353 | PTPRE | 0.780 | 0.00000055 | 0.11 |
| 8151149 | ARFGEF1 | 0.779 | 0.00000116 | 0.11 |
| 8059596 | TRIP12 | 0.779 | 0.00000176 | 0.13 |
| 7953291 | CD9 | 0.779 | 0.00002865 | 0.37 |
| 8138670 | HNRNPA2B1 | 0.778 | 0.00000409 | 0.18 |
| 7987048 | MTMR10 | 0.778 | 0.00000185 | 0.13 |
| 8115562 | RNF145 | 0.777 | 0.00000352 | 0.18 |
| 7995631 | RBL2 | 0.776 | 0.00000169 | 0.13 |
| 8060418 | SIRPA | 0.776 | 0.00000908 | 0.25 |
| 8054135 | MGAT4A | 0.775 | 0.00000538 | 0.21 |
| 8065776 | NCOA6 | 0.774 | 0.00000894 | 0.25 |
| 7922889 | IVNS1ABP | 0.774 | 0.00000403 | 0.18 |
| 8093976 | TBC1D14 | 0.772 | 0.00000388 | 0.18 |
| 7957277 | ZDHHC17 | 0.772 | 0.00000587 | 0.22 |
| 7969651 | DNAJC3 | 0.771 | 0.00000952 | 0.25 |
| 8120992 | ZNF292 | 0.769 | 0.00000454 | 0.19 |
| 8128394 | PNISR | 0.768 | 0.00000889 | 0.25 |
| 7974066 | PNN | 0.767 | 0.00001815 | 0.31 |
| 8073733 | NUP50 | 0.763 | 0.00000827 | 0.25 |
| 8174119 | ZMAT1 | 0.763 | 0.00002377 | 0.33 |
| 8022441 | ROCK1 | 0.762 | 0.00001478 | 0.29 |
| 7950409 | KCNE3 | 0.762 | 0.00002144 | 0.31 |
| 8013965 | SSH2 | 0.761 | 0.00002132 | 0.31 |
| 8079140 | SNRK | 0.761 | 0.00001898 | 0.31 |
| 8126018 | STK38 | 0.761 | 0.00000914 | 0.25 |
| 8068238 | IFNAR2 | 0.761 | 0.00001059 | 0.25 |
| 7989224 | ADAM10 | 0.759 | 0.00001028 | 0.25 |
| 8009205 | DDX42 | 0.759 | 0.00001432 | 0.29 |
| 8048980 | CAB39 | 0.759 | 0.00001656 | 0.31 |
| 8144317 | KBTBD11 | 0.758 | 0.00001927 | 0.31 |
| 8066417 | SERINC3 | 0.757 | 0.00000657 | 0.23 |
| 8119000 | MAPK14 | 0.757 | 0.00028250 | 0.89 |
| 8098177 | KLHL2 | 0.756 | 0.00013219 | 0.67 |
| 8050128 | KIDINS220 | 0.756 | 0.00001988 | 0.31 |
| 8157534 | CNTRL | 0.755 | 0.00001872 | 0.31 |
| 8161701 | TMEM2 | 0.754 | 0.00005385 | 0.48 |
| 8112687 | COL4A3BP | 0.754 | 0.00001227 | 0.27 |
| 7999044 | CREBBP | 0.752 | 0.00003472 | 0.40 |
| 8171762 | RPS6KA3 | 0.752 | 0.00001432 | 0.29 |
| 8163775 | MEGF9 | 0.751 | 0.00004637 | 0.46 |
| 8167971 | MIR223 | 0.751 | 0.00005968 | 0.50 |
| 8023882 | ZNF516 | 0.751 | 0.00004656 | 0.46 |
| 7986132 | MAN2A2 | 0.751 | 0.00007207 | 0.53 |
| 7948667 | AHNAK | 0.751 | 0.00008217 | 0.54 |
| 8079462 | NBEAL2 | 0.750 | 0.00001967 | 0.31 |
| 8099410 | BOD1L | 0.750 | 0.00029880 | 0.92 |
| 8031737 | ZNF548 | 0.750 | 0.00004914 | 0.47 |
| 7988921 | MYO5A | 0.750 | 0.00001438 | 0.29 |

TABLE 22
Prediction performance of ASD55 using various machine learning
algorithms. ASD55 denotes the genes in a classifier developed on P1
with 55 genes (Table 21). The average prediction performances
from 100-repeated leave-group-out cross validations using the P1
dataset are shown. For each prediction instance, 20% of ASD
(N = 13) and 20% of controls (N = 7) were randomly selected
for a testing set, and the other 80% of samples served as a training
set. This procedure was repeated 100 times to calculate the average
performance of ASD55 with 6 machine learning algorithms listed
below. The overall performance of PLS was comparable to the
other 5 methods. The sensitivities were relatively higher than the
specificities across different methods except for the Naïve Bayes
classifier. (AUC: Area under the receiver operation characteristics
curve, ACC: Accuracy, SENS: Sensitivity, SPEC: Specificity,
PPV: Positive Predictive Value, NPV: Negative Predictive Value)

| | | ACC | SENS | SPEC | PPV | NPV |
|---|---|---|---|---|---|---|
| Machine learning method | AUC | (%) | (%) | (%) | (%) | (%) |
| Partial Least Squares | 0.851 | 77.4 | 85.5 | 62.3 | 81.3 | 72.1 |
| Logistic Regression | 0.761 | 71.4 | 76.5 | 61.2 | 79.8 | 56.7 |
| Naïve Bayes | 0.805 | 74.4 | 73.8 | 75.6 | 85.8 | 59.0 |
| kNN (k = 5) | 0.763 | 77.3 | 90.0 | 51.9 | 78.9 | 72.1 |
| Random Forest | 0.771 | 74.2 | 87.8 | 47.1 | 76.9 | 66.1 |
| Support Vector Machine | 0.765 | 79.5 | 85.5 | 67.6 | 84.1 | 70.0 |

TABLE 23
Functional enrichment of genes in ASD55. The term categories are presented as defined in DAVID.

| Term | | | EASE | FDR | |
|---|---|---|---|---|---|
| Category | Term | Count | score P | (%) | Genes |
| UP_TISSUE | Epithelium | 17 | 0.001577 | 1.642572 | ZNF516, CEP110, |
| | | | | | CREBBP, HNRNPA2B1, |
| | | | | | SSH2, IVNS1ABP, |
| | | | | | ARFGEF1, PNN, SFRS18, |
| | | | | | RPS6KA3, MAPK14, |
| | | | | | COL4A3BP, NCOA6, |
| | | | | | NUP50, AHNAK, TRIP12, |
| | | | | | DDX42 |
| GOTERM_BP_FAT | GO: 0006796~phosphate | 10 | 0.001781 | 2.418818 | RPS6KA3, ADAM10, |
| | metabolic process | | | | PTPRE, ROCK1, STK38, |
| | | | | | SNRK, MAPK14, |
| | | | | | MTMR10, COL4A3BP, |
| | | | | | SSH2 |
| GOTERM_BP_FAT | GO: 0006793~phosphorus | 10 | 0.001781 | 2.418818 | RPS6KA3, ADAM10, |
| | metabolic process | | | | PTPRE, ROCK1, STK38, |
| | | | | | SNRK, MAPK14, |
| | | | | | MTMR10, COL4A3BP, |
| | | | | | SSH2 |
| GOTERM_BP_FAT | GO: 0006468~protein amino | 8 | 0.003086 | 4.157323 | RPS6KA3, ADAM10, |
| | acid phosphorylation | | | | PTPRE, ROCK1, STK38, |
| | | | | | SNRK, MAPK14, |
| | | | | | COL4A3BP |
| UP_TISSUE | Bone marrow | 8 | 0.004121 | 4.24187 | ZNF516, ZDHHC17, |
| | | | | | PTPRE, SNRK, NCOA6, |
| | | | | | KIAA0247, TRIP12, SLA |
| GOTERM_BP_FAT | GO: 0016310~phosphorylation | 8 | 0.008293 | 10.81146 | RPS6KA3, ADAM10, |
| | | | | | PTPRE, ROCK1, STK38, |
| | | | | | SNRK, MAPK14, |
| | | | | | COL4A3BP |
| UP_TISSUE | Blood | 7 | 0.011362 | 11.30151 | IFNAR2, SFRS18, SULF2, |
| | | | | | UTY, IVNS1ABP, DNAJC3, |
| | | | | | KCNE3 |
| UP_TISSUE | Placenta | 17 | 0.017419 | 16.84134 | ZNF292, PTPRE, STK38, |
| | | | | | RBL2, CEP110, SIRPA, |
| | | | | | PNN, SERINC3, SFRS18, |
| | | | | | RPS6KA3, MAPK14, |
| | | | | | COL4A3BP, MTMR10, |
| | | | | | KIAA0247, NBEAL2, |
| | | | | | AHNAK, RNF145 |
| KEGG_PATHWAY | hsa04350: TGF~beta signaling | 3 | 0.023044 | 19.20807 | RBL2, ROCK1, CREBBP |
| | pathway | | | | |
| UP_TISSUE | Human rectum tumor | 2 | 0.039277 | 34.33012 | IVNS1ABP, RNF145 |
| GOTERM_BP_FAT | GO: 0009615~response to | 3 | 0.041138 | 43.85211 | IFNAR2, IVNS1ABP, |
| | virus | | | | DNAJC3 |
| KEGG_PATHWAY | hsa04722:Neurotrophin | 3 | 0.04433 | 33.95553 | RPS6KA3, MAPK14, |
| | signaling pathway | | | | KIDINS220 |
| UP_TISSUE | Liver | 11 | 0.059278 | 47.34047 | MAN2A2, MGAT4A, |
| | | | | | ADAM10, ROCK1, SNRK, |
| | | | | | SULF2, MAPK14, |
| | | | | | CEP110, HNRNPA2B1, |
| | | | | | SIRPA, AHNAK |
| UP_TISSUE | Platelet | 5 | 0.069066 | 52.81508 | CD9, ADAM10, UTY, |
| | | | | | MAPK14, SLA |
| UP_TISSUE | Brain | 29 | 0.071092 | 53.88205 | ZMAT1, MYO5A, ZNF292, |
| | | | | | MEGF9, UTY, SSH2, |
| | | | | | ZNF12, IVNS1ABP, |
| | | | | | ARFGEF1, KLHL2, SLA, |
| | | | | | PNN, SFRS18, SNRK, |
| | | | | | TBC1D14, NUP50, |
| | | | | | NBEAL2, RNF145, PTPRE, |
| | | | | | ROCK1, HNRNPA2B1, |
| | | | | | CREBBP, KIDINS220, |
| | | | | | SIRPA, TMEM2, |
| | | | | | RPS6KA3, ZDHHC17, |
| | | | | | SULF2, KBTBD11 |
| GOTERM_BP_FAT | GO: 0016311~dephosphorylation | 3 | 0.075833 | 66.16086 | PTPRE, MTMR10, SSH2 |
| UP_TISSUE | Trachea | 4 | 0.092261 | 63.79246 | MGAT4A, UTY, DNAJC3, |
| | | | | | DDX42 |
| GOTERM_BP_FAT | GO: 0001701~in utero | 3 | 0.095245 | 74.72167 | ADAM10, COL4A3BP, |
| | embryonic development | | | | NCOA6 |
| GOTERM_BP_FAT | GO: 0007243~protein kinase | 4 | 0.095559 | 74.84185 | IFNAR2, RPS6KA3, |
| | cascade | | | | STK38, MAPK14 |

TABLE 24
Pathways enriched with age-correlated genes in ASD.

| | | EASE | FDR | |
|---|---|---|---|---|
| KEGG pathways | Count | score P | (%) | Genes |
| One carbon pool by folate | 7 | 0.000002 | 0.002 | MTHFD1, TYMS, SHMT2, DHFR, ATIC, |
| | | | | MTHFD1L, GART |
| Cell cycle | 10 | 0.001034 | 1.181 | CDK1, CCNE1, CCNB2, BUB1, BUB1B, |
| | | | | CDC16, ATR, SMC1A, ATM, MCM6 |
| Oocyte meiosis | 9 | 0.002019 | 2.294 | CDK1, CCNE1, CCNB2, MOS, BUB1, |
| | | | | PPP3CB, CDC16, PPP1CC, SMC1A |
| Primary immunodeficiency | 5 | 0.006123 | 6.807 | RFX5, ICOS, TNFRSF13B, IGLL1, CD79A |
| B cell receptor signaling | 6 | 0.010567 | 11.480 | HRAS, CD81, PPP3CB, CD22, MALT1, |
| pathway | | | | CD79A |
| p53 signaling pathway | 6 | 0.011629 | 12.565 | CDK1, CCNE1, CCNB2, RRM2, ATR, ATM |

### ABBREVIATIONS

The meanings of certain abbreviations used in the specification are provided below.
ASD—autism spectrum disorders
ROC—receiver operating characteristic
AUC—area under the receiver operating characteristic curve
CI—confidence interval
DSM-IV-TR—Diagnostic and Statistical Manual of Mental Disorders, 4th edition, Text Revision
ADI-R—autism diagnostic interview-revised
ADOS—autism diagnostic observation schedule
CMA—chromosomal microarray analysis
U133p2—Affymetrix HG-U133 Plus 2.0 array
GeneST—Affymetrix Human Gene 1.0 ST array
FDR—false discovery rate
qRT-PCR—quantitative realtime polymerase chain reaction
PLS—partial least squares
ASD245—a prediction model with 245 genes

### KEGG—Kyoto Encyclopedia of Genes and Genomes

### OMIM—Online Mendelian Inheritance in Man

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of characterizing the autism spectrum disorder status of an individual in need thereof, the method comprising:
(a) subjecting a clinical sample obtained from the individual to a gene expression analysis, wherein the gene expression analysis comprises determining expression levels of a plurality of autism spectrum disorder-associated genes in the clinical sample using an expression level determining system, wherein the autism spectrum disorder-associated genes comprise at least ten genes selected from Table 4, 5, 6, 8, 9, 10, 11, 13, 14, 15, 16, 17, 18, 19, 20, 21, 23, or 24; and
(b) determining the autism spectrum disorder status of the individual based on the expression levels of the plurality of autism spectrum disorder-associated genes.

2. The method of claim 1, wherein step (b) comprises comparing each expression level determined in (a) with an appropriate reference level, and the autism spectrum disorder status of the individual is determined based on the results of the comparison.

3. The method of claim 1 or 2 further comprising obtaining the clinical sample from the individual.

4. The method of any preceding claim, further comprising diagnosing autism spectrum disorder in the individual based on the autism spectrum disorder status.

5. The method of any preceding claim, wherein the autism spectrum disorder-associated genes comprise at least one of: LRRC6, SULF2, and YES1.

6. The method of any one of claims 1 to 5, wherein a higher level of at least one autism spectrum disorder-associated gene selected from: ZNF12, RBL2, ZNF292, IVNS1ABP, ZFP36L2, ARFGEF1, UTY, SLA, KIAA0247, HNRNPA2B1, RNF145, PTPRE, SFRS18, ZNF238, TRIP12, PNN, ZDHHC17, MLL3, MTMR10, STK38, SERINC3, NIPBL, TIGD1, DDX42, NUP50, CAB39, ROCK1, SULF2, FABP2, KIDINS220, NCOA6, SIRPA, PCSK5, ADAM10, ZNF33A, ZMAT1, C10orf28, MGAT4A, CEP110, ZZEF1, CREBZF, DOCK11, ATRN, COL4A3BP, FAM133A, TTC14, TMEM30A, MYO5A, KDM2A, ZCCHC14, RNF44, ZBTB44, CLTC, UTRN, ATXN7, PPP1R12A, LBR, TBC1D14, SPATA13, HK2, CREBBP, MED23, ZFYVE16, PAN3, RBBP6, AVL9, ZNF354A, ACTR2, TMBIM1, RPS6KA3, DNMBP, NBEAL2, MYSM1, TMEM2, SNRK, KIAA1109, HECA, DNAJC3, KIF5B, POLR2B, ANTXR2, VPS13C, MANBA, NIN, LRRC6, and YES1 compared with an appropriate reference level indicates that the individual has autism spectrum disorder.

7. The method of any one of claims 1 to 6, wherein a lower level of STXBP6 compared with an appropriate reference level indicates that the individual has autism spectrum disorder.

8. The method of claim 1 or 3, wherein step (b) comprises applying an autism spectrum disorder-classifier to the expression levels to determine the autism spectrum disorder status of the individual.

9. The method of any preceding claim, wherein the autism spectrum disorder-associated genes comprise at least one gene selected from each of at least two of the following KEGG pathways: Neurotrophin signaling pathway, Long-term potentiation, mTOR signaling pathway, Progesterone-mediated oocyte maturation, Regulation of actin cytoskeleton, Fc gamma R-mediated phagocytosis, Renal cell carcinoma, Chemokine signaling pathway, Type II diabetes mellitus, Non-small cell lung cancer, Colorectal cancer, ErbB signaling pathway, Prostate cancer, and Glioma.

10. The method of claim 9, wherein the autism spectrum disorder-associated genes comprise at least one gene selected from each of the KEGG pathways.

11. The method of any preceding claim, wherein the autism spectrum disorder-associated genes comprise at least two different genes selected from at least two of the following sets:
(i) MAPK1, RPS6KA3, YWHAG, CRKL, MAP2K1, PIK3CB, PIK3CD, SH2B3, MAPK8, KIDINS220;
(ii) MAPK1, RPS6KA3, GNAQ, MAP2K1, CREBBP, PPP3CB, PPP1R12A;
(iii) MAPK1, RPS6KA3, PIK3CB, PIK3CD, CAB39, RICTOR;
(iv) IGF1R, MAPK1, RPS6KA3, MAP2K1, PIK3CB, PIK3CD, MAPK8;
(v) GNA13, MAPK1, CRKL, ROCK1, MAP2K1, PIK3CB, PIK3CD, SSH2, PPP1R12A, IQGAP2, ITGB2;
(vi) MAPK1, PTPRC, DOCK2, CRKL, MAP2K1, PIK3CB, PIK3CD;
(vii) MAPK1, CRKL, MAP2K1, PIK3CB, PIK3CD, CREBBP;
(viii) MAPK1, DOCK2, CRKL, ROCK1, MAP2K1, PIK3CB, PREX1, PIK3CD, CCR2, CCR10;
(ix) MAPK1, PIK3CB, PIK3CD, HK2, MAPK8;
(x) MAPK1, RASSF5, MAP2K1, PIK3CB, PIK3CD;
(xi) IGF1R, MAPK1, MAP2K1, PIK3CB, PIK3CD, MAPK8;
(xi) MAPK1, CRKL, MAP2K1, PIK3CB, PIK3CD, MAPK8;
(xiii) IGF1R, MAPK1, MAP2K1, PIK3CB, PIK3CD, CREBBP; and
(xiv) IGF1R, MAPK1, MAP2K1, PIK3CB, PIK3CD.

12. The method of any preceding claim, wherein the autism spectrum disorder genes comprise at least one gene selected from Table 9.

13. The method of claim 12, wherein the autism spectrum disorder is autistic disorder (AUT).

14. The method of one of claims 1 to 11, wherein the autism spectrum disorder genes comprise at least one gene selected from Table 10.

15. The method of claim 14, wherein the autism spectrum disorder is pervasive developmental disorder—not otherwise specified (PDDNOS).

16. The method of any one of claims 1 to 11, wherein the autism spectrum disorder genes comprise at least one gene selected from Table 11.

17. The method of claim 16, wherein the autism spectrum disorder is Asperger's disorder (ASP).

18. The method of any one of claims 1 to 11, wherein the clinical sample is a sample of peripheral blood, brain tissue, or spinal fluid.

19. The method of any one of claims 1 to 18, wherein each expression level is a level of an RNA encoded by an autism spectrum disorder-associated gene of the plurality.

20. The method of any one of claims 1 to 19, wherein the expression level determining system comprises a hybridization-based assay for determining the level of the RNA in the clinical sample.

21. The method of claim 20, wherein the hybridization-based assay is an oligonucleotide array assay, an oligonucleotide conjugated bead assay, a molecular inversion probe assay, a serial analysis of gene expression (SAGE) assay, or an RT-PCR assay.

22. The method of any one of claims 1 to 18, wherein each expression level is a level of a protein encoded by an autism spectrum disorder-associated gene of the plurality.

23. The method of any one of claim 1 to 18, or 22, wherein the expression level determining system comprises an antibody-based assay for determining the level of the protein in the clinical sample.

24. The method of claim 23, wherein the antibody-based assay is an antibody array assay, an antibody conjugated-bead assay, an enzyme-linked immuno-sorbent (ELISA) assay, or an immunoblot assay.

25. A method of characterizing the autism spectrum disorder status in an individual in need thereof, the method comprising:
(a) subjecting a clinical sample obtained from the individual to a gene expression analysis, wherein the gene expression analysis comprises determining expression levels of a plurality of autism spectrum disorder-associated genes in the clinical sample using an expression level determining system, wherein the autism spectrum disorder-associated genes comprise at least ten genes selected from Table 4, 5, 6, 8, 9, 10, 11, 13, 14, 15, 16, 17, 18, 19, 20, 21, 23, or 24; and
(b) applying an autism spectrum disorder-classifier to the expression levels, wherein the autism spectrum disorder-classifier characterizes the autism spectrum disorder status of the individual based on the expression levels.

26. The method of claim 25, further comprising diagnosing autism spectrum disorder in the individual based on the autism spectrum disorder status.

27. The method of claim 25 or 26, wherein the autism spectrum disorder-classifier is based on an algorithm selected from logistic regression, partial least squares, linear discriminant analysis, quadratic discriminant analysis, neural network, naïve Bayes, C4.5 decision tree, k-nearest neighbor, random forest, and support vector machine.

28. The method of any one of claims 25 to 27, wherein the autism spectrum disorder-classifier has an accuracy of at least 65%.

29. The method of any one of claims 25 to 27, wherein the autism spectrum disorder-classifier has an accuracy in a range of about 65% to 90%.

30. The method of any one of claims 25 to 29, wherein the autism spectrum disorder-classifier has a sensitivity of at least 65%.

31. The method of any one of claims 25 to 29, wherein the autism spectrum disorder-classifier has a sensitivity in a range of about 65% to about 95%.

32. The method of any one of claims 25 to 31, wherein the autism spectrum disorder-classifier has a specificity of at least 65%.

33. The method of any one of claims 25 to 31, wherein the autism spectrum disorder-classifier has a specificity in range of about 65% to about 85%.

34. The method of any one of claims 25 to 33, wherein the autism spectrum disorder-classifier is trained on a data set comprising expression levels of the plurality of autism spectrum disorder-associated genes in clinical samples obtained from a plurality of individuals identified as having autism spectrum disorder.

35. The method of claim 34, wherein the interquartile range of ages of the plurality of individuals identified as having autism spectrum disorder is from about 2 years to about 10 years.

36. The method of any one of claims 25 to 34, wherein the autism spectrum disorder-classifier is trained on a data set comprising expression levels of the plurality of autism spectrum disorder-associated genes in clinical samples obtained from a plurality of individuals identified as not having autism spectrum disorder.

37. The method of claim 36, wherein the interquartile range of ages of the plurality of individuals identified as not having autism spectrum disorder is from about 2 years to about 10 years.

38. The method of any one of claims 25 to 37, wherein the autism spectrum disorder-classifier is trained on a data set consisting of expression levels of the plurality of autism spectrum disorder-associated genes in clinical samples obtained from a plurality of male individuals.

39. The method of any one of claims 25 to 38, wherein the autism spectrum disorder-classifier is trained on a data set comprising expression levels of the plurality of autism spectrum disorder-associated genes in clinical samples obtained from a plurality of individuals identified as having autism spectrum disorder.

40. The method of claim 39, wherein the individuals were identified as having autism spectrum disorder based on DSM-IV-TR criteria.

41. The method of any one of claims 25 to 40, wherein the autism spectrum disorder-associated genes comprise at least one of: LRRC6, SULF2, and YES1.

42. The method of any one of claims 25 to 41, wherein the autism spectrum disorder-associated genes comprise at least one gene selected from each of at least two of the following KEGG pathways: Neurotrophin signaling pathway, Long-term potentiation, mTOR signaling pathway, Progesterone-mediated oocyte maturation, Regulation of actin cytoskeleton, Fc gamma R-mediated phagocytosis, Renal cell carcinoma, Chemokine signaling pathway, Type II diabetes mellitus, Non-small cell lung cancer, Colorectal cancer, ErbB signaling pathway, Prostate cancer, and Glioma.

43. The method of claim 42, wherein the autism spectrum disorder-associated genes comprise at least one gene selected from each of the KEGG pathways.

44. The method of any one of claims 25 to 43, wherein the autism spectrum disorder-associated genes comprise at least two different genes selected from at least two of the following sets:
(i) MAPK1, RPS6KA3, YWHAG, CRKL, MAP2K1, PIK3CB, PIK3CD, SH2B3, MAPK8, KIDINS220;
(ii) MAPK1, RPS6KA3, GNAQ, MAP2K1, CREBBP, PPP3CB, PPP1R12A;
(iii) MAPK1, RPS6KA3, PIK3CB, PIK3CD, CAB39, RICTOR;
(iv) IGF1R, MAPK1, RPS6KA3, MAP2K1, PIK3CB, PIK3CD, MAPK8;
(v) GNA13, MAPK1, CRKL, ROCK1, MAP2K1, PIK3CB, PIK3CD, SSH2, PPP1R12A, IQGAP2, ITGB2;
(vi) MAPK1, PTPRC, DOCK2, CRKL, MAP2K1, PIK3CB, PIK3CD;
(vii) MAPK1, CRKL, MAP2K1, PIK3CB, PIK3CD, CREBBP;
(viii) MAPK1, DOCK2, CRKL, ROCK1, MAP2K1, PIK3CB, PREX1, PIK3CD, CCR2, CCR10;
(ix) MAPK1, PIK3CB, PIK3CD, HK2, MAPK8;
(x) MAPK1, RASSF5, MAP2K1, PIK3CB, PIK3CD;
(xi) IGF1R, MAPK1, MAP2K1, PIK3CB, PIK3CD, MAPK8;
(xi) MAPK1, CRKL, MAP2K1, PIK3CB, PIK3CD, MAPK8;
(xiii) IGF1R, MAPK1, MAP2K1, PIK3CB, PIK3CD, CREBBP; and
(xiv) IGF1R, MAPK1, MAP2K1, PIK3CB, PIK3CD.

45. The method of any one of claims 25 to 44, wherein the clinical sample is a sample of peripheral blood, brain tissue, or spinal fluid.

46. The method of any one of claims 25 to 45, wherein each expression level is a level of an RNA encoded by an autism spectrum disorder-associated gene of the plurality.

47. The method of any one of claims 25 to 46, wherein the expression level determining system comprises a hybridization-based assay for determining the level of the RNA in the clinical sample.

48. The method of claim 47, wherein the hybridization-based assay is an oligonucleotide array assay, an oligonucleotide conjugated bead assay, a molecular inversion probe assay, a serial analysis of gene expression (SAGE) assay, or an RT-PCR assay.

49. The method of any one of claims 25 to 45, wherein each expression level is a level of a protein encoded by an autism spectrum disorder-associated gene of the plurality.

50. The method of any one of claim 25 to 45, or 49, wherein the expression level determining system comprises an antibody-based assay for determining the level of the protein in the clinical sample.

51. The method of claim 50, wherein the antibody-based assay is an antibody array assay, an antibody conjugated-bead assay, an enzyme-linked immuno-sorbent (ELISA) assay, or an immunoblot assay.

52. An array consisting essentially of oligonucleotide probes that hybridize to nucleic acids having sequence correspondence to mRNAs of at least ten autism spectrum disorder-associated genes selected from Table 6.

53. An array consisting essentially of antibodies that bind specifically to proteins encoded by at least ten autism spectrum disorder-associated genes selected from Table 6.

54. A method of monitoring progression of an autism spectrum disorder in an individual in need thereof, the method comprising:
(a) obtaining a clinical sample from the individual;
(b) determining expression levels of a plurality of autism spectrum disorder-associated genes in the clinical sample using an expression level determining system,
(c) comparing each expression level determined in (b) with an appropriate reference level, wherein the results of the comparison are indicative of the extent of progression of the autism spectrum disorder in the individual.

55. A method of monitoring progression of an autism spectrum disorder in an individual in need thereof, the method comprising:
(a) obtaining a first clinical sample from the individual,
(b) determining expression levels of a plurality of autism spectrum disorder-associated genes in the first clinical sample using an expression level determining system,
(c) obtaining a second clinical sample from the individual,
(d) determining expression levels of the plurality of autism spectrum disorder-associated genes in the second clinical sample using an expression level determining system,
(e) comparing the expression level of each autism spectrum disorder-associated gene determined in (b) with the expression level determined in (d) of the same autism spectrum disorder associated-gene,
wherein the results of comparing in (e) are indicative of the extent of progression of the autism spectrum disorder in the individual.

56. The method of claim 54 or 55, wherein the autism spectrum disorder-associated genes comprise at least ten genes selected from Table 6.

57. A method of monitoring progression of an autism spectrum disorder in an individual in need thereof, the method comprising:
(a) obtaining a first clinical sample from the individual,
(b) obtaining a second clinical sample from the individual,
(c) determining the expression level of an autism spectrum disorder-associated gene in the first clinical sample using an expression level determining system,
(d) determining the expression level of the autism spectrum disorder-associated gene in the second clinical sample using an expression level determining system,
(e) comparing the expression level determined in (c) with the expression level determined in (d),
(f) repeating (c)-(e) for at least one other autism spectrum disorder-associated gene,
wherein the results of comparing in (e) for the at least two autism spectrum-associated genes are indicative of the extent of progression of the autism spectrum disorder in the individual.

58. A method of monitoring progression of an autism spectrum disorder in an individual in need thereof, the method comprising:
(a) obtaining a first clinical sample from the individual,
(b) obtaining a second clinical sample from the individual,
(c) determining a first expression pattern comprising expression levels of at least two autism spectrum disorder-associated genes in the first clinical sample using an expression level determining system,
(d) determining a second expression pattern comprising expression levels of at least two autism spectrum disorder-associated genes in the second clinical sample using an expression level determining system,
(e) comparing the first expression pattern with the second expression pattern,
wherein the results of comparing in (e) are indicative of the extent of progression of the autism spectrum disorder in the individual.

59. The method of any one of claims 55 to 58, wherein the time between obtaining the first clinical sample and obtaining the second clinical sample is a time sufficient for a change in the severity of the autism spectrum disorder to occur in the individual.

60. The method of any one of claims 55 to 58, wherein between obtaining the first clinical sample and obtaining the second clinical sample the individual is treated for the autism spectrum associated disorder.

61. A method of assessing the efficacy of a treatment for an autism spectrum disorder in an individual in need thereof, the method comprising:
(a) obtaining a clinical sample from the individual,
(b) administering a treatment to the individual for the autism spectrum disorder,
(c) determining an expression pattern comprising expression levels of at least two autism spectrum disorder-associated genes in the clinical sample,
(e) comparing the expression pattern with an appropriate reference expression pattern, wherein the appropriate reference expression pattern comprises expression levels of the at least two autism spectrum disorder-associated genes in a clinical sample obtained from an individual who does not have the autism spectrum disorder,
wherein the results of the comparison in (c) are indicative of the efficacy of the treatment.

62. A method of assessing the efficacy of a treatment for an autism spectrum disorder in an individual in need thereof, the method comprising:
(a) obtaining a first clinical sample from the individual,
(b) administering a treatment to the individual for the autism spectrum disorder,
(c) obtaining a second clinical sample from the individual after having administered the treatment to the individual,
(d) determining a first expression pattern comprising expression levels of at least two autism spectrum disorder-associated genes in the first clinical sample,
(e) comparing the first expression pattern with an appropriate reference expression pattern, wherein the appropriate reference expression pattern comprises expression levels of the at least two autism spectrum disorder-associated genes in a clinical sample obtained from an individual who does not have the autism spectrum disorder,
(f) determining a second expression pattern comprising expression levels of at least two autism spectrum disorder-associated genes in the second clinical sample, and
(g) comparing the second expression pattern with the appropriate reference expression pattern, wherein a difference between the second expression pattern and the appropriate reference expression pattern that is less than the difference between the first expression pattern and the appropriate reference pattern is indicative of the treatment being effective.

63. A method for selecting an appropriate dosage of a treatment for an autism spectrum associated disorder in an individual in need thereof, the method comprising:
(a) administering a first dosage of a treatment for an autism spectrum associated disorder to the individual,
(b) assessing the efficacy of the first dosage of the treatment, in part, by determining at least one expression pattern comprising expression levels of at least two autism spectrum disorder-associated genes in a clinical sample obtained from the individual,
(c) administering a second dosage of a treatment for an autism spectrum associated disorder in the individual:
(d) assessing the efficacy of the second dosage of the treatment, in part, by determining at least one expression pattern comprising expression levels of at least two autism spectrum disorder-associated genes in a clinical sample obtained from the individual,
wherein the appropriate dosage is selected as the dosage administered in (a) or (c) that has the greatest efficacy.

64. The method of claim 63, wherein the efficacy is assessed in (b) and (d) according to the method of claim 61.

65. A method for selecting an appropriate dosage of a treatment for an autism spectrum associated disorder in an individual in need thereof, the method comprising:
(a) administering a dosage of a treatment for an autism spectrum associated disorder to the individual;
(b) assessing the efficacy of the dosage of the treatment, in part, by determining at least one expression pattern comprising expression levels of at least two autism spectrum disorder-associated genes in a clinical sample obtained from the individual, and
(c) selecting the dosage as being appropriate for the treatment for the autism spectrum associated disorder in the individual, if the efficacy determined in (b) is at or above a threshold level, wherein the threshold level is an efficacy level at or above which a treatment substantially improves at least one symptom of an autism spectrum disorder.

66. A method for identifying an agent useful for treating an autism spectrum associated disorder in an individual in need thereof, the method comprising:
(a) contacting an autism spectrum associated disorder-cell with a test agent,
(b) determining at least one expression pattern comprising expression levels of at least two autism spectrum disorder-associated genes in the autism spectrum disorder-associated cell,
(c) comparing the at least one expression pattern with a test expression pattern, and
(d) identifying the agent as being useful for treating the autism spectrum associated disorder based on the comparison in (c).

67. The method of claim 66, wherein test expression pattern is an expression pattern indicative of an individual who does not have the autism spectrum disorder, and wherein a decrease in a difference between the at least one expression pattern and the test expression pattern resulting from contacting the autism spectrum disorder-associated cell with the test agent identifies the test agent as being useful for the treatment of the autism spectrum associated disorder.

68. The method of claim 66 or 67, wherein the autism spectrum disorder-associated cell is contacted with the test agent in (a) in vivo.

69. The method of claim 66 or 67, wherein the autism spectrum disorder-associated cell is contacted with the test agent in (a) in vitro.

70. The method of any preceding claim, wherein the autism spectrum disorder is autistic disorder (AUT), pervasive developmental disorder—not otherwise specified (PDDNOS), or Asperger's disorder (ASP).

71. The method of any one of claims 54 to 70, wherein the autism spectrum disorder-associated genes are selected from Table 4, 5, 6, 8, 9, 10, or 11.
